(19) United States
(12) Patent Application Publication
(10) Pub. No.: US 2006/0292099 A1
(43) Pub. Date: Dec. 28, 2006

(54) Treatment of eye disorders with sirtuin modulators

(76) Inventors: Michael Milburn, Cary, NC (US); Christoph H. Westphal, Brookline, MA (US); David J. Livingston, Barrington, RI (US); Peter Elliott, Marlborough, MA (US); Philip Lambert, Northborough, MA (US); Karl D. Normington, Acton, MA (US)

Correspondence Address: FISH & NEAVE IP GROUP;ROPES & GRAY LLP, ONE INTERNATIONAL PLACE, BOSTON, MA, 02110-2624, US

(21) Appl. No.: 11/440,584

(22) Filed: May 24, 2006

Related U.S. Application Data
(60) Provisional application No. 60/684,252, filed on May 25, 2005.
(60) Provisional application No. 60/788,358, filed on Mar. 30, 2006.

(51) Int. Cl. A61K 8/00

(52) U.S. Cl. 424/70.1

(57) ABSTRACT

Sirtuin modulators, particularly sirtuin activators, are useful in treating vision impairment. In general, the sirtuin modulators inhibit the progression of vision impairment resulting from various eye disorders. The invention also includes pharmaceutically acceptable formulations of sirtuin modulators, particular ophthalmically acceptable formulations.

### RELATED APPLICATIONS

This application claims the benefit of U.S. Provisional Application Nos. 60/684,252, filed May 25, 2005, 60/731,550, filed Oct. 28, 2005, and 60/788,358, filed Mar. 30, 2006. The contents of these applications are incorporated herein by reference in their entirety.

### BACKGROUND OF THE INVENTION

According to a study sponsored by the National Eye Institute, vision loss is becoming a major public health problem as the population ages. It reports that blindness or low vision affects 3.3 million Americans of age 40 and over. By 2020, it projects that this number will increase to 5.5 million.

The study found that vision loss and blindness are strongly age-linked. Although people age 80 and over account for over 8% of the overall U.S. population, they represent 69% of the blind population. The most common eye diseases in Americans age 40 and over are age-related macular degeneration, glaucoma, cataracts and diabetic retinopathy. The causes for these diseases are varied, and include injury, exposure to toxins, underlying health conditions (e.g., diabetes, arteriosclerosis), and genetic factors (e.g., overproduction of aqueous humor). With the exception of cataracts, where the lens can be removed and replaced, there is no cure for these diseases and vision loss is generally permanent. The extent of permanent vision loss is largely dependent upon the extent of damage to one or both of the optic nerves and the retina.

Thus, there is a need for protective compounds that inhibit, reduce, or otherwise treat vision impairment or progression. These protective compounds would be useful in the context of injuries arising from impact or toxic chemicals including counteracting toxic side-effects associated with certain chemotherapeutic regimes, or improving quality of life in populations experiencing progressive vision impairment.

### SUMMARY OF THE INVENTION

The present invention relates to the use of protective agents to treat (including inhibit or reduce) vision impairment, particularly vision impairment resulting from damage to the retina or optic nerve. More specifically, the present invention relates to the use of sirtuin modulators (e.g., direct or indirect sirtuin activators (STACs) or inhibitors) to treat vision impairment. While the efficacy of sirtuin modulators disclosed herein may be due to their anti-apoptotic and anti-aging properties, the efficacy may also be due to another mechanism.

Accordingly, one aspect of the present invention describes a method for treating vision impairment by administering to a patient a therapeutic dosage of sirtuin modulator selected from a compound disclosed herein, or a pharmaceutically acceptable salt, prodrug or a metabolic derivative thereof.

In certain aspects of the invention, the vision impairment is caused by damage to the optic nerve or central nervous system. In particular embodiments, optic nerve damage is caused by high intraocular pressure, such as that created by glaucoma. In other particular embodiments, optic nerve damage is caused by swelling of the nerve, which is often associated with an infection or an immune (e.g., autoimmune) response, such as that which occurs in optic neuritis or multiple sclerosis. In further particular embodiment, optic nerve damage is caused by ischemia, generally caused by a deficiency in the blood supply, such as anterior ischemic optic neuropathy.

In certain aspects of the invention, the vision impairment is caused by retinal damage. In particular embodiments, retinal damage is caused by disturbances in blood flow to the retina (e.g., arteriosclerosis). In particular embodiments, retinal damage is caused by disrupton of the macula (e.g., exudative or non-exudative macular degeneration). The axons of the retinal ganglion cells (RGC's) comprise the optic nerve, so damage to the retinal ganglion cell body can lead to damage of the optic nerve.

In certain aspects of the invention, the sirtuin modulators can be used to inhibit (e.g., treat prophylactically) damage, disease or general aging of the eye that can ultimately lead to vision impairment. Damage to the eye can be secondary to another disease or treatment by another medicament for that disease. Damage can also be secondary to surgical procedures either directly on the eye or elsewhere on a patient. In addition, prevention of the effects of general aging as well as overuse of the eye would be beneficial to patients as eye function declines.

Furthermore, an improvement in the present invention relates to methods for augmenting treatments which require administration of a chemotherapeutic agent that has a vision impairing side effect. The improvement includes administering prophylacticaly or therapeutically an effective amount of a sirtuin modulator to treat the vision impairing side effects of the chemotherapeutic drug, preferably without impairing its efficacy. The sirtuin modulator and chemotherapeutic agent may be provided in various modes including administration prior to, simultaneously with, or subsequent to administration of the chemotherapeutic agent. The sirtuin modulator and chemotherapeutic agent may also be provided in various forms including but not limited to a single pharmaceutical preparation, e.g. as a single dosage form, or a kit in which each is provided in separate dosages, along with instructions for co-administering the two agents.

The present invention also relates to methods for conducting pharmaceutical business comprising manufacturing, testing, marketing, distributing, and licensing preparations or kits for administering a sirtuin modulator and optionally additional agents.

Another aspect of the present invention provides a composition that includes nanoparticles comprising a sirtuin modulator, or a pharmaceutically acceptable salt, prodrug or metabolic derivative thereof. Such particles typically have a mean diameter of 50 nm to 500 nm, such as 100 nm to 200 nm.

A further aspect of the present invention provides a composition that includes a cyclodextrin and a sirtuin modulator, or a pharmaceutically acceptable salt, prodrug or metabolic derivative thereof. Such compositions are advantageously liquids or lyophilized powders (e.g., water-soluble powders).

The invention also provides fast melt tablets containing a sirtuin modulator, or a pharmaceutically acceptable salt, prodrug or metabolic derivative thereof. Such tablets typically have an oral dissolution time of less than 1 minute, such as less than 30 seconds.

In addition, the invention provides implantable devices that contain a sirtuin modulator, or a pharmaceutically acceptable salt, prodrug or metabolic derivative thereof. In particular embodiments, the devices are suitable for implantation in the eye. These devices typically provide extended release of the sirtuin modulator, for example, release for at least 1 month or for at least one year (e.g., 6 months to 2 years). These devices can be biodegradable or non-biodegradable (e.g., a replacement lens).

In another aspect of the invention, the invention provides a pharmaceutical composition comprising a micronized sirtuin modulator or a pharmaceutically acceptable salt, prodrug or a metabolic derivative thereof, where particles of the micronized sirtuin modulator have an average diameter of less than about 30 microns.

The invention further includes the use of the compositions disclosed herein in the manufacture of a medicament for treating vision impairment.

### BRIEF DESCRIPTION OF THE DRAWINGS

FIG. 1 shows plant polyphenol sirtuin 1 (SIRT1) activators.

FIG. 2 shows stilbene and chalcone SIRT1 activators.

FIG. 3 shows flavone SIRT1 activators.

FIG. 4 shows flavone SIRT1 modulators FIG. 5 shows isoflavone, flavanone and anthocyanidin SIRT1 modulators.

FIG. 6 shows catechin (Flavan-3-ol) SIRT1 modulators.

FIG. 7 shows free radical protective SIRT1 modulators.

FIG. 8 shows SIRT1 modulators.

FIG. 9 shows SIRT1 modulators.

FIG. 10 shows resveratrol analog SIRT1 activators.

FIG. 11 shows resveratrol analog SIRT1 activators.

FIG. 12 shows resveratrol analog SIRT1 activators.

FIG. 13 shows resveratrol analog SIRT1 modulators.

FIG. 14 shows resveratrol analog SIRT1 modulators.

FIGS. 15A-G shows sirtuin activators.

FIG. 16 shows sirtuin inhibitors.

FIG. 17A shows the change in the average clinical experimental autoimmune encephalomyelitis (EAE) score over time after immunization with Proteolipid Protein (PLP), and FIG. 17B shows the percentage of eyes from EAE mice that developed optic neuritis.

FIG. 18 shows that there is a significant decrease in retinal ganglion cells (RGCs) over time in optic neuritis eyes, as compared to control eyes and eyes of EAE that did not develop optic neuritis.

FIG. 19 shows that nicotinamide riboside is effective preserving RCGs in an acute optic neuritis model.

FIG. 20 shows fluorogold-labeled RGCs (A) of eye with optic neuritis treated with placebo (PBS) (representative of Group 3 in Example 8) and (B) of eye with optic neuritis treated with nicotinamide riboside (representative of Group 5, Example 8).

FIG. 21 shows a schematic outline of the experiment described in Example 9.

FIG. 22 shows the RGC numbers in eyes from all treatment groups in Example 9.

FIG. 23 shows a comparison of the plasma profile from unprocessed resveratrol in methylcellulose (MC) (Res; mean particle size approx. 13 um) to micronized resveratrol (Micro; mean particle size approx. 1.0 um) in either methylcellulose (MC) or HPMC/DOSS after oral gavage in mice at the doses shown.

FIG. 24 shows a comparison of the plasma profile from micronized resveratrol (Micro; mean particle size approx. 1.0 um) in ether Tween 80 or HPMC/DOSS after oral gavage in mice at the doses shown.

FIG. 25 shows a comparison of the plasma profile from micronized resveratrol in HPMC/DOSS following either oral gavage or intraperitoneal injection in mice at the doses shown.

FIG. 26 shows a comparison of various formulations of resveratrol in rats following intraperitoneal administration at the indicated doses.

FIG. 27 shows a comparison of various formulations of resveratrol in rats following oral administration at the indicated doses.

### DETAILED DESCRIPTION OF THE INVENTION

A. Overview

The present invention discloses compositions and methods for treating eye disorders that lead to vision impairment or loss of vision (blindness). In particular, the present invention discloses methods for treating vision impairment due to damage to the retina or optic nerve.

B. Definitions

The term “vision impairment” refers to diminished vision, which is often only partially reversible or irreversible upon treatment (e.g., surgery). Particularly severe vision impairment is termed “blindness” or “vision loss”, which refers to a complete loss of vision, vision worse than 20/200 that cannot be improved with corrective lenses, or a visual field of less than 20 degrees diameter (10 degrees radius).

As used herein, the term “inhibiting” means to reduce the risk of occurrence of an abnormal biological or a medical event, such as vision loss, in a cell, a tissue, a system, animal or human.

The term “treating” refers to: inhibiting a disease, disorder or condition from occurring in a cell, a tissue, a system, animal or human which may be predisposed to the disease, disorder and/or condition but has not yet been diagnosed as having it; stabilizing a disease, disorder or condition, i.e., arresting its development; and relieving one or more symptoms of the disease, disorder or condition, i.e., causing regression of the disease, disorder and/or condition.

As used herein, a therapeutic that “inhibits” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “as valence and stability permits” in reference to compounds disclosed herein refers to compounds that have in vitro or in vivo half-lives at room temperature of at least 12 hours, or at least 24 hours, and are preferably capable of being stored at 0° C. for a week without decomposing by more than about 10%.

The terms “half-life” or “half-lives” refer to the time required for half of a quantity of a substance to be converted to another chemically distinct species in vitro or in vivo.

The term “prodrug” refers to any compound that is converted to a more pharmacologically active compound under physiological conditions (i.e., in vivo). A common method for making a prodrug is to select moieties that are hydrolyzed under physiological conditions to provide the desired biologically active drug.

The term “metabolic derivative” refers to a compound derived by one or more in vitro or in vivo enzymatic transformations on the parent compound.

“Sirtuin modulator” refers to a compound that up regulates (e.g., activate or stimulate), down regulates (e.g., inhibit or suppress) or otherwise changes a functional property or biological activity of a sirtuin protein. Sirtuin modulators may act to modulate a sirtuin protein either directly or indirectly. In certain embodiments, a sirtuin modulator may be a sirtuin activator or a sirtuin inhibitor.

“Sirtuin” refers to a member of the sirtuin deacetylase protein family, or preferably to the sir2 family, which include yeast Sir2 (GenBank Accession No. P53685), C. elegans Sir-2.1 (GenBank Accession No. NP_{—}501912), and human SIRT1 (GenBank Accession No. NM_{—}012238 and NP_{—}036370 (or AF083106)) and SIRT2 (GenBank Accession No. NM_{—}012237, NM-030593, NP_{—}036369, NP_{—}085096, and AF083107) proteins. Other family members include the four additional yeast Sir2-like genes termed “HST genes” (homologues of Sir two) HST1, HST2, HST3 and HST4, and the five other human homologues hSIRT3, hSIRT4, hSIRT5, hSIRT6 and hSIRT7 (Brachmann et al. (1995) Genes Dev. 9:2888 and Frye et al. (1999) BBRC 260:273). Preferred sirtuins are those that share more similarities with SIRT1, i.e., hSIRT1, and/or Sir2 than with SIRT2, such as those members having at least part of the N-terminal sequence present in SIRT1 and absent in SIRT2 such as SIRT3 has.

“SIRT1 protein” refers to a member of the sir2 family of sirtuin deacetylases. In one embodiment, a SIRT1 protein includes yeast Sir2 (GenBank Accession No. P53685), C. elegans Sir-2.1 (GenBank Accession No. NP_{—}501912), human SIRT1 (GenBank Accession No. NM_{—}012238 and NP_{—}036370 (or AF083106)), human SIRT2 (GenBank Accession No. NM_{—}012237, NM_{—}030593, NP_{—}036369, NP_{—}085096, and AF083107) proteins, and equivalents and fragments thereof. In another embodiment, a SIRT1 protein includes a polypeptide comprising a sequence consisting of, or consisting essentially of, the amino acid sequence set forth in GenBank Accession Nos. NP 036370, NP_{—}501912, NP_{—}085096, NP_{—}036369, and P53685. SIRT1 proteins include polypeptides comprising all or a portion of the amino acid sequence set forth in GenBank Accession Nos. NP_{—}036370, NP_{—}501912, NP_{—}085096, NP_{—}036369, and P53685; the amino acid sequence set forth in GenBank Accession Nos. NP_{—}036370, NP_{—}501912, NP_{—}085096, NP_{—}036369, and P53685 with 1 to about 2, 3, 5, 7, 10, 15, 20, 30, 50, 75 or more conservative amino acid substitutions; an amino acid sequence that is at least 60%, 70%, 80%, 90%, 95%, 96%, 97%, 98%, or 99% identical to GenBank Accession Nos. NP_{—}036370, NP_{—}501912, NP_{—}085096, NP_{—}036369, and P53685 and functional fragments thereof. Polypeptides of the invention also include homologs (e.g., orthologs and paralogs), variants, or fragments, of GenBank Accession Nos. NP_{—}036370, NP_{—}501912, NP_{—}085096, NP_{—}036369, and P53685.

“Sirtuin activator” refers to a compound that increases the level of a sirtuin protein and/or increases at least one activity of a sirtuin protein. In an exemplary embodiment, a sirtuin activator may increase at least one biological activity of a sirtuin protein by at least about 10%, 25%, 50%, 75%, 100%, or more. Exemplary biological activities of sirtuin proteins include deacetylation, e.g., of histones and p53; extending lifespan; increasing genomic stability; silencing transcription; and controlling the segregation of oxidized proteins between mother and daughter cells.

“Sirtuin inhibitor” refers to a compound that decreases the level of a sirtuin protein and/or decreases at least one activity of a sirtuin protein. In an exemplary embodiment, a sirtuin inhibitor may decrease at least one biological activity of a sirtuin protein by at least about 10%, 25%, 50%, 75%, 100%, or more. Exemplary biological activities of sirtuin proteins include deacetylation, e.g., of histones and p53; extending lifespan; increasing genomic stability; silencing transcription; and controlling the segregation of oxidized proteins between mother and daughter cells.

The term “ED₅₀” means the dose of a drug that produces 50% of its maximum response or effect.

For purposes of this invention, the chemical elements are identified in accordance with the Periodic Table of the Elements, CAS version, Handbook of Chemistry and Physics, 67th Ed., 1986-87, inside cover.

C. Exemplary Embodiments

Sirtuin modulators are useful in the context of injuries arising from neurotoxic (e.g., toxic to the optic nerve or the regions of the brain processing visual input) chemicals including counteracting toxic side-effects associated with certain chemotherapeutic regimes, vascular disorders (e.g., arteriosclerosis, neovascularization such as that associated with diabetes), increased ophthalmic pressure (caused by, e.g., certain drugs, surgery, glaucoma, inflammation), hereditary predisposition, infection and/or immune and autoimmune disorders or improving quality of life in aging populations experiencing progressive vision impairment. The present invention contemplates uses of such sirtuin modulators both for vision loss and vision impairment.

Accordingly, in one embodiment, the present invention describes a method for treating vision impairment due to a condition disclosed herein comprising administering to a patient a sirtuin modulator.

In one embodiment, the sirtuin modulator is a sirtuin activator. Examples of sirtuin activators include resveratrol and analogs thereof and nicotinamide riboside and analogs thereof, particularly phosphorylated analogs thereof. Prodrugs of each of these activators are also suitable for use in the invention.

In another embodiment, the sirtuin modulator is a sirtuin inhibitor.

In one embodiment, exemplary sirtuin activators are those described in Howitz et al. (2003) Nature 425: 191 and include, for example, resveratrol (3,5,4′-Trihydroxy-trans-stilbene), butein (3,4,2′,4′-Tetrahydroxychalcone), piceatannol (3,5,3′,4′-Tetrahydroxy-trans-stilbene), isoliquiritigenin (4,2′,4′-Trihydroxychalcone), fisetin (3,7,3′,4′-Tetrahyddroxyflavone), quercetin (3,5,7,3′,4′-Pentahydroxyflavone), Deoxyrhapontin (3,5-Dihydroxy-4′-methoxystilbene 3-O-β-D-glucoside); trans-Stilbene; Rhapontin (3,3′,5-Trihydroxy-4′-methoxystilbene 3-O-β-D-glucoside); cis-Stilbene; Butein (3,4,2′,4′-Tetrahydroxychalcone); 3,4,2′4′6′-Pentahydroxychalcone; Chalcone; 7,8,3′,4′-Tetrahydroxyflavone; 3,6,2′,3′-Tetrahydroxyflavone; 4′-Hydroxyflavone; 5,4′-Dihydroxyflavone; 5,7-Dihydroxyflavone; Morin (3,5,7,2′,4′-Pentahydroxyflavone); Flavone; 5-Hydroxyflavone; (−)-Epicatechin (Hydroxy Sites: 3,5,7,3′,4′); (−)-Catechin (Hydroxy Sites: 3,5,7,3′,4′); (−)-Gallocatechin (Hydroxy Sites: 3,5,7,3′,4′,5′) (+)-Catechin (Hydroxy Sites: 3,5,7,3′,4′); 5,7,3′,4′,5′-pentahydroxyflavone; Luteolin (5,7,3′,4′-Tetrahydroxyflavone); 3,6,3′,4′-Tetrahydroxyflavone; 7,3′,4′,5′-Tetrahydroxyflavone; Kaempferol (3,5,7,4′-Tetrahydroxyflavone); 6-Hydroxyapigenin (5,6,7,4′-Tetrahydoxyflavone); Scutellarein); Apigenin (5,7,4′-Trihydroxyflavone); 3,6,2′,4′-Tetrahydroxyflavone; 7,4′-Dihydroxyflavone; Daidzein (7,4′-Dihydroxyisoflavone); Genistein (5,7,4′-Trihydroxyflavanone); Naringenin (5,7,4′-Trihydroxyflavanone); 3,5,7,3′,4′-Pentahydroxyflavanone; Flavanone; Pelargonidin chloride (3,5,7,4′-Tetrahydroxyflavylium chloride); Hinokitiol (b-Thujaplicin; 2-hydroxy-4-isopropyl-2,4,6-cycloheptatrien-1-one); L-(+)-Ergothioneine ((S)-a-Carboxy-2,3-dihydro-N,N,N-trimethyl-2-thioxo-1H-imidazole-4-ethanaminium inner salt); Caffeic Acid Phenyl Ester; MCI-186 (3-Methyl-1-phenyl-2-pyrazolin-5-one); HBED (N,N′-Di-(2-hydroxybenzyl)ethylenediamine-N,N′-diacetic acid•H2O); Ambroxol (trans-4-(2-Amino-3,5-dibromobenzylamino) cyclohexane•HCl; and U-83836E ((−)-2-((4-(2,6-di-1-Pyrrolidinyl-4-pyrimidinyl)-1-piperzainyl)methyl)-3,4-dihydro-2,5,7,8-tetramethyl-2H-1-benzopyran-6-ol•2HCl). Analogs and derivatives thereof can also be used.

Other sirtuin activators may have any of formulas 1-25, 30, 32-65, and 69-76 below, and include pharmaceutically acceptable salts, prodrugs or metabolic derivatives thereof.

In one embodiment, a sirtuin activator is a stilbene or chalcone compound of formula 1: 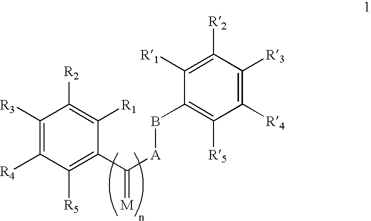
wherein, independently for each occurrence,

R₁, R₂, R₃, R₄, R₅, R′₁, R′₂, R′₃, R′₄, and R′₅ represent H, alkyl, aryl, heteroaryl, aralkyl, alkaryl, heteroaralkyl, halide, NO₂, SR, OR, N(R)₂, or carboxyl;

R represents H, alkyl, aryl, heteroaryl, or aralkyl;

M represents O, NR, or S;

A-B represents a bivalent alkyl, alkenyl, alkynyl, amido, sulfonamido, diazo, ether, alkylamino, alkylsulfide, hydroxylamine, or hydrazine group; and

n is 0 or 1.

In a further embodiment, a sirtuin activator is a compound of formula 1 and the attendant definitions, wherein n is 0. In a further embodiment, a sirtuin activator is a compound of formula 1 and the attendant definitions, wherein n is 1. In a further embodiment, a sirtuin activator is a compound of formula 1 and the attendant definitions, wherein A-B is ethenyl. In a further embodiment, a sirtuin activator is a compound of formula 1 and the attendant definitions, wherein A-B is —CH₂CH(Me)CH(Me)CH₂—. In a further embodiment, a sirtuin activator is a compound of formula 1 and the attendant definitions, wherein M is O. In a further embodiment, the methods comprises a compound of formula 1 and the attendant definitions, wherein R₁, R₂, R₃, R₄, R₅, R′₁, R′₂, R′₃, R′₄, and R′₅ are H. In a further embodiment, a sirtuin activator is a compound of formula 1 and the attendant definitions, wherein R₂, R₄, and R′₃ are OH. In a further embodiment, a sirtuin activator is a compound of formula 1 and the attendant definitions, wherein R₂, R₄, R′₂ and R′₃ are OH. In a further embodiment, a sirtuin activator is a compound of formula 1 and the attendant definitions, wherein R₃, R₅, R′₂ and R′₃ are OH. In a further embodiment, a sirtuin activator is a compound of formula 1 and the attendant definitions, wherein R₁, R₃, R₅, R′₂ and R′₃ are OH. In a further embodiment, a sirtuin activator is a compound of formula 1 and the attendant definitions, wherein R₂ and R′₂ are OH; R₄ is O-β-D-glucoside; and R′₃ is OCH₃. In a further embodiment, a sirtuin activator is a compound of formula 1 and the attendant definitions, wherein R₂ is OH; R₄ is O-β-D-glucoside; and R′₃ is OCH₃.

In a further embodiment, a sirtuin activator is a compound of formula 1 and the attendant definitions, wherein n is 0; A-B is ethenyl; and R₁, R₂, R₃, R₄, R₅, R′₁, R′₂, R′₃, R′₄, and R′₅ are H (trans stilbene). In a further embodiment, a sirtuin activator is a compound of formula 1 and the attendant definitions, wherein n is 1; A-B is ethenyl; M is O; and R₁, R₂, R₃, R₄, R₅, R′₁, R′₂, R′₃, R′₄, and R′₅ are H (chalcone). In a further embodiment, a sirtuin activator is a compound of formula 1 and the attendant definitions, wherein n is 0; A-B is ethenyl; R₂, R₄, and R′₃ are OH; and R₁, R₃, R₅, R′₁, R′₂, R′₄, and R′₅ are H (resveratrol). In a further embodiment, a sirtuin activator is a compound of formula 1 and the attendant definitions, wherein n is 0; A-B is ethenyl; R₂, R₄, R′₂ and R′₃ are OH; and R₁, R₃, R₅, R′₁, R′₄ and R′₅ are H (piceatannol). In a further embodiment, a sirtuin activator is a compound of formula 1 and the attendant definitions, wherein n is 1; A-B is ethenyl; M is O; R₃, R₅, R′₂ and R′₃ are OH; and R₁, R₂, R₄, R′₁, R′₄, and R′₅ are H (butein). In a further embodiment, a sirtuin activator is a compound of formula 1 and the attendant definitions, wherein n is 1; A-B is ethenyl; M is O; R₁, R₃, R₅, R′₂ and R′₃ are OH; and R₂, R₄, R′₁, R′₄, and R′₅ are H (3,4,2′,4′,6′-pentahydroxychalcone). In a further embodiment, a sirtuin activator is a compound of formula 1 and the attendant definitions, wherein n is 0; A-B is ethenyl; R₂ and R′₂ are OH, R₄ is O-β-D-glucoside, R′₃ is OCH₃; and R₁, R₃, R₅, R′₁, R′₄, and R′₅ are H (rhapontin). In a further embodiment, a sirtuin activator is a compound of formula 1 and the attendant definitions, wherein n is 0; A-B is ethenyl; R₂ is OH, R₄ is O-β-D-glucoside, R′₃ is OCH₃; and R₁, R₃, R₅, R′₁, R′₂, R′₄, and R′₅ are H (deoxyrhapontin). In a further embodiment, a sirtuin activator is a compound of formula 1 and the attendant definitions, wherein n is 0; A-B is —CH₂CH(Me)CH(Me)CH₂—; R₂, R₃, R′₂, and R′₃ are OH; and R₁, R₄, R₅, R′₁, R′₄, and R′₅ are H (NDGA).

In another embodiment, a sirtuin activator is a flavanone compound of formula 2:

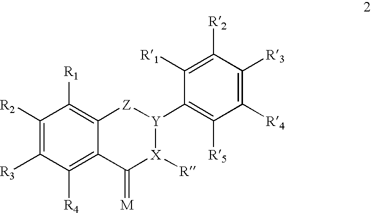

wherein, independently for each occurrence,

R₁, R₂, R₃, R₄, R′₁, R′₂, R′₃, R′₄, R′₅, and R″ represent H, alkyl, aryl, heteroaryl, aralkyl, alkaryl, heteroaralkyl, halide, NO₂, SR, OR, N(R)₂, or carboxyl;

R represents H, alkyl, aryl, heteroaryl, or aralkyl;

M represents H₂, O, NR, or S;

Z represents CR, O, NR, or S;

X represents CR or N; and

Y represents CR or N.

In a further embodiment, a sirtuin activator is a compound of formula 2 and the attendant definitions, wherein X and Y are both CH. In a further embodiment, a sirtuin activator is a compound of formula 2 and the attendant definitions, wherein M is O. In a further embodiment, a sirtuin activator is a compound of formula 2 and the attendant definitions, wherein M is H₂. In a further embodiment, a sirtuin activator is a compound of formula 2 and the attendant definitions, wherein Z is O. In a further embodiment, a sirtuin activator is a compound of formula 2 and the attendant definitions, wherein R″ is H. In a further embodiment, a sirtuin activator is a compound of formula 2 and the attendant definitions, wherein R″ is OH. In a further embodiment, a sirtuin activator is a compound of formula 2 and the attendant definitions, wherein R″ is an alkoxycarbonyl. In a further embodiment, a sirtuin activator is a compound of formula 2 and the attendant definitions, wherein R₁ is 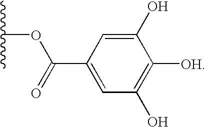
In a further embodiment, a sirtuin activator is a compound of formula 2 and the attendant definitions, wherein R₁, R₂, R₃, R₄, R′₁, R′₂, R′₃, R′₄, R′₅ and R″ are H. In a further embodiment, a sirtuin activator is a compound of formula 2 and the attendant definitions, wherein R₂, R₄, and R′₃ are OH. In a further embodiment, a sirtuin activator is a compound of formula 2 and the attendant definitions, wherein R₄, R′₂, R′₃, and R″ are OH. In a further embodiment, a sirtuin activator is a compound of formula 2 and the attendant definitions, wherein R₂, R₄, R′₂, R′₃, and R″ are OH. In a further embodiment, a sirtuin activator is a compound of formula 2 and the attendant definitions, wherein R₂, R₄, R′₂, R′₃, R′₄, and R″ are OH.

In a further embodiment, a sirtuin activator is a compound of formula 2 and the attendant definitions, wherein X and Y are CH; M is O; Z and O; R″ is H; and R₁, R₂, R₃, R₄, R′₁, R′₂, R′₃, R′₄, R′₅ and R″ are H (flavanone). In a further embodiment, a sirtuin activator is a compound of formula 2 and the attendant definitions, wherein X and Y are CH; M is O; Z and O; R″ is H; R₂, R₄, and R′₃ are OH; and R₁, R₃, R′₁, R′₂, R′₄, and R′₅ are H (naringenin). In a further embodiment, a sirtuin activator is a compound of formula 2 and the attendant definitions, wherein X and Y are CH; M is O; Z and O; R″ is OH; R₂, R₄, R′₂, and R′₃ are OH; and R₁, R₃, R′₁, R′₄, and R′₅ are H (3,5,7,3′,4′-pentahydroxyflavanone). In a further embodiment, a sirtuin activator is a compound of formula 2 and the attendant definitions, wherein X and Y are CH; M is H₂; Z and 0; R″ is OH; R₂, R₄, R′₂, and R′₃, are OH; and R₁, R₃, R′₁, R′₄ and R′₅ are H (epicatechin). In a further embodiment, a sirtuin activator is a compound of formula 2 and the attendant definitions, wherein X and Y are CH; M is H₂; Z and 0; R″ is OH; R₂, R₄, R′₂, R′₃, and R′₄ are OH; and R₁, R₃, R′₁, and R′₅ are H (gallocatechin). In a further embodiment, a sirtuin activator is a compound of formula 2 and the attendant definitions, wherein X and Y are CH; M is H₂; Z and O; R″ is 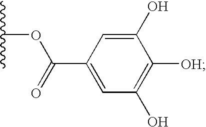
R₂, R₄, R′₂, R′₃, R′₄, and R″ are OH; and R₁, R₃, R′₁, and R′₅ are H (epigallocatechin gallate).

In another embodiment, a sirtuin activator is an isoflavanone compound of formula 3:

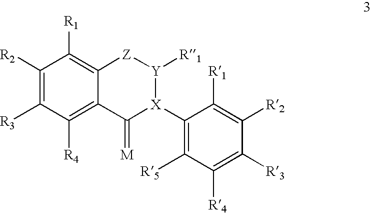

wherein, independently for each occurrence,

R₁, R₂, R₃, R₄, R′₁, R′₂, R′₃, R′₄, R′₅, and R″, represent H, alkyl, aryl, heteroaryl, aralkyl, alkaryl, heteroaralkyl, halide, NO₂, SR, OR, N(R)₂, or carboxyl;

R represents H, alkyl, aryl, heteroaryl, or aralkyl;

M represents H₂, O, NR, or S;

Z represents C(R)₂, O, NR, or S;

X represents CR or N; and

Y represents CR or N.

In another embodiment, a sirtuin activator is a flavone compound of formula 4:

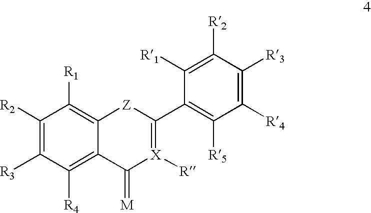

wherein, independently for each occurrence,

R₁, R₂, R₃, R₄, R′₁, R′₂, R′₃, R′₄, and R′₅, represent H, alkyl, aryl, heteroaryl, aralkyl, alkaryl, heteroaralkyl, halide, NO₂, SR, OR, N(R)₂, or carboxyl;

R represents H, alkyl, aryl, heteroaryl, or aralkyl;

M represents H₂, O, NR, or S;

Z represents CR, O, NR, or S; and

X represents CR″ or N, wherein

R″ is H, alkyl, aryl, heteroaryl, alkaryl, heteroaralkyl, halide, NO₂, SR, OR, N(R)₂, or carboxyl.

In a further embodiment, a sirtuin activator is a compound of formula 4 and the attendant definitions, wherein X is C. In a further embodiment, a sirtuin activator is a compound of formula 4 and the attendant definitions, wherein X is CR. In a further embodiment, a sirtuin activator is a compound of formula 4 and the attendant definitions, wherein Z is O. In a further embodiment, a sirtuin activator is a compound of formula 4 and the attendant definitions, wherein M is O. In a further embodiment, a sirtuin activator is a compound of formula 4 and the attendant definitions, wherein R″ is H. In a further embodiment, a sirtuin activator is a compound of formula 4 and the attendant definitions, wherein R″ is OH. In a further embodiment, a sirtuin activator is a compound of formula 4 and the attendant definitions, wherein R₁, R₂, R₃, R₄, R′₁, R′₂, R′₃, R′₄, and R′₅ are H. In a further embodiment, a sirtuin activator is a compound of formula 4 and the attendant definitions, wherein R₂, R′₂, and R′₃ are OH. In a further embodiment, a sirtuin activator is a compound of formula 4 and the attendant definitions, wherein R₂, R₄, R′₂, R′₃, and R′₄ are OH. In a further embodiment, a sirtuin activator is a compound of formula 4 and the attendant definitions, wherein R₂, R₄, R′₂, and R′₃ are OH. In a further embodiment, a sirtuin activator is a compound of formula 4 and the attendant definitions, wherein R₃, R′₂, and R′₃ are OH. In a further embodiment, a sirtuin activator is a compound of formula 4 and the attendant definitions, wherein R₂, R₄, R′₂, and R′₃ are OH. In a further embodiment, a sirtuin activator is a compound of formula 4 and the attendant definitions, wherein R₂, R′₂, R′₃, and R′₄ are OH. In a further embodiment, a sirtuin activator is a compound of formula 4 and the attendant definitions, wherein R₂, R₄, and R′₃ are OH. In a further embodiment, a sirtuin activator is a compound of formula 4 and the attendant definitions, wherein R₂, R₃, R₄, and R′₃ are OH. In a further embodiment, a sirtuin activator is a compound of formula 4 and the attendant definitions, wherein R₂, R₄, and R′₃ are OH. In a further embodiment, a sirtuin activator is a compound of formula 4 and the attendant definitions, wherein R₃, R′₁, and R′₃ are OH. In a further embodiment, a sirtuin activator is a compound of formula 4 and the attendant definitions, wherein R₂ and R′₃ are OH. In a further embodiment, a sirtuin activator is a compound of formula 4 and the attendant definitions, wherein R₁, R₂, R′₂, and R′₃ are OH. In a further embodiment, a sirtuin activator is a compound of formula 4 and the attendant definitions, wherein R₃, R′₁, and R′₂ are OH. In a further embodiment, a sirtuin activator is a compound of formula 4 and the attendant definitions, wherein R′₃ is OH. In a further embodiment, a sirtuin activator is a compound of formula 4 and the attendant definitions, wherein R₄ and R′₃ are OH. In a further embodiment, a sirtuin activator is a compound of formula 4 and the attendant definitions, wherein R₂ and R₄ are OH. In a further embodiment, a sirtuin activator is a compound of formula 4 and the attendant definitions, wherein R₂, R₄, R′₁, and R′₃ are OH. In a further embodiment, a sirtuin activator is a compound of formula 4 and the attendant definitions, wherein R₄ is OH. In a further embodiment, a sirtuin activator is a compound of formula 4 and the attendant definitions, wherein R₂, R₄, R′₂, R′₃, and R′₄ are OH. In a further embodiment, a sirtuin activator is a compound of formula 4 and the attendant definitions, wherein R₂, R′₂, R′₃, and R′₄ are OH. In a further embodiment, a sirtuin activator is a compound of formula 4 and the attendant definitions, wherein R₁, R₂, R₄, R′₂, and R′₃ are OH.

In a further embodiment, a sirtuin activator is a compound of formula 4 and the attendant definitions, wherein X is CH; Z is O; M is O; and R₁, R₂, R₃, R₄, R′₁, R′₂, R′₃, R′₄, and R′₅ are H (flavone). In a further embodiment, a sirtuin activator is a compound of formula 4 and the attendant definitions, wherein X is COH; Z is O; M is O; R₂, R′₂, and R′₃ are OH; and R₁, R₃, R₄, R′₁, R′₄, and R′₅ are H (fisetin). In a further embodiment, a sirtuin activator is a compound of formula 4 and the attendant definitions, wherein X is CH; Z is O; M is O; R₂, R₄, R′₂, R′₃, and R′₄ are OH; and R₁, R₃, R′₁, and R′₅ are H (5,7,3′,4′,5′-pentahydroxyflavone). In a further embodiment, a sirtuin activator is a compound of formula 4 and the attendant definitions, wherein X is CH; Z is O; M is O; R₂, R₄, R′₂, and R′₃ are OH; and R₁, R₃, R′₁, R′₄, and R′₅ are H (luteolin). In a further embodiment, a sirtuin activator is a compound of formula 4 and the attendant definitions, wherein X is COH; Z is O; M is O; R₃, R′₂, and R′₃ are OH; and R₁, R₂, R₄, R′, R′₄, and R′₅ are H (3,6,3′,4′-tetrahydroxyflavone). In a further embodiment, a sirtuin activator is a compound of formula 4 and the attendant definitions, wherein X is COH; Z is O; M is O; R₂, R₄, R′₂, and R′₃ are OH; and R₁, R₃, R′₁, R′₄, and R′₅ are H (quercetin). In a further embodiment, a sirtuin activator is a compound of formula 4 and the attendant definitions, wherein X is CH; Z is O; M is O; R₂, R′₂, R′₃, and R′₄ are OH; and R₁, R₃, R₄, R′₁, and R′₅ are H. In a further embodiment, a sirtuin activator is a compound of formula 4 and the attendant definitions, wherein X is COH; Z is O; M is O; R₂, R₄, and R′₃ are OH; and R₁, R₃, R′₁, R′₂, R′₄, and R′₅ are H. In a further embodiment, a sirtuin activator is a compound of formula 4 and the attendant definitions, wherein X is CH; Z is O; M is O; R₂, R₃, R₄, and R′₃ are OH; and R₁, R′₁, R′₂, R′₄, and R′₅ are H. In a further embodiment, a sirtuin activator is a compound of formula 4 and the attendant definitions, wherein X is CH; Z is O; M is O; R₂, R₄, and R′₃ are OH; and R₁, R₃, R′₁, R′₂, R′₄, and R′₅ are H. In a further embodiment, a sirtuin activator is a compound of formula 4 and the attendant definitions, wherein X is COH; Z is O; M is O; R₃, R′₁, and R′₃ are OH; and R₁, R₂, R₄, R′₂, R′₄, and R′₅ are H. In a further embodiment, a sirtuin activator is a compound of formula 4 and the attendant definitions, wherein X is CH; Z is O; M is O; R₂ and R′₃ are OH; and R₁, R₃, R₄, R′₁, R′₂, R′₄, and R′₅ are H. In a further embodiment, a sirtuin activator is a compound of formula 4 and the attendant definitions, wherein X is COH; Z is O; M is O; R₁, R₂, R′₂, and R′₃ are OH; and R₁, R₂, R₄, R′₃, R′₄, and R′₅ are H. In a further embodiment, a sirtuin activator is a compound of formula 4 and the attendant definitions, wherein X is COH; Z is O; M is O; R₃, R′₁, and R′₂ are OH; and R₁, R₂, R₄; R′₃, R′₄, and R′₅ are H. In a further embodiment, a sirtuin activator is a compound of formula 4 and the attendant definitions, wherein X is CH; Z is O; M is O; R′₃ is OH; and R₁, R₂, R₃, R₄, R′₁, R′₂, R′₄, and R′₅ are H. In a further embodiment, a sirtuin activator is a compound of formula 4 and the attendant definitions, wherein X is CH; Z is O; M is O; R₄ and R′₃ are OH; and R₁, R₂, R₃, R′₁, R′₂, R′₄, and R′₅ are H. In a further embodiment, a sirtuin activator is a compound of formula 4 and the attendant definitions, wherein X is CH; Z is O; M is O; R₂ and R₄ are OH; and R₁, R₃, R′₁, R′₂, R′₃, R′₄, and R′₅ are H. In a further embodiment, a sirtuin activator is a compound of formula 4 and the attendant definitions, wherein X is COH; Z is O; M is O; R₂, R₄, R′₁, and R′₃ are OH; and R₁, R₃, R′₂, R′₄, and R′₅ are H. In a further embodiment, a sirtuin activator is a compound of formula 4 and the attendant definitions, wherein X is CH; Z is O; M is O; R₄ is OH; and R₁, R₂, R₃, R′₁, R′₂, R′₃, R′₄, and R′₅ are H. In a further embodiment, a sirtuin activator is a compound of formula 4 and the attendant definitions, wherein X is COH; Z is O; M is O; R₂, R₄, R′₂, R′₃, and R′₄ are OH; and R₁, R₃, R′₁, and R′₅ are H. In a further embodiment, a sirtuin activator is a compound of formula 4 and the attendant definitions, wherein X is COH; Z is O; M is O; R₂, R′₂, R′₃, and R′₄ are OH; and R₁, R₃, R₄, R′₁, and R′₅ are H. In a further embodiment, a sirtuin activator is a compound of formula 4 and the attendant definitions, wherein X is COH; Z is O; M is O; R₁, R₂, R₄, R′₂, and R′₃ are OH; and R₃, R′₁, R′₄, and R′₅ are H.

In another embodiment, a sirtuin activator is an isoflavone compound of formula 5:

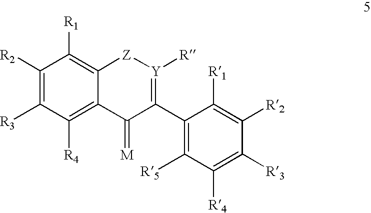

wherein, independently for each occurrence,

R₁, R₂, R₃, R₄, R′₁, R′₂, R′₃, R′₄, and R′₅, represent H, alkyl, aryl, heteroaryl, aralkyl, alkaryl, heteroaralkyl, halide, NO₂, SR, OR, N(R)₂, or carboxyl;

R represents H, alkyl, aryl, heteroaryl, or aralkyl;

M represents H₂, O, NR, or S;

Z represents C(R)₂, O, NR, or S; and

Y represents CR″ or N, wherein

R″ represents H, alkyl, aryl, heteroaryl, alkaryl, heteroaralkyl, halide, NO₂, SR, OR, N(R)₂, or carboxyl.

In a further embodiment, a sirtuin activator is a compound of formula 5 and the attendant definitions, wherein Y is CR″. In a further embodiment, a sirtuin activator is a compound of formula 5 and the attendant definitions, wherein Y is CH. In a further embodiment, a sirtuin activator is a compound of formula 5 and the attendant definitions, wherein Z is O. In a further embodiment, a sirtuin activator is a compound of formula 5 and the attendant definitions, wherein M is O. In a further embodiment, a sirtuin activator is a compound of formula 5 and the attendant definitions, wherein R₂ and R′₃ are OH. In a further embodiment, a sirtuin activator is a compound of formula 5 and the attendant definitions, wherein R₂, R₄, and R′₃ are OH.

In a further embodiment, a sirtuin activator is a compound of formula 5 and the attendant definitions, wherein Y is CH; Z is O; M is O; R₂ and R′₃ are OH; and R₁, R₃, R₄, R′₁, R′₂, R′₄, and R′₅ are H. In a further embodiment, a sirtuin activator is a compound of formula 5 and the attendant definitions, wherein Y is CH; Z is O; M is O; R₂, R₄, and R′₃ are OH; and R₁, R₃, R′₁, R′₂, R′₄, and R′₅ are H.

In another embodiment, a sirtuin activator is an anthocyanidin compound of formula 6:

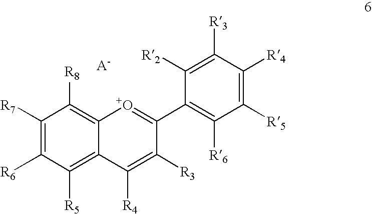

wherein, independently for each occurrence,

R₃, R₄, R₅, R₆, R₇, R₈, R′₂, R′₃, R′₄, R′₅, and R′₆ represent H, alkyl, aryl, heteroaryl, aralkyl, alkaryl, heteroaralkyl, halide, NO₂, SR, OR, N(R)₂, or carboxyl;

R represents H, alkyl, aryl, heteroaryl, or aralkyl; and

A⁻ represents an anion selected from the following: Cl⁻, Br⁻, or I⁻.

In a further embodiment, a sirtuin activator is a compound of formula 6 and the attendant definitions, wherein A⁻ is Cl⁻. In a further embodiment, a sirtuin activator is a compound of formula 6 and the attendant definitions, wherein R₃, R₅, R₇, and R′₄ are OH. In a further embodiment, a sirtuin activator is a compound of formula 6 and the attendant definitions, wherein R₃, R₅, R₇, R′₃, and R′₄ are OH. In a further embodiment, a sirtuin activator is a compound of formula 6 and the attendant definitions, wherein R₃, R₅, R₇, R′₃, R′₄, and R′₅ are OH.

In a further embodiment, a sirtuin activator is a compound of formula 6 and the attendant definitions, wherein A⁻ is Cl⁻; R₃, R₅, R₇, and R′₄ are OH; and R₄, R₆, R₈, R′₂, R′₃, R′₅, and R′₆ are H. In a further embodiment, a sirtuin activator is a compound of formula 6 and the attendant definitions, wherein A⁻ is Cl⁻; R₃, R₅, R₇, R′₃, and R′₄ are OH; and R₄, R₆, R₈, R′₂, R′₅, and R′₆ are H. In a further embodiment, a sirtuin activator is a compound of formula 6 and the attendant definitions, wherein A⁻ is Cl⁻; R₃, R₅, R₇, R′₃, R′₄, and R′₅ are OH; and R₄, R₆, R′₁, R′₂, and R′₆ are H.

In a further embodiment, a sirtuin activator is a stilbene, chalcone, or flavone compound represented by formula 7:

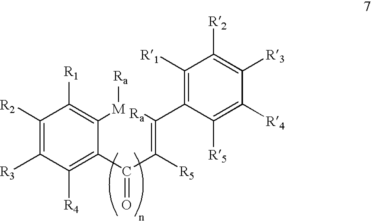

wherein, independently for each occurrence,

M is absent or O;

R₁, R₂, R₃, R₄, R₅, R′₁, R′₂, R′₃, R′₄, and R′₅ represent H, alkyl, aryl, heteroaryl, aralkyl, alkaryl, heteroaralkyl, halide, NO₂, SR, OR, N(R)₂, or carboxyl;

Rₐ represents H or the two instances of Rₐ form a bond;

R represents H, alkyl, aryl, heteroaryl, aralkyl; and

n is 0 or 1.

In a further embodiment, a sirtuin activator is an activating compound represented by formula 7 and the attendant definitions, wherein n is 0. In a further embodiment, a sirtuin activator is an activating compound represented by formula 7 and the attendant definitions, wherein n is 1. In a further embodiment, a sirtuin activator is an activating compound represented by formula 7 and the attendant definitions, wherein M is absent. In a further embodiment, a sirtuin activator is an activating compound represented by formula 7 and the attendant definitions, wherein M is O. In a further embodiment, a sirtuin activator is an activating compound represented by formula 7 and the attendant definitions, wherein Rₐ is H. In a further embodiment, a sirtuin activator is an activating compound represented by formula 7 and the attendant definitions, wherein M is O and the two Rₐ form a bond.

In a further embodiment, a sirtuin activator is an activating compound represented by formula 7 and the attendant definitions, wherein R₅ is H. In a further embodiment, a sirtuin activator is an activating compound represented by formula 7 and the attendant definitions, wherein R₅ is OH. In a further embodiment, a sirtuin activator is an activating compound represented by formula 7 and the attendant definitions, wherein R₁, R₃, and R′₃ are OH. In a further embodiment, a sirtuin activator is an activating compound represented by formula 7 and the attendant definitions, wherein R₂, R₄, R′₂, and R′₃ are OH. In a further embodiment, a sirtuin activator is an activating compound represented by formula 7 and the attendant definitions, wherein R₂, R′₂, and R′₃ are OH. In a further embodiment, a sirtuin activator is an activating compound represented by formula 7 and the attendant definitions, wherein R₂ and R₄ are OH.

In a further embodiment, a sirtuin activator is a compound represented by formula 7 and the attendant definitions, wherein n is 0; M is absent; Rₐ is H; R₅ is H; R₁, R₃, and R′₃ are OH; and R₂, R₄, R′₁, R′₂, R′₄, and R′₅ are H. In a further embodiment, a sirtuin activator is an activating compound represented by formula 7 and the attendant definitions, wherein n is 1; M is absent; Rₐ is H; R₅ is H; R₂, R₄, R′₂, and R′₃ are OH; and R₁, R₃, R′₁, R′₄, and R′₅ are H. In a further embodiment, a sirtuin activator is an activating compound represented by formula 7 and the attendant definitions, wherein n is 1; M is O; the two Rₐ form a bond; R₅ is OH; R₂, R′₂, and R′₃ are OH; and R₁, R₃, R₄, R′₁, R′₄, and R′₅ are H.

Other sirtuin activators include compounds having a formula selected from the group consisting of formulas 8-25 and 30 set forth below.

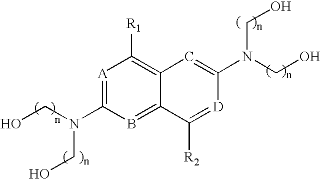

8
R₁, R₂ = H, aryl, heterocycle, small alkyl
A,B,C,D = CR₁,N
n = 0,1,2,3

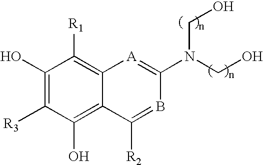

9
R₁, R₂ = H, aryl, heterocycle, small alkyl
R₃ = H, small alkyl
A,B = CR₁,N
n = 0,1,2,3

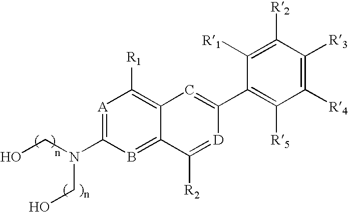

10
R₁, R₂ = H, aryl, heterocycle, small alkyl
R′₁-R′₅ = H, OH
A,B,C,D = CR₁,N
n = 0,1,2,3

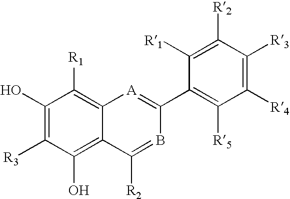

11
R₁, R₂ = H, aryl, heterocycle, small alkyl
R₃ = H, small alkyl
R′₁-R′₅ = H, OH
A,B = CR₁,N
n = 0,1,2,3

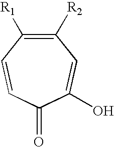

12
R₁, R₂ = H, alkyl, alkenyl

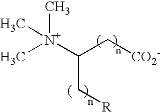

13
R = Heterocycle, aryl
n = 0-10

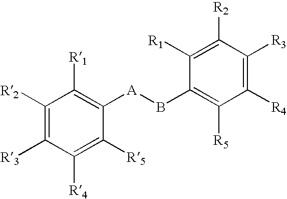

14

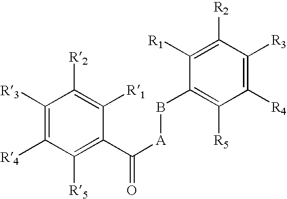

15

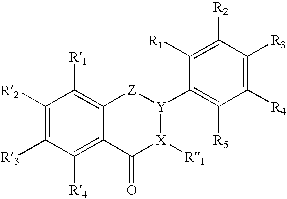

16

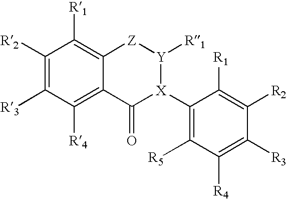

17

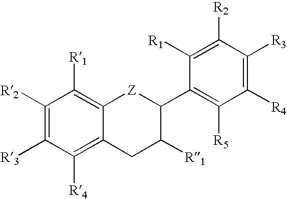

18
R₁ = H, halogen,NO₂,SR(R = H,alkyl,aryl),OR(R = H, alkyl, aryl), NRR′(R,R′ = alkyl,aryl), alkyl, aryl, carboxy
R₂ = H, halogen,NO₂,SR(R = H,alkyl,aryl),OR(R = H, alkyl, aryl), NRR′(R,R′ = alkyl,aryl), alkyl, aryl, carboxy
R₃ = H, halogen,NO₂,SR(R = H,alkyl,aryl),OR(R = H, alkyl, aryl), NRR′(R,R′ = alkyl,aryl), alkyl, aryl, carboxy
R₄ = H, halogen,NO₂,SR(R = H,alkyl,aryl),OR(R = H, alkyl, aryl), NRR′(R,R′ = alkyl,aryl), alkyl, aryl, carboxy
R₅ = H, halogen,NO₂,SR(R = H,alkyl,aryl),OR(R = H, alkyl, aryl), NRR′(R,R′ = alkyl,aryl), alkyl, aryl, carboxy
R′₁ = H, halogen,NO₂,SR(R = H,alkyl,aryl),OR(R = H, alkyl, aryl), NRR′(R,R′ = alkyl,aryl), alkyl, aryl, carboxy
R′₂ = H, halogen,NO₂,SR(R = H,alkyl,aryl),OR(R = H, alkyl, aryl), NRR′(R,R′ = alkyl,aryl), alkyl, aryl, carboxy
R′₃ = H, halogen,NO₂,SR(R = H,alkyl,aryl),OR(R = H, alkyl, aryl), NRR′(R,R′ = alkyl,aryl), alkyl, aryl, carboxy
R′₄ = H, halogen,NO₂,SR(R = H,alkyl,aryl),OR(R = H, alkyl, aryl), NRR′(R,R′ = alkyl,aryl), alkyl, aryl, carboxy
R′₅ = H, halogen,NO₂,SR(R = H,alkyl,aryl),OR(R = H, alkyl, aryl), NRR′(R,R′ = alkyl,aryl), alkyl, aryl, carboxy
R″₁ = H, halogen,NO₂,SR(R = H,alkyl,aryl),OR(R = H, alkyl, aryl), NRR′(R,R′ = alkyl,aryl), alkyl, aryl, carboxy
A-B = ethene,ethyne,amide,sulfonamide,diazo,alkyl ether,alkyl amine,alkyl sulfide,hydroxyamine,hydrazine
X = CR,N
Y = CR,N
Z = O,S,C(R)₂,NR
R = H, alkyl, aryl, aralkyl

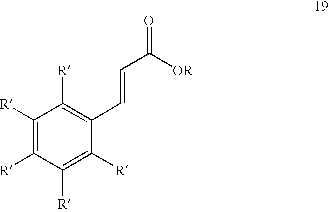

wherein, independently for each occurrence,

R═H, alkyl, aryl, heterocyclyl, heteroaryl, or aralkyl; and

R′═H, halogen, NO₂, SR, OR, NR₂, alkyl, aryl, or carboxy.

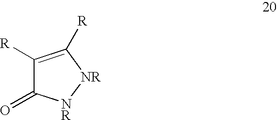

wherein, independently for each occurrence,

R═H, alkyl, aryl, heterocyclyl, heteroaryl, or aralkyl.

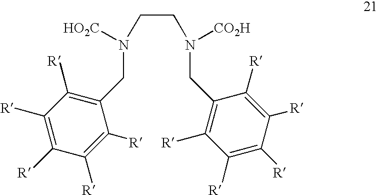

wherein, independently for each occurrence,

R′═H, halogen, NO₂, SR, OR, NR₂, alkyl, aryl, aralkyl, or carboxy; and

R═H, alkyl, aryl, heterocyclyl, heteroaryl, or aralkyl.

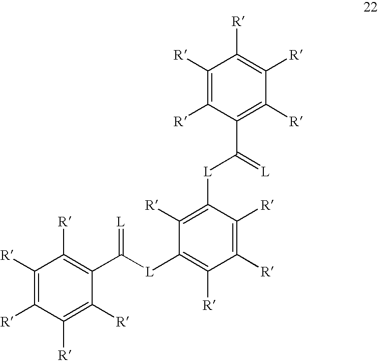

wherein, independently for each occurrence,

L represents CR₂, O, NR, or S;

R represents H, alkyl, aryl, aralkyl, or heteroaralkyl; and

R′ represents H, halogen, NO₂, SR, OR, NR₂, alkyl, aryl, aralkyl, or carboxy.

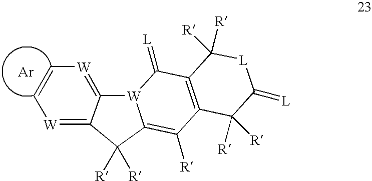

wherein, independently for each occurrence,

L represents CR₂, O, NR, or S;

W represents CR or N;

R represents H, alkyl, aryl, aralkyl, or heteroaralkyl;

Ar represents a fused aryl or heteroaryl ring; and

R′ represents H, halogen, NO₂, SR, OR, NR₂, alkyl, aryl, aralkyl, or carboxy.

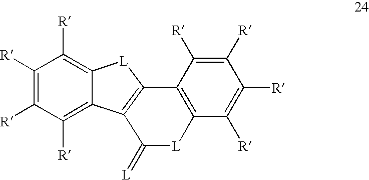

wherein, independently for each occurrence,

L represents CR₂, O, NR, or S;

R represents H, alkyl, aryl, aralkyl, or heteroaralkyl; and

R′ represents H, halogen, NO₂, SR, OR, NR₂, alkyl, aryl, aralkyl, or carboxy.

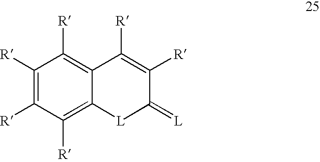

wherein, independently for each occurrence,

L represents CR₂, O, NR, or S;

R represents H, alkyl, aryl, aralkyl, or heteroaralkyl; and

R′ represents H, halogen, NO₂, SR, OR, NR₂, alkyl, aryl, aralkyl, or carboxy.

In a further embodiment, a sirtuin activator is a stilbene, chalcone, or flavone compound represented by formula 30:

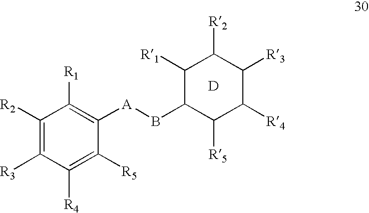

wherein, independently for each occurrence,

D is a phenyl or cyclohexyl group;

R₁, R₂, R₃, R₄, R₅, R′₁, R′₂, R′₃, R′₄, and R′₅ represent H, alkyl, aryl, heteroaryl, alkaryl, heteroaralkyl, halide, NO₂, SR, OR, N(R)₂, carboxyl, azide, ether; or any two adjacent R or R′ groups taken together form a fused benzene or cyclohexyl group;

R represents H, alkyl, aryl, or aralkyl; and

A-B represents an ethylene, ethenylene, or imine group;

provided that when A-B is ethenylene, D is phenyl, and R′₃ is H: R₃ is not OH when R₁, R₂, R₄, and R₅ are H; and R₂ and R₄ are not OMe when R₁, R₃, and R₅ are H; and R₃ is not OMe when R₁, R₂, R₄, and R₅ are H.

In a further embodiment, a sirtuin activator is a compound represented by formula 30 and the attendant definitions, wherein D is a phenyl group.

In a further embodiment, a sirtuin activator is a compound represented by formula 30 and the attendant definitions, wherein A-B is an ethenylene or imine group.

In a further embodiment, a sirtuin activator is a compound represented by formula 30 and the attendant definitions, wherein A-B is an ethenylene group.

In a further embodiment, a sirtuin activator is a compound represented by formula 30 and the attendant definitions, wherein R₂ is OH.

In a further embodiment, a sirtuin activator is a compound represented by formula 30 and the attendant definitions, wherein R₄ is OH

In a further embodiment, a sirtuin activator is a compound represented by formula 30 and the attendant definitions, wherein R₂ and R₄ are OH.

In a further embodiment, a sirtuin activator is a compound represented by formula 30 and the attendant definitions, wherein D is a phenyl group; and A-B is an ethenylene group.

In a further embodiment, a sirtuin activator is a compound represented by formula 30 and the attendant definitions, wherein D is a phenyl group; A-B is an ethenylene group; and R₂ and R₄ are OH.

In a further embodiment, a sirtuin activator is a compound represented by formula 30 and the attendant definitions, wherein A-B is ethenylene; D is a phenyl ring; R₂ and R₄ are OH; and R′₃ is Cl.

In a further embodiment, a sirtuin activator is a compound represented by formula 30 and the attendant definitions, wherein A-B is ethenylene; D is a phenyl ring; R₂ and R₄ are OH; and R′₃ is OH.

In a further embodiment, a sirtuin activator is a compound represented by formula 30 and the attendant definitions, wherein A-B is ethenylene; D is a phenyl ring; R₂ and R₄ are OH; and R′₃ is H.

In a further embodiment, a sirtuin activator is a compound represented by formula 30 and the attendant definitions, wherein A-B is ethenylene; D is a phenyl ring; R₂ and R₄ are OH; and R′₃ is CH₂CH₃.

In a further embodiment, a sirtuin activator is a compound represented by formula 30 and the attendant definitions, wherein A-B is ethenylene; D is a phenyl ring; R₂ and R₄ are OH; and R′₃ is F.

In a further embodiment, a sirtuin activator is a compound represented by formula 30 and the attendant definitions, wherein A-B is ethenylene; D is a phenyl ring; R₂ and R₄ are OH; and R′₃ is Me.

In a further embodiment, a sirtuin activator is a compound represented by formula 30 and the attendant definitions, wherein A-B is ethenylene; D is a phenyl ring; R₂ and R₄ are OH; and R′₃ is an azide.

In a further embodiment, a sirtuin activator is a compound represented by formula 30 and the attendant definitions, wherein A-B is ethenylene; D is a phenyl ring; R₂ and R₄ are OH; and R′₃ is SMe.

In a further embodiment, a sirtuin activator is a compound represented by formula 30 and the attendant definitions, wherein A-B is ethenylene; D is a phenyl ring; R₂ and R₄ are OH; and R′₃ is NO₂.

In a further embodiment, a sirtuin activator is a compound represented by formula 30 and the attendant definitions, wherein A-B is ethenylene; D is a phenyl ring; R₂ and R₄ are OH; and R′₃ is CH(CH₃)₂.

In a further embodiment, a sirtuin activator is a compound represented by formula 30 and the attendant definitions, wherein A-B is ethenylene; D is a phenyl ring; R₂ and R₄ are OH; and R′₃ is OMe.

In a further embodiment, a sirtuin activator is a compound represented by formula 30 and the attendant definitions, wherein A-B is ethenylene; D is a phenyl ring; R₂ and R₄ are OH; R′₂ is OH; and R′₃ is OMe.

In a further embodiment, a sirtuin activator is a compound represented by formula 30 and the attendant definitions, wherein A-B is ethenylene; D is a phenyl ring; R₂ is OH; R₄ is carboxyl; and R′₃ is OH.

In a further embodiment, a sirtuin activator is a compound represented by formula 30 and the attendant definitions, wherein A-B is ethenylene; D is a phenyl ring; R₂ and R₄ are OH; and R′₃ is carboxyl.

In a further embodiment, a sirtuin activator is a compound represented by formula 30 and the attendant definitions, wherein A-B is ethenylene; D is a phenyl ring; R₂ and R₄ are OH; and R′₃ and R′₄ taken together form a fused benzene ring.

In a further embodiment, a sirtuin activator is a compound represented by formula 30 and the attendant definitions, wherein A-B is ethenylene; D is a phenyl ring; and R₄ is OH.

In a further embodiment, a sirtuin activator is a compound represented by formula 30 and the attendant definitions, wherein A-B is ethenylene; D is a phenyl ring; R₂ and R₄ are OCH₂OCH₃; and R′₃ is SMe.

In a further embodiment, a sirtuin activator is a compound represented by formula 30 and the attendant definitions, wherein A-B is ethenylene; D is a phenyl ring; R₂ and R₄ are OH; and R′₃ is carboxyl.

In a further embodiment, a sirtuin activator is a compound represented by formula 30 and the attendant definitions, wherein A-B is ethenylene; D is a cyclohexyl ring; and R₂ and R₄ are OH.

In a further embodiment, a sirtuin activator is a compound represented by formula 30 and the attendant definitions, wherein A-B is ethenylene; D is a phenyl ring; and R₃ and R₄ are OMe.

In a further embodiment, a sirtuin activator is a compound represented by formula 30 and the attendant definitions, wherein A-B is ethenylene; D is a phenyl ring; R₂ and R₄ are OH; and R′₃ is OH.

In another embodiment, a sirtuin activator is a compound of formula 32: 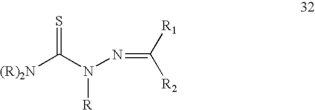
wherein, independently for each occurrence:

R is H, or a substituted or unsubstituted alkyl, aryl, aralkyl, heterocyclyl, heterocyclylalkyl, heteroaryl, or heteroaralkyl; and

R₁ and R₂ are a substituted or unsubstituted alkyl, aryl, aralkyl, heterocyclyl, heterocyclylalkyl, heteroaryl, or heteroaralkyl.

In a further embodiment, a sirtuin activator is a compound of formula 32 and the attendant definitions wherein R is H.

In a further embodiment, a sirtuin activator is a compound of formula 32 and the attendant definitions wherein R₁ is 3-hydroxyphenyl.

In a further embodiment, a sirtuin activator is a compound of formula 32 and the attendant definitions wherein R₂ is methyl.

In a further embodiment, a sirtuin activator is a compound of formula 32 and the attendant definitions wherein R is H and R₁ is 3-hydroxyphenyl.

In a further embodiment, a sirtuin activator is a compound of formula 32 and the attendant definitions wherein R is H, R₁ is 3-hydroxyphenyl, and R₂ is methyl.

In another embodiment, a sirtuin activator is a compound of formula 33: 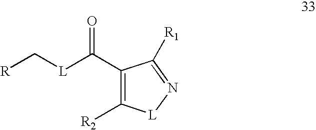
wherein, independently for each occurrence:

R is H, or a substituted or unsubstituted alkyl, alkenyl, or alkynyl;

R₁ and R₂ are a substituted or unsubstituted alkyl, aryl, aralkyl, heterocyclyl, heterocyclylalkyl, heteroaryl, or heteroaralkyl; and

L is O, S, or NR.

In a further embodiment, a sirtuin activator is a compound of formula 33 and the attendant definitions wherein R is alkynyl.

In a further embodiment, a sirtuin activator is a compound of formula 33 and the attendant definitions wherein R₁ is 2,6-dichlorophenyl.

In a further embodiment, a sirtuin activator is a compound of formula 33 and the attendant definitions wherein R₂ is methyl.

In a further embodiment, a sirtuin activator is a compound of formula 33 and the attendant definitions wherein L is O.

In a further embodiment, a sirtuin activator is a compound of formula 33 and the attendant definitions wherein R is alkynyl and R₁ is 2,6-dichlorophenyl.

In a further embodiment, a sirtuin activator is a compound of formula 33 and the attendant definitions wherein R is alkynyl, R₁ is 2,6-dichlorophenyl, and R₂ is methyl.

In a further embodiment, a sirtuin activator is a compound of formula 33 and the attendant definitions wherein R is alkynyl, R₁ is 2,6-dichlorophenyl, R₂ is methyl, and L is O.

In another embodiment, a sirtuin activator is a compound of formula 34: 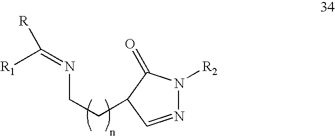
wherein, independently for each occurrence:

R, R₁, and R₂ are H, or a substituted or unsubstituted alkyl, aryl, aralkyl, heterocyclyl, heterocyclylalkyl, heteroaryl, or heteroaralkyl; and

n is an integer from 0 to 5 inclusive.

In a further embodiment, a sirtuin activator is a compound of formula 34 and the attendant definitions wherein R is 3,5-dichloro-2-hydroxyphenyl.

In a further embodiment, a sirtuin activator is a compound of formula 34 and the attendant definitions wherein R₁ is H.

In a further embodiment, a sirtuin activator is a compound of formula 34 and the attendant definitions wherein R₂ is H.

In a further embodiment, a sirtuin activator is a compound of formula 34 and the attendant definitions wherein n is 1.

In a further embodiment, a sirtuin activator is a compound of formula 34 and the attendant definitions wherein R is 3,5-dichloro-2-hydroxyphenyl and R₁ is H.

In a further embodiment, a sirtuin activator is a compound of formula 34 and the attendant definitions wherein R is 3,5-dichloro-2-hydroxyphenyl, R₁ is H, and R₂ is H.

In a further embodiment, a sirtuin activator is a compound of formula 34 and the attendant definitions wherein R is 3,5-dichloro-2-hydroxyphenyl, R₁ is H, R₂ is H, and n is 1.

In another embodiment, a sirtuin activator is a compound of formula 35: 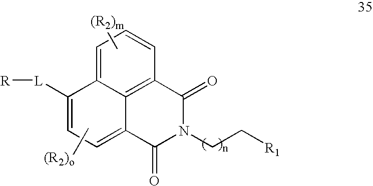
wherein, independently for each occurrence:

R is H or a substituted or unsubstituted alkyl, aryl, aralkyl, heterocyclyl, heterocyclylalkyl, heteroaryl, or heteroaralkyl;

R₁ is a substituted or unsubstituted alkyl, aryl, aralkyl, heterocyclyl, heterocyclylalkyl, heteroaryl, or heteroaralkyl;

R₂ is hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, heteroaralkyl;

L is O, NR, or S;

m is an integer from 0 to 3 inclusive;

n is an integer from 0 to 5 inclusive; and

o is an integer from 0 to 2 inclusive.

In a further embodiment, a sirtuin activator is a compound of formula 35 and the attendant definitions wherein R is phenyl.

In a further embodiment, a sirtuin activator is a compound of formula 35 and the attendant definitions wherein R₁ is pyridine.

In a further embodiment, a sirtuin activator is a compound of formula 35 and the attendant definitions wherein L is S.

In a further embodiment, a sirtuin activator is a compound of formula 35 and the attendant definitions wherein m is 0.

In a further embodiment, a sirtuin activator is a compound of formula 35 and the attendant definitions wherein n is 1.

In a further embodiment, a sirtuin activator is a compound of formula 35 and the attendant definitions wherein o is 0.

In a further embodiment, a sirtuin activator is a compound of formula 35 and the attendant definitions wherein R is phenyl and R₁ is pyridine.

In a further embodiment, a sirtuin activator is a compound of formula 35 and the attendant definitions wherein R is phenyl, R₁ is pyridine, and L is S.

In a further embodiment, a sirtuin activator is a compound of formula 35 and the attendant definitions wherein R is phenyl, R₁ is pyridine, L is S, and m is 0.

In a further embodiment, a sirtuin activator is a compound of formula 35 and the attendant definitions wherein R is phenyl, R₁ is pyridine, L is S, m is 0, and n is 1.

In a further embodiment, a sirtuin activator is a compound of formula 35 and the attendant definitions wherein R is phenyl, R₁ is pyridine, L is S, m is 0, n is 1, and o is 0.

In another embodiment, a sirtuin activator is a compound of formula 36: 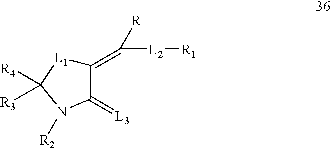
wherein, independently for each occurrence:

R, R₃, and R₄ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, heteroaralkyl;

R₁ and R₂ are H or a substituted or unsubstituted alkyl, aryl, aralkyl, heterocyclyl, heterocyclylalkyl, heteroaryl, heteroaralkyl;

L₁ is O, NR₁, S, C(R)₂, or SO₂; and

L₂ and L₃ are O, NR₁, S, or C(R)₂.

In a further embodiment, a sirtuin activator is a compound of formula 36 and the attendant definitions wherein R is H.

In a further embodiment, a sirtuin activator is a compound of formula 36 and the attendant definitions wherein R₁ is 4-chlorophenyl.

In a further embodiment, a sirtuin activator is a compound of formula 36 and the attendant definitions wherein R₂ is 4-chlorophenyl.

In a further embodiment, a sirtuin activator is a compound of formula 36 and the attendant definitions wherein R₃ is H.

In a further embodiment, a sirtuin activator is a compound of formula 36 and the attendant definitions wherein R₄ is H.

In a further embodiment, a sirtuin activator is a compound of formula 36 and the attendant definitions wherein L₁ is SO₂.

In a further embodiment, a sirtuin activator is a compound of formula 36 and the attendant definitions wherein L₂ is NH.

In a further embodiment, a sirtuin activator is a compound of formula 36 and the attendant definitions wherein L₃ is O.

In a further embodiment, a sirtuin activator is a compound of formula 36 and the attendant definitions wherein R is H and R₁ is 4-chlorophenyl.

In a further embodiment, a sirtuin activator is a compound of formula 36 and the attendant definitions wherein R is H, R₁ is 4-chlorophenyl, and R₂ is 4-chlorophenyl.

In a further embodiment, a sirtuin activator is a compound of formula 36 and the attendant definitions wherein R is H, R₁ is 4-chlorophenyl, R₂ is 4-chlorophenyl, and R₃ is H.

In a further embodiment, a sirtuin activator is a compound of formula 36 and the attendant definitions wherein R is H, R₁ is 4-chlorophenyl, R₂ is 4-chlorophenyl, R₃ is H, and R₄ is H.

In a further embodiment, a sirtuin activator is a compound of formula 36 and the attendant definitions wherein R is H, R₁ is 4-chlorophenyl, R₂ is 4-chlorophenyl, R₃ is H, R₄ is H, and L₁ is SO₂

In a further embodiment, a sirtuin activator is a compound of formula 36 and the attendant definitions wherein R is H, R₁ is 4-chlorophenyl, R₂ is 4-chlorophenyl, R₃ is H, R₄ is H, L₁ is SO₂, and L₂ is NH.

In a further embodiment, a sirtuin activator is a compound of formula 36 and the attendant definitions wherein R is H, R₁ is 4-chlorophenyl, R₂ is 4-chlorophenyl, R₃ is H, R₄ is H, L₁ is SO₂, L₂ is NH, and L₃ is O.

In another embodiment, a sirtuin activator is a compound of formula 37: 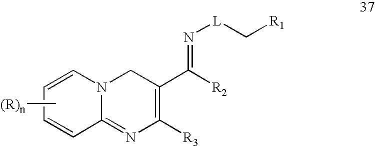
wherein, independently for each occurrence:

R is hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, heteroaralkyl;

R₁ is H or a substituted or unsubstituted alkyl, aryl, aralkyl, heterocyclyl, heterocyclylalkyl, heteroaryl, heteroaralkyl;

R₂ and R₃ are H or a substituted or unsubstituted alkyl, aryl, aralkyl, heterocyclyl, heterocyclylalkyl, heteroaryl, heteroaralkyl;

L is O, NR₁, or S; and

n is an integer from 0 to 4 inclusive.

In a further embodiment, a sirtuin activator is a compound of formula 37 and the attendant definitions wherein R is methyl.

In a further embodiment, a sirtuin activator is a compound of formula 37 and the attendant definitions wherein n is 1.

In a further embodiment, a sirtuin activator is a compound of formula 37 and the attendant definitions wherein R₁ is 3-fluorophenyl.

In a further embodiment, a sirtuin activator is a compound of formula 37 and the attendant definitions wherein R₂ is H.

In a further embodiment, a sirtuin activator is a compound of formula 37 and the attendant definitions wherein R₃ is 4-chlorophenyl.

In a further embodiment, a sirtuin activator is a compound of formula 37 and the attendant definitions wherein L is O.

In a further embodiment, a sirtuin activator is a compound of formula 37 and the attendant definitions wherein R is methyl and n is 1.

In a further embodiment, a sirtuin activator is a compound of formula 37 and the attendant definitions wherein R is methyl, n is 1, and R₁ is 3-fluorophenyl.

In a further embodiment, a sirtuin activator is a compound of formula 37 and the attendant definitions wherein R is methyl, n is 1, R₁ is 3-fluorophenyl, and R₂ is H.

In a further embodiment, a sirtuin activator is a compound of formula 37 and the attendant definitions wherein R is methyl, n is 1, R₁ is 3-fluorophenyl, R₂ is H, and R₃ is 4-chlorophenyl.

In another embodiment, a sirtuin activator is a compound of formula 38: 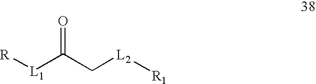
wherein, independently for each occurrence:

R and R₁ are H or a substituted or unsubstituted alkyl, aryl, aralkyl, heterocyclyl, heterocyclylalkyl, heteroaryl, or heteroaralkyl; and

L₁ and L₂ are O, NR, or S.

In a further embodiment, a sirtuin activator is a compound of formula 38 and the attendant definitions wherein R is 3-methoxyphenyl.

In a further embodiment, a sirtuin activator is a compound of formula 38 and the attendant definitions wherein R₁ is 4-t-butylphenyl.

In a further embodiment, a sirtuin activator is a compound of formula 38 and the attendant definitions wherein L₁ is NH.

In a further embodiment, a sirtuin activator is a compound of formula 38 and the attendant definitions wherein L₂ is O.

In a further embodiment, a sirtuin activator is a compound of formula 38 and the attendant definitions wherein R is 3-methoxyphenyl and R₁ is 4-t-butylphenyl.

In a further embodiment, a sirtuin activator is a compound of formula 38 and the attendant definitions wherein R is 3-methoxyphenyl, R₁ is 4-t-butylphenyl, and L₁ is NH.

In a further embodiment, a sirtuin activator is a compound of formula 38 and the attendant definitions wherein R is 3-methoxyphenyl, R₁ is 4-t-butylphenyl, L₁ is NH, and L₂ is O.

In another embodiment, a sirtuin activator is a compound of formula 39: 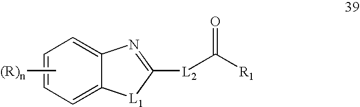
wherein, independently for each occurrence:

R is H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₁ is H or a substituted or unsubstituted alkyl, aryl, alkaryl, heterocyclyl, heterocyclylalkyl, heteroaryl, or heteroaralkyl;

L₁ and L₂ are O, NR, or S; and

n is an integer from 0 to 4 inclusive.

In a further embodiment, a sirtuin activator is a compound of formula 39 and the attendant definitions wherein R is methyl.

In a further embodiment, a sirtuin activator is a compound of formula 39 and the attendant definitions wherein n is 1.

In a further embodiment, a sirtuin activator is a compound of formula 39 and the attendant definitions wherein R₁ is 3,4,5-trimethoxyphenyl.

In a further embodiment, a sirtuin activator is a compound of formula 39 and the attendant definitions wherein L₁ is S.

In a further embodiment, a sirtuin activator is a compound of formula 39 and the attendant definitions wherein L₂ is NH.

In a further embodiment, a sirtuin activator is a compound of formula 39 and the attendant definitions wherein R is methyl and n is 1.

In a further embodiment, a sirtuin activator is a compound of formula 39 and the attendant definitions wherein R is methyl, n is 1, and R₁ is 3,4,5-trimethoxyphenyl.

In a further embodiment, a sirtuin activator is a compound of formula 39 and the attendant definitions wherein R is methyl, n is 1, R₁ is 3,4,5-trimethoxyphenyl, and L₁ is S.

In a further embodiment, a sirtuin activator is a compound of formula 39 and the attendant definitions wherein R is methyl, n is 1, R₁ is 3,4,5-trimethoxyphenyl, L₁ is S, and L₂ is NH.

In another embodiment, a sirtuin activator is a compound of formula 40: 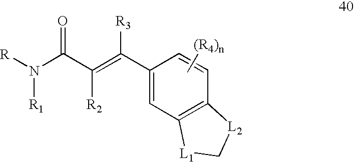
wherein, independently for each occurrence:

R, R₁, R₂, R₃ are H or a substituted or unsubstituted alkyl, aryl, alkaryl, heterocyclyl, heterocyclylalkyl, heteroaryl, or heteroaralkyl;

R₄ is hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁ and L₂ are O, NR, or S; and

n is an integer from 0 to 3 inclusive.

In a further embodiment, a sirtuin activator is a compound of formula 40 and the attendant definitions wherein R is H.

In a further embodiment, a sirtuin activator is a compound of formula 40 and the attendant definitions wherein R₁ is perfluorophenyl.

In a further embodiment, a sirtuin activator is a compound of formula 40 and the attendant definitions wherein R₂ is H.

In a further embodiment, a sirtuin activator is a compound of formula 40 and the attendant definitions wherein R₃ is H.

In a further embodiment, a sirtuin activator is a compound of formula 40 and the attendant definitions wherein L₁ is O.

In a further embodiment, a sirtuin activator is a compound of formula 40 and the attendant definitions wherein L₂ is O.

In a further embodiment, a sirtuin activator is a compound of formula 40 and the attendant definitions wherein n is O.

In a further embodiment, a sirtuin activator is a compound of formula 40 and the attendant definitions wherein R is H and R₁ is perfluorophenyl.

In a further embodiment, a sirtuin activator is a compound of formula 40 and the attendant definitions wherein R is H, R₁ is perfluorophenyl, and R₂ is H.

In a further embodiment, a sirtuin activator is a compound of formula 40 and the attendant definitions R is H, R₁ is perfluorophenyl, R₂ is H, and R₃ is H.

In a further embodiment, a sirtuin activator is a compound of formula 40 and the attendant definitions wherein R is H, R₁ is perfluorophenyl, R₂ is H, R₃ is H, and L₁ is O.

In a further embodiment, a sirtuin activator is a compound of formula 40 and the attendant definitions wherein R is H, R₁ is perfluorophenyl, R₂ is H, R₃ is H, L₁ is O, and L₂ is O.

In a further embodiment, a sirtuin activator is a compound of formula 40 and the attendant definitions wherein R is H, R₁ is perfluorophenyl, R₂ is H, R₃ is H, L₁ is O, L₂ is O, and n is 0.

In another embodiment, a sirtuin activator is a compound of formula 41: 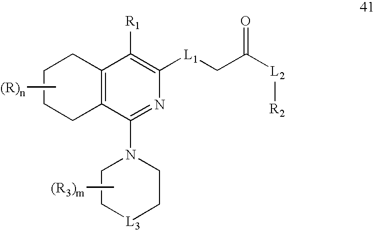
wherein, independently for each occurrence:

R, R₁, and R₃ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₂ is H or a substituted or unsubstituted alkyl, aryl, aralkyl, heterocyclyl, heterocyclylalkyl, heteroaryl, or heteroaralkyl;

L₁, L₂, and L₃ are O, NR₂, or S; and

m and n are integers from 0 to 8 inclusive.

In a further embodiment, a sirtuin activator is a compound of formula 41 and the attendant definitions wherein n is 0.

In a further embodiment, a sirtuin activator is a compound of formula 41 and the attendant definitions wherein R₁ is cyano.

In a further embodiment, a sirtuin activator is a compound of formula 41 and the attendant definitions wherein R₂ is ethyl.

In a further embodiment, a sirtuin activator is a compound of formula 41 and the attendant definitions wherein m is 0.

In a further embodiment, a sirtuin activator is a compound of formula 41 and the attendant definitions wherein L₁ is S.

In a further embodiment, a sirtuin activator is a compound of formula 41 and the attendant definitions wherein L₂ is O.

In a further embodiment, a sirtuin activator is a compound of formula 41 and the attendant definitions wherein L₃ is O.

In a further embodiment, a sirtuin activator is a compound of formula 41 and the attendant definitions wherein n is 0 and R₁ is cyano.

In a further embodiment, a sirtuin activator is a compound of formula 41 and the attendant definitions wherein n is 0, R₁ is cyano, and R₂ is ethyl.

In a further embodiment, a sirtuin activator is a compound of formula 41 and the attendant definitions wherein n is 0, R₁ is cyano, R₂ is ethyl, and m is 0.

In a further embodiment, a sirtuin activator is a compound of formula 41 and the attendant definitions wherein n is 0, R₁ is cyano, R₂ is ethyl, m is 0, and L₁ is S.

In a further embodiment, a sirtuin activator is a compound of formula 41 and the attendant definitions wherein n is 0, R₁ is cyano, R₂ is ethyl, m is 0, L₁ is S, and L₂ is O.

In a further embodiment, a sirtuin activator is a compound of formula 41 and the attendant definitions wherein n is 0, R₁ is cyano, R₂ is ethyl, m is 0, L₁ is S, L₂ is O, and L₃ is O.

In another embodiment, a sirtuin activator is a compound of formula 42: 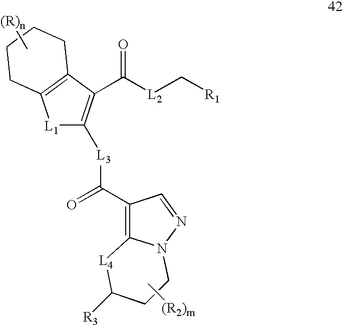
wherein, independently for each occurrence:

R and R₂ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₁ and R₃ are H or a substituted or unsubstituted alkyl, aryl, aralkyl, heterocyclyl, heterocyclylalkyl, heteroaryl, or heteroaralkyl;

L₁, L₂, L₃, and L₄ are O, NR₁, or S;

m is an integer from 0 to 6 inclusive; and

n is an integer from 0 to 8 inclusive.

In a further embodiment, a sirtuin activator is a compound of formula 42 and the attendant definitions wherein n is 0.

In a further embodiment, a sirtuin activator is a compound of formula 42 and the attendant definitions wherein R₁ is methyl.

In a further embodiment, a sirtuin activator is a compound of formula 42 and the attendant definitions wherein R₂ is CF₃ and m is 1.

In a further embodiment, a sirtuin activator is a compound of formula 42 and the attendant definitions wherein R₃ is 4-methylphenyl.

In a further embodiment, a sirtuin activator is a compound of formula 42 and the attendant definitions wherein L₁ is S.

In a further embodiment, a sirtuin activator is a compound of formula 42 and the attendant definitions wherein L₂ is 0.

In a further embodiment, a sirtuin activator is a compound of formula 42 and the attendant definitions wherein L₃ is NR₁.

In a further embodiment, a sirtuin activator is a compound of formula 42 and the attendant definitions wherein L₄ is NR₁.

In a further embodiment, a sirtuin activator is a compound of formula 42 and the attendant definitions wherein n is 0 and R₁ is methyl.

In a further embodiment, a sirtuin activator is a compound of formula 42 and the attendant definitions wherein n is 0, R₁ is methyl, R₂ is CF₃, and m is 1.

In a further embodiment, a sirtuin activator is a compound of formula 42 and the attendant definitions wherein n is 0, R₁ is methyl, R₂ is CF₃, m is 1; and R₃ is 4-methylphenyl.

In a further embodiment, a sirtuin activator is a compound of formula 42 and the attendant definitions wherein n is 0, R₁ is methyl, R₂ is CF₃, m is 1; R₃ is 4-methylphenyl; and L₁ is S.

In a further embodiment, a sirtuin activator is a compound of formula 42 and the attendant definitions wherein n is 0, R₁ is methyl, R₂ is CF₃, m is 1; R₃ is 4-methylphenyl; L₁ is S, and L₂ is O.

In a further embodiment, a sirtuin activator is a compound of formula 42 and the attendant definitions wherein n is 0, R₁ is methyl, R₂ is CF₃, m is 1; R₃ is 4-methylphenyl; L₁ is S, L₂ is O; and L₃ is NR₁.

In a further embodiment, a sirtuin activator is a compound of formula 42 and the attendant definitions wherein n is 0, R₁ is methyl, R₂ is CF₃, m is 1; R₃ is 4-methylphenyl; L₁ is S, L₂ is O; L₃ is NR₁, and L₄ is NR₁.

In another embodiment, a sirtuin activator is a compound of formula 43: 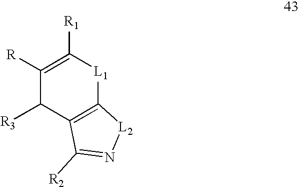
wherein, independently for each occurrence:

R and R₁ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₂ and R₃ are H or a substituted or unsubstituted alkyl, aryl, aralkyl, heterocyclyl, heterocyclylalkyl, heteroaryl, or heteroaralkyl; and

L₁ and L₂ are O, NR₂, or S.

In a further embodiment, a sirtuin activator is a compound of formula 43 and the attendant definitions wherein R is cyano.

In a further embodiment, a sirtuin activator is a compound of formula 43 and the attendant definitions wherein R₁ is NH₂.

In a further embodiment, a sirtuin activator is a compound of formula 43 and the attendant definitions wherein R₂ is 4-bromophenyl.

In a further embodiment, a sirtuin activator is a compound of formula 43 and the attendant definitions wherein R₃ is 3-hydroxy-4-methoxyphenyl.

In a further embodiment, a sirtuin activator is a compound of formula 43 and the attendant definitions wherein L₁ is O.

In a further embodiment, a sirtuin activator is a compound of formula 43 and the attendant definitions wherein L₂ is NR₂.

In a further embodiment, a sirtuin activator is a compound of formula 43 and the attendant definitions wherein R is cyano and R₁ is NH₂.

In a further embodiment, a sirtuin activator is a compound of formula 43 and the attendant definitions wherein R is cyano, R₁ is NH₂, and R₂ is 4-bromophenyl.

In a further embodiment, a sirtuin activator is a compound of formula 43 and the attendant definitions wherein R is cyano, R₁ is NH₂, R₂ is 4-bromophenyl, and R₃ is 3-hydroxy-4-methoxyphenyl.

In a further embodiment, a sirtuin activator is a compound of formula 43 and the attendant definitions wherein R is cyano, R₁ is NH₂, R₂ is 4-bromophenyl, R₃ is 3-hydroxy-4-methoxyphenyl, and L₁ is O.

In a further embodiment, a sirtuin activator is a compound of formula 43 and the attendant definitions wherein R is cyano, R₁ is NH₂, R₂ is 4-bromophenyl, R₃ is 3-hydroxy-4-methoxyphenyl, L₁ is O, and L₂ is NR₂.

In another embodiment, a sirtuin activator is a compound of formula 44: 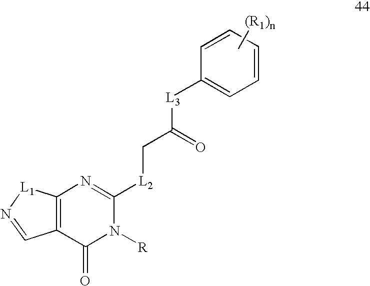
wherein, independently for each occurrence:

R is H or a substituted or unsubstituted alkyl, aryl, aralkyl, heterocyclyl, heterocyclylalkyl, heteroaryl, or heteroaralkyl;

R₁ is hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁, L₂, and L₃ are O, NR, or S; and

n is an integer from 0 to 5 inclusive.

In a further embodiment, a sirtuin activator is a compound of formula 44 and the attendant definitions wherein R is 3-trifluoromethylphenyl.

In a further embodiment, a sirtuin activator is a compound of formula 44 and the attendant definitions wherein R₁ is C(O)OCH₃.

In a further embodiment, a sirtuin activator is a compound of formula 44 and the attendant definitions wherein L₁ is NR.

In a further embodiment, a sirtuin activator is a compound of formula 44 and the attendant definitions wherein L₂ is S.

In a further embodiment, a sirtuin activator is a compound of formula 44 and the attendant definitions wherein L₃ is NR.

In a further embodiment, a sirtuin activator is a compound of formula 44 and the attendant definitions wherein n is 2.

In a further embodiment, a sirtuin activator is a compound of formula 44 and the attendant definitions wherein R is 3-trifluoromethylphenyl and R₁ is C(O)OCH₃.

In a further embodiment, a sirtuin activator is a compound of formula 44 and the attendant definitions wherein R is 3-trifluoromethylphenyl, R₁ is C(O)OCH₃, and L₁ is NR.

In a further embodiment, a sirtuin activator is a compound of formula 44 and the attendant definitions wherein R is 3-trifluoromethylphenyl, R₁ is C(O)OCH₃, L₁ is NR, and L₂ is S.

In a further embodiment, a sirtuin activator is a compound of formula 44 and the attendant definitions wherein R is 3-trifluoromethylphenyl, R₁ is C(O)OCH₃, L₁ is NR, L₂ is S, and L₃ is NR.

In a further embodiment, a sirtuin activator is a compound of formula 44 and the attendant definitions wherein R is 3-trifluoromethylphenyl, R₁ is C(O)OCH₃, L₁ is NR, L₂ is S, L₃ is NR, and n is 2.

In another embodiment, a sirtuin activator is a compound of formula 45: 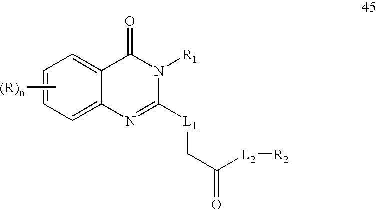
wherein, independently for each occurrence:

R is hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₁ and R₂ are H or a substituted or unsubstituted alkyl, aryl, aralkyl, heterocyclyl, heterocyclylalkyl, heteroaryl, or heteroaralkyl;

L₁ and L₂ are O, NR₁, or S; and

n is an integer from 0 to 4 inclusive.

In a further embodiment, a sirtuin activator is a compound of formula 45 and the attendant definitions wherein n is 0.

In a further embodiment, a sirtuin activator is a compound of formula 45 and the attendant definitions wherein R₁ is 2-tetrahydrofuranylmethyl.

In a further embodiment, a sirtuin activator is a compound of formula 45 and the attendant definitions wherein R₂ is —CH₂CH₂C₆H₄SO₂NH₂.

In a further embodiment, a sirtuin activator is a compound of formula 45 and the attendant definitions wherein L₁ is S.

In a further embodiment, a sirtuin activator is a compound of formula 45 and the attendant definitions wherein L₂ is NR₁.

In a further embodiment, a sirtuin activator is a compound of formula 45 and the attendant definitions wherein n is 0 and R₁ is 2-tetrahydrofuranylmethyl.

In a further embodiment, a sirtuin activator is a compound of formula 45 and the attendant definitions wherein n is 0, R₁ is 2-tetrahydrofuranylmethyl, and R₂ is —CH₂CH₂C₆H₄SO₂NH₂.

In a further embodiment, a sirtuin activator is a compound of formula 45 and the attendant definitions wherein n is 0, R₁ is 2-tetrahydrofuranylmethyl, R₂ is —CH₂CH₂C₆H₄SO₂NH₂, and L₁ is S.

In a further embodiment, a sirtuin activator is a compound of formula 45 and the attendant definitions wherein n is 0, R₁ is 2-tetrahydrofuranylmethyl, R₂ is —CH₂CH₂C₆H₄SO₂NH₂, L₁ is S₂ and L₂ is NR₁.

In another embodiment, a sirtuin activator is a compound of formula 46: 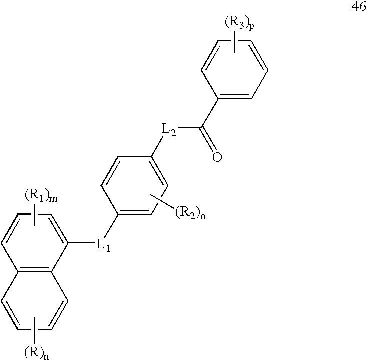
wherein, independently for each occurrence:

R, R₁, R₂, and R₃ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁ and L₂ are O, NR₄, or S;

R₄ is H or a substituted or unsubstituted alkyl, aryl, aralkyl, heterocyclyl, heterocyclylalkyl, heteroaryl, or heteroaralkyl;

n is an integer from 0 to 4 inclusive;

m is an integer from 0 to 3 inclusive;

o is an integer from 0 to 4 inclusive; and

p is an integer from 0 to 5 inclusive.

In a further embodiment, a sirtuin activator is a compound of formula 46 and the attendant definitions wherein n is 0.

In a further embodiment, a sirtuin activator is a compound of formula 46 and the attendant definitions wherein m is 1.

In a further embodiment, a sirtuin activator is a compound of formula 46 and the attendant definitions wherein R₁ is Cl.

In a further embodiment, a sirtuin activator is a compound of formula 46 and the attendant definitions wherein o is 1.

In a further embodiment, a sirtuin activator is a compound of formula 46 and the attendant definitions wherein R₂ is Cl.

In a further embodiment, a sirtuin activator is a compound of formula 46 and the attendant definitions wherein p is 3.

In a further embodiment, a sirtuin activator is a compound of formula 46 and the attendant definitions wherein R₃ is OH or 1.

In a further embodiment, a sirtuin activator is a compound of formula 46 and the attendant definitions wherein n is 0, and m is 1.

In a further embodiment, a sirtuin activator is a compound of formula 46 and the attendant definitions wherein n is 0, m is 1, and o is 1.

In a further embodiment, a sirtuin activator is a compound of formula 46 and the attendant definitions wherein n is 0, m is 1, o is 1, and R₁ is Cl.

In a further embodiment, a sirtuin activator is a compound of formula 46 and the attendant definitions wherein n is 0, m is 1, o is 1, R₁ is Cl, and p is 3.

In a further embodiment, a sirtuin activator is a compound of formula 46 and the attendant definitions wherein n is 0, m is 1, o is 1, R₁ is Cl, p is 3, and R₂ is OH or I.

In another embodiment, a sirtuin activator is a compound of formula 47: 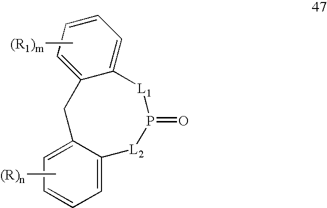
wherein, independently for each occurrence:

R and R₁ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁ and L₂ are O, NR₄, or S;

R₄ is H or a substituted or unsubstituted alkyl, aryl, aralkyl, heterocyclyl, heterocyclylalkyl, heteroaryl, or heteroaralkyl; and

m and n are integers from 0 to 4 inclusive.

In a further embodiment, a sirtuin activator is a compound of formula 47 and the attendant definitions wherein n is 2.

In a further embodiment, a sirtuin activator is a compound of formula 47 and the attendant definitions wherein R is methyl or t-butyl.

In a further embodiment, a sirtuin activator is a compound of formula 47 and the attendant definitions wherein m is 2.

In a further embodiment, a sirtuin activator is a compound of formula 47 and the attendant definitions wherein R₁ is methyl or t-butyl.

In a further embodiment, a sirtuin activator is a compound of formula 47 and the attendant definitions wherein L₁ is O.

In a further embodiment, a sirtuin activator is a compound of formula 47 and the attendant definitions wherein L₂ is O.

In a further embodiment, a sirtuin activator is a compound of formula 47 and the attendant definitions wherein n is 2 and R is methyl or t-butyl.

In a further embodiment, a sirtuin activator is a compound of formula 47 and the attendant definitions wherein n is 2, R is methyl or t-butyl, and m is 2.

In a further embodiment, a sirtuin activator is a compound of formula 47 and the attendant definitions wherein n is 2, R is methyl or t-butyl, m is 2, and R₁ is methyl or t-butyl.

In a further embodiment, a sirtuin activator is a compound of formula 47 and the attendant definitions wherein n is 2, R is methyl or t-butyl, m is 2, R₁ is methyl or t-butyl, and L₁ is O.

In a further embodiment, a sirtuin activator is a compound of formula 47 and the attendant definitions wherein n is 2, R is methyl or t-butyl, m is 2, R₁ is methyl or t-butyl, L₁ is O, and L₂ is O.

In another embodiment, a sirtuin activator is a compound of formula 48: 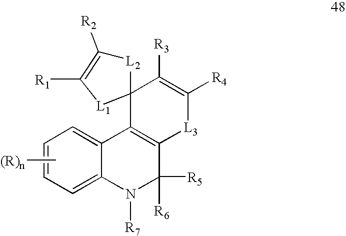
wherein, independently for each occurrence:

R, R₁, R₂, R₃, R₄, R₅, and R₆ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₇ is H or a substituted or unsubstituted alkyl, acyl, aryl, aralkyl, heterocyclyl, heterocyclylalkyl, heteroaryl, or heteroaralkyl;

L₁, L₂, and L₃ are O, NR₇, or S and n is an integer from 0 to 4 inclusive.

In a further embodiment, a sirtuin activator is a compound of formula 48 and the attendant definitions wherein n is 1.

In a further embodiment, a sirtuin activator is a compound of formula 48 and the attendant definitions wherein R is methyl.

In a further embodiment, a sirtuin activator is a compound of formula 48 and the attendant definitions wherein R₁ is C(O)OCH₃.

In a further embodiment, a sirtuin activator is a compound of formula 48 and the attendant definitions wherein R₂ is C(O)OCH₃.

In a further embodiment, a sirtuin activator is a compound of formula 48 and the attendant definitions wherein R₃ is C(O)OCH₃.

In a further embodiment, a sirtuin activator is a compound of formula 48 and the attendant definitions wherein R₄ is C(O)OCH₃.

In a further embodiment, a sirtuin activator is a compound of formula 48 and the attendant definitions wherein R₅ is methyl.

In a further embodiment, a sirtuin activator is a compound of formula 48 and the attendant definitions wherein R₆ is methyl.

In a further embodiment, a sirtuin activator is a compound of formula 48 and the attendant definitions wherein R₇ is C(O)CF₃.

In a further embodiment, a sirtuin activator is a compound of formula 48 and the attendant definitions wherein L₁ is S.

In a further embodiment, a sirtuin activator is a compound of formula 48 and the attendant definitions wherein L₂ is S.

In a further embodiment, a sirtuin activator is a compound of formula 48 and the attendant definitions wherein L₃ is S.

In a further embodiment, a sirtuin activator is a compound of formula 48 and the attendant definitions wherein n is 1 and R is methyl.

In a further embodiment, a sirtuin activator is a compound of formula 48 and the attendant definitions wherein n is 1, R is methyl, and R₁ is C(O)OCH₃.

In a further embodiment, a sirtuin activator is a compound of formula 48 and the attendant definitions wherein n is 1, R is methyl, R₁ is C(O)OCH₃, and R₂ is C(O)OCH₃.

In a further embodiment, a sirtuin activator is a compound of formula 48 and the attendant definitions wherein n is 1, R is methyl, R₁ is C(O)OCH₃, R₂ is C(O)OCH₃, and R₃ is C(O)OCH₃.

In a further embodiment, a sirtuin activator is a compound of formula 48 and the attendant definitions wherein n is 1, R is methyl, R₁ is C(O)OCH₃, R₂ is C(O)OCH₃, R₃ is C(O)OCH₃, and R₄ is C(O)OCH₃.

In a further embodiment, a sirtuin activator is a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, and R₅ is methyl.

In a further embodiment, a sirtuin activator is a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, R₅ is methyl, and R₆ is methyl.

In a further embodiment, a sirtuin activator is a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, R₅ is methyl, R₆ is methyl, and R₇ is C(O)CF₃.

In a further embodiment, a sirtuin activator is a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, R₅ is methyl, R₆ is methyl, R₇ is C(O)CF₃, and L₁ is S.

In a further embodiment, a sirtuin activator is a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, R₅ is methyl, R₆ is methyl, R₇ is C(O)CF₃, L₁ is S, and L₂ is S.

In a further embodiment, a sirtuin activator is a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, R₅ is methyl, R₆ is methyl, R₇ is C(O)CF₃, L₁ is S, L₂ is S, and L₃ is S.

In another embodiment, a sirtuin activator is a compound of formula 49: 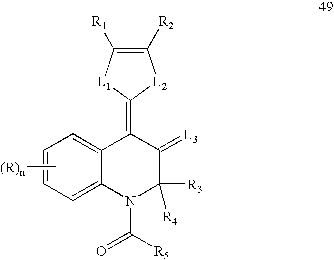
wherein, independently for each occurrence:

R, R₁, R₂, R₃, R₄, and R₅ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁, L₂, and L₃ are O, NR₆, or S;

R₆ is H or a substituted or unsubstituted alkyl, aryl, aralkyl, heterocyclyl, heterocyclylalkyl, heteroaryl, or heteroaralkyl; and

n is an integer from 0 to 4 inclusive.

In a further embodiment, a sirtuin activator is a compound of formula 49 and the attendant definitions wherein n is 1.

In a further embodiment, a sirtuin activator is a compound of formula 49 and the attendant definitions wherein R is methyl.

In a further embodiment, a sirtuin activator is a compound of formula 49 and the attendant definitions wherein R₁ is C(O)OCH₃.

In a further embodiment, a sirtuin activator is a compound of formula 49 and the attendant definitions wherein R₂ is C(O)OCH₃.

In a further embodiment, a sirtuin activator is a compound of formula 49 and the attendant definitions wherein R₃ is methyl.

In a further embodiment, a sirtuin activator is a compound of formula 49 and the attendant definitions wherein R₄ is methyl.

In a further embodiment, a sirtuin activator is a compound of formula 49 and the attendant definitions wherein R₅ is CH₂CH(CH₃)₂.

In a further embodiment, a sirtuin activator is a compound of formula 49 and the attendant definitions wherein L₁ is S.

In a further embodiment, a sirtuin activator is a compound of formula 49 and the attendant definitions wherein L₂ is S.

In a further embodiment, a sirtuin activator is a compound of formula 49 and the attendant definitions wherein L₃ is S.

In a further embodiment, a sirtuin activator is a compound of formula 49 and the attendant definitions wherein n is 1 and R is methyl.

In a further embodiment, a sirtuin activator is a compound of formula 49 and the attendant definitions wherein n is 1, R is methyl, and R₁ is C(O)OCH₃.

In a further embodiment, a sirtuin activator is a compound of formula 49 and the attendant definitions wherein n is 1, R is methyl, R₁ is C(O)OCH₃, and R₂ is C(O)OCH₃.

In a further embodiment, a sirtuin activator is a compound of formula 49 and the attendant definitions wherein n is 1, R is methyl, R₁ is C(O)OCH₃, R₂ is C(O)OCH₃, and R₃ is methyl.

In a further embodiment, a sirtuin activator is a compound of formula 49 and the attendant definitions wherein n is 1, R is methyl, R₁ is C(O)OCH₃, R₂ is C(O)OCH₃, R₃ is methyl, and R₄ is methyl.

In a further embodiment, a sirtuin activator is a compound of formula 49 and the attendant definitions wherein n is 1, R is methyl, R₁ is C(O)OCH₃, R₂ is C(O)OCH₃, R₃ is methyl, R₄ is methyl, and R₅ is CH₂CH(CH₃)₂.

In a further embodiment, a sirtuin activator is a compound of formula 49 and the attendant definitions wherein n is 1, R is methyl, R₁ is C(O)OCH₃, R₂ is C(O)OCH₃, R₃ is methyl, R₄ is methyl, R₅ is CH₂CH(CH₃)₂, and L₁ is S.

In a further embodiment, a sirtuin activator is a compound of formula 49 and the attendant definitions wherein n is 1, R is methyl, R₁ is C(O)OCH₃, R₂ is C(O)OCH₃, R₃ is methyl, R₄ is methyl, R₅ is CH₂CH(CH₃)₂, and L₁ is S.

In a further embodiment, a sirtuin activator is a compound of formula 49 and the attendant definitions wherein n is 1, R is methyl, R₁ is C(O)OCH₃, R₂ is C(O)OCH₃, R₃ is methyl, R₄ is methyl, R₅ is CH₂CH(CH₃)₂, L₁ is S, and L₂ is S.

In a further embodiment, a sirtuin activator is a compound of formula 49 and the attendant definitions wherein n is 1, R is methyl, R₁ is C(O)OCH₃, R₂ is C(O)OCH₃, R₃ is methyl, R₄ is methyl, R₅ is CH₂CH(CH₃)₂, L₁ is S, and L₂ is S.

In a further embodiment, a sirtuin activator is a compound of formula 49 and the attendant definitions wherein n is 1, R is methyl, R₁ is C(O)OCH₃, R₂ is C(O)OCH₃, R₃ is methyl, R₄ is methyl, R₅ is CH₂CH(CH₃)₂, L₁ is S, L₂ is S, and L₃ is S.

In another embodiment, a sirtuin activator is a compound of formula 50: 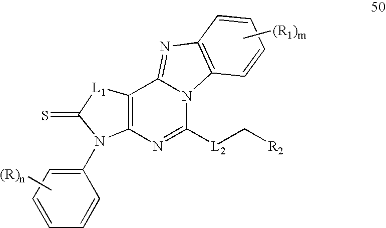
wherein, independently for each occurrence:

R and R₁ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₂ is H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁ and L₂ are O, NR₃, or S;

R₃ is H or a substituted or unsubstituted alkyl, aryl, aralkyl, heterocyclyl, heterocyclylalkyl, heteroaryl, or heteroaralkyl;

n is an integer from 0 to 5 inclusive; and

m is an integer from 0 to 4 inclusive.

In a further embodiment, a sirtuin activator is a compound of formula 50 and the attendant definitions wherein n is 1.

In a further embodiment, a sirtuin activator is a compound of formula 50 and the attendant definitions wherein R is CO₂Et.

In a further embodiment, a sirtuin activator is a compound of formula 50 and the attendant definitions wherein m is 0.

In a further embodiment, a sirtuin activator is a compound of formula 50 and the attendant definitions wherein R₂ is cyano.

In a further embodiment, a sirtuin activator is a compound of formula 50 and the attendant definitions wherein L₁ is S.

In a further embodiment, a sirtuin activator is a compound of formula 50 and the attendant definitions wherein L₂ is S.

In a further embodiment, a sirtuin activator is a compound of formula 50 and the attendant definitions wherein n is 1 and R is CO₂Et.

In a further embodiment, a sirtuin activator is a compound of formula 50 and the attendant definitions wherein n is 1, R is CO₂Et, and m is 0.

In a further embodiment, a sirtuin activator is a compound of formula 50 and the attendant definitions wherein n is 1, R is CO₂Et, m is 0, and R₂ is cyano.

In a further embodiment, a sirtuin activator is a compound of formula 50 and the attendant definitions wherein n is 1, R is CO₂Et, m is 0, R₂ is cyano, and L₁ is S.

In a further embodiment, a sirtuin activator is a compound of formula 50 and the attendant definitions wherein n is 1, R is CO₂Et, m is 0, R₂ is cyano, L₁ is S, and L₂ is S.

In another embodiment, a sirtuin activator is a compound of formula 51: 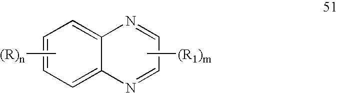
wherein, independently for each occurrence:

R and R₁ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

n is an integer from 0 to 4 inclusive; and

m is an integer from 0 to 2 inclusive.

In a further embodiment, a sirtuin activator is a compound of formula 51 and the attendant definitions wherein n is 2.

In a further embodiment, a sirtuin activator is a compound of formula 51 and the attendant definitions wherein R is Cl or trifluoromethyl.

In a further embodiment, a sirtuin activator is a compound of formula 51 and the attendant definitions wherein m is 2.

In a further embodiment, a sirtuin activator is a compound of formula 51 and the attendant definitions wherein R₁ is phenyl.

In a further embodiment, a sirtuin activator is a compound of formula 51 and the attendant definitions wherein n is 2 and R is Cl or trifluoromethyl.

In a further embodiment, a sirtuin activator is a compound of formula 51 and the attendant definitions wherein n is 2, R is Cl or trifluoromethyl, and m is 2.

In a further embodiment, a sirtuin activator is a compound of formula 51 and the attendant definitions wherein n is 2, R is Cl or trifluoromethyl, m is 2, and R₁ is phenyl.

In a further embodiment, a sirtuin activator is a compound of formula 51 and the attendant definitions wherein n is 1.

In a further embodiment, a sirtuin activator is a compound of formula 51 and the attendant definitions wherein R is F.

In a further embodiment, a sirtuin activator is a compound of formula 51 and the attendant definitions wherein R₁ is 4-methylphenyl.

In a further embodiment, a sirtuin activator is a compound of formula 51 and the attendant definitions wherein n is 1 and R is F.

In a further embodiment, a sirtuin activator is a compound of formula 51 and the attendant definitions wherein n is 1, R is F, and m is 2.

In a further embodiment, a sirtuin activator is a compound of formula 51 and the attendant definitions wherein n is 1, R is F, m is 2, and R₁ is 4-methylphenyl.

In another embodiment, a sirtuin activator is a compound of formula 52: 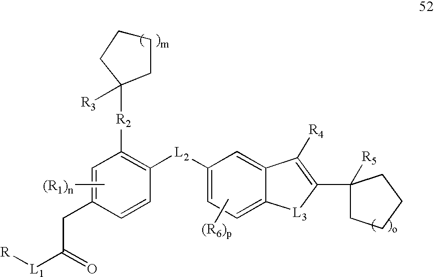
wherein, independently for each occurrence:

R is H or a substituted or unsubstituted alkyl, aryl, aralkyl, heterocyclyl, heterocyclylalkyl, heteroaryl, or heteroaralkyl;

R₁ and R₆ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₂ is alkylene, alkenylene, or alkynylene;

R₃, R₄, and R₅ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁, L₂, and L₃ are O, NR, or S;

n and p are integers from 0 to 3 inclusive; and

m and o are integers from 0 to 2 inclusive.

In a further embodiment, a sirtuin activator is a compound of formula 52 and the attendant definitions wherein R is CH₂CH₂OH.

In a further embodiment, a sirtuin activator is a compound of formula 52 and the attendant definitions wherein n is 1.

In a further embodiment, a sirtuin activator is a compound of formula 52 and the attendant definitions wherein R₁ is I.

In a further embodiment, a sirtuin activator is a compound of formula 52 and the attendant definitions wherein R₂ is alkynylene.

In a further embodiment, a sirtuin activator is a compound of formula 52 and the attendant definitions wherein m is 1.

In a further embodiment, a sirtuin activator is a compound of formula 52 and the attendant definitions wherein R₃ is OH.

In a further embodiment, a sirtuin activator is a compound of formula 52 and the attendant definitions wherein R₄ is C(O)OEt.

In a further embodiment, a sirtuin activator is a compound of formula 52 and the attendant definitions wherein o is 1.

In a further embodiment, a sirtuin activator is a compound of formula 52 and the attendant definitions wherein R₅ is OH.

In a further embodiment, a sirtuin activator is a compound of formula 52 and the attendant definitions wherein p is 0.

In a further embodiment, a sirtuin activator is a compound of formula 52 and the attendant definitions wherein L₁ is NH.

In a further embodiment, a sirtuin activator is a compound of formula 52 and the attendant definitions wherein L₂ is O.

In a further embodiment, a sirtuin activator is a compound of formula 52 and the attendant definitions wherein L₃ is O.

In a further embodiment, a sirtuin activator is a compound of formula 52 and the attendant definitions wherein R is CH₂CH₂OH and n is 1.

In a further embodiment, a sirtuin activator is a compound of formula 52 and the attendant definitions wherein R is CH₂CH₂OH, n is 1, and R₁ is I.

In a further embodiment, a sirtuin activator is a compound of formula 52 and the attendant definitions wherein R is CH₂CH₂OH, n is 1, R₁ is I, and R₂ is alkynylene.

In a further embodiment, a sirtuin activator is a compound of formula 52 and the attendant definitions wherein R is CH₂CH₂OH, n is 1, R₁ is I, R₂ is alkynylene, and m is 1.

In a further embodiment, a sirtuin activator is a compound of formula 52 and the attendant definitions wherein R is CH₂CH₂OH, n is 1, R₁ is I, R₂ is alkynylene, m is 1, and R₃ is OH.

In a further embodiment, a sirtuin activator is a compound of formula 52 and the attendant definitions wherein R is CH₂CH₂OH, n is 1, R₁ is I, R₂ is alkynylene, m is 1, R₃ is OH, and R₄ is C(O)OEt.

In a further embodiment, a sirtuin activator is a compound of formula 52 and the attendant definitions wherein R is CH₂CH₂OH, n is 1, R₁ is I, R₂ is alkynylene, m is 1, R₃ is OH, R₄ is C(O)OEt, and o is 1.

In a further embodiment, a sirtuin activator is a compound of formula 52 and the attendant definitions wherein R is CH₂CH₂OH, n is 1, R₁ is I, R₂ is alkynylene, m is 1, R₃ is OH, R₄ is C(O)OEt, o is 1, and R₅ is OH.

In a further embodiment, a sirtuin activator is a compound of formula 52 and the attendant definitions wherein R is CH₂CH₂OH, n is 1, R₁ is I, R₂ is alkynylene, m is 1, R₃ is OH, R₄ is C(O)OEt, o is 1, R₅ is OH, and p is 0.

In a further embodiment, a sirtuin activator is a compound of formula 52 and the attendant definitions wherein R is CH₂CH₂OH, n is 1, R₁ is 1, R₂ is alkynylene, m is 1, R₃ is OH, R₄ is C(O)OEt, o is 1, R₅ is OH, p is 0, and L₁ is NH.

In a further embodiment, a sirtuin activator is a compound of formula 52 and the attendant definitions wherein R is CH₂CH₂OH, n is 1, R₁ is 1, R₂ is alkynylene, m is 1, R₃ is OH, R₄ is C(O)OEt, o is 1, R₅ is OH, p is 0, L₁ is NH, and L₂ is 0.

In a further embodiment, a sirtuin activator is a compound of formula 52 and the attendant definitions wherein R is CH₂CH₂OH, n is 1, R₁ is I, R₂ is alkynylene, m is 1, R₃ is OH, R₄ is C(O)OEt, o is 1, R₅ is OH, p is 0, L₁ is NH, L₂ is O, and L₃ is O.

In another embodiment, a sirtuin activator is a compound of formula 53: 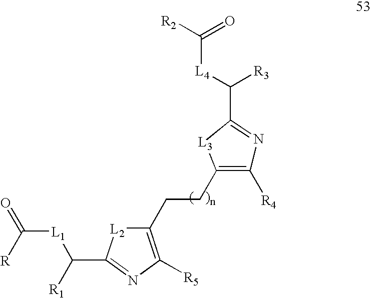
wherein, independently for each occurrence:

R, R₁, R₂, R₃, R₄, and R₅ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁, L₂, L₃, and L₄ are O, NR₆, or S;

R₆ is and H, or a substituted or unsubstituted alkyl, aryl, aralkyl, heterocyclyl, heterocyclylalkyl, heteroaryl, or heteroaralkyl; and

n is an integer from 0 to 5 inclusive.

In a further embodiment, a sirtuin activator is a compound of formula 53 and the attendant definitions wherein R is O-t-butyl.

In a further embodiment, a sirtuin activator is a compound of formula 53 and the attendant definitions wherein R₁ is t-butyl.

In a further embodiment, a sirtuin activator is a compound of formula 53 and the attendant definitions wherein R₂ is O-t-butyl.

In a further embodiment, a sirtuin activator is a compound of formula 53 and the attendant definitions wherein R₃ is t-butyl.

In a further embodiment, a sirtuin activator is a compound of formula 53 and the attendant definitions wherein R₄ is C(O)OMe.

In a further embodiment, a sirtuin activator is a compound of formula 53 and the attendant definitions wherein R₅ is C(O)OMe.

In a further embodiment, a sirtuin activator is a compound of formula 53 and the attendant definitions wherein L₁ is NH.

In a further embodiment, a sirtuin activator is a compound of formula 53 and the attendant definitions wherein L₂ is O.

In a further embodiment, a sirtuin activator is a compound of formula 53 and the attendant definitions wherein L₃ is O.

In a further embodiment, a sirtuin activator is a compound of formula 53 and the attendant definitions wherein L₄ is NH.

In a further embodiment, a sirtuin activator is a compound of formula 53 and the attendant definitions wherein n is 1.

In a further embodiment, a sirtuin activator is a compound of formula 53 and the attendant definitions wherein R is O-t-butyl and R₁ is t-butyl.

In a further embodiment, a sirtuin activator is a compound of formula 53 and the attendant definitions wherein R is O-t-butyl, R₁ is t-butyl, and R₂ is O-t-butyl.

In a further embodiment, a sirtuin activator is a compound of formula 53 and the attendant definitions wherein R is O-t-butyl, R₁ is t-butyl, R₂ is O-t-butyl, and R₃ is t-butyl.

In a further embodiment, a sirtuin activator is a compound of formula 53 and the attendant definitions wherein R is O-t-butyl, R₁ is t-butyl, R₂ is O-t-butyl, R₃ is t-butyl, and R₄ is C(O)OMe.

In a further embodiment, a sirtuin activator is a compound of formula 53 and the attendant definitions wherein R is O-t-butyl, R₁ is t-butyl, R₂ is O-t-butyl, R₃ is t-butyl, R₄ is C(O)OMe, and R₅ is C(O)OMe.

In a further embodiment, a sirtuin activator is a compound of formula 53 and the attendant definitions wherein R is O-t-butyl, R₁ is t-butyl, R₂ is O-t-butyl, R₃ is t-butyl, R₄ is C(O)OMe, R₅ is C(O)OMe, and L₁ is NH.

In a further embodiment, a sirtuin activator is a compound of formula 53 and the attendant definitions wherein R is O-t-butyl, R₁ is t-butyl, R₂ is O-t-butyl, R₃ is t-butyl, R₄ is C(O)OMe, R₅ is C(O)OMe, L₁ is NH, and L₂ is O.

In a further embodiment, a sirtuin activator is a compound of formula 53 and the attendant definitions wherein R is O-t-butyl, R₁ is t-butyl, R₂ is O-t-butyl, R₃ is t-butyl, R₄ is C(O)OMe, R₅ is C(O)OMe, L₁ is NH, L₂ is O, and L₃ is O.

In a further embodiment, a sirtuin activator is a compound of formula 53 and the attendant definitions wherein R is O-t-butyl, R₁ is t-butyl, R₂ is O-t-butyl, R₃ is t-butyl, R₄ is C(O)OMe, R₅ is C(O)OMe, L₁ is NH, L₂ is O, L₃ is O, and L₄ is NH.

In a further embodiment, a sirtuin activator is a compound of formula 53 and the attendant definitions wherein R is O-t-butyl, R₁ is t-butyl, R₂ is O-t-butyl, R₃ is t-butyl, R₄ is C(O)OMe, R₅ is C(O)OMe, L₁ is NH, L₂ is O, L₃ is O, L₄ is NH, and n is 1.

In another embodiment, a sirtuin activator is a compound of formula 54: 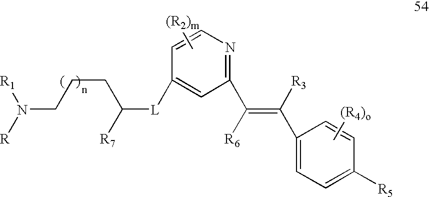
wherein, independently for each occurrence:

R and R₁ are H or a substituted or unsubstituted alkyl, aryl, aralkyl, heterocyclyl, heterocyclylalkyl, heteroaryl, or heteroaralkyl;

R₂, R₄, and R₅ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₃, R₆, and R₇ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L is O, NR, or S;

n and o are integers from 0 to 4 inclusive; and

m is an integer from 0 to 3 inclusive.

In a further embodiment, a sirtuin activator is a compound of formula 54 and the attendant definitions wherein R is ethyl.

In a further embodiment, a sirtuin activator is a compound of formula 54 and the attendant definitions wherein R₁ is ethyl.

In a further embodiment, a sirtuin activator is a compound of formula 54 and the attendant definitions wherein m is 0.

In a further embodiment, a sirtuin activator is a compound of formula 54 and the attendant definitions wherein R₃ is H.

In a further embodiment, a sirtuin activator is a compound of formula 54 and the attendant definitions wherein o is 0.

In a further embodiment, a sirtuin activator is a compound of formula 54 and the attendant definitions wherein R₅ is Cl.

In a further embodiment, a sirtuin activator is a compound of formula 54 and the attendant definitions wherein R₆ is H.

In a further embodiment, a sirtuin activator is a compound of formula 54 and the attendant definitions wherein R₇ is methyl.

In a further embodiment, a sirtuin activator is a compound of formula 54 and the attendant definitions wherein L is NH.

In a further embodiment, a sirtuin activator is a compound of formula 54 and the attendant definitions wherein n is 1.

In a further embodiment, a sirtuin activator is a compound of formula 54 and the attendant definitions wherein R is ethyl and R₁ is ethyl.

In a further embodiment, a sirtuin activator is a compound of formula 54 and the attendant definitions wherein R is ethyl, R₁ is ethyl, and m is 0.

In a further embodiment, a sirtuin activator is a compound of formula 54 and the attendant definitions wherein R is ethyl, R₁ is ethyl, m is 0, and R₃ is H.

In a further embodiment, a sirtuin activator is a compound of formula 54 and the attendant definitions wherein R is ethyl, R₁ is ethyl, m is 0, R₃ is H, and o is 0.

In a further embodiment, a sirtuin activator is a compound of formula 54 and the attendant definitions wherein R is ethyl, R₁ is ethyl, m is 0, R₃ is H, o is 0, and R₅ is Cl.

In a further embodiment, a sirtuin activator is a compound of formula 54 and the attendant definitions wherein R is ethyl, R₁ is ethyl, m is 0, R₃ is H, o is 0, R₅ is Cl, and R₆ is H.

In a further embodiment, a sirtuin activator is a compound of formula 54 and the attendant definitions wherein R is ethyl, R₁ is ethyl, m is 0, R₃ is H, o is 0, R₅ is Cl, R₆ is H, and R₇ is methyl.

In a further embodiment, a sirtuin activator is a compound of formula 54 and the attendant definitions wherein R is ethyl, R₁ is ethyl, m is 0, R₃ is H, o is 0, R₅ is Cl, R₆ is H, R₇ is methyl, and L is NH.

In a further embodiment, a sirtuin activator is a compound of formula 54 and the attendant definitions wherein R is ethyl, R₁ is ethyl, m is 0, R₃ is H, o is 0, R₅ is C₁, R₆ is H, R₇ is methyl, L is NH, and n is 1.

In another embodiment, a sirtuin activator is a compound of formula 55: 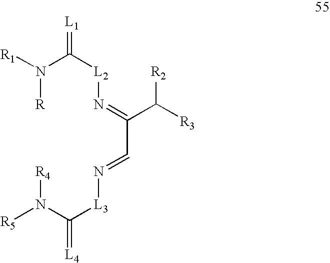
wherein, independently for each occurrence:

R, R₁, R₄, and R₅ are H or a substituted or unsubstituted alkyl, aryl, aralkyl, heterocyclyl, heterocyclylalkyl, heteroaryl, or heteroaralkyl;

R₂ and R₃ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl; and

L₁, L₂, L₃, and L₄ are O, NR, or S.

In a further embodiment, a sirtuin activator is a compound of formula 55 and the attendant definitions wherein R is H.

In a further embodiment, a sirtuin activator is a compound of formula 55 and the attendant definitions wherein R₁ is H.

In a further embodiment, a sirtuin activator is a compound of formula 55 and the attendant definitions wherein R₂ is OEt.

In a further embodiment, a sirtuin activator is a compound of formula 55 and the attendant definitions wherein R₃ is methyl.

In a further embodiment, a sirtuin activator is a compound of formula 55 and the attendant definitions wherein R₄ is H.

In a further embodiment, a sirtuin activator is a compound of formula 55 and the attendant definitions wherein R₅ is H.

In a further embodiment, a sirtuin activator is a compound of formula 55 and the attendant definitions wherein L₁ is S.

In a further embodiment, a sirtuin activator is a compound of formula 55 and the attendant definitions wherein L₂ is NH.

In a further embodiment, a sirtuin activator is a compound of formula 55 and the attendant definitions wherein L₃ is NH.

In a further embodiment, a sirtuin activator is a compound of formula 55 and the attendant definitions wherein L₄ is S.

In a further embodiment, a sirtuin activator is a compound of formula 55 and the attendant definitions wherein R is H and R₁ is H.

In a further embodiment, a sirtuin activator is a compound of formula 55 and the attendant definitions wherein R is H, R₁ is H, and R₂ is OEt.

In a further embodiment, a sirtuin activator is a compound of formula 55 and the attendant definitions wherein R is H, R₁ is H, R₂ is OEt, and R₃ is methyl.

In a further embodiment, a sirtuin activator is a compound of formula 55 and the attendant definitions wherein R is H, R₁ is H, R₂ is OEt, R₃ is methyl, and R₄ is H.

In a further embodiment, a sirtuin activator is a compound of formula 55 and the attendant definitions wherein R is H, R₁ is H, R₂ is OEt, R₃ is methyl, R₄ is H, and R₅ is H.

In a further embodiment, a sirtuin activator is a compound of formula 55 and the attendant definitions wherein R is H, R₁ is H, R₂ is OEt, R₃ is methyl, R₄ is H, R₅ is H, and L₁ is S.

In a further embodiment, a sirtuin activator is a compound of formula 55 and the attendant definitions wherein R is H, R₁ is H, R₂ is OEt, R₃ is methyl, R₄ is H, R₅ is H, L₁ is S, and L₂ is NH.

In a further embodiment, a sirtuin activator is a compound of formula 55 and the attendant definitions wherein R is H, R₁ is H, R₂ is OEt, R₃ is methyl, R₄ is H, R₅ is H, L₁ is S, L₂ is NH, and L₃ is NH.

In a further embodiment, a sirtuin activator is a compound of formula 55 and the attendant definitions wherein R is H, R₁ is H, R₂ is OEt, R₃ is methyl, R₄ is H, R₅ is H, L₁ is S, L₂ is NH, L₃ is NH, and L₄ is S.

In another embodiment, a sirtuin activator is a compound of formula 56: 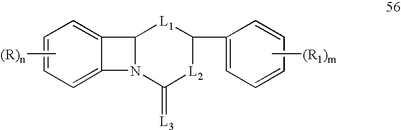
wherein, independently for each occurrence:

R and R₁ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁, L₂, and L₃ are O, NR₂, or S;

R₂ is H or a substituted or unsubstituted alkyl, aryl, aralkyl, heterocyclyl, heterocyclylalkyl, heteroaryl, or heteroaralkyl;

n is an integer from 0 to 4 inclusive; and

m is an integer from 0 to 5 inclusive.

In a further embodiment, a sirtuin activator is a compound of formula 56 and the attendant definitions wherein n is 0.

In a further embodiment, a sirtuin activator is a compound of formula 56 and the attendant definitions wherein m is 0.

In a further embodiment, a sirtuin activator is a compound of formula 56 and the attendant definitions wherein L₁ is NH.

In a further embodiment, a sirtuin activator is a compound of formula 56 and the attendant definitions wherein L₂ is S.

In a further embodiment, a sirtuin activator is a compound of formula 56 and the attendant definitions wherein L₃ is S.

In a further embodiment, a sirtuin activator is a compound of formula 56 and the attendant definitions wherein m is 0 and n is 0.

In a further embodiment, a sirtuin activator is a compound of formula 56 and the attendant definitions wherein m is 0, n is 0, and L₁ is NH.

In a further embodiment, a sirtuin activator is a compound of formula 56 and the attendant definitions wherein m is 0, n is 0, L₁ is NH, and L₂ is S.

In a further embodiment, a sirtuin activator is a compound of formula 56 and the attendant definitions wherein m is 0, n is 0, L₁ is NH, L₂ is S, and L₃ is S.

In another embodiment, a sirtuin activator is a compound of formula 57: 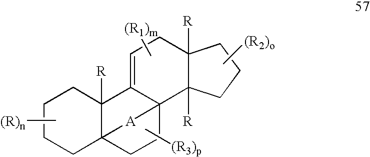
wherein, independently for each occurrence:

R, R₁, R₂, and R₃ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

A is alkylene, alkenylene, or alkynylene;

n is an integer from 0 to 8 inclusive;

m is an integer from 0 to 3 inclusive;

o is an integer from 0 to 6 inclusive; and

p is an integer from 0 to 4 inclusive.

In a further embodiment, a sirtuin activator is a compound of formula 57 and the attendant definitions wherein n is 2.

In a further embodiment, a sirtuin activator is a compound of formula 57 and the attendant definitions wherein R is OH or methyl.

In a further embodiment, a sirtuin activator is a compound of formula 57 and the attendant definitions wherein m is 1.

In a further embodiment, a sirtuin activator is a compound of formula 57 and the attendant definitions wherein R₁ is methyl.

In a further embodiment, a sirtuin activator is a compound of formula 57 and the attendant definitions wherein o is 1.

In a further embodiment, a sirtuin activator is a compound of formula 57 and the attendant definitions wherein R₂ is C(O)CH₃.

In a further embodiment, a sirtuin activator is a compound of formula 57 and the attendant definitions wherein p is 2.

In a further embodiment, a sirtuin activator is a compound of formula 57 and the attendant definitions wherein R₃ is CO₂H.

In a further embodiment, a sirtuin activator is a compound of formula 57 and the attendant definitions wherein A is alkenylene.

In a further embodiment, a sirtuin activator is a compound of formula 57 and the attendant definitions wherein n is 2 and R is OH or methyl.

In a further embodiment, a sirtuin activator is a compound of formula 57 and the attendant definitions wherein n is 2, R is OH or methyl, and m is 1.

In a further embodiment, a sirtuin activator is a compound of formula 57 and the attendant definitions wherein n is 2, R is OH or methyl, m is 1, and R₁ is methyl.

In a further embodiment, a sirtuin activator is a compound of formula 57 and the attendant definitions wherein n is 2, R is OH or methyl, m is 1, R₁ is methyl, and o is 1.

In a further embodiment, a sirtuin activator is a compound of formula 57 and the attendant definitions wherein n is 2, R is OH or methyl, m is 1, R₁ is methyl, o is 1, and R₂ is C(O)CH₃.

In a further embodiment, a sirtuin activator is a compound of formula 57 and the attendant definitions wherein n is 2, R is OH or methyl, m is 1, R₁ is methyl, o is 1, R₂ is C(O)CH₃, and p is 2.

IIn a further embodiment, a sirtuin activator is a compound of formula 57 and the attendant definitions wherein n is 2, R is OH or methyl, m is 1, R₁ is methyl, o is 1, R₂ is C(O)CH₃, p is 2, and R₃ is CO₂H.

In a further embodiment, a sirtuin activator is a compound of formula 57 and the attendant definitions wherein n is 2, R is OH or methyl, m is 1, R₁ is methyl, o is 1, R₂ is C(O)CH₃, p is 2, R₃ is CO₂H, and A is alkenylene.

In another embodiment, a sirtuin activator is a compound of formula 58: 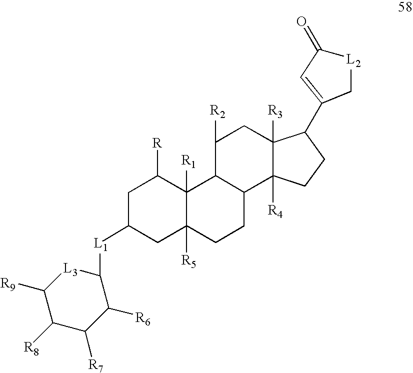
wherein, independently for each occurrence:

R, R₁, R₂, R₃, R₄, R₅, R₆, R₇, R₈, and R₉ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁, L₂, and L₃ are O, NR₁₀, or S; and

R₁₀ is H or a substituted or unsubstituted alkyl, aryl, aralkyl, heterocyclyl, heterocyclylalkyl, heteroaryl, or heteroaralkyl.

In a further embodiment, a sirtuin activator is a compound of formula 58 and the attendant definitions wherein R is OH.

In a further embodiment, a sirtuin activator is a compound of formula 58 and the attendant definitions wherein R₁ is CH₂OH.

In a further embodiment, a sirtuin activator is a compound of formula 58 and the attendant definitions wherein R₂ is OH.

In a further embodiment, a sirtuin activator is a compound of formula 58 and the attendant definitions wherein R₃ is methyl.

In a further embodiment, a sirtuin activator is a compound of formula 58 and the attendant definitions wherein R₄ is OH.

In a further embodiment, a sirtuin activator is a compound of formula 58 and the attendant definitions wherein R₅ is OH.

In a further embodiment, a sirtuin activator is a compound of formula 58 and the attendant definitions wherein R₆ is OH.

In a further embodiment, a sirtuin activator is a compound of formula 58 and the attendant definitions wherein R₇ is OH.

In a further embodiment, a sirtuin activator is a compound of formula 58 and the attendant definitions wherein R₈ is OH.

In a further embodiment, a sirtuin activator is a compound of formula 58 and the attendant definitions wherein R₉ is methyl.

In a further embodiment, a sirtuin activator is a compound of formula 58 and the attendant definitions wherein L₁ is O.

In a further embodiment, a sirtuin activator is a compound of formula 58 and the attendant definitions wherein L₂ is O.

In a further embodiment, a sirtuin activator is a compound of formula 58 and the attendant definitions wherein L₃ is O.

In a further embodiment, a sirtuin activator is a compound of formula 58 and the attendant definitions wherein R is OH and R₁ is CH₂OH.

In a further embodiment, a sirtuin activator is a compound of formula 58 and the attendant definitions wherein R is OH, R₁ is CH₂OH, and R₂ is OH.

In a further embodiment, a sirtuin activator is a compound of formula 58 and the attendant definitions wherein R is OH, R₁ is CH₂OH, R₂ is OH, and R₃ is methyl.

In a further embodiment, a sirtuin activator is a compound of formula 58 and the attendant definitions wherein R is OH, R₁ is CH₂OH, R₂ is OH, R₃ is methyl, and R₄ is OH.

In a further embodiment, a sirtuin activator is a compound of formula 58 and the attendant definitions wherein R is OH, R₁ is CH₂OH, R₂ is OH, R₃ is methyl, R₄ is OH, and R₅ is OH.

In a further embodiment, a sirtuin activator is a compound of formula 58 and the attendant definitions wherein R is OH, R₁ is CH₂OH, R₂ is OH, R₃ is methyl, R₄ is OH, R₅ is OH, and R₆ is OH.

In a further embodiment, a sirtuin activator is a compound of formula 58 and the attendant definitions wherein R is OH, R₁ is CH₂OH, R₂ is OH, R₃ is methyl, R₄ is OH, R₅ is OH, R₆ is OH, and R₇ is OH.

In a further embodiment, a sirtuin activator is a compound of formula 58 and the attendant definitions wherein R is OH, R₁ is CH₂OH, R₂ is OH, R₃ is methyl, R₄ is OH, R₅ is OH, R₆ is OH, R₇ is OH, and R₈ is OH.

In a further embodiment, a sirtuin activator is a compound of formula 58 and the attendant definitions wherein R is OH, R₁ is CH₂OH, R₂ is OH, R₃ is methyl, R₄ is OH, R₅ is OH, R₆ is OH, R₇ is OH, R₈ is OH, and R₉ is methyl.

In a further embodiment, a sirtuin activator is a compound of formula 58 and the attendant definitions wherein R is OH, R₁ is CH₂OH, R₂ is OH, R₃ is methyl, R₄ is OH, R₅ is OH, R₆ is OH, R₇ is OH, R₈ is OH, R₉ is methyl, and L₁ is O.

In a further embodiment, a sirtuin activator is a compound of formula 58 and the attendant definitions wherein R is OH, R₁ is CH₂OH, R₂ is OH, R₃ is methyl, R₄ is OH, R₅ is OH, R₆ is OH, R₇ is OH, R₈ is OH, R₉ is methyl, L₁ is O, and L₂ is O.

In a further embodiment, a sirtuin activator is a compound of formula 58 and the attendant definitions wherein R is OH, R₁ is CH₂OH, R₂ is OH, R₃ is methyl, R₄ is OH, R₅ is OH, R₆ is OH, R₇ is OH, R₈ is OH, R₉ is methyl, L₁ is O, L₂ is O, and L₃ is O.

In another embodiment, a sirtuin activator is a compound of formula 59: 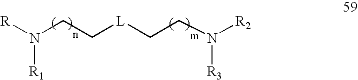
wherein, independently for each occurrence:

R, R₁, R₂, and R₃ are H or a substituted or unsubstituted alkyl, aryl, aralkyl, heterocyclyl, heterocyclylalkyl, heteroaryl, or heteroaralkyl;

L is O, NR, S, or Se; and

n and m are integers from 0 to 5 inclusive.

In a further embodiment, a sirtuin activator is a compound of formula 59 and the attendant definitions wherein R is H.

In a further embodiment, a sirtuin activator is a compound of formula 59 and the attendant definitions wherein R₁ is H.

In a further embodiment, a sirtuin activator is a compound of formula 59 and the attendant definitions wherein R₂ is H.

In a further embodiment, a sirtuin activator is a compound of formula 59 and the attendant definitions wherein R₃ is H.

In a further embodiment, a sirtuin activator is a compound of formula 59 and the attendant definitions wherein L is Se.

In a further embodiment, a sirtuin activator is a compound of formula 59 and the attendant definitions wherein n is 1.

In a further embodiment, a sirtuin activator is a compound of formula 59 and the attendant definitions wherein m is 1.

In a further embodiment, a sirtuin activator is a compound of formula 59 and the attendant definitions wherein R is H and R₁ is H.

In a further embodiment, a sirtuin activator is a compound of formula 59 and the attendant definitions wherein R is H, R₁ is H, and R₂ is H.

In a further embodiment, a sirtuin activator is a compound of formula 59 and the attendant definitions wherein R is H, R₁ is H, R₂ is H, and R₃ is H.

In a further embodiment, a sirtuin activator is a compound of formula 59 and the attendant definitions wherein R is H, R₁ is H, R₂ is H, R₃ is H, and L is Se.

In a further embodiment, a sirtuin activator is a compound of formula 59 and the attendant definitions wherein R is H, R₁ is H, R₂ is H, R₃ is H, L is Se, and n is 1.

In a further embodiment, a sirtuin activator is a compound of formula 0.59 and the attendant definitions wherein R is H, R₁ is H, R₂ is H, R₃ is H, L is Se, n is 1, and m is 1.

In another embodiment, a sirtuin activator is a compound of formula 60: 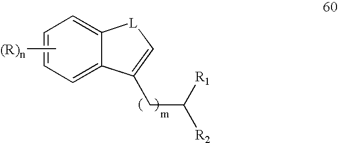
wherein, independently for each occurrence:

R is hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₁ and R₂ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L is O, NR₃, S, or SO₂;

R₃ is H or a substituted or unsubstituted alkyl, aryl, aralkyl, heterocyclyl, heterocyclylalkyl, heteroaryl, or heteroaralkyl;

n is an integer from 0 to 4 inclusive; and m is an integer from 1 to 5 inclusive.

In a further embodiment, a sirtuin activator is a compound of formula 60 and the attendant definitions wherein n is 1.

In a further embodiment, a sirtuin activator is a compound of formula 60 and the attendant definitions wherein R is Cl.

In a further embodiment, a sirtuin activator is a compound of formula 60 and the attendant definitions wherein R₁ is NH₂.

In a further embodiment, a sirtuin activator is a compound of formula 60 and the attendant definitions wherein R₂ is CO₂H.

In a further embodiment, a sirtuin activator is a compound of formula 60 and the attendant definitions wherein L is SO₂.

In a further embodiment, a sirtuin activator is a compound of formula 60 and the attendant definitions wherein m is 1.

In a further embodiment, a sirtuin activator is a compound of formula 60 and the attendant definitions wherein n is 1 and R is Cl.

In a further embodiment, a sirtuin activator is a compound of formula 60 and the attendant definitions wherein n is 1, R is Cl, and R₁ is NH₂.

In a further embodiment, a sirtuin activator is a compound of formula 60 and the attendant definitions wherein n is 1, R is Cl, R₁ is NH₂, and R₂ is CO₂H.

In a further embodiment, a sirtuin activator is a compound of formula 60 and the attendant definitions wherein n is 1, R is Cl, R₁ is NH₂, R₂ is CO₂H, and L is SO₂.

In a further embodiment, a sirtuin activator is a compound of formula 60 and the attendant definitions wherein n is 1, R is Cl, R₁ is NH₂, R₂ is CO₂H, L is SO₂, and m is 1.

In another embodiment, a sirtuin activator is a compound of formula 61: 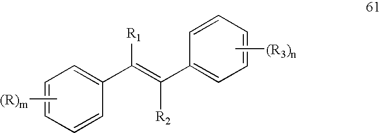
wherein, independently for each occurrence:

R, R₁, R₂, and R₃ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

n and m are integers from 0 to 5 inclusive.

In a further embodiment, a sirtuin activator is a compound of formula 61 and the attendant definitions wherein n is 2.

In a further embodiment, a sirtuin activator is a compound of formula 61 and the attendant definitions wherein R is 3-hydroxy and 5-hydroxy.

In a further embodiment, a sirtuin activator is a compound of formula 61 and the attendant definitions wherein R₁ is H.

In a further embodiment, a sirtuin activator is a compound of formula 61 and the attendant definitions wherein R₂ is H.

In a further embodiment, a sirtuin activator is a compound of formula 61 and the attendant definitions wherein m is 0.

In a further embodiment, a sirtuin activator is a compound of formula 61 and the attendant definitions wherein m is 1.

In a further embodiment, a sirtuin activator is a compound of formula 61 and the attendant definitions wherein R₃ is 4-hydroxy.

In a further embodiment, a sirtuin activator is a compound of formula 61 and the attendant definitions wherein R₃ is 4-methoxy.

In a further embodiment, a sirtuin activator is a compound of formula 61 and the attendant definitions wherein n is 2 and R is 3-hydroxy and 5-hydroxy.

In a further embodiment, a sirtuin activator is a compound of formula 61 and the attendant definitions wherein n is 2, R is 3-hydroxy and 5-hydroxy, and R₁ is H.

In a further embodiment, a sirtuin activator is a compound of formula 61 and the attendant definitions wherein n is 2, R is 3-hydroxy and 5-hydroxy, R₁ is H, and R₂ is H.

In a further embodiment, a sirtuin activator is a compound of formula 61 and the attendant definitions wherein n is 2, R is 3-hydroxy and 5-hydroxy, R₁ is H, R₂ is H, and m is 0.

In a further embodiment, a sirtuin activator is a compound of formula 61 and the attendant definitions wherein n is 2, R is 3-hydroxy and 5-hydroxy, R₁ is H, R₂ is H, and m is 1.

In a further embodiment, a sirtuin activator is a compound of formula 61 and the attendant definitions wherein n is 2, R is 3-hydroxy and 5-hydroxy, R₁ is H, R₂ is H, m is 1, and R₃ is 4-hydroxy.

In a further embodiment, a sirtuin activator is a compound of formula 61 and the attendant definitions wherein n is 2, R is 3-hydroxy and 5-hydroxy, R₁ is H, R₂ is H, m is 1, and R₃ is 4-methoxy.

In another embodiment, a sirtuin activator is a compound of formula 62: 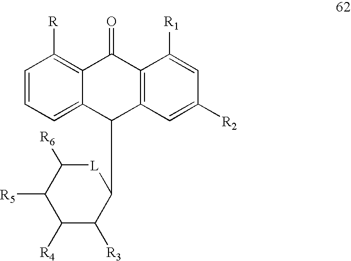
wherein, independently for each occurrence:

R, R₁, R₂, R₃, R₄, R₅, and R₆ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L is O, NR₇, or S; and

R₇ is H or a substituted or unsubstituted alkyl, aryl, aralkyl, heterocyclyl, heterocyclylalkyl, heteroaryl, or heteroaralkyl.

In a further embodiment, a sirtuin activator is a compound of formula 62 and the attendant definitions wherein R is OH.

In a further embodiment, a sirtuin activator is a compound of formula 62 and the attendant definitions wherein R₁ is OH.

In a further embodiment, a sirtuin activator is a compound of formula 62 and the attendant definitions wherein R₂ is CH₂OH.

In a further embodiment, a sirtuin activator is a compound of formula 62 and the attendant definitions wherein R₃ is OH.

In a further embodiment, a sirtuin activator is a compound of formula 62 and the attendant definitions wherein R₄ is OH.

In a further embodiment, a sirtuin activator is a compound of formula 62 and the attendant definitions wherein R₅ is OH.

In a further embodiment, a sirtuin activator is a compound of formula 62 and the attendant definitions wherein R₆ is CH₂OH.

In a further embodiment, a sirtuin activator is a compound of formula 62 and the attendant definitions wherein L is O.

In a further embodiment, a sirtuin activator is a compound of formula 62 and the attendant definitions wherein R is OH and R₁ is OH.

In a further embodiment, a sirtuin activator is a compound of formula 62 and the attendant definitions wherein R is OH, R₁ is OH, and R₂ is CH₂OH.

In a further embodiment, a sirtuin activator is a compound of formula 62 and the attendant definitions wherein R is OH, R₁ is OH, R₂ is CH₂OH, and R₃ is OH.

In a further embodiment, a sirtuin activator is a compound of formula 62 and the attendant definitions wherein R is OH, R₁ is OH, R₂ is CH₂OH, R₃ is OH, and R₄ is OH.

In a further embodiment, a sirtuin activator is a compound of formula 62 and the attendant definitions wherein R is OH, R₁ is OH, R₂ is CH₂OH, R₃ is OH, R₄ is OH, and R₅ is OH.

In a further embodiment, a sirtuin activator is a compound of formula 62 and the attendant definitions wherein R is OH, R₁ is OH, R₂ is CH₂OH, R₃ is OH, R₄ is OH, R₅ is OH, and R₆ is CH₂OH.

In a further embodiment, a sirtuin activator is a compound of formula 62 and the attendant definitions wherein R is OH, R₁ is OH, R₂ is CH₂OH, R₃ is OH, R₄ is OH, R₅ is OH, R₆ is CH₂OH, and L is O.

In another embodiment, a sirtuin activator is a compound of formula 63: 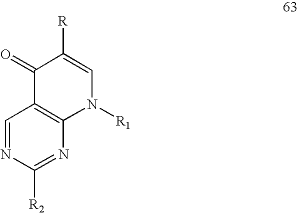
wherein, independently for each occurrence:

R, R₁₁, and R₂ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl.

In a further embodiment, a sirtuin activator is a compound of formula 63 and the attendant definitions wherein R is CO₂H.

In a further embodiment, a sirtuin activator is a compound of formula 63 and the attendant definitions wherein R₁ is ethyl.

In a further embodiment, a sirtuin activator is a compound of formula 63 and the attendant definitions wherein R₂ is N-1-pyrrolidine.

In a further embodiment, a sirtuin activator is a compound of formula 63 and the attendant definitions wherein R is CO₂H and R₁ is ethyl.

In a further embodiment, a sirtuin activator is a compound of formula 63 and the attendant definitions wherein R is CO₂H and R₂ is N-1-pyrrolidine.

In a further embodiment, a sirtuin activator is a compound of formula 63 and the attendant definitions wherein R₁ is ethyl and R₂ is N-1-pyrrolidine.

In a further embodiment, a sirtuin activator is a compound of formula 63 and the attendant definitions wherein R is CO₂H, R₁ is ethyl, and R₂ is N-1-pyrrolidine.

In another embodiment, a sirtuin activator is a compound of formula 64: 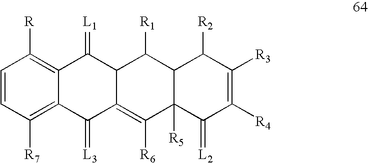
wherein, independently for each occurrence:

R, R₁, R₂, R₃, R₄, R₅, R₆, and R₇ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁, L₂, and L₃ are CH₂, O, NR₈, or S; and

R₈ is H or a substituted or unsubstituted alkyl, aryl, aralkyl, heterocyclyl, heterocyclylalkyl, heteroaryl, or heteroaralkyl.

In a further embodiment, a sirtuin activator is a compound of formula 64 and the attendant definitions wherein R is Cl.

In a further embodiment, a sirtuin activator is a compound of formula 64 and the attendant definitions wherein R is H.

In a further embodiment, a sirtuin activator is a compound of formula 64 and the attendant definitions wherein R₁ is OH.

In a further embodiment, a sirtuin activator is a compound of formula 64 and the attendant definitions wherein R₂ is N(Me)₂.

In a further embodiment, a sirtuin activator is a compound of formula 64 and the attendant definitions wherein R₃ is OH.

In a further embodiment, a sirtuin activator is a compound of formula 64 and the attendant definitions wherein R₄ is C(O)NH₂.

In a further embodiment, a sirtuin activator is a compound of formula 64 and the attendant definitions wherein R₅ is OH.

In a further embodiment, a sirtuin activator is a compound of formula 64 and the attendant definitions wherein R₆ is OH.

In a further embodiment a sirtuin activator is a compound of formula 64 and the attendant definitions wherein R₇ is OH.

In a further embodiment, a sirtuin activator is a compound of formula 64 and the attendant definitions wherein L₁ is CH₂.

In a further embodiment, a sirtuin activator is a compound of formula 64 and the attendant definitions wherein L₂ is O.

In a further embodiment, a sirtuin activator is a compound of formula 64 and the attendant definitions wherein L₃ is O.

In a further embodiment, a sirtuin activator is a compound of formula 64 and the attendant definitions wherein R is Cl and R₁ is OH.

In a further embodiment, a sirtuin activator is a compound of formula 64 and the attendant definitions wherein R is Cl, R₁ is OH, and R₂ is N(Me)₂.

In a further embodiment, a sirtuin activator is a compound of formula 64 and the attendant definitions wherein R is Cl, R₁ is OH, R₂ is N(Me)₂, and R₃ is OH.

In a further embodiment, a sirtuin activator is a compound of formula 64 and the attendant definitions wherein R is Cl, R₁ is OH, R₂ is N(Me)₂, R₃ is OH, and R₄ is C(O)NH₂.

In a further embodiment, a sirtuin activator is a compound of formula 64 and the attendant definitions wherein R is Cl, R₁ is OH, R₂ is N(Me)₂, R₃ is OH, R₄ is C(O)NH₂, and R₅ is OH.

In a further embodiment, a sirtuin activator is a compound of formula 64 and the attendant definitions wherein R is Cl, R₁ is OH, R₂ is N(Me)₂, R₃ is OH, R₄ is C(O)NH₂, R₅ is OH, and R₆ is OH.

In a further embodiment, a sirtuin activator is a compound of formula 64 and the attendant definitions wherein R is Cl, R₁ is OH, R₂ is N(Me)₂, R₃ is OH, R₄ is C(O)NH₂, R₅ is OH, R₆ is OH, and R₇ is OH.

In a further embodiment, a sirtuin activator is a compound of formula 64 and the attendant definitions wherein R is Cl, R₁ is OH, R₂ is N(Me)₂, R₃ is OH, R₄ is C(O)NH₂, R₅ is OH, R₆ is OH, R₇ is OH, and L₁ is CH₂.

In a further embodiment, a sirtuin activator is a compound of formula 64 and the attendant definitions wherein R is Cl, R₁ is OH, R₂ is N(Me)₂, R₃ is OH, R₄ is C(O)NH₂, R₅ is OH, R₆ is OH, R₇ is OH, L₁ is CH₂, and L₂ is O.

In a further embodiment, a sirtuin activator is a compound of formula 64 and the attendant definitions wherein R is Cl, R₁ is OH, R₂ is N(Me)₂, R₃ is OH, R₄ is C(O)NH₂, R₅ is OH, R₆ is OH, R₇ is OH, L₁ is CH₂, L₂ is O, and L₃ is O.

In a further embodiment, a sirtuin activator is a compound of formula 64 and the attendant definitions wherein R is H and R₁ is OH.

In a further embodiment, a sirtuin activator is a compound of formula 64 and the attendant definitions wherein R is H, R₁ is OH, and R₂ is N(Me)₂.

In a further embodiment, a sirtuin activator is a compound of formula 64 and the attendant definitions wherein R is H, R₁ is OH, R₂ is N(Me)₂, and R₃ is OH.

In a further embodiment, a sirtuin activator is a compound of formula 64 and the attendant definitions wherein R is H, R₁ is OH, R₂ is N(Me)₂, R₃ is OH, and R₄ is C(O)NH₂.

In a further embodiment, a sirtuin activator is a compound of formula 64 and the attendant definitions wherein R is H, R₁ is OH, R₂ is N(Me)₂, R₃ is OH, R₄ is C(O)NH₂, and R₅ is OH.

In a further embodiment, a sirtuin activator is a compound of formula 64 and the attendant definitions wherein R is H, R₁ is OH, R₂ is N(Me)₂, R₃ is OH, R₄ is C(O)NH₂, R₅ is OH, and R₆ is OH.

In a further embodiment, a sirtuin activator is a compound of formula 64 and the attendant definitions wherein R is H, R₁ is OH, R₂ is N(Me)₂, R₃ is OH, R₄ is C(O)NH₂, R₅ is OH, R₆ is OH, and R₇ is OH.

In a further embodiment, a sirtuin activator is a compound of formula 64 and the attendant definitions wherein R is H, R₁ is OH, R₂ is N(Me)₂, R₃ is OH, R₄ is C(O)NH₂, R₅ is OH, R₆ is OH, R₇ is OH, and L₁ is CH₂.

In a further embodiment, a sirtuin activator is a compound of formula 64 and the attendant definitions wherein R is H, R₁ is OH, R₂ is N(Me)₂, R₃ is OH, R₄ is C(O)NH₂, R₅ is OH, R₆ is OH, R₇ is OH, L₁ is CH₂, and L₂ is O.

In a further embodiment, a sirtuin activator is a compound of formula 64 and the attendant definitions wherein R is H, R₁ is OH, R₂ is N(Me)₂, R₃ is OH, R₄ is C(O)NH₂, R₅ is OH, R₆ is OH, R₇ is OH, L₁ is CH₂, L₂ is O, and L₃ is O.

In another embodiment, a sirtuin activator is a compound of formula 65: 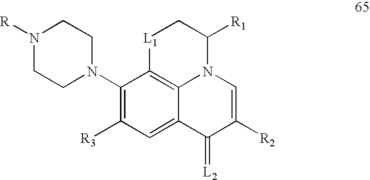
wherein, independently for each occurrence:

R is H or a substituted or unsubstituted alkyl, aryl, aralkyl, heterocyclyl, heterocyclylalkyl, heteroaryl, or heteroaralkyl;

R₁, R₂, and R₃ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl; and

L₁ and L₂ are O, NR, or S.

In a further embodiment, a sirtuin activator is a compound of formula 65 and the attendant definitions wherein R is methyl.

In a further embodiment, a sirtuin activator is a compound of formula 65 and the attendant definitions wherein R₁ is methyl.

In a further embodiment, a sirtuin activator is a compound of formula 65 and the attendant definitions wherein R₂ is CO₂H.

In a further embodiment, a sirtuin activator is a compound of formula 65 and the attendant definitions wherein R₃ is F.

In a further embodiment, a sirtuin activator is a compound of formula 65 and the attendant definitions wherein L₁ is O.

In a further embodiment, a sirtuin activator is a compound of formula 65 and the attendant definitions wherein L₂ is O.

In a further embodiment, a sirtuin activator is a compound of formula 65 and the attendant definitions wherein R is methyl and R₁ is methyl.

In a further embodiment, a sirtuin activator is a compound of formula 65 and the attendant definitions wherein R is methyl, R₁ is methyl, and R₂ is CO₂H.

In a further embodiment, a sirtuin activator is a compound of formula 65 and the attendant definitions wherein R is methyl, R₁ is methyl, R₂ is CO₂H, and R₃ is F.

In a further embodiment, a sirtuin activator is a compound of formula 65 and the attendant definitions wherein R is methyl, R₁ is methyl, R₂ is CO₂H, R₃ is F, and L₁ is O.

In a further embodiment, a sirtuin activator is a compound of formula 65 and the attendant definitions wherein R is methyl, R₁ is methyl, R₂ is CO₂H, R₃ is F, L₁ is O, and L₂ is O.

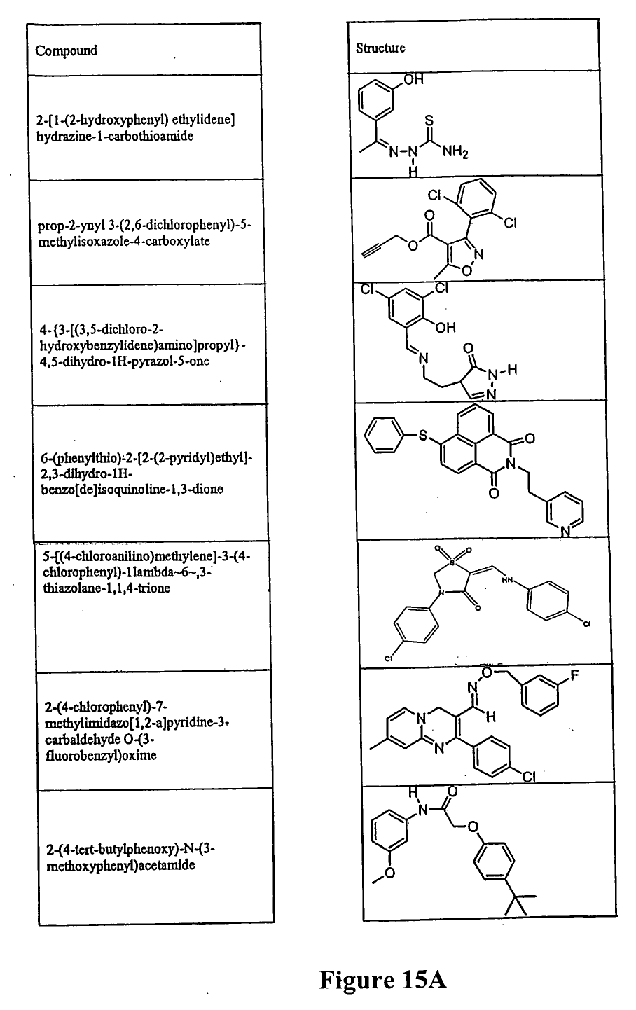

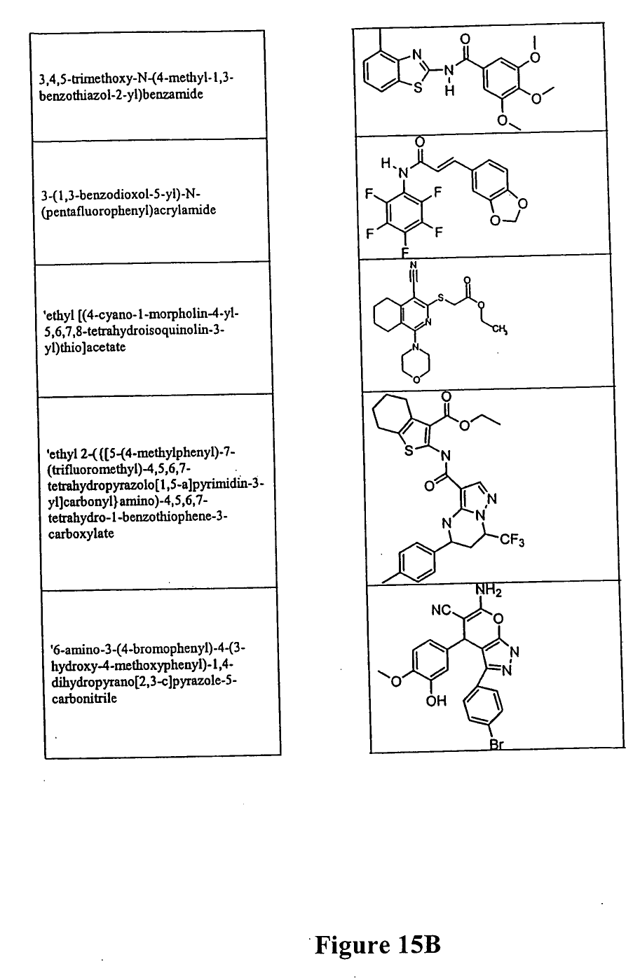

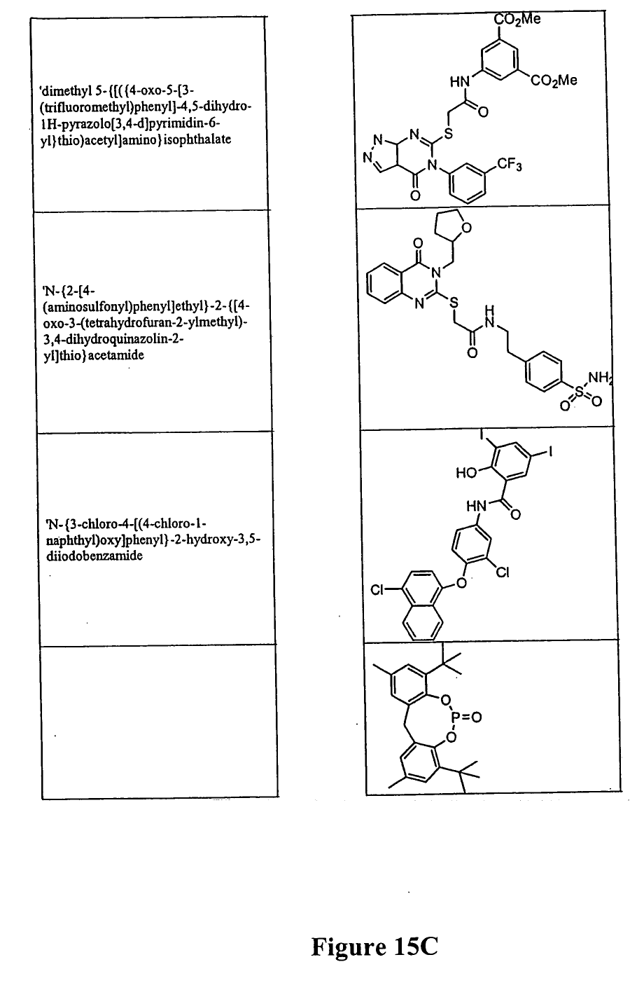

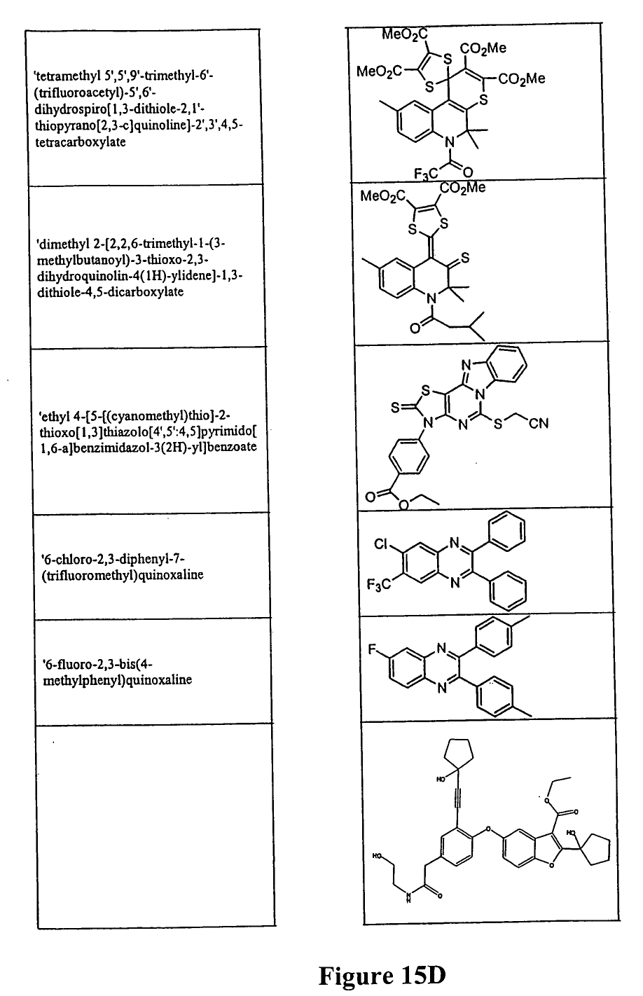

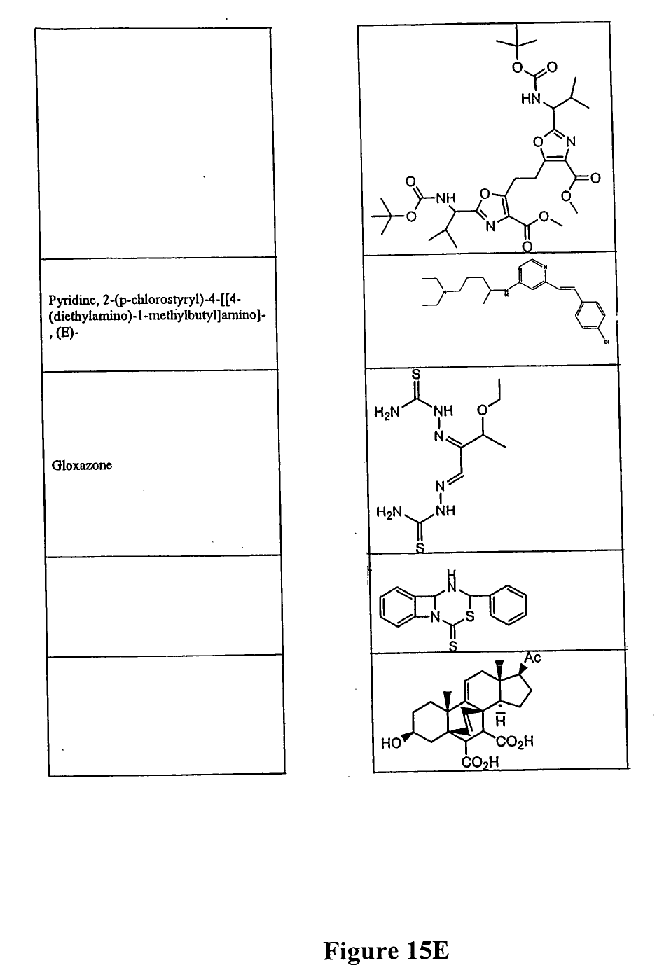

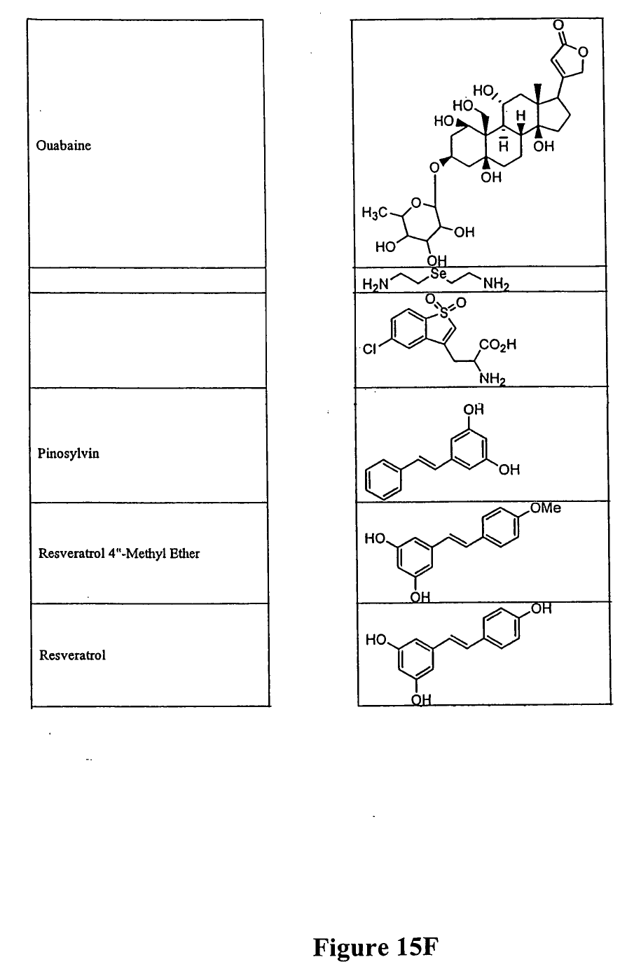

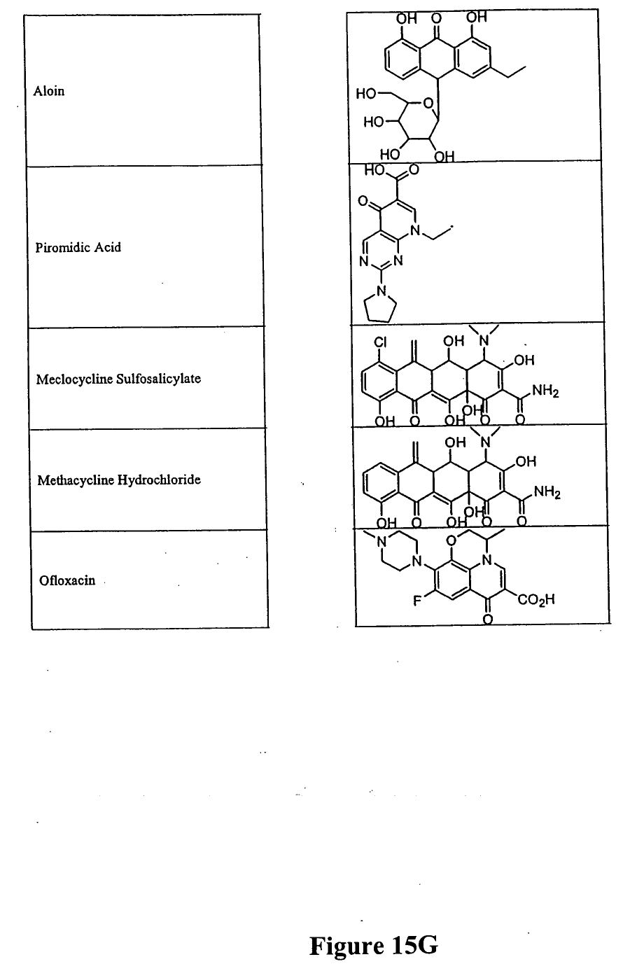

A preferred compound of formula 8 is Dipyridamole; a preferred compound of formula 12 is Hinokitiol; a preferred compound of formula 13 is L-(+)-Ergothioneine; a preferred compound of formula 19 is Caffeic Acid Phenol Ester; a preferred compound of formula 20 is MCI-186 and a preferred compound of formula 21 is HBED. Activating compounds may also be oxidized forms of the compounds of FIGS. 15A-G.

Also included are pharmaceutically acceptable addition salts and complexes of the compounds of formulas 1-25, 30, 32-65, and 69-76. In cases wherein the compounds may have one or more chiral centers, unless specified, the compounds contemplated herein may be a single stereoisomer or racemic mixtures of stereoisomers.

In one embodiment, a sirtuin activator is a stilbene, chalcone, or flavone compound represented by formula 7: 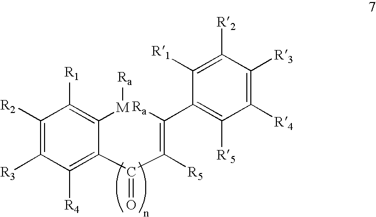
wherein, independently for each occurrence,

M is absent or O;

R₁, R₂, R₃, R′₃, R′₄, and R′₅ represent H, alkyl, aryl, heteroaryl, aralkyl, alkaryl, heteroaralkyl, halide, NO₂, SR, OR, N(R)₂, or carboxyl;

Rₐ represents H or the two instances of Rₐ form a bond;

R represents H, alkyl, or aryl; and

n is O or 1.

In a further embodiment, a sirtuin activator is a compound represented by formula 7 and the attendant definitions, wherein n is 0. In a further embodiment, a sirtuin activator is a compound represented by formula 7 and the attendant definitions, wherein n is 1. In a further embodiment, a sirtuin activator is a compound represented by formula 7 and the attendant definitions, wherein M is absent. In a further embodiment, a sirtuin activator is a compound represented by formula 7 and the attendant definitions, wherein M is O. In a further embodiment, a sirtuin activator is a compound represented by formula 7 and the attendant definitions, wherein Rₐ is H. In a further embodiment, a sirtuin activator is a compound represented by formula 7 and the attendant definitions, wherein M is O and the two Rₐ form a bond. In a further embodiment, a sirtuin activator is a compound represented by formula 7 and the attendant definitions, wherein R₅ is H. In a further embodiment, a sirtuin activator is a compound represented by formula 7 and the attendant definitions, wherein R₅ is OH.

In a further embodiment, a sirtuin activator is a compound represented by formula 7 and the attendant definitions, wherein R₁, R₃, and R′₃ are OH. In a further embodiment, a sirtuin activator is a compound represented by formula 7 and the attendant definitions, wherein R₂, R₄, R′₂, and R′₃ are OH. In a further embodiment, a sirtuin activator is a compound represented by formula 7 and the attendant definitions, wherein R₂, R′₂, and R′₃ are OH.

In a further embodiment, a sirtuin activator is a compound represented by formula 7 and the attendant definitions, wherein n is 0; M is absent; Rₐ is H; R₅ is H; R₁, R₃, and R′₃ are OH; and R₂, R₄, R′₁, R′₂, R′₄, and R′₅ are H. In a further embodiment, a sirtuin activator is a compound represented by formula 7 and the attendant definitions, wherein n is 1; M is absent; Rₐ is H; R₅ is H; R₂, R₄, R′₂, and R′₃ are OH; and R₁, R₃, R′₁, R′₄, and R′₅ are H. In a further embodiment, a sirtuin activator is a compound represented by formula 7 and the attendant definitions, wherein n is 1; M is O; the two Rₐ form a bond; R₅ is OH; R₂, R′₂, and R′₃ are OH; and R₁, R₃, R₄, R′₁, R′₄, and R′₅ are H.

In another embodiment, exemplary sirtuin activators are isonicotinamide analogs, such as, for example, the isonicotinamide analogs described in U.S. Pat. Nos. 5,985,848; 6,066,722; 6,228,847; 6,492,347; 6,803,455; and U.S. Patent Publication Nos. 2001/0019823; 2002/0061898; 2002/0132783; 2003/0149261; 2003/0229033; 2003/0096830; 2004/0053944; 2004/0110772; and 2004/0181063, the disclosures of which are hereby incorporated by reference in their entirety. In an exemplary emobidment, sirtuin activators may be an isonicotinamide analog having any of formulas 69-72 below. In one embodiment, a sirtuin activator is an isonicotinamide analog compound of formula 69:

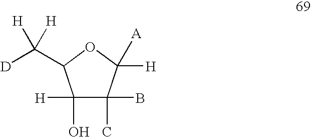

Wherein A is a nitrogen-, oxygen-, or sulfur-linked aryl, alkyl, cyclic, or heterocyclic group. The A moieties thus described, optionally have leaving group characteristics. In embodiments encompassed herein, A is further substituted with an electron contributing moiety. B and C are both hydrogen, or one of B or C is a halogen, amino, or thiol group and the other of B or C is hydrogen; and D is a primary alcohol, a hydrogen, or an oxygen, nitrogen, carbon, or sulfur linked to phosphate, a phosphoryl group, a pyrophosphoryl group, or adenosine monophosphate through a phosphodiester or carbon-, nitrogen-, or sulfur-substituted phosphodiester bridge, or to adenosine diphosphate through a phosphodiester or carbon-, nitrogen-, or sulfur-substituted pyrophosphodiester bridge.

In one example, A is a substituted N-linked aryl or heterocyclic group, an O-linked aryl or heterocyclic group having the formula —O—Y, or an S-linked aryl or heterocyclic group having the formula —O—Y; both B and C are hydrogen, or one of B or C is a halogen, amino, or thiol group and the other of B or C is hydrogen; and D is a primary alcohol or hydrogen. Nonlimiting preferred examples of A are set forth below, where each R is H or an electron-contributing moiety and Z is an alkyl, aryl, hydroxyl, OZ′ where Z′ is an alkyl or aryl, amino, NHZ′ where Z′ is an alkyl or aryl, or NHZ′Z″ where Z′ and Z″ are independently an alkyl or aryl.

Examples of A Include i-xiv Below: 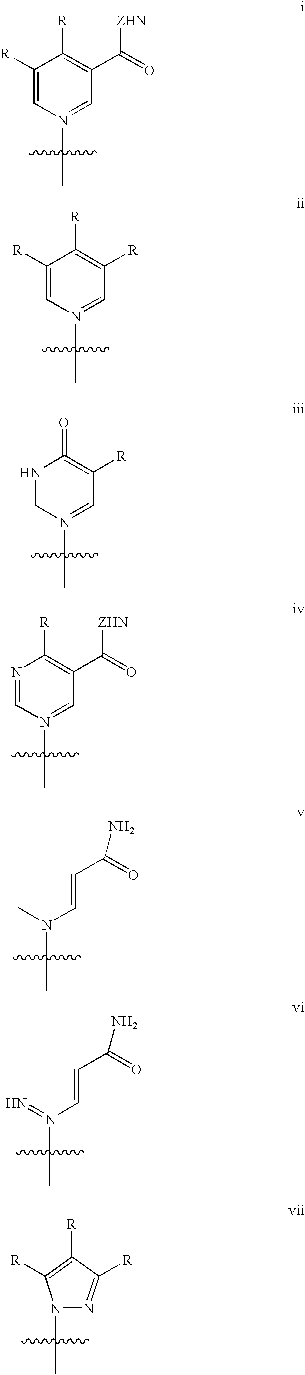 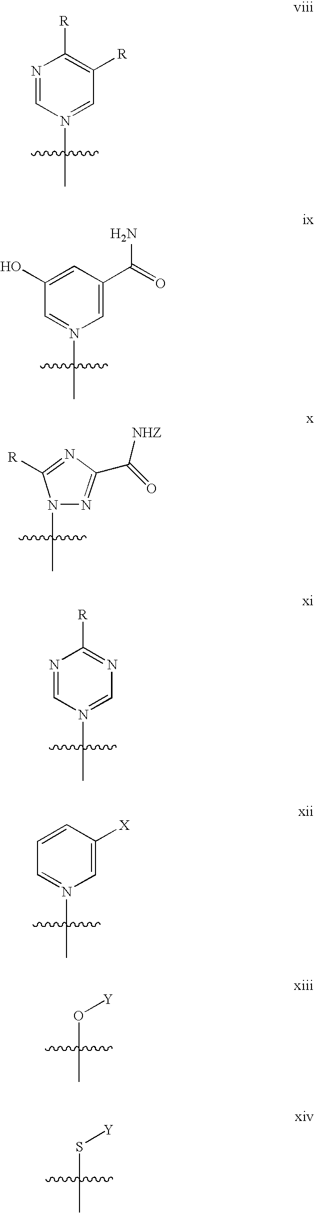
where Y is a group consistent with a leaving group function.

Examples of Y include, but are not limited to, xv-xxvii below:

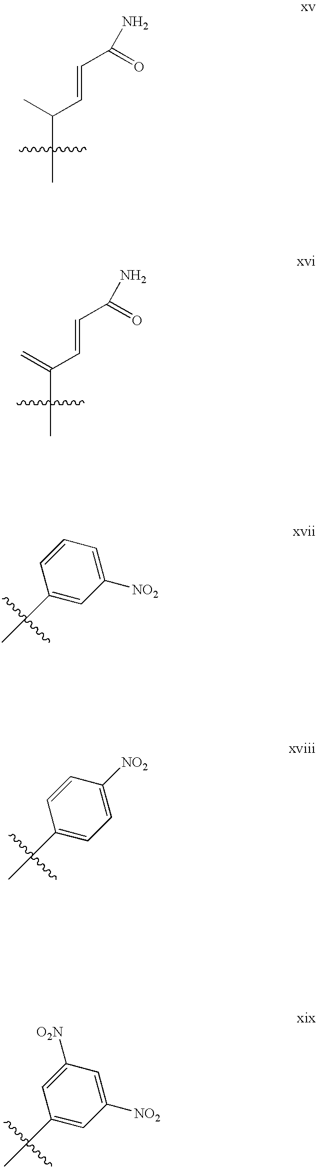

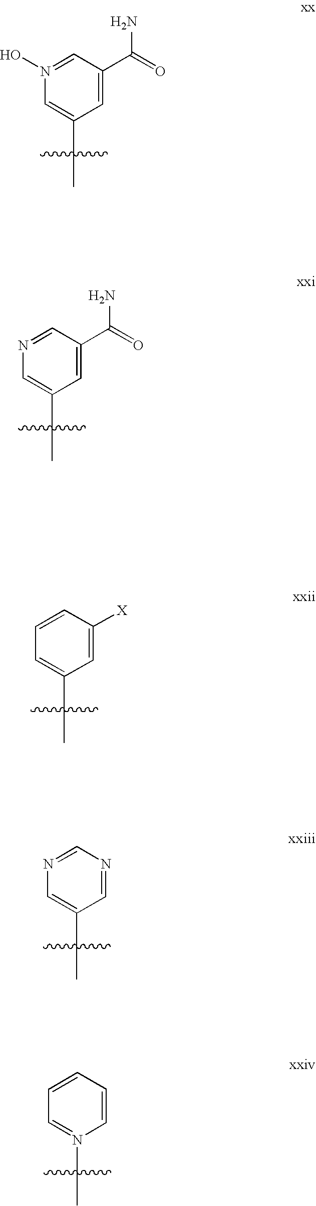

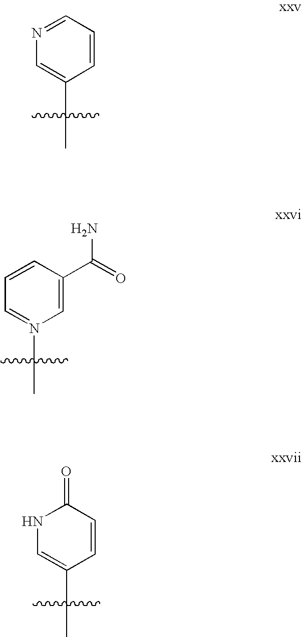

Wherein, for i-xxvii, X is halogen, thiol, or substituted thiol, amino or substituted amino, oxygen or substituted oxygen, or aryl or alkyl groups or heterocycles.

In certain embodiments, A is a substituted nicotinamide group (i above, where Z is H), a substituted pyrazolo group (vii above), or a substituted 3-carboxamid-imidazolo group (x above, where Z is H). Additionally, both B and C may be hydrogen, or one of B or C is a halogen, amino, or thiol group and the other of B or C is hydrogen; and D is a primary alcohol or hydrogen.

In other embodiments, one of B or C may be halogen, amino, or thiol group when the other of B or C is a hydrogen. Furthermore, D may be a hydrogen or an oxygen, nitrogen, carbon, or sulfur linked to phosphate, a phosphoryl group, a pyrophosphoryl group, or adenosine monophosphate through a phosphodiester or carbon-, nitrogen-, or sulfur-substituted phosphodiester bridge, or to adenosine diphosphate through a phosphodiester or carbon-, nitrogen-, or sulfur-substituted pyrophosphodiester bridge. Analogues of adenosine monophosphate or adenosine diphosphate also can replace the adenosine monophosphate or adenosine diphosphate groups.

In some embodiments, A has two or more electron contributing moieties.

In other embodiments, a sirtuin activator is an isonicotinamide analog compound of formulas 70, 71, or 72 below. 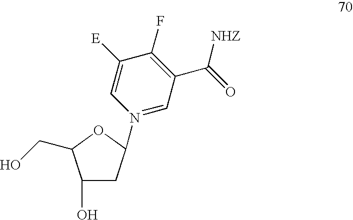
wherein Z is an alkyl, aryl, hydroxyl, OZ′ where Z′ is an alkyl or aryl, amino, NHZ′ where Z′ is an alkyl or aryl, or NHZ′Z″ where Z′ and Z″ are independently an alkyl or aryl; E and F are independently H, CH₃, OCH₃, CH₂CH₃, NH₂, OH, NHCOH, NHCOCH₃, N(CH₃)₂, C(CH₃)₂, an aryl or a C3-C10 alkyl, preferably provided that, when one of of E or F is H, the other of E or F is not H; 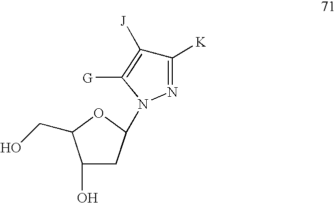
wherein G, J or K is CONHZ, Z is an alkyl, aryl, hydroxyl, OZ′ where Z′ is an alkyl or aryl, amino, NHZ′ where Z′ is an alkyl or aryl, or NHZ′Z″ where Z′ and Z″ are independently an alkyl or aryl, and the other two of G, J and K is independently CH₃, OCH₃, CH₂CH₃, NH₂, OH, NHCOH, NHCOCH₃; 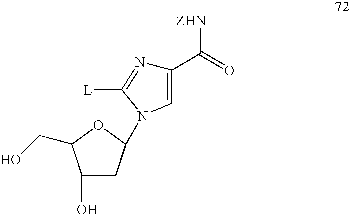
wherein Z is an alkyl, aryl, hydroxyl, OZ′ where Z′ is an alkyl or aryl, amino, NHZ′ where Z′ is an alkyl or aryl, or NHZ′Z″ where Z′ and Z″ are independently an alkyl or aryl; and L is CH₃, OCH₃, CH₂CH₃, NH₂, OH, NHCOH, NHCOCH₃.

In an exemplary embodiment, the compound is formula 70 above, wherein E and F are independently H, CH₃, OCH₃, or OH, preferably provided that, when one of E or F is H, the other of E or F is not H.

In another exemplary embodiment, the compound is β-1′-5-methyl-nicotinamide-2′-deoxyribose, β-D-1′-5-methyl-nico-tinamide-2′-deoxyribofuranoside, β-1′-4,5-dimethyl-nicotinamide-2′-de-oxyribose or β-D-1′-4,5-dimethyl-nicotinamide-2′-deoxyribofuranoside.

In yet another embodiment, the compound is β-1′-5-methyl-nicotinamide-2′-deoxyribose.

Without being bound to any particular mechanism, it is believed that the electron-contributing moiety on A stabilizes the compounds of the invention such that they are less susceptible to hydrolysis from the rest of the compound. This improved chemical stability improves the value of the compound, since it is available for action for longer periods of time in biological systems due to resistance to hydrolytic breakdown. The skilled artisan could envision many electron-contributing moieties that would be expected to serve this stabilizing function. Non-limiting examples of suitable electron contributing moieties are methyl, ethyl, O-methyl, amino, NMe₂, hydroxyl, CMe₃, aryl and alkyl groups. Preferably, the electron-contributing moiety is a methyl, ethyl, O-methyl, amino group. In the most preferred embodiments, the electron-contributing moiety is a methyl group.

The compounds of formulas 69-72 are useful both in free form and in the form of salts. The term “pharmaceutically acceptable salts” is intended to apply to non-toxic salts derived from inorganic or organic acids and includes, for example, salts derived from the following acids: hydrochloric, sulfuric, phosphoric, acetic, lactic, fumaric, succinic, tartaric, gluconic, citric, methanesulfonic, and p-toluenesulfonic acids. “Pharmaceutically acceptable salts” also include hydrates, solvates, co-crystals and polymorphs of sirtuin modulators.

Also provided are compounds of formulas 69-72 that are the tautomers, pharmaceutically-acceptable salts, esters, and pro-drugs of the inhibitor compounds disclosed herein.

The biological availability of the compounds of formulas 69-72 can be enhanced by conversion into a pro-drug form. Such a pro-drug can have improved lipophilicity relative to the unconverted compound, and this can result in enhanced membrane permeability. One particularly useful form of pro-drug is an ester derivative. Its utility relies upon the action of one or more of the ubiquitous intracellular lipases to catalyse the hydrolysis of ester groups, to release the active compound at or near its site of action. In one form of pro-drug, one or more hydroxy groups in the compound can be O-acylated, to make an acylate derivative.

Pro-drug forms of a 5-phosphate ester derivative of compounds of formulas 69-72 can also be made. These may be particularly useful, since the anionic nature of the 5-phosphate may limit its ability to cross cellular membranes. Conveniently, such a 5-phosphate derivative can be converted to an uncharged bis(acyloxymethyl)ester derivative. The utility of such a pro-drug relies upon the action of one or more of the ubiquitous intracellular lipases to catalyse the hydrolysis of ester groups, releasing a molecule of formaldehyde and a compound of the present invention at or near its site of action. Specific examples of the utility of, and general methods for making, such acyloxymethyl ester pro-drug forms of phosphorylated carbohydrate derivatives have been described (Kang et al., 1998; Jiang et al., 1998; Li et al., 1997; Kruppa et al., 1997).

In another embodiment, exemplary sirtuin activators are O-acetyl-ADP-ribose analogs, including 2′-O-acetyl-ADP-ribose and 3′-O-acetyl-ADP-ribose, and analogs thereof. Exemplary O-acetyl-ADP-ribose analogs are described, for example, in U.S. Patent Publication Nos. 2004/0053944; 2002/0061898; and 2003/0149261, the disclosures of which are hereby incorporated by reference in their entirety. In an exemplary emobidment, sirtuin activators may be an O-acetyl-ADP-ribose analog having any of formulas 73-76 below. In one embodiment, a sirtuin activator is an O-acetyl-ADP-ribose analog compound of formula 73: 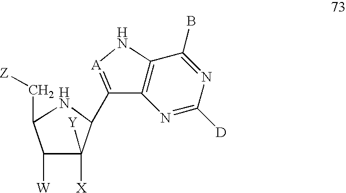
wherein:

A is selected from N, CH and CR, where R is selected from halogen, optionally substituted alkyl, aralkyl and aryl, OH, NH₂, NHR¹, NR¹R² and SR³, where R¹, R² and R³ are each optionally substituted alkyl, aralkyl or aryl groups;

B is selected from OH, NH₂, NHR⁴, H and halogen, where R⁴ is an optionally substituted alkyl, aralkyl or aryl group;

D is selected from OH, NH₂, NHR⁵, H, halogen and SCH₃, where R⁵ is an optionally substituted alkyl, aralkyl or aryl group;

X and Y are independently selected from H, OH and halogen, with the proviso that when one of X and Y is hydroxy or halogen, the other is hydrogen;

Z is OH, or, when X is hydroxy, Z is selected from hydrogen, halogen, hydroxy, SQ and OQ, where Q is an optionally substituted alkyl, aralkyl or aryl group; and

W is OH or H, with the proviso that when W is OH, then A is CR where R is as defined above;

or a tautomer thereof; or a pharmaceutically acceptable salt thereof; or an ester thereof; or a prodrug thereof.

In certain embodiments, when B is NHR⁴ and/or D is NHR⁵, then R⁴ and/or R⁵ are C1-C4 alkyl.

In other embodiments, when one or more halogens are present they are chosen from chlorine and fluorine.

In another embodiment, when Z is SQ or OQ, Q is C1-C5 alkyl or phenyl.

In an exemplary embodiment, D is H, or when D is other than H, B is OH.

In another embodiment, B is OH, D is H, OH or NH₂, X is OH or H, Y is H, most preferably with Z as OH, H, or methylthio, especially OH.

In certain embodiments W is OH, Y is H, X is OH, and A is CR where R is methyl or halogen, preferably fluorine.

In other embodiments, W is H, Y is H, X is OH and A is CH.

In other embodiments, a sirtuin activator is an O-acetyl-ADP-ribose analog compound of formula 74:

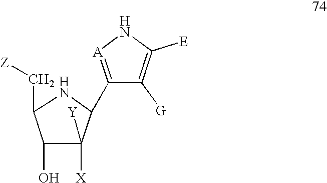

wherein A, X, Y, Z and R are defined for compounds of formula (73) where first shown above; E is chosen from CO₂H or a corresponding salt form, CO₂R, CN, CONH₂, CONHR or CONR₂; and G is chosen from NH₂, NHCOR, NHCONHR or NHCSNHR; or a tautomer thereof, or a pharmaceutically acceptable salt thereof, or an ester thereof, or a prodrug thereof.

In certain embodiments, E is CONH₂ and G is NH₂.

In other embodiments, E is CONH₂, G is NH₂, X is OH or H, is H, most preferable with Z as OH, H or methylthio, especially OH.

Exemplary sirtuin activators include the following:
(1S)-1,4-dideoxy-1-C-(4-hydroxypyrrolo[3,2-d]pyrimidin-7-yl)-1,4-imino-D-ribitol
(1S)-1-C-(2-amino-4-hydroxypyrrolo[3,2-d]pyrimidin-7-yl)-1,4-dideoxy-1,4-imino-D-ribitol
(1R)-1-C-(4-hydroxypyrrolo[3,2-d]pyrimidin-7-yl)-1,4-imino-1,2,4-trideoxy-D-erythro-pentitol
(1 S)-1-C-(4-hydroxypyrrolo[3,2-d]pyrimidin-7-yl)-1,4-imino-1,4,5-trideoxy-D-ribitol
(1S)-1,4-dideoxy-1-C-(4-hydroxypyrrolo[3,2-d]pyrimidin-7-yl)-1,4-imino-5-methylthio-D-ribitol
(1 S)-1,4-dideoxy-1-C-(2,4-dihydroxypyrrolo[3,2-d]pyrimidin-7-yl)-1,4-imino-D-ribitol
(1R)-1-C-(2,4-dihydroxypyrrolo[3,2-d]pyrimidin-7-yl)-1,4-imino-1,2,4-trideoxy-D-erthro-pentitol
(1S)-1-C-(2,4-dihydroxypyrrolo[3,2-d]pyrimidin-7-yl)-1,4-imino-1,4,5-trideoxy-D-ribitol
(1S)-1,4-dideoxy-1-C-(2,4-dihydroxypyrrolo[3,2-d]pyrimidin-7-yl)-1,4-imino-5-ethylthio-D-ribitol
(1R)-1-C-(2-amino-4-hydroxypyrrolo[3,2-d]pyrimidin-7-yl)-1,4-imino-1,2,4-trideoxy-D-erythro-pentitol
(1S)-1-C-(2-amino-4-hydroxypyrrolo[3,2-d]pyrimidin-7-yl)-1,4-imino-1,4,5-trideoxy-D-ribitol
(1S)-1-C-(2-amino-4-hydroxypyrrolo[3,2-d]pyrimidin-7-yl)-1,4-dideoxy-1,4-imino-5-methylthio-D-ribitol
(1S)-1,4-dideoxy-1-C-(7-hydroxypyrazolo[4,3-d]pyrimidin-3-yl)-1,4-imino-D-ribitol
(1R)-1-C-(7-hydroxypyrazolo[4,3-d]pyrimidin-3-yl)-1,4-imino-1,2,4-trideoxy-D-erythro-pentitol
(1S)-1-C-(7-hydroxypyrazolo[4,3-d]pyrimidin-3-yl)-1,4-imino-1,4,5-trideoxy-D-ribitol
(1S)-1,4-dideoxy-1-C-(7-hydroxypyrazolo[4,3-d]pyrimidin-3-yl)-1,4-imino-5-ethylthio-D-ribitol
(1S)-1,4-dideoxy-1-C-(5,7-dihydroxypyrazolo[4,3-d]pyrimidin-3-yl)-1,4-imino-D-ribitol
(1R)-1-C-(5,7-dihydroxypyrazolo[4,3-d]pyrimidin-3-yl)-1,4-imino-1,2,4-trideoxy-D-erythro-pentitol
(1S)-1-C-(5,7-dihydroxypyrazolo[4,3-d]pyrimidin-3-yl)-1,4-imino-1,4,5-trideoxy-D-ribitol
(1S)-1,4-dideoxy-1-C-(5,7-dihydroxypyrazolo[4,3-d]pyrimidin-3-yl)-1,4-imino-5-methylthio-D-ribitol
(1 S)-1-C-(5-amino-7-hydroxypyrazolo[4,3-d]pyrimidin-3-yl)-1,4-dideoxy-1,4-imino-D-ribitol
(1R)-1-C-(S-amino-7-hydroxypyrazolo[4,3-d]pyrimidin-3-yl)-1,4-imino-1,2,4-trideoxy-D-erythro-pentitol
(1 S)-1-C-(5-amino-7-hydroxypyrazolo[4,3-d]pyrimidin-3-yl)-1,4-imino-1,4,5-trideoxy-D-ribitol
(1S)-1-C-(5-amino-7-hydroxypyrazolo[4,3-d]pyrimidin-3-yl)-1,4-dideoxy-1,4-imino-5-methylthio-D-ribitol
(1 S)-1-C-(3-amino-2-carboxamido-4-pyrrolyl)-1,4-dideoxy-1,4-imino-D-ribitol.
(1S)-1,4-dideoxy-1-C-(4-hydroxypyrrolo[3,2-d]pyrimidin-7-yl)-1,4-imino-D-ribitol 5-phosphate
(1S)-1-C-(2-amino-4-hydroxypyrrolo[3,2-d]pyrimidin-7-yl)-1,4-imino-D-ribitol 5-phosphate
(1S)-1-C-(3-amino-2-carboxamido-4-pyrrolyl)-1,4-dideoxy-1,4-imino-D-ribitol

In yet other embodiments, sirtuin activators are O-acetyl-ADP-ribose analog compounds of formula 75 and 76, their tautomers and pharmaceutically acceptable salts.

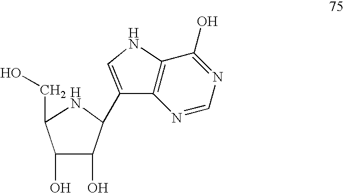

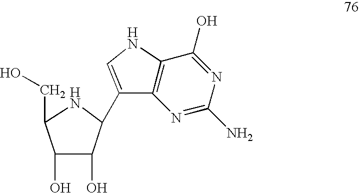

The biological availability of a compound of formula (73) or formula (74) can be enhanced by conversion into a pro-drug form. Such a pro-drug can have improved lipophilicity relative to the compound of formula (73) or formula (74), and this can result in enhanced membrane permeability. One particularly useful form of a pro-drug is an ester derivative. Its utility relies upon the action of one or more of the ubiquitous intracellular lipases to catalyse the hydrolysis of these ester group(s), to release the compound of formula (73) and formula (74) at or near its site of action.

In one form of a prodrug, one or more of the hydroxy groups in a compound of formula (73) or formula (74) can be O-acylated, to make, for example a 5-O-butyrate or a 2,3-di-O-butyrate derivative.

Prodrug forms of 5-phosphate ester derivative of a compounds of formula (73) or formula (74) can also be made and may be particularly useful, since the anionic nature of the 5-phosphate may limit its ability to cross cellular membranes. Conveniently, such a 5-phosphate derivative can be converted to an uncharged bis(acyloxymethyl)ester derivative. The utility of such a pro-drug relies upon the action of one or more of the ubiquitous intracellular lipases to catalyse the hydrolysis of these ester group(s), releasing a molecule of formaldehyde and the compound of formula (73) or formula (74) at or near its site of action.

In an exemplary embodiment, analogs of 2′-AADPR or 3′-AADPR that are designed to have increased stability from esterase action through the use of well-known substitutes for ester oxygen atoms that are subject to esterase attack. The esterase-labile oxygen atoms in 2′-AADPR and 3′-AADPR would be understood to be the ester oxygen linking the acetate group with the ribose, and the ester oxygen between the two phosphorus atoms. As is known in the art, substitution of either or both of these ester oxygen atoms with a CF₂, a NH, or a S would be expected to provide a 2′-AADPR or 3′-AADPR analog that is substantially more stable due to increased resistance to esterase action.

Thus, in some embodiments, the invention is directed to analogs 2′-O-acetyl-ADP-ribose or 3′-O-acetyl-ADP-ribose exhibiting increased stability in cells. The preferred analogs comprise a CF₂, a NH, or a S instead of the acetyl ester oxygen or the oxygen between two phosphorus atoms. The most preferred substitute is CF₂. Replacement of the acetyl ester oxygen is particularly preferred. In other preferred embodiments, both the ester oxygen and the oxygen between the two phosphorus atoms are independently substituted with a CF₂, a NH, or a S.

In another embodiment, the present invention relates to sirtuin-inhibitory compounds. Exemplary sirtuin inhibitory compounds include compounds that inhibit the activity of a class III histone deacetylase, such as, for example, nicotinamide (NAM), suranim; NF023 (a G-protein antagonist); NF279 (a purinergic receptor antagonist); Trolox (6-hydroxy-2,5,7,8,tetramethylchroman-2-carboxylic acid); (−)-epigallocatechin (hydroxy on sites 3,5,7,3′,4′, 5′); (−)-epigallocatechin gallate (Hydroxy sites 5,7,3′,4′,5′ and gallate ester on 3); cyanidin choloride (3,5,7,3′,4′-pentahydroxyflavylium chloride); delphinidin chloride (3,5,7,3′,4′,5′-hexahydroxyflavylium chloride); myricetin (cannabiscetin; 3,5,7,3′,4′,5′-hexahydroxyflavone); 3,7,3′,4′,5′-pentahydroxyflavone; and gossypetin (3,5,7,8,3′,4′-hexahydroxyflavone), all of which are further described in Howitz et al. (2003) Nature 425:191. Other inhibitors, such as sirtinol and splitomicin, are described in Grozinger et al. (2001) J. Biol. Chem. 276:38837, Dedalov et al. (2001) PNAS 98:15113 and Hirao et al. (2003) J. Biol. Chem 278: 52773. Analogs and derivatives of these compounds can also be used.

A sirtuin inhibitory compound may have a formula selected from the group of formulas 26-29, 31, and 66-68: 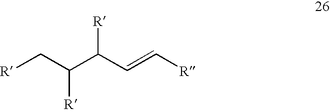
wherein, independently for each occurrence,

R′ represents H, halogen, NO₂, SR, OR, NR₂, alkyl, aryl, aralkyl, or carboxy;

R represents H, alkyl, aryl, aralkyl, or heteroaralkyl; and

R″ represents alkyl, alkenyl, or alkynyl;

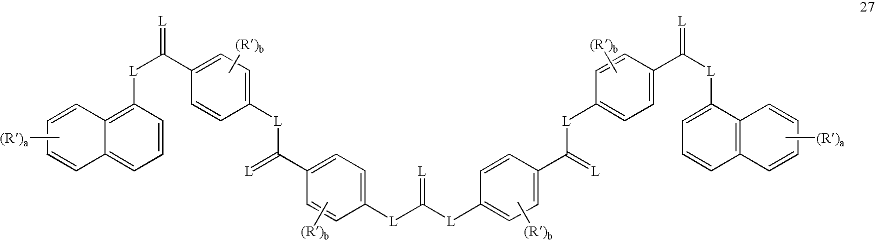

wherein, independently for each occurrence,

L represents O, NR, or S;

R represents H, alkyl, aryl, aralkyl, or heteroaralkyl;

R′ represents H, halogen, NO₂, SR, SO₃, OR, NR₂, alkyl, aryl, aralkyl, or carboxy;

a represents an integer from 1 to 7 inclusive; and

b represents an integer from 1 to 4 inclusive;

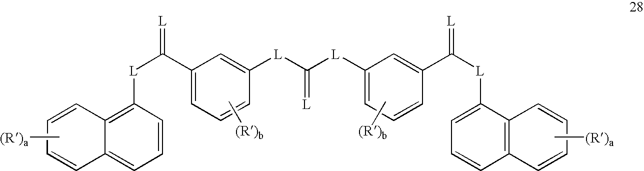

wherein, independently for each occurrence, L represents O, NR, or S;

R represents H, alkyl, aryl, aralkyl, or heteroaralkyl;

R′ represents H, halogen, NO₂, SR, SO₃, OR, NR₂, alkyl, aryl, or carboxy;

a represents an integer from 1 to 7 inclusive; and

b represents an integer from 1 to 4 inclusive;

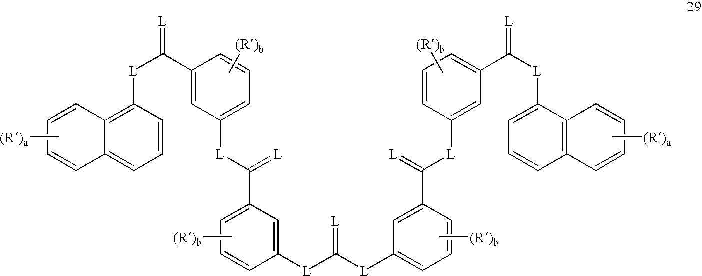

wherein, independently for each occurrence,

L represents O, NR, or S;

R represents H, alkyl, aryl, aralkyl, or heteroaralkyl;

R′ represents H, halogen, NO₂, SR, SO₃, OR, NR₂, alkyl, aryl, aralkyl, or carboxy;

a represents an integer from 1 to 7 inclusive; and

b represents an integer from 1 to 4 inclusive;

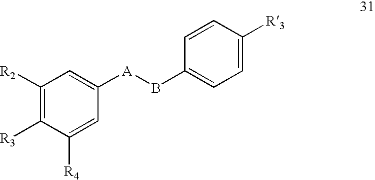

wherein, independently for each occurrence,

R₂, R₃, and R₄ are H, OH, or O-alkyl;

R′₃ is H or NO₂; and

A-B is an ethenylene or amido group.

In a further embodiment, the inhibiting compound is represented by formula 31 and the attendant definitions, wherein R₃ is OH, A-B is ethenylene, and R′₃ is H.

In a further embodiment, the inhibiting compound is represented by formula 31 and the attendant definitions, wherein R₂ and R₄ are OH, A-B is an amido group, and R′₃ is H.

In a further embodiment, the inhibiting compound is represented by formula 31 and the attendant definitions, wherein R₂ and R₄ are OMe, A-B is ethenylene, and R′₃ is NO₂.

In a further embodiment, the inhibiting compound is represented by formula 31 and the attendant definitions, wherein R₃ is OMe, A-B is ethenylene, and R′₃ is H.

In another embodiment, a sirtuin inhibitor is a compound of formula 66:

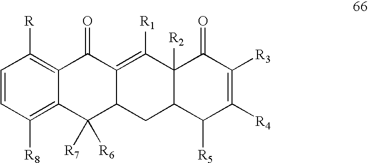

wherein, independently for each occurrence:

R, R₁, R₂, R₃, R₄, R₅, R₆, R₇, and R₈ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl.

In a further embodiment, a sirtuin inhibitor is a compound of formula 66 and the attendant definitions wherein R is OH.

In a further embodiment, a sirtuin inhibitor is a compound of formula 66 and the attendant definitions wherein R₁ is OH.

In a further embodiment, a sirtuin inhibitor is a compound of formula 66 and the attendant definitions wherein R₂ is OH.

In a further embodiment, a sirtuin inhibitor is a compound of formula 66 and the attendant definitions wherein R₃ is C(O)NH₂.

In a further embodiment, a sirtuin inhibitor is a compound of formula 66 and the attendant definitions wherein R₄ is OH.

In a further embodiment, a sirtuin inhibitor is a compound of formula 66 and the attendant definitions wherein R₅ is NMe₂.

In a further embodiment, a sirtuin inhibitor is a compound of formula 66 and the attendant definitions wherein R₆ is methyl.

In a further embodiment, a sirtuin inhibitor is a compound of formula 66 and the attendant definitions wherein R₇ is OH.

In a further embodiment, a sirtuin inhibitor is a compound of formula 66 and the attendant definitions wherein R₈ is Cl.

In a further embodiment, a sirtuin inhibitor is a compound of formula 66 and the attendant definitions wherein R is OH and R₁ is OH.

In a further embodiment, a sirtuin inhibitor is a compound of formula 66 and the attendant definitions wherein R is OH, R₁ is OH, and R₂ is OH.

In a further embodiment, a sirtuin inhibitor is a compound of formula 66 and the attendant definitions wherein R is OH, R₁ is OH, R₂ is OH, and R₃ is C(O)NH₂.

In a further embodiment, a sirtuin inhibitor is a compound of formula 66 and the attendant definitions wherein R is OH, R₁ is OH, R₂ is OH, R₃ is C(O)NH₂, and R₄ is OH.

In a further embodiment, a sirtuin inhibitor is a compound of formula 66 and the attendant definitions wherein R is OH, R₁ is OH, R₂ is OH, R₃ is C(O)NH₂, R₄ is OH, and R₅ is NMe₂.

In a further embodiment, a sirtuin inhibitor is a compound of formula 66 and the attendant definitions wherein R is OH, R₁ is OH, R₂ is OH, R₃ is C(O)NH₂, R₄ is OH, R₅ is NMe₂, and R₆ is methyl.

In a further embodiment, a sirtuin inhibitor is a compound of formula 66 and the attendant definitions wherein R is OH, R₁ is OH, R₂ is OH, R₃ is C(O)NH₂, R₄ is OH, R₅ is NMe₂, R₆ is methyl, and R₇ is OH.

In a further embodiment, a sirtuin inhibitor is a compound of formula 66 and the attendant definitions wherein R is OH, R₁ is OH, R₂ is OH, R₃ is C(O)NH₂, R₄ is OH, R₅ is NMe₂, R₆ is methyl, R₇ is OH, and R₈ is Cl.

In another embodiment, a sirtuin inhibitor is a compound of formula 67: 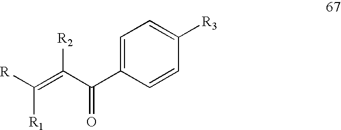
wherein, independently for each occurrence:

R, R₁, R₂, and R₃ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl.

In a further embodiment, a sirtuin inhibitor is a compound of formula 67 and the attendant definitions wherein R is Cl.

In a further embodiment, a sirtuin inhibitor is a compound of formula 67 and the attendant definitions wherein R₁ is H.

In a further embodiment, a sirtuin inhibitor is a compound of formula 67 and the attendant definitions wherein R₂ is H.

In a further embodiment, a sirtuin inhibitor is a compound of formula 67 and the attendant definitions wherein R₃ is Br.

In a further embodiment, a sirtuin inhibitor is a compound of formula 67 and the attendant definitions wherein R is Cl and R₁ is H.

In a further embodiment, a sirtuin inhibitor is a compound of formula 67 and the attendant definitions wherein R is Cl, R₁ is H, and R₂ is H.

In a further embodiment, a sirtuin inhibitor is a compound of formula 67 and the attendant definitions wherein R is Cl, R₁ is H, R₂ is H, and R₃ is Br.

In another embodiment, a sirtuin inhibitor is a compound of formula 68: 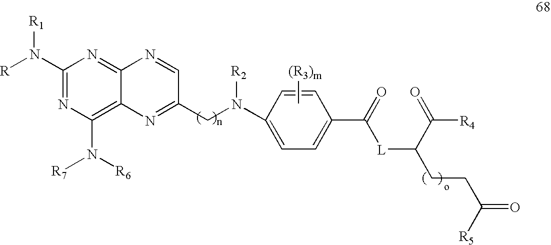
wherein, independently for each occurrence:

R, R₁, R₂, R₆, and R₇ are H or a substituted or unsubstituted alkyl, aryl, aralkyl, heterocyclyl, heterocyclylalkyl, heteroaryl, or heteroaralkyl;

R₃, R₄, and R₅ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L is O, NR, or S;

m is an integer from 0 to 4 inclusive; and

n and o are integers from 0 to 6 inclusive.

In a further embodiment, a sirtuin inhibitor is a compound of formula 68 and the attendant definitions wherein R is H.

In a further embodiment, a sirtuin inhibitor is a compound of formula 68 and the attendant definitions wherein R₁ is H.

In a further embodiment, a sirtuin inhibitor is a compound of formula 68 and the attendant definitions wherein R₂ is methyl.

In a further embodiment, a sirtuin inhibitor is a compound of formula 68 and the attendant definitions wherein m is 0.

In a further embodiment, a sirtuin inhibitor is a compound of formula 68 and the attendant definitions wherein R₄ is OH.

In a further embodiment, a sirtuin inhibitor is a compound of formula 68 and the attendant definitions wherein R₅ is OH.

In a further embodiment, a sirtuin inhibitor is a compound of formula 68 and the attendant definitions wherein R₆ is H.

In a further embodiment, a sirtuin inhibitor is a compound of formula 68 and the attendant definitions wherein R₇ is H.

In a further embodiment, a sirtuin inhibitor is a compound of formula 68 and the attendant definitions wherein L is NH.

In a further embodiment, a sirtuin inhibitor is a compound of formula 68 and the attendant definitions wherein n is 1.

In a further embodiment, a sirtuin inhibitor is a compound of formula 68 and the attendant definitions wherein o is 1.

In a further embodiment, a sirtuin inhibitor is a compound of formula 68 and the attendant definitions wherein R is H and R₁ is H.

In a further embodiment, a sirtuin inhibitor is a compound of formula 68 and the attendant definitions wherein R is H, R₁ is H, and R₂ is methyl.

In a further embodiment, a sirtuin inhibitor is a compound of formula 68 and the attendant definitions wherein R is H, R₁ is H, R₂ is methyl, and m is 0.

In a further embodiment, a sirtuin inhibitor is a compound of formula 68 and the attendant definitions wherein R is H, R₁ is H, R₂ is methyl, m is 0, and R₄ is OH.

In a further embodiment, a sirtuin inhibitor is a compound of formula 68 and the attendant definitions wherein R is H, R₁ is H, R₂ is methyl, m is 0, R₄ is OH, and R₅ is OH.

In a further embodiment, a sirtuin inhibitor is a compound of formula 68 and the attendant definitions wherein R is H, R₁ is H, R₂ is methyl, m is 0, R₄ is OH, R₅ is OH, and R₆ is H.

In a further embodiment, a sirtuin inhibitor is a compound of formula 68 and the attendant definitions wherein R is H, R₁ is H, R₂ is methyl, m is 0, R₄ is OH, R₅ is OH, R₆ is H, and R₇ is H.

In a further embodiment, a sirtuin inhibitor is a compound of formula 68 and the attendant definitions wherein R is H, R₁ is H, R₂ is methyl, m is 0, R₄ is OH, R₅ is OH, R₆ is H, R₇ is H, and L is NH.

In a further embodiment, a sirtuin inhibitor is a compound of formula 68 and the attendant definitions wherein R is H, R₁ is H, R₂ is methyl, m is 0, R₄ is OH, R₅ is OH, R₆ is H, R₇ is H, L is NH, and n is 1.

In a further embodiment, a sirtuin inhibitor is a compound of formula 68 and the attendant definitions wherein R is H, R₁ is H, R₂ is methyl, m is 0, R₄ is OH, R₅ is OH, R₆ is H, R₇ is H, L is NH, n is 1, and o is 1.

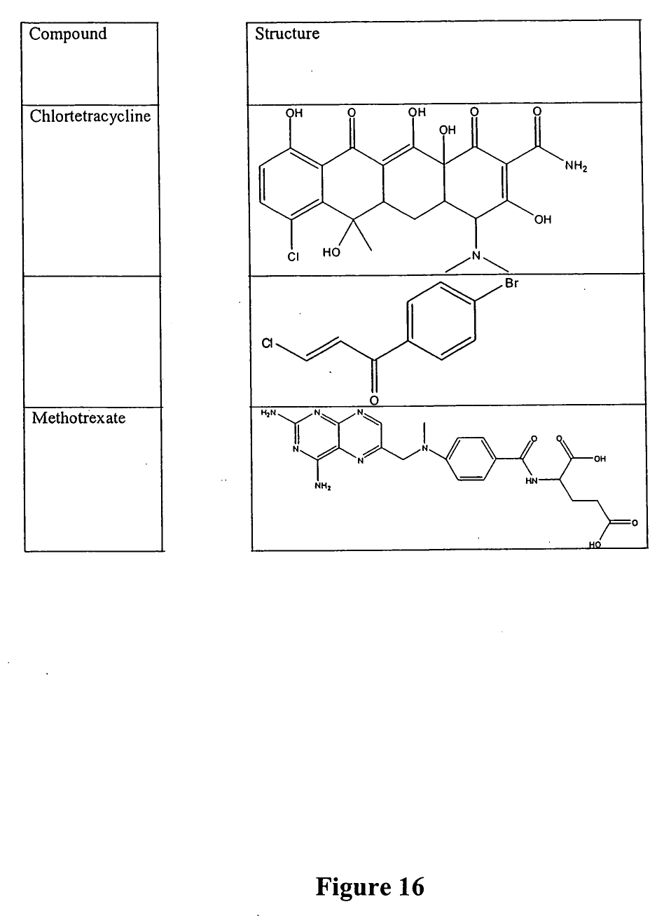

Inhibitory compounds may also be oxidized forms of compounds of FIG. 16. An oxidized form of chlortetracyclin may be an activator.

In one embodiment, sirtuin modulators for use in the invention are represented by Formula 77 or 78: 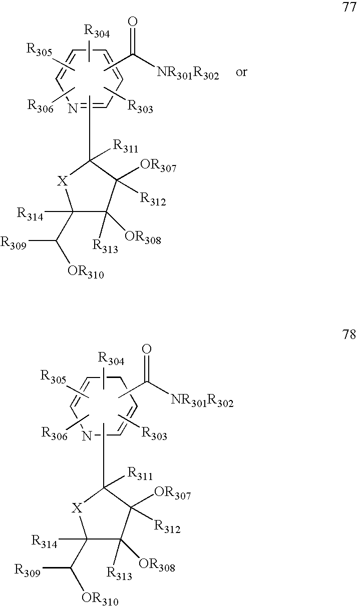
or a pharmaceutically acceptable salt thereof, where:

R₃₀₁ and R₃₀₂ are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₃₀₁ and R₃₀₂ taken together with the atom to which they are attached form a substituted or unsubstituted non-aromatic heterocyclic group;

R₃₀₃, R₃₀₄, R₃₀₅ and R₃₀₆ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′, —NO₂ and —NRC(O)R′;

R₃₀₇, R₃₀₈ and R₃₁₀ are independently selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR;

R₃₀₉ is selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′ and —NRC(O)R′;

R₃₁₁, R₃₁₂, R₃₁₃ and R₃₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)_{N}NRR′, —NRR′, —NRC(O)OR′, —NO₂ and —NRC(O)R′;

R and R′ are independently —H, a substituted or unsubstituted alkyl group, a substituted or unsubstituted aryl group or a substituted or unsubstituted non-aromatic heterocyclic group;

X is O or S; and

n is 1 or 2.

A group of suitable compounds encompassed by Formulas 77 and 78 is represented by Structural Formulas 79 and 80:

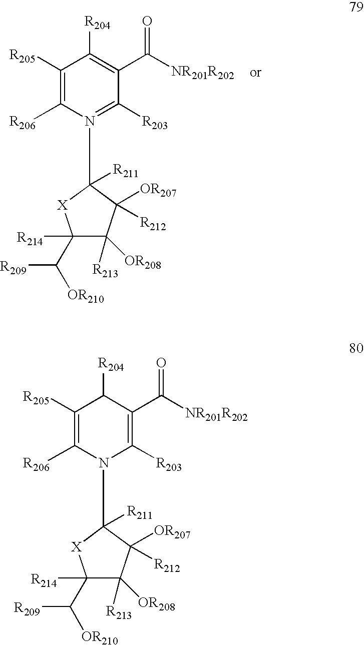

or a pharmaceutically acceptable salt thereof, where:

R₂₀₁ and R₂₀₂ are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₂₀₁ and R₂₀₂ taken together with the atom to which they are attached form a substituted or unsubstituted non-aromatic heterocyclic group;

R₂₀₃, R₂₀₄, R₂₀₅ and R₂₀₆ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′, —NO₂ and —NRC(O)R′;

R₂₀₇, R₂₀₈ and R₂₁₀ are independently selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR;

R₂₀₉ is selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′ and —NRC(O)R′;

R₂₁₁, R₂₁₂, R₂₁₃ and R₂₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)ₙNRR′, —NRR′, —NRC(O)OR′, —NO₂ and —NRC(O)R′;

R and R′ are independently —H, a substituted or unsubstituted alkyl group, a substituted or unsubstituted aryl group or a substituted or unsubstituted non-aromatic heterocyclic group;

X is O or S, preferably O; and

n is 1 or 2.

In a particular group of compounds represented by Formula 79 or 80, at least one of R₂₀₇, R₂₀₈ and R₂₁₀ is a substituted or unsubstituted alkyl group, a substituted or unsubstituted aryl group, —C(O)R, —C(O)OR, —C(O)NHR, —C(S)R, —C(S)OR or —C(O)SR. Typically, at least one of R₂₀₇, R₂₀₈ and R₂₀ is —C(O)R or —C(O)OR. More typically, at least one of R₂₀₇, R₂₀₈ and R₂₁₀ is —C(O)R. In such compounds, R is preferably a substituted or unsubstituted alkyl, particularly an unsubstituted alkyl group such as methyl or ethyl.

In another particular group of compounds represented by Formula 79 or 80, R₂₀₄ is a halogen (e.g., fluorine, bromine, chlorine) or hydrogen (including a deuterium and/or tritium isotope). Suitable compounds include those where at least one of R₂₀₇, R₂₀₈ and R₂₁₀ is a substituted or unsubstituted alkyl group, a substituted or unsubstituted aryl group, —C(O)R, —C(O)OR, —C(O)NHR, —C(S)R, —C(S)OR or —C(O)SR and R₂₀₄ is a halogen or hydrogen.

Typically, for compounds represented by Formulas 79 and 80, R₂₀₃-R₂₀₆ are —H. In addition, R₂₀₉ and R₂₁₁-R₂₉₄ are typically —H. Particular compounds represented by Formulas 79 and 80 are selected such that R₂₀₃-R₂₀₆, R₂₀₉ and R₂₁₁-R₂₁₄ are all —H. For these compounds, R₂₀₄, R₂₀₇, R₂₀₉ and R₂₁₀ have the values described above. In an exemplary embodiment, R₂₀₁-R₂₁₄ are each —H.

R₂₀, and R₂₀₂ are typically —H or a substituted or unsubstituted alkyl group, more typically —H. In compounds having these values of R₂₀, and R₂₀₂, R₂₀₃-R₂₀₆, R₂₀₉ and R₂₁₁-R₂₉₄ typically have the values described above.

In certain methods of the invention, at least one of R₂₀₁-R₂₉₄ is not —H when X is O.

In certain methods of the invention, R₂₀₆ is not —H or —NH₂ when R₂₀₁-R₂₀₅ and R₂₀₇-R₂₁₄ are each —H.

In one embodiment, a sirtuin modulator is represented by Formula 81 or 82:

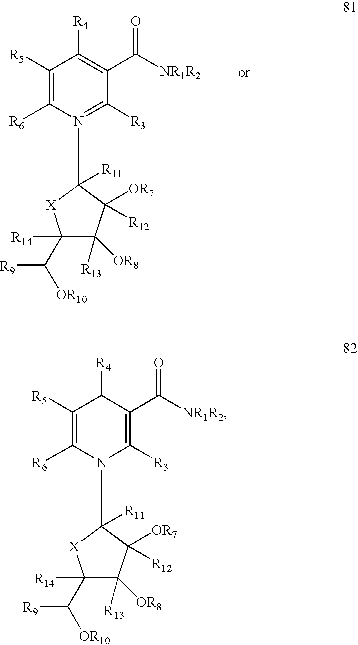

or a pharmaceutically acceptable salt thereof, wherein:

R₁ and R₂ are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₁ and R₂ taken together with the atom to which they are attached form a substituted or unsubstituted non-aromatic heterocyclic group, provided that when one of R₁ and R₂ is —H, the other is not an alkyl group substituted by —C(O)OCH₂CH₃;

R₃, R₄ and R₅ are independently selected from the group consisting of—H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′, —NO₂ and —NRC(O)R′;

R₆ is selected from the group consisting of—H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRC(O)OR′, —NO₂ and —NRC(O)R′;

R₇, R₈ and R₁₀ are independently selected from the group consisting of—H, a substituted or unsubstituted alkyl group, a substituted or unsubstituted aryl group, —C(O)R, —C(O)OR, —C(O)NHR, —C(S)R, —C(S)OR and —C(O)SR;

R₉ selected from the group consisting of—H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′ and —NRC(O)R′;

R₁, R₁₂, R₁₃ and R₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)ₙNRR′, —NRR′, —NRC(O)OR′, —NO₂ and —NRC(O)R′;

R and R′ are independently —H, a substituted or unsubstituted alkyl group, a substituted or unsubstituted aryl group or a substituted or unsubstituted non-aromatic heterocyclic group;

X is O or S, preferably O ; and

n is 1 or 2,

provided that R₁-R₁₄ are not each —H and that R₁-R₉ and R₁₁-R₁₄ are not each —H when R₁₀ is —C(O)C₆H₅.

In certain embodiments, R₁ is —H.

In certain embodiments, R₇, R₈ and R₁₀ are independently —H, —C(O)R or —C(O)OR, typically —H or —C(O)R such as —H or —C(O)CH₃. In particular embodiments, R₁ is —H and R₇, R₈ and R₁₀ are independently —H, —C(O)R or —C(O)OR.

In certain embodiments, R₉ is —H. In particular embodiments, R₉ is —H when R₁ is —H and/or R₇, R₈ and R₁₀ are independently —H, —C(O)R or —C(O)OR.

In certain embodiments, R₂ is —H. In particular embodiments, R₂ is —H when R₉ is —H, R₁ is —H and/or R₇, R₈ and R₁₀ are independently —H, —C(O)R or —C(O)OR.

Typically, R₂ is —H when R₉ is —H, R₁ is —H and R₇, R₈ and R₁₀ are independently —H, —C(O)R or —C(O)OR.

In certain embodiments, R₄ is —H or a halogen, such as deuterium or fluorine.

In one embodiment, a sirtuin modulator is represented by Formula 83 or 84:

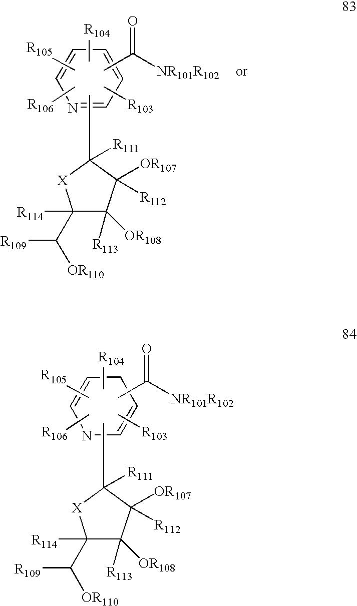

or a pharmaceutically acceptable salt thereof, wherein:

R₁₀₇, and R₁₀₂ are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₁₀₇, and R₁₀₂ taken together with the atom to which they are attached form a substituted or unsubstituted non-aromatic heterocyclic group;

R₁₀₃, R₁₀₄, R₁₀₅ and R₁₀₆ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′, —NO₂ and —NRC(O)R′; R₁₀₇ and R₁₀₈ are selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR, wherein at least one of R₁₀₇ and R₁₀₈ is a substituted or unsubstituted alkyl group, a substituted or unsubstituted aryl group, —C(O)R, —C(O)OR, —C(O)NHR, —C(S)R, —C(S)OR or —C(O)SR;

R₁₀₉ is selected from the group consisting of—H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′ and —NRC(O)R′;

R₁₁₀ is selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR, provided that R₁₁₀ is not —C(O)C₆H₅;

R₁₁₁, R₁₁₂, R₁₁₃ and R₁₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)ₙNRR′, —NRR′, —NRC(O)OR′, —NO₂ and —NRC(O)R′;

R and R′ are independently —H, a substituted or unsubstituted alkyl group, a substituted or unsubstituted aryl group or a substituted or unsubstituted non-aromatic heterocyclic group;

X is O or S; and

n is 1 or 2.

In another embodiment, a sirtuin modulator is represented by Formula 85 or 86:

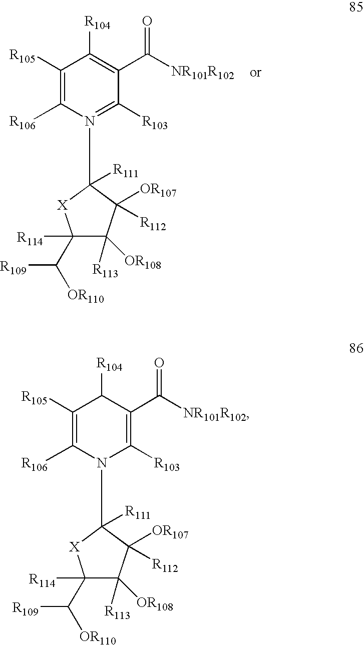

or a pharmaceutically acceptable salt thereof, where:

R₁₀₁, and R₁₀₂ are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₁₀₁, and R₁₀₂ taken together with the atom to which they are attached form a substituted or unsubstituted non-aromatic heterocyclic group;

R₁₀₃, R₁₀₄, R₁₀₅ and R₁₀₆ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′, —NO₂ and —NRC(O)R′;

R₁₀₇ and R₁₀₈ are selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR, wherein at least one of R₁₀₇ and R₁₀₈ is a substituted or unsubstituted alkyl group, a substituted or unsubstituted aryl group, —C(O)R, —C(O)OR, —C(O)NHR, —C(S)R, —C(S)OR or —C(O)SR;

R₁₀₉ is selected from the group consisting of—H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′ and —NRC(O)R′;

R₁₁₀ is selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR, provided that R₁₁₀ is not —C(O)C₆H₅;

R₁₁₁, R₁₁₂, R₁₁₃ and R₁₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)ₙNRR′, —NRR′, —NRC(O)OR′, —NO₂ and —NRC(O)R′;

R and R′ are independently —H, a substituted or unsubstituted alkyl group, a substituted or unsubstituted aryl group or a substituted or unsubstituted non-aromatic heterocyclic group;

X is O or S; and

n is 1 or 2.

For compounds represented by Formulas 83-86, typically at least one of R₁₀₇ and R₁₀₈ is —C(O)R, such as —C(O)CH₃. In particular embodiments, R₁₀₇, R₁₀₈ and R₁₁₀ are independently —H or —C(O)R (e.g., —C(O)CH₃).

In certain embodiments, such as when R₁₀₇, R₁₀₈ and R₁₁₀ have the values described above, R₁₀₁ and R₁₀₂ are each —H.

In certain embodiments, R₁₀₉ is —H.

In certain embodiments, R₁₀₃-R₁₀₆ are each —H.

In certain embodiments, R₁₁₁-R₁₁₄ are each —H.

In particular embodiments, R₁₀₇, R₁₀₈ and R₁₁₀ have the values described above and R₁₀₁-R₁₀₆, R₁₀₉ and R₁₁₁-R₁₁₄ are each —H.

In certain embodiments, R₁₀₄ is —H or a halogen, typically deuterium or fluorine. The remaining values are as described above.

For sirtuin modulators represented by Formula 87 or 88:

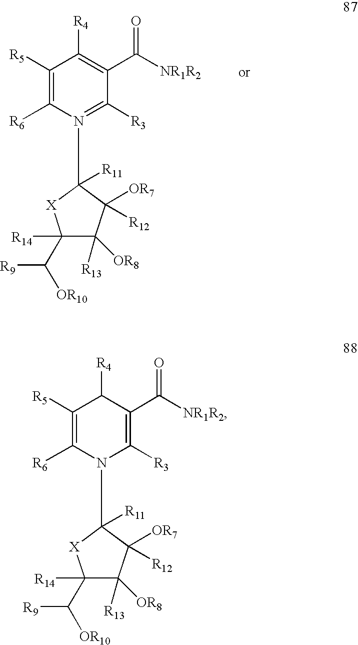

R₄ in certain embodiments is —H (e.g., deuterium, tritium) or a halogen (e.g., fluorine, bromine, chlorine).

In embodiments of the invention where R₁-R₆ can each be —H, they typically are each —H. In embodiments of the invention where one of R₁-R₆ is not —H, typically the remaining values are each —H and the non —H value is a substituted or unsubstituted alkyl group or a halogen (R₁ and R₂ are typically a substituted or unsubstituted alkyl group).

In certain embodiments, R₁₁-R₁₄ are each —H. When R₁₁-R₁₄ are each —H, R₁-R₆ typically have the values described above.

In certain embodiments, R₉ is —H. When R₉ is —H, typically R₁₁-R₁₄ are each —H and R₁-R₆ have the values described above.

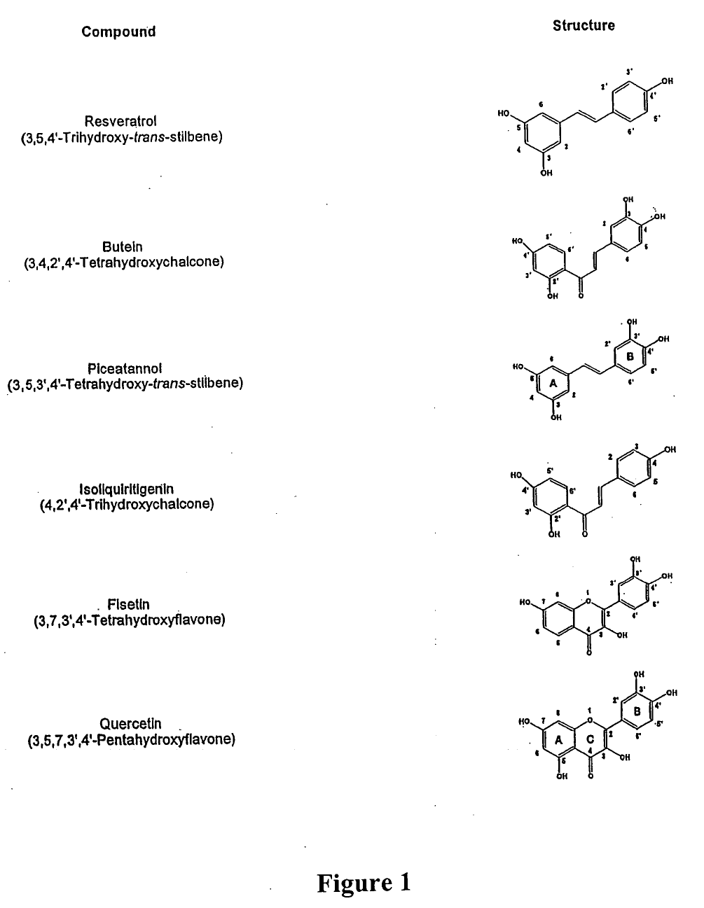

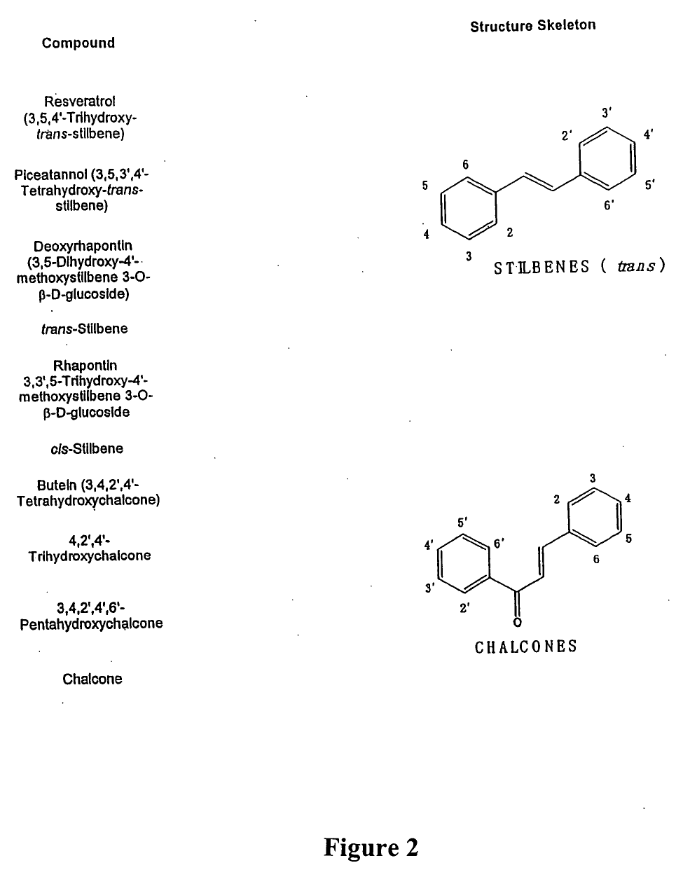

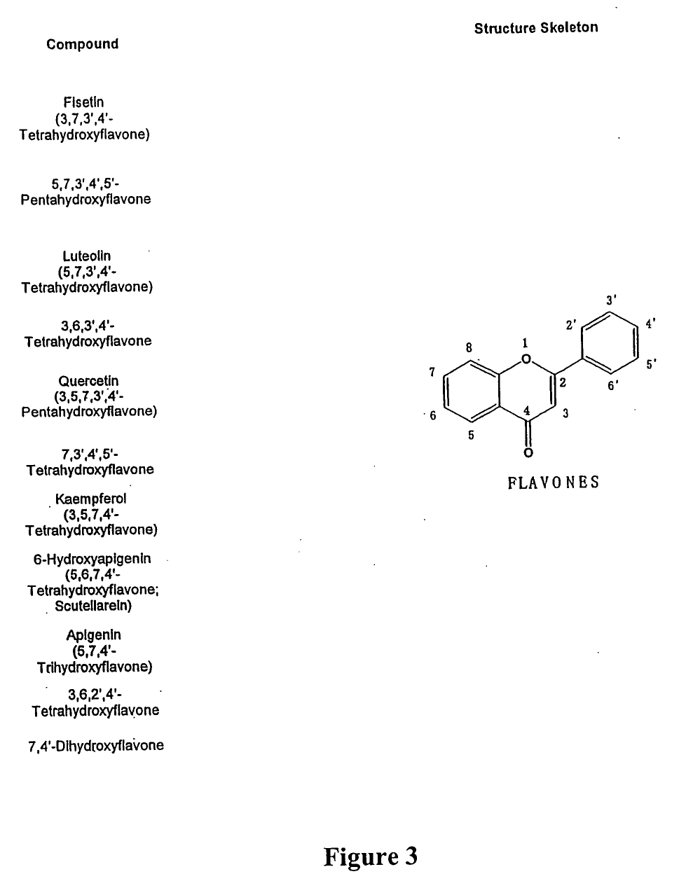

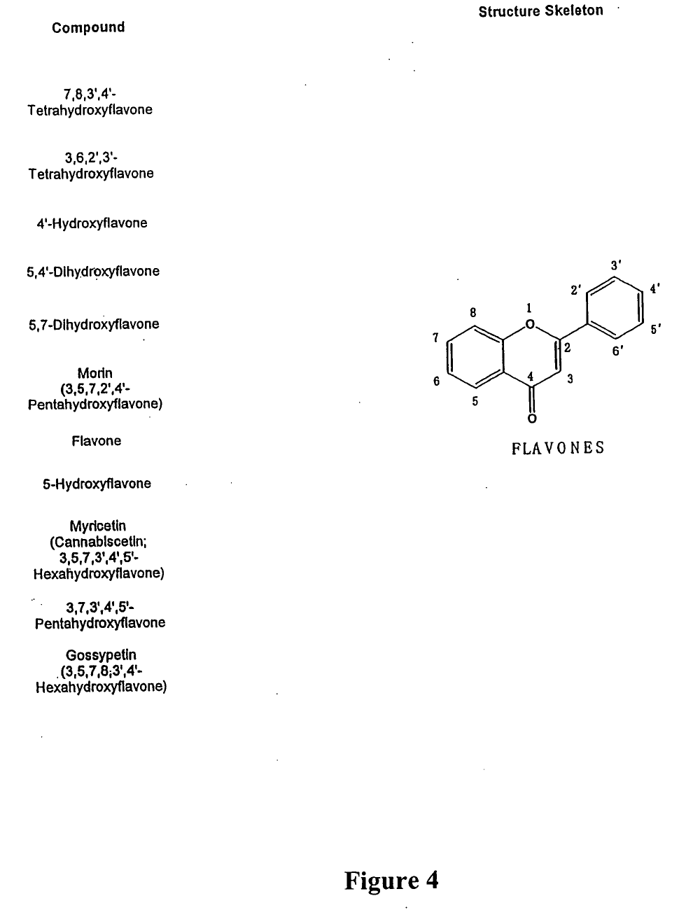

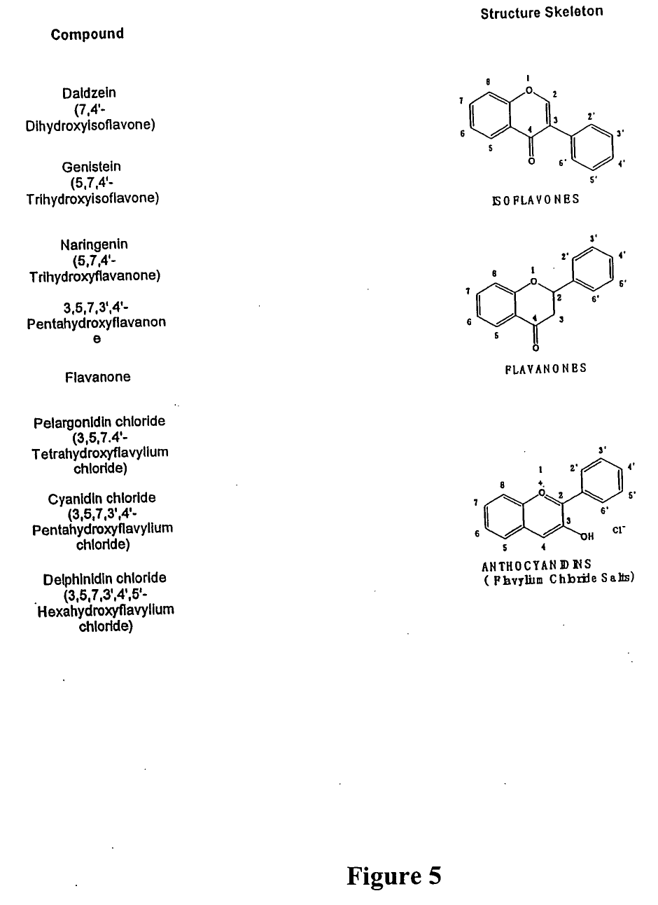

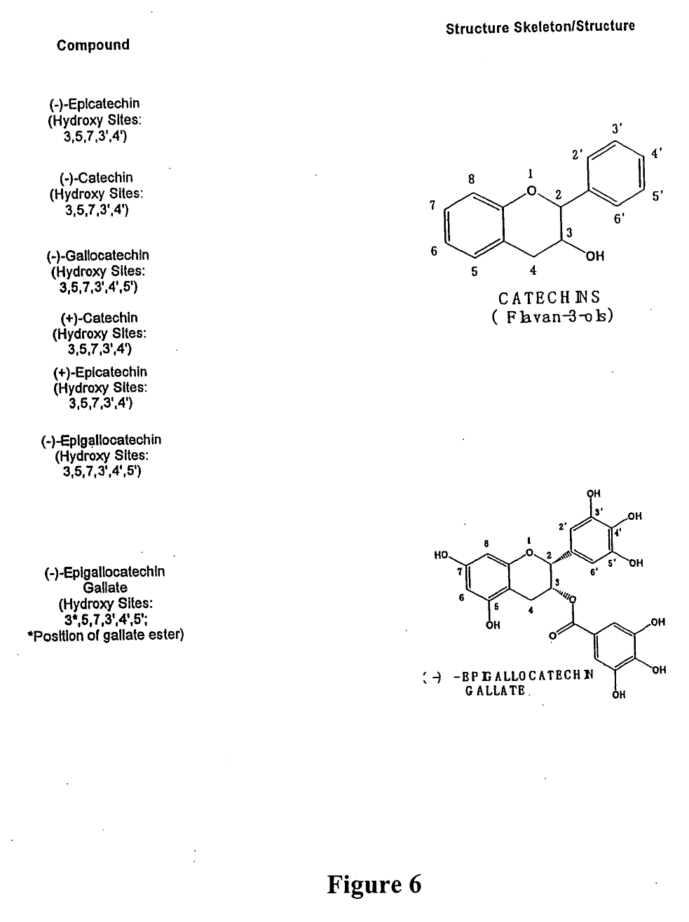

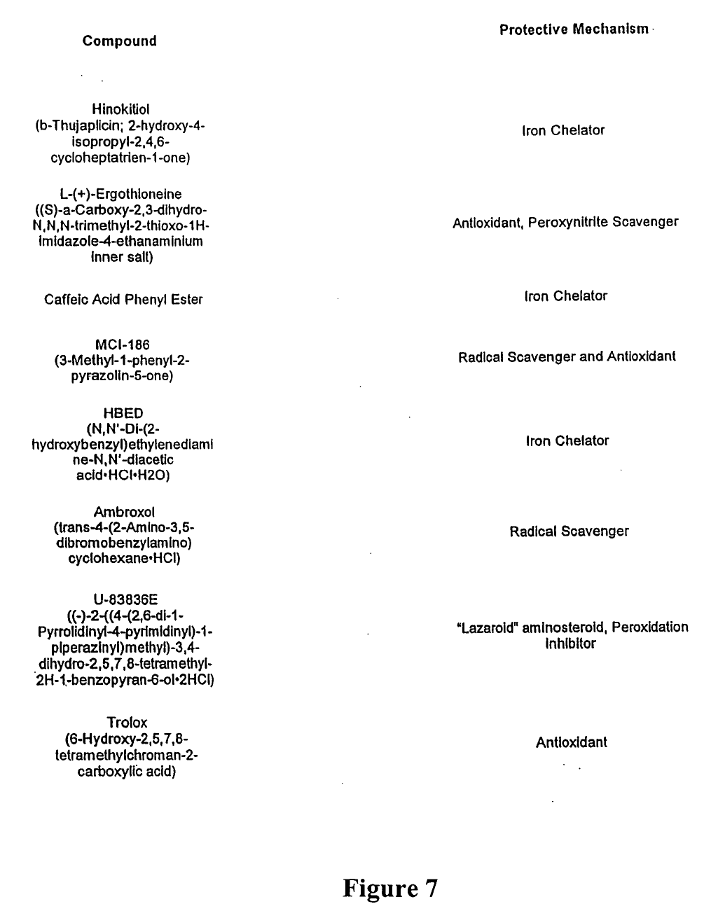

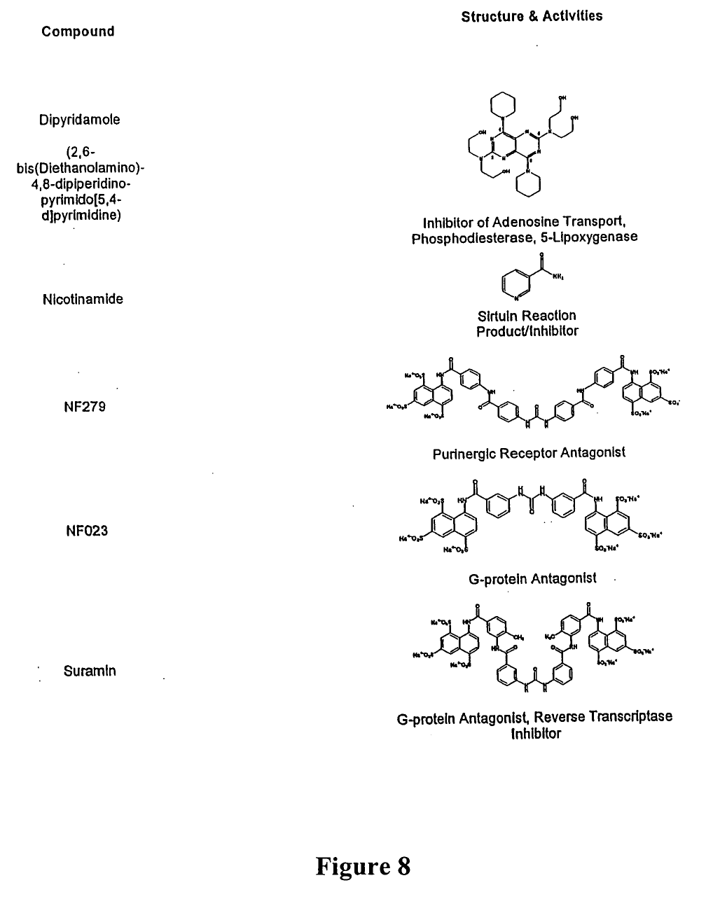

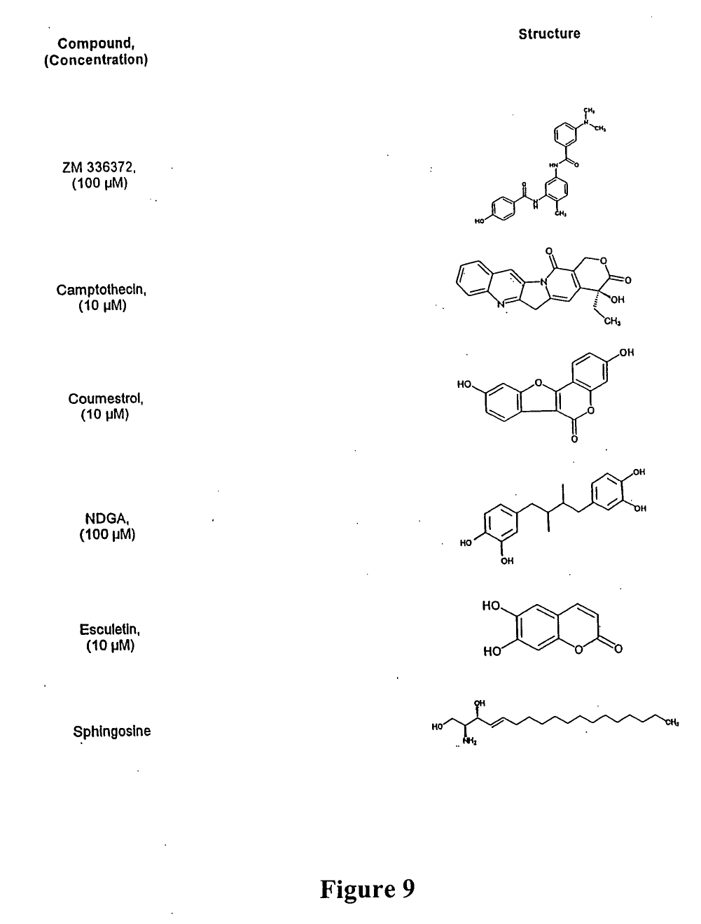

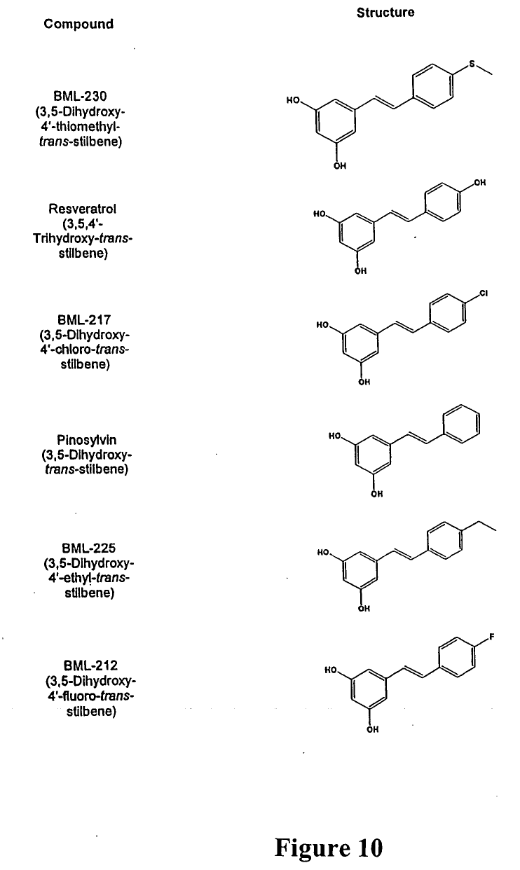

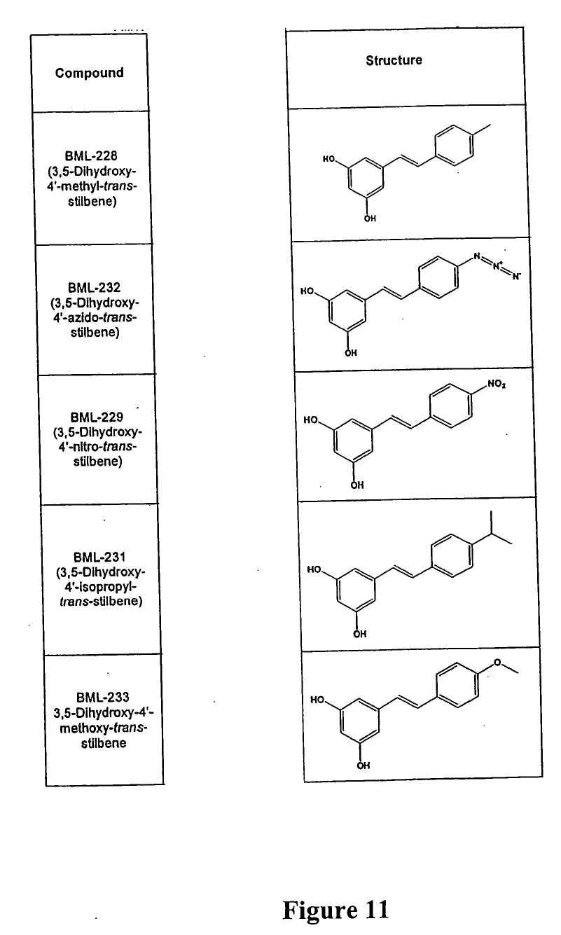

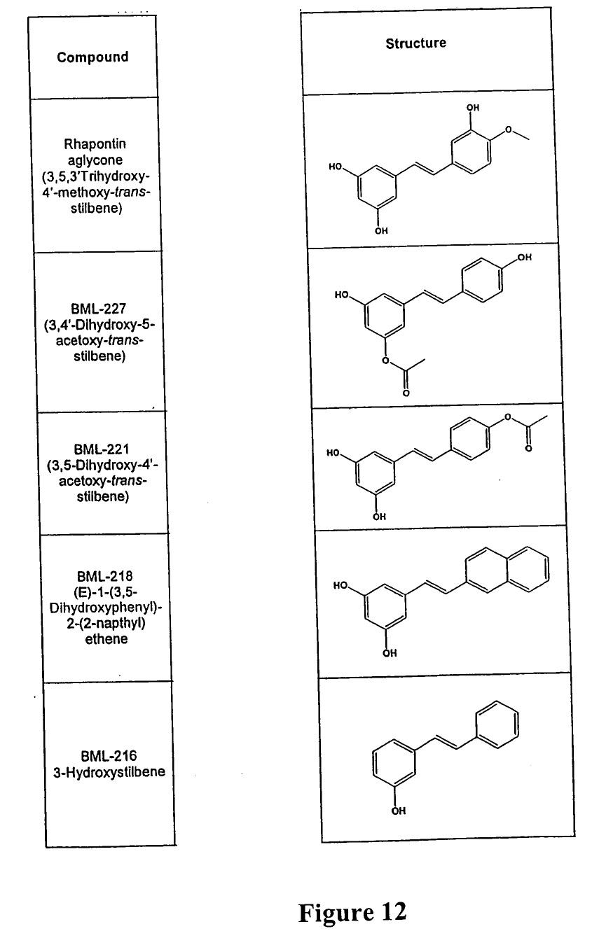

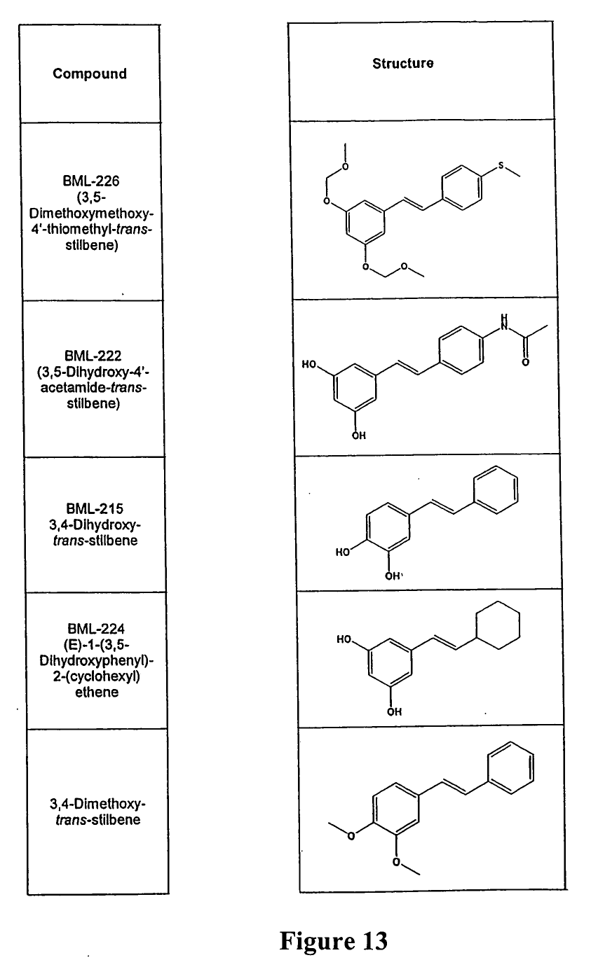

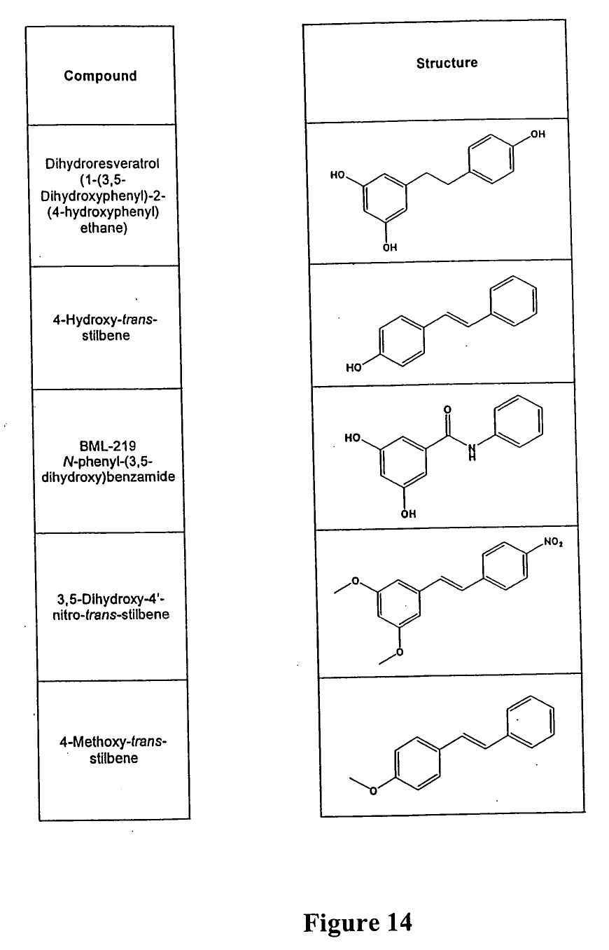

Specific examples of sirtuin modulators (e.g., sirtuin activators and sirtuin inhibitors) are shown in FIGS. 1-16.

In certain embodiments, sirtuin modulators of the invention exclude compounds encompassed by Formulae 77-88.

In certain embodiments, sirtuin modulators of the invention exclude one or more compounds disclosed by U.S. Provisional Application No. 60/667,179, filed Mar. 30, 2005.

Also included are pharmaceutically acceptable addition salts and complexes of the sirtuin modulators described herein. In cases wherein the compounds may have one or more chiral centers, unless specified, the compounds contemplated herein may be a single stereoisomer or racemic mixtures of stereoisomers.

The compounds and salts thereof described herein also include their corresponding hydrates (e.g., hemihydrate, monohydrate, dihydrate, trihydrate, tetrahydrate) and solvates. Suitable solvents for preparation of solvates and hydrates can generally be selected by a skilled artisan.

The compounds and salts thereof can be present in amorphous or crystalline (including co-crystalline and polymorph) forms.

Sirtuin modulating compounds also include the related secondary metabolites, such as phosphate, sulfate, acyl (e.g., acetyl, fatty acid acyl) and sugar (e.g., glucurondate, glucose) derivatives (e.g., of hydroxyl groups), particularly the sulfate, acyl and sugar derivatives. In other words, substituent groups —OH also include —OSO₃⁻ M⁺ and —OPO₄²⁻ M²⁺, where M⁺ and M⁺ are a suitable cation or pair of cations (preferably H⁺, NH₄⁺ or an alkali metal ion such as Na⁺ or K⁺) and sugars such as

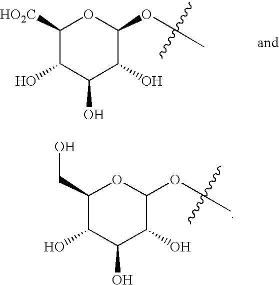

These groups are generally cleavable to —OH by hydrolysis or by metabolic (e.g., enzymatic) cleavage.

In cases in which the sirtuin modulators have unsaturated carbon-carbon double bonds, both the cis (Z) and trans (E) isomers are contemplated herein. In cases wherein the compounds may exist in tautomeric forms, such as keto-enol tautomers, such as 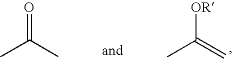
each tautomeric form is contemplated as being included within the methods presented herein, whether existing in equilibrium or locked in one form by appropriate substitution with R′. The meaning of any substituent at any one occurrence is independent of its meaning, or any other substituent's meaning, at any other occurrence.

Also included in the methods presented herein are prodrugs of the sirtuin modulators described herein. Prodrugs are considered to be any covalently bonded carriers that release the active parent drug in vivo.

Analogs and derivatives of the sirtuin modulators described herein can also be used for activating a member of the sirtuin protein family. For example, derivatives or analogs may make the compounds more stable or improve their ability to traverse cell membranes or being phagocytosed or pinocytosed. Exemplary derivatives include glycosylated derivatives, as described, e.g., in U.S. Pat. No. 6,361,815 for resveratrol. Other derivatives of resveratrol include cis- and trans-resveratrol and conjugates thereof with a saccharide, such as to form a glucoside (see, e.g., U.S. Pat. No. 6,414,037). Glucoside polydatin, referred to as piceid or resveratrol 3-O-beta-D-glucopyranoside, can also be used. Saccharides to which compounds may be conjugated include glucose, galactose, maltose, lactose and sucrose. Glycosylated stilbenes are further described in Regev-Shoshani et al. Biochemical J. (published on Apr. 16, 2003 as BJ20030141). Other derivatives of compounds described herein are esters, amides and prodrugs. Esters of resveratrol are described, e.g., in U.S. Pat. No. 6,572,882. Resveratrol and derivatives thereof can be prepared as described in the art, e.g., in U.S. Pat. Nos. 6,414,037; 6,361,815; 6,270,780; 6,572,882; and Brandolini et al. (2002) J. Agric. Food. Chem. 50:7407. Derivatives of hydroxyflavones are described, e.g., in U.S. Pat. No. 4,591,600. Resveratrol and other activating compounds can also be obtained commercially, e.g., from Sigma.

In certain embodiments, if a sirtuin modulator occurs naturally, it may be at least partially isolated from its natural environment prior to use. For example, a plant polyphenol may be isolated from a plant and partially or significantly purified prior to use in the methods described herein. A modulating compound may also be prepared synthetically, in which case it would be free of other compounds with which it is naturally associated. In an illustrative embodiment, a modulating composition comprises, or a modulating compound is associated with, less than about 50%, 10%, 1%, 0.1%, 10-2% or 10-3% of a compound with which it is naturally associated.

In certain embodiments, the subject sirtuin modulators, such as SIRT1 activators, do not have any substantial ability to inhibit P13-kinase, inhibit aldoreductase and/or inhibit tyrosine protein kinases at concentrations (e.g., in vivo) effective for modulating the deacetylase activity of the sirtuin, e.g., SIRT1. For instance, in preferred embodiments the sirtuin modulator is chosen to have an EC₅₀ for modulating sirtuin deacetylase activity that is at least 5 fold less than the EC₅₀ for inhibition of one or more of aldoreductase and/or tyrosine protein kinases, and even more preferably at least 10 fold, 100 fold or even 1000 fold less. Methods for assaying P13-Kinase activity, aldose reductase activity, and tyrosine kinase activity are well known in the art and kits to perform such assays may be purchased commercially. See e.g., U.S. Patent Publication No. 2003/0158212 for P13-kinase assays; U.S. Patent Publication No. 2002/20143017 for aldose reductase assays; tyrosine kinase assay kits may be purchased commercially, for example, from Promega (Madison, Wis.; world wide web at promega.com), Invitrogen (Carlsbad, Calif.; world wide web at invitrogen.com) or Molecular Devices (Sunnyvale, Calif.; world wide web at moleculardevices.com).

In certain embodiments, the subject sirtuin modulators do not have any substantial ability to transactivate EGFR tyrosine kinase activity at concentrations (e.g., in vivo) effective for activating the deacetylase activity of the sirtuin. For instance, in preferred embodiments the sirtuin modulator is chosen to have an EC₅₀ for modulating sirtuin deacetylase activity that is at least 5 fold less than the EC₅₀ for transactivating EGFR tyrosine kinase activity, and even more preferably at least 10 fold, 100 fold or even 1000 fold less. Methods for assaying transactivation of EGFR tyrosine kinase activity are well known in the art, see e.g., Pai et al. Nat. Med. 8: 289-93 (2002) and Vacca et al. Cancer Research 60: 5310-5317 (2000).

In certain embodiments, the subject sirtuin modulators do not have any substantial ability to cause coronary dilation at concentrations (e.g., in vivo) effective for activating the deacetylase activity of the sirtuin. For instance, in preferred embodiments the sirtuin modulator is chosen to have an EC₅₀ for modulating sirtuin deacetylase activity that is at least 5 fold less than the EC₅₀ for coronary dilation, and even more preferably at least 10 fold, 100 fold or even 1000 fold less. Methods for assaying vasodilation are well known in the art, see e.g., U.S. Patent Publication No. 2004/0236153.

In certain embodiments, the subject sirtuin modulators do not have any substantial spasmolytic activity at concentrations (e.g., in vivo) effective for modulating the deacetylase activity of the sirtuin. For instance, in preferred embodiments the sirtuin modulator is chosen to have an EC₅₀ for modulating sirtuin deacetylase activity that is at least 5 fold less than the EC₅₀ for spasmolytic effects (such as on gastrointestinal muscle), and even more preferably at least 10 fold, 100 fold or even 1000 fold less. Methods for assaying spasmolytic activity are well known in the art, see e.g., U.S. Patent Publication No. 2004/0248987.

In certain embodiments, the subject sirtuin modulators do not have any substantial ability to inhibit hepatic cytochrome P450 1B1 (CYP) at concentrations (e.g., in vivo) effective for modulating the deacetylase activity of the sirtuin. For instance, in preferred embodiments the sirtuin modulator is chosen to have an EC₅₀ for modulating sirtuin deacetylase activity that is at least 5 fold less than the EC₅₀ for inhibition of P450 1B1, and even more preferably at least 10 fold, 100 fold or even 1000 fold less. Methods for assaying cytochrome P450 activity are well known in the art and kits to perform such assays may be purchased commercially. See e.g., U.S. Pat. Nos. 6,420,131 and 6,335,428 and Promega (Madison, Wis.; world wide web at promega.com).

In certain embodiments, the subject sirtuin modulators do not have any substantial ability to inhibit nuclear factor-kappaB (NF-κB) at concentrations (e.g., in vivo) effective for modulating the deacetylase activity of the sirtuin. For instance, in preferred embodiments the sirtuin modulator is chosen to have an EC₅₀ for modulating sirtuin deacetylase activity that is at least 5 fold less than the EC₅₀ for inhibition of NF-κB, and even more preferably at least 10 fold, 100 fold or even 1000 fold less. Methods for assaying NF-κB activity are well known in the art and kits to perform such assays may be purchased commercially (e.g., from Oxford Biomedical Research (Ann Arbor, Mich.)).

In certain embodiments, the subject sirtuin modulators do not have any substantial ability to inhibit a histone deacetylase (HDACs) class I, a HDAC class II, or HDACs I and II, at concentrations (e.g., in vivo) effective for modulating the deacetylase activity of the sirtuin. For instance, in preferred embodiments the sirtuin modulator is chosen to have an EC₅₀ for modulating sirtuin deacetylase activity that is at least 5 fold less than the EC₅₀ for inhibition of an HDAC I and/or HDAC II, and even more preferably at least 10 fold, 100 fold or even 1000 fold less. Methods for assaying HDAC I and/or HDAC II activity are well known in the art and kits to perform such assays may be purchased commercially. See e.g., BioVision, Inc. (Mountain View, Calif.; world wide web at biovision.com) and Thomas Scientific (Swedesboro, N.J.; world wide web at tomassci.com).

In certain embodiments, the subject sirtuin modulators do not have any substantial ability to activate SIRT1 orthologs in lower eukaryotes, particularly yeast or human pathogens, at concentrations (e.g., in vivo) effective for modulating the deacetylase activity of human SIRT1. For instance, in preferred embodiments the SIRT1 modulator is chosen to have an EC₅₀ for modulating human SIRT1 deacetylase activity that is at least 5 fold less than the EC₅₀ for activating yeast Sir2 (such as Candida, S. cerevisiae, etc), and even more preferably at least 10 fold, 100 fold or even 1000 fold less.

In certain embodiments, the SIRT1 modulating compounds may have the ability to modulate one or more sirtuin protein homologs, such as, for example, one or more of human SIRT1, SIRT2, SIRT3, SIRT4, SIRT5, SIRT6, or SIRT7. In other embodiments, a SIRT1 modulator does not have any substantial ability to modulate other sirtuin protein homologs, such as, for example, one or more of human SIRT2, SIRT3, SIRT4, SIRT5, SIRT6, or SIRT7, at concentrations (e.g., in vivo) effective for modulating the deacetylase activity of human SIRT1. For instance, the SIRT1 modulator may be chosen to have an EC₅₀ for modulating human SIRT1 deacetylase activity that is at least 5 fold less than the EC₅₀ for modulating one or more of human SIRT2, SIRT3, SIRT4, SIRT5, SIRT6, or SIRT7, and even more preferably at least 10 fold, 100 fold or even 1000 fold less.

In other embodiments, the subject sirtuin modulators do not have any substantial ability to inhibit protein kinases; to phosphorylate mitogen activated protein (MAP) kinases; to inhibit the catalytic or transcriptional activity of cyclo-oxygenases, such as COX-2; to inhibit nitric oxide synthase (iNOS); or to inhibit platelet adhesion to type I collagen at concentrations (e.g., in vivo) effective for activating the deacetylase activity of the sirtuin. For instance, in preferred embodiments, the sirtuin modulator is chosen to have an EC₅₀ for modulating sirtuin deacetylase activity that is at least 5 fold less than the EC₅₀ for performing any of these activities, and even more preferably at least 10 fold, 100 fold or even 1000 fold less. Methods for assaying protein kinase activity, cyclo-oxygenase activity, nitric oxide synthase activity, and platelet adhesion activity are well known in the art and kits to perform such assays may be purchased commercially. See e.g., Promega (Madison, Wis.; world wide web at promega.com), Invitrogen (Carlsbad, Calif.; world wide web at invitrogen.com); Molecular Devices (Sunnyvale, Calif.; world wide web at moleculardevices.com) or Assay Designs (Ann Arbor, Mich.; world wide web at assaydesigns.com) for protein kinase assay kits; Amersham Biosciences (Piscataway, N.J.; world wide web at amershambiosciences.com) for cyclo-oxygenase assay kits; Amersham Biosciences (Piscataway, N.J.; world wide web at amershambiosciences.com) and R&D Systems (Minneapolis, Minn.; world wide web at mdsystems.com) for nitric oxide synthase assay kits; and U.S. Pat. Nos. 5,321,010; 6,849,290; and 6,774,107 for platelet adhesion assays.

One aspect of the present invention is a method for inhibiting, reducing or otherwise treating vision impairment by administering to a patient a therapeutic dosage of sirtuin modulator selected from a compound disclosed herein, or a pharmaceutically acceptable salt, prodrug or a metabolic derivative thereof.

In certain aspects of the invention, the vision impairment is caused by damage to the optic nerve or central nervous system. In particular embodiments, optic nerve damage is caused by high intraocular pressure, such as that created by glaucoma. In other particular embodiments, optic nerve damage is caused by swelling of the nerve, which is often associated with an infection or an immune (e.g., autoimmune) response such as in optic neuritis.

Glaucoma describes a group of disorders which are associated with a visual field defect, cupping of the optic disc, and optic nerve damage. These are commonly referred to as glaucomatous optic neuropathies. Most glaucomas are usually, but not always, associated with a rise in intraocular pressure. Exemplary forms of glaucoma include Glaucoma and Penetrating Keratoplasty, Acute Angle Closure, Chronic Angle Closure, Chronic Open Angle, Angle Recession, Aphakic and Pseudophakic, Drug-Induced, Hyphema, Intraocular Tumors, Juvenile, Lens-Particle, Low Tension, Malignant, Neovascular, Phacolytic, Phacomorphic, Pigmentary, Plateau Iris, Primary Congenital, Primary Open Angle, Pseudoexfoliation, Secondary Congenital, Adult Suspect, Unilateral, Uveitic, Ocular Hypertension, Ocular Hypotony, Posner-Schlossman Syndrome and Scleral Expansion Procedure in Ocular Hypertension & Primary Open-angle Glaucoma.

Intraocular pressure can also be increased by various surgical procedures, such as phacoemulsification (i.e., cataract surgery) and implanation of structures such as an artificial lens. In addition, spinal surgeries in particular, or any surgery in which the patient is prone for an extended period of time can lead to increased interoccular pressure.

Optic neuritis (ON) is inflammation of the optic nerve and causes acute loss of vision. It is highly associated with multiple sclerosis (MS) as 15-25% of MS patients initially present with ON, and 50-75% of ON patients are diagnosed with MS. ON is also associated with infection (e.g., viral infection, meningitis, syphilis), inflammation (e.g., from a vaccine), infiltration and ischemia.

Another condition leading to optic nerve damage is anterior ischemic optic neuropathy (AION). There are two types of AION. Arteritic AION is due to giant cell arteritis (vasculitis) and leads to acute vision loss. Non-arteritic AION encompasses all cases of ischemic optic neuropathy other than those due to giant cell arteritis. The pathophysiology of AION is unclear although it appears to incorporate both inflammatory and ischemic mechanisms.

Other damage to the optic nerve is typically associated with demyleination, inflammation, ischemia, toxins, or trauma to the optic nerve. Exemplary conditions where the optic nerve is damaged include Demyelinating Optic Neuropathy (Optic Neuritis, Retrobulbar Optic Neuritis), Optic Nerve Sheath Meningioma, Adult Optic Neuritis, Childhood Optic Neuritis, Anterior Ischemic Optic Neuropathy, Posterior Ischemic Optic Neuropathy, Compressive Optic Neuropathy, Papilledema, Pseudopapilledema and Toxic/Nutritional Optic Neuropathy.

Other neurological conditions associated with vision loss, albeit not directly associated with damage to the optic nerve, include Amblyopia, Bells Palsy, Chronic Progressive External Ophthalmoplegia, Multiple Sclerosis, Pseudotumor Cerebri and Trigeminal Neuralgia.

In certain aspects of the invention, the vision impairment is caused by retinal damage. In particular embodiments, retinal damage is caused by disturbances in blood flow to the eye (e.g., arteriosclerosis, vasculitis). In particular embodiments, retinal damage is caused by disrupton of the macula (e.g., exudative or non-exudative macular degeneration).

Exemplary retinal diseases include Exudative Age Related Macular Degeneration, Nonexudative Age Related Macular Degeneration, Retinal Electronic Prosthesis and RPE Transplantation Age Related Macular Degeneration, Acute Multifocal Placoid Pigment Epitheliopathy, Acute Retinal Necrosis, Best Disease, Branch Retinal Artery Occlusion, Branch Retinal Vein Occlusion, Cancer Associated and Related Autoimmune Retinopathies, Central Retinal Artery Occlusion, Central Retinal Vein Occlusion, Central Serous Chorioretinopathy, Eales Disease, Epimacular Membrane, Lattice Degeneration, Macroaneurysm, Diabetic Macular Edema, Irvine-Gass Macular Edema, Macular Hole, Subretinal Neovascular Membranes, Diffuse Unilateral Subacute Neuroretinitis, Nonpseudophakic Cystoid Macular Edema, Presumed Ocular Histoplasmosis Syndrome, Exudative Retinal Detachment, Postoperative Retinal Detachment, Proliferative Retinal Detachment, Rhegmatogenous Retinal Detachment, Tractional Retinal Detachment, Retinitis Pigmentosa, CMV Retinitis, Retinoblastoma, Retinopathy of Prematurity, Birdshot Retinopathy, Background Diabetic Retinopathy, Proliferative Diabetic Retinopathy, Hemoglobinopathies Retinopathy, Purtscher Retinopathy, Valsalva Retinopathy, Juvenile Retinoschisis, Senile Retinoschisis, Terson Syndrome and White Dot Syndromes.

Other exemplary diseases include ocular bacterial infections (e.g. conjunctivitis, keratitis, tuberculosis, syphilis, gonorrhea), viral infections (e.g. Ocular Herpes Simplex Virus, Varicella Zoster Virus, Cytomegalovirus retinitis, Human Immunodeficiency Virus (HIV)) as well as progressive outer retinal necrosis secondary to HIV or other HIV-associated and other immunodeficiency-associated ocular diseases. In addition, ocular diseases include fungal infections (e.g. Candida choroiditis, histoplasmosis), protozoal infections (e.g. toxoplasmosis) and others such as ocular toxocariasis and sarcoidosis.

One aspect of the invention is a method for inhibiting, reducing or treating vision impairment in a subject undergoing treatment with a chemotherapeutic drug (e.g., a neurotoxic drug, a drug that raises intraocular pressure such as a steroid), by administering to the subject in need of such treatment a therapeutic dosage of a sirtuin modulator disclosed herein.

Another aspect of the invention is a method for inhibiting, reducing or treating vision impairment in a subject undergoing surgery, including ocular or other surgeries performed in the prone position such as spinal cord surgery, by administering to the subject in need of such treatment a therapeutic dosage of a sirtuin modulator disclosed herein. Ocular surgeries include cataract, iridotomy and lens replacements.

Another aspect of the invention is the treatment, including inhibition and prophylactic treatment, of age-related ocular diseases including cataracts, dry eye, retinal damage and the like, by administering to the subject in need of such treatment a therapeutic dosage of a sirtuin modulator disclosed herein.

The formation of cataracts is associated with several biochemical changes in the lens of the eye, such as decreased levels of antioxidants ascorbic acid and glutathione, increased lipid, amino acid and protein oxidation, increased sodium and calcium, loss of amino acids and decreased lens metabolism. The lens, which lacks blood vessels, is suspended in extracellular fluids in the anterior part of the eye. Nutrients, such as ascorbic acid, glutathione, vitamin E, selenium, bioflavonoids and carotenoids are required to maintain the transparency of the lens. Low levels of selenium results in an increase of free radical-inducing hydrogen peroxide, which is neutralized by the selenium-dependent antioxidant enzyme glutathione peroxidase. Lens-protective glutathione peroxidase is also dependent on the amino acids methionine, cysteine, glycine and glutamic acid.

Cataracts can also develop due to an inability to properly metabolize galactose found in dairy products that contain lactose, a disaccharide composed of the monosaccharide galactose and glucose. Cataracts can be prevented, delayed, slowed and possibly even reversed if detected early and metabolically corrected.

Retinal damage is attributed, inter alia, to free radical initiated reactions in glaucoma, diabetic retinopathy and age-related macular degeneration (AMD). The eye is a part of the central nervous system and has limited regenerative capability. The retina is composed of numerous nerve cells which contain the highest concentration of polyunsaturated fatty acids (PFA) and subject to oxidation. Free radicals are generated by UV light entering the eye and mitochondria in the rods and cones, which generate the energy necessary to transform light into visual impulses. Free radicals cause peroxidation of the PFA by hydroxyl or superoxide radicals which in turn propagate additional free radicals. The free radicals cause temporary or permanent damage to retinal tissue.

Glaucoma is usually viewed as a disorder that causes an elevated intraocular pressure (IOP) that results in permanent damage to the retinal nerve fibers, but a sixth of all glaucoma cases do not develop an elevated IOP. This disorder is now perceived as one of reduced vascular perfusion and an increase in neurotoxic factors. Recent studies have implicated elevated levels of glutamate, nitric oxide and peroxynitirite in the eye as the causes of the death of retinal ganglion cells. Neuroprotective agents may be the future of glaucoma care. For example, nitric oxide synthase inhibitors block the formation of peroxynitrite from nitric oxide and superoxide. In a recent study, animals treated with aminoguanidine, a nitric oxide synthase inhibitor, had a reduction in the loss of retinal ganglion cells. It was concluded that nitric oxide in the eye caused cytotoxicity in many tissues and neurotoxicity in the central nervous system.

Diabetic retinopathy occurs when the underlying blood vessels develop microvascular abnormalities consisting primarily of microaneurysms and intraretinal hemorrhages. Oxidative metabolites are directly involved with the pathogenesis of diabetic retinopathy and free radicals augment the generation of growth factors that lead to enhanced proliferative activity. Nitric oxide produced by endothelial cells of the vessels may also cause smooth muscle cells to relax and result in vasodilation of segments of the vessel. Ischemia and hypoxia of the retina occur after thickening of the arterial basement membrane, endothelial proliferation and loss of pericytes. The inadequate oxygenation causes capillary obliteration or nonperfusion, arteriolar-venular shunts, sluggish blood flow and an impaired ability of RBCs to release oxygen. Lipid peroxidation of the retinal tissues also occurs as a result of free radical damage.

The macula is responsible for our acute central vision and composed of light-sensing cells (cones) while the underlying retinal pigment epithelium (RPE) and choroid nourish and help remove waste materials. The RPE nourishes the cones with the vitamin A substrate for the photosensitive pigments and digests the cones shed outer tips. RPE is exposed to high levels of UV radiation, and secretes factors that inhibit angiogenesis. The choroid contains a dense vascular network that provides nutrients and removes the waste materials.

In AMD, the shed cone tips become indigestible by the RPE, where the cells swell and die after collecting too much undigested material. Collections of undigested waste material, called drusen, form under the RPE. Photoxic damage also causes the accumulation of lipofuscin in RPE cells. The intracellular lipofuscin and accumulation of drusen in Bruch's membrane interferes with the transport of oxygen and nutrients to the retinal tissues, and ultimately leads to RPE and photoreceptor dysfunction. In exudative AMD, blood vessels grow from the choriocapillaris through defects in Bruch's membrane and may grow under the RPE, detaching it from the choroid, and leaking fluid or bleeding.

Macular pigment, one of the protective factors that prevent sunlight from damaging the retina, is formed by the accumulation of nutritionally derived carotenoids, such as lutein, the fatty yellow pigment that serves as a delivery vehicle for other important nutrients and zeaxanthin. Antioxidants such as vitamins C and E, beta-carotene and lutein, as well as zinc, selenium and copper, are all found in the healthy macula. In addition to providing nourishment, these antioxidants protect against free radical damage that initiates macular degeneration.

Another aspect of the invention is the prevention or treatment of damage to the eye caused by stress, chemical insult or radiation, by administering to the subject in need of such treatment a therapeutic dosage of a sirtuin modulator disclosed herein. Radiation or electromagnetic damage to the eye can include that caused by CRT's or exposure to sunlight or UV.

In certain aspects of the invention, the invention excludes the treatment of one or more of the following conditions: cataracts, retinopathy, retinitis pigmentosa, ocular neuritis and vascular disease of capillary beds of the eye. The invention contemplates the exclusion of any one or more of the above-listed conditions, including any combination thereof.

In certain aspects of the invention, the invention excludes the treatment of vision conditions associated with one or more of the following conditions: insulin resistance, diabetes, obesity (including metabolic syndrome), cell death and/or dysfunction, aging, blood coagulation disorders, cardiovascular disease, stress, cancer, inflammation, neurodegeneration, viral disease and fungal diseases. The invention contemplates the exclusion of vision conditions associated with any one or more of the above-listed conditions, including any combination thereof.

In certain embodiments of the invention, the invention excludes treatment or prevention of one or more vision conditions disclosed by U.S. Provisional Application No. 60/667,179, filed Mar. 30, 2006.

Another aspect of the invention is a pharmaceutical dosage form comprising a therapeutically effective amount of a sirtuin modulator, or a pharmaceutically acceptable salt, prodrug or metabolic derivative thereof. In one embodiment, the dosage form is a tablet, capsule or an oral solution. In another embodiment, the dosage may be adapted for intravenous infusion, parenteral delivery or oral delivery. Preferably, the dosage form is suitable for ophthalmic administration, such as a solution, gel or cream or an implantable device.

In another embodiment, the therapeutically effective amount of the sirtuin modulator is in the range of from about 0.1 mg/kg body weight to about 500 mg/kg body weight, from about 1 mg/kg body weight to about 400 mg/kg body weight, from about 10 mg/kg body weight to about 100 mg/kg body weight, or even from about 10 mg/kg body weight to about 75 mg/kg body weight.

Another aspect of the present invention is a method for conducting a pharmaceutical business, comprising:

a. manufacturing a preparation of any of the sirtuin modulators disclosed herein; and

b. marketing to healthcare providers the benefits of using the preparation or kit in the treatment of vision impairment.

In certain embodiments, the invention provides a method for conducting a pharmaceutical business, comprising:

a. providing a distribution network for selling said preparation; and

b. providing instruction material to patients or physicians for using the preparation or kit to treat vision impairment.

In certain embodiments, the invention also provides a method for conducting a pharmaceutical business, comprising:

a. determining an appropriate formulation and dosage of a sirtuin modulator for the treatment of vision impairment;

b. conducting therapeutic profiling of formulations identified in step (a), for efficacy and toxicity in animals; and

c. providing a distribution network for selling a preparation identified in step (b) as having an acceptable therapeutic profile.

In still further embodiments, the method includes an additional step of providing a sales group for marketing the preparation to healthcare providers.

In yet other embodiments, the invention provides a method for conducting a pharmaceutical business, comprising:

a. determining an appropriate formulation and dosage of a sirtuin modulator to be administered in the treatment of vision impairment; and

b. licensing, to a third party, the rights for further development and sale of the formulation.

D. Exemplary Formulations

In another aspect, the present invention provides pharmaceutical compositions. The composition for use in the subject method may be conveniently formulated for administration with a biologically acceptable medium, such as water, buffered saline (e.g., phosphate-buffered saline), polyol (for example, glycerol, propylene glycol, liquid polyethylene glycol and the like) or suitable mixtures thereof. The optimum concentration of the active ingredient(s) in the chosen medium can be determined empirically, according to procedures well known to medicinal chemists. As used herein, “biologically acceptable medium” includes solvents, dispersion media, and the like which may be appropriate for the desired route of administration of the pharmaceutical preparation. Except insofar as any conventional media or agent is incompatible with the treating vision impairment, its use in the pharmaceutical preparation of the invention is contemplated. Suitable vehicles and their formulation inclusive of other proteins are described, for example, in the book Remington's Pharmaceutical Sciences (Remington's Pharmaceutical Sciences. Mack Publishing Company, Easton, Pa., USA 1985). These vehicles include injectable “deposit formulations”.

Exemplary formuations of the invention include nicotinamide riboside dissolved in phosphate-buffered saline (PBS), reservatrol together with beta-cyclodextrin (e.g., 10-20 mM or 14-16 mM resveratrol in 5-15% (about 10%) beta-cyclodextrin), and resveratrol nanoparticles together with a cellulose derivative (e.g., hydroxypropylmethylcellulose (HPMC)) and dioctyl sodium sulfosuccinate (DOSS) (e.g., 15-25% resveratrol nanoparticles, 1-1.5% HPMC, 0.01-0.10% DOSS). Each of these formulations can optionally include additional active agents, buffers (e.g., PBS), preservatives and the like. Preferably, such formulations are isotonic.

Pharmaceutical formulations of the present invention can also include veterinary compositions, e.g., pharmaceutical preparations of a sirtuin modulator suitable for veterinary uses, e.g., for the treatment of livestock or domestic animals, e.g., dogs.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a drug at a particular target site.

Methods of introduction may additional be provided by non-biodegradable devices. In particular, a sirtuin modulator can be administered via an implantable lens. The sirtuin modulator can be coated on the lens, dispersed throughout the lens or both.

The preparations of the present invention may be given intraocularly (e.g., intravitreally), orally, parenterally, topically, or rectally. They are, of course, given by forms suitable for each administration route. For example, they are administered in tablets or capsule form, by injection, inhalation, eye lotion, ointment, suppository, controlled release patch, etc.; administration by injection, infusion or inhalation; topical by lotion or ointment; and rectal by suppositories. Oral and topical administrations are preferred.

The phrases “parenteral administration” or “administered parenterally” as used herein mean modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracistemally and topically, as by powders, ointments or drops, including ophthalmically, buccally and sublingually.

A sirtuin modulator may be administered topically to the eye or eye lid, for example, using drops, an ointment, a cream, a gel, a suspension, etc. The agent(s) may be formulated with excipients such as methylcellulose, hydroxypropyl methylcellulose, hydroxypropyl cellulose, polyvinyl pyrrolidine, neutral poly(meth)acrylate esters, and other viscosity-enhancing agents. The agent(s) may be injected into the eye, for example, injection under the conjunctiva or tenon capsule, intravitreal injection, or retrobulbar injection. The agent(s) may be administered with a slow release drug delivery system, such as polymers, matrices, microcapsules, or other delivery systems formulated from, for example, glycolic acid, lactic acid, combinations of glycolic and lactic acid, liposomes, silicone, polyanhydride polyvinyl acetate alone or in combination with polyethylene glycol, etc. The delivery device can be implanted intraocularly, for example, implanted under the conjunctiva, implanted in the wall of the eye, sutured to the sclera, for long-term drug delivery.

There are used for an ophthalmic composition customary pharmaceutically acceptable excipients and additives known to the person skilled in the art, for example those of the type mentioned below, especially carriers, stabilizers, solubilizers, tonicity enhancing agents, buffer substances, preservatives, thickeners, complexing agents and other excipients. Examples of such additives and excipients can be found in U.S. Pat. Nos. 5,891,913, 5,134,124 and 4,906,613.

Formulations of the present invention in an embodiment are prepared, for example by mixing the active agent with the corresponding excipients and/or additives to form corresponding ophthalmic compositions. The active agent is preferably administered in the form of eye drops, the active agent being conventionally dissolved, for example, in a carrier. The solution is, where appropriate, adjusted and/or buffered to the desired pH and, where appropriate, a stabilizer, a solubilizer or a tonicity enhancing agent is added. Where appropriate, preservatives and/or other excipients are added to an ophthalmic formulation of the invention.

Carriers used in accordance to an embodiment of the present invention are typically suitable for topical or general administration, and are for example water, aqueous solutions such as phosphate-buffered saline, mixtures of water and water-miscible solvents, such as C1- to C7-alkanols, vegetable oils or mineral oils including from about 0.5% to about 5% by weight hydroxyethylcellulose, ethyl oleate, carboxymethylcellulose, polyvinylpyrrolidone and other non-toxic water-soluble polymers for ophthalmic uses, such as, for example, cellulose derivatives, such as methylcellulose, alkali metal salts of carboxymethylcellulose, hydroxymethylcellulose, hydroxyethylcellulose, methylhydroxypropylcellulose and hydroxypropylcellulose, acrylates or methacrylates, such as salts of polyacrylic acid or ethyl acrylate, polyacrylamides, natural products, such as gelatin, alginates, pectins, tragacanth, karaya gum, xanthan gum, carrageenin, agar and acacia, starch derivatives, such as starch acetate and hydroxypropyl starch, and also other synthetic products, such as polyvinyl alcohol, polyvinylpyrrolidone, polyvinyl methyl ether, polyethylene oxide, preferably cross-linked polyacrylic acid, such as neutral Carbopol, or mixtures of those polymers. Preferred carriers include, for example, water, cellulose derivatives, such as methylcellulose, alkali metal salts of carboxymethylcellulose, hydroxymethylcellulose, hydroxyethylcellulose, methylhydroxypropylcellulose and hydroxypropylcellulose, neutral Carbopol, or mixtures thereof. The concentration of the carrier ranges, for example, from about 1 to about 100,000 times the concentration of the active ingredient.

The solubilizers used for an ophthalmic composition of the present invention in an embodiment include, for example, tyloxapol, fatty acid glycerol poly-lower alkylene glycol esters, fatty acid poly-lower alkylene glycol esters, polyethylene glycols, glycerol ethers vitamin E and vitamin E derivatives, such as Vitamin E Tocopherol Polyethylene Glycol 1000 Succinate (TPGS) or mixtures of those compounds. A specific example of a solubilizer is a reaction product of castor oil and ethylene oxide. Reaction products of castor oil and ethylene oxide have proved to be particularly good solubilizers that are tolerated extremely well by the eye. The concentration used depends especially on the concentration of the active ingredient. The amount added is typically sufficient to solubilize the active ingredient. For example, the concentration of the solubilizer ranges from about 0.1 to about 5000 times the concentration of the active ingredient pursuant to an embodiment of the present invention.

According to an embodiment of the present invention lower alkylene means linear or branched alkylene with up to and including seven carbon atoms. Examples are methylene, ethylene, 1,3-propylene, 1,2-propylene, 1,5-pentylene, 2,5 hexylene, 1,7-heptylene and the like. Lower alkylene is preferably, such as linear or branched alkylene, with up to and including four carbon atoms.

Examples of buffer substances are acetate, ascorbate, borate, hydrogen carbonate/carbonate, citrate, gluconate, lactate, phosphate, propionate, perborate TRIS (tromethamine) buffers and the like. Tromethamine and borate buffer are preferred buffers. The amount of buffer substance added is, for example, that necessary to ensure and maintain a physiologically tolerable pH range. The pH range is typically in the range of from about 5 to about 9, preferably from about 6 to about 8.2 and more preferably from about 6.8 to about 8.1.

Tonicity enhancing agents are, for example, ionic compounds, such as alkali metal or alkaline earth metal halides, such as, for example, CaCl₂, KBr, KCi, LiCI, NaI, NaBr or NaCl, or boric acid and the like. Non-ionic tonicity enhancing agents are, for example, urea, glycerol, sorbitol, mannitol, propylene glycol, dextrose and the like. For example, sufficient tonicity enhancing agent is added to impart to the ready-for-use ophthalmic composition an osmolality of approximately front about 50 mOsmol to about 1000 mOsmol, preferred from about 100 mOsmol to about 400 mOsmol, more preferred from about 200 mOsmol to about 400 mOsmol and even more preferred from about 280 mOsmol to about 350 mOsmol.

Examples of preservatives are quaternary ammonium salts, such as cetrimide, benzalkonium chloride or benzoxonium chloride, alkyl-mercury salts of thiosalicylic acid, such as, for example, thimerosal, phenylmercuric nitrate, phenylmercuric acetate or phenylmercuric borate, parabens, such as, for example, methylparaben or propylparaben, alcohols, such as, for example, chlorobutanol, benzyl alcohol or phenyl ethanol, guanidine derivatives, such as, for example, chlorohexidine or polyhexamethylene biguanide, or sorbic acid and the like. Where appropriate, a sufficient amount of preservative is added to the ophthalmic composition to ensure protection against secondary contaminations during use caused by bacteria and fungi.

Ophthalmic formulations of the present invention can also include, for example, non-toxic excipients, such as, for example, emulsifiers, wetting agents or fillers, such as, for example, the polyethylene glycols designated 200, 300, 400 and 600, or Carbowax designated 1000, 1500, 4000, 6000 and 10,000 and the like. Other excipients that may be used if desired are listed below but they are not intended to limit in any way the scope of the possible excipients. They include complexing agents, such as disodium-EDTA or EDTA; antioxidants, such as ascorbic acid, acetylcysteine, cysteine, sodium hydrogen sulfite, butyl-hydroxyanisole, butyl-hydroxytoluene or alpha-tocopherol acetate; stabilizers, such as a cyclodextrin, thiourea, thiosorbitol, sodium dioctyl sulfosuccinate or monothioglycerol vitamin E and vitamin E derivatives, such as Vitamin E Tocopherol Polyethylene Glycol 1000 Succinate (TPGS); or other excipients, such as, for example, lauric acid sorbitol ester, triethanol amine oleate or palmitic acid ester and the like. Preferred excipients are complexing agents, such as disodium-EDTA and stabilizers, such as a cyclodextrin and the like. Other preferred excipients include penetration enhancers such as benzalkonium chloride, Brij polymers such as PEG lauryl ether, and also dodecylmaltoside. The amount and type of excipient added is in accordance with the particular requirements and is generally in the range of from approximately 0.0001% by weight to approximately 90% by weight.

As indicated above a simple formulation of the present invention according to an embodiment includes an aqueous solvent which may be sterile water suitable for administration to the eye having an active agent dissolved, suspended or emulsified therein. However, preferred formulations of the present invention include the active agent dissolved in a formulation which is referred to in the art as an artificial tear formulation. Such artificial tear formulations are disclosed and described within U.S. Pat. Nos. 5,895,654; 5,627,611; and 5,591,426 as well as patents and publications cited and referred to in these patents, all of which are intended to be incorporated herein by reference.

Artificial tear formulations of the present invention in an embodiment promote good wettability and spread. Further, the artificial tear formulations preferably have good retention and stability on the eye and do not cause significant discomfort to the user. An exemplary artificial tear composition of the present invention includes:

(1) polyvinylpyrrolidone, preferably in the amount of about 0.1 to 5% by weight of said solution;

(2) benzalkonium chloride, preferably in an amount of about 0.01% to about 0.10% by weight;

(3) hydroxypropyl methylcellulose, preferably in an amount of about 0.2% to about 1.5% by weight of said solution; and

(4) glycerin, preferably in an amount of about 0.2% to about 1.0% by weight of said solution, wherein the composition is an aqueous solution having isotonic properties.

Those skilled in the art will recognize that a wide range of different formulations and artificial tear formulations which can be utilized in connection with the present invention.

Additional ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatable with such fluids.

Formulations of the present invention can be administered in a manner generally known to those skilled in the art. In an embodiment, the formulation is administered using an eyedropper. The eyedropper can be constructed in any suitable way.

It may be desirable to utilize a measured dose eyedropper of the type described within U.S. Pat. No. 5,514,118 or an illuminated eyedropper device of the type described in U.S. Pat. No. 5,584,823. A range of other eye droppers can also be utilized of the type described within the following U.S. Pat. Nos. 5,059,188; 4,834,727; 4,629,456; and 4,515,295. The patents cited here which disclose eyedroppers are incorporated herein by reference as are the various patents and publications cited and discussed within these patents.

Compositions usable for injection into the vitreous body contain a physiologically tolerable carrier together with the relevant agent as described herein, dissolved or dispersed therein as an active ingredient. As used with respect to the vitrous body, the term “pharmaceutically acceptable” refers to compositions, carriers, diluents and reagents which represent materials that are capable of administration into the vitreous body of a mammal without the production of undesirable physiological effects. The preparation of an injectable pharmacological composition typically contains active ingredients dissolved or dispersed therein. The preparation can also be emulsified. The active ingredient can be mixed with excipients which are pharmaceutically acceptable and compatible with the active ingredient and in amounts suitable for use in the therapeutic methods described herein. Suitable excipients are, for example, water, saline, sorbitol, glycerol or the like and combinations thereof. In addition, if desired, the composition can contain minor amounts of auxiliary substances such as wetting or emulsifying agents, pH buffering agents, and the like which enhance the effectiveness of the active ingredient. The composition can also contain viscosity enhancing agents like hyaluronic acid. The therapeutic composition of the present invention can include pharmaceutically acceptable salts of the components therein. Pharmaceutically acceptable salts include the acid addition salts that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Particularly preferred is the HCl salt.

Depending from the application form the active compound liberates in an immediate or a sustained release manner. A sustained release ophthalmic formulation is preferred when it is desirable to reduce the injection frequency.

One possibility to achieve sustained release kinetics is by micronizing the active compound. A micronized liquid dispersion, generally formed by either milling or precipitation, can be dried prior to formulating the composition into a solid dose form for administration. Typically, micronized active compounds are formulated as a freely-flowing liquid (e.g., not a gel, paste or gum) or as a solid dosage form (e.g., a tablet or capsule). Such formulations are preferably safe for internal use (e.g., ingestion, injection).

Powders comprising micronized drug can be made by spray-drying aqueous dispersions of a micronized drug to form a dry powder which consists of aggregated drug particles. Alternatively, the aqueous dispersion of drug can contain a dissolved diluent, such as lactose or mannitol, which when spray dried forms diluent particles, each of which contains at least one embedded drug particle.

Micronized drug dispersions can also be freeze-dried to obtain powders suitable for formulation into solid dose forms. Such powders comprise aggregated micronized drug particles. Freeze dried powders can also be obtained by freeze drying aqueous dispersions of drug, which additionally contain a dissolved diluent such as lactose or mannitol. In these instances the freeze dried powders consist of particles of diluent, each of which contains at least one embedded drug particle.

Other known methods of processing liquid dispersions, and which can be employed in the present invention, include granulation, including but not limited to high shear granulation, fluid bed granulation, roto granulation, and melt granulation. Additional methods such as spray coating and extrusion spherization can also be used. Any other conventional method for drying or otherwise processing a liquid dispersion can also be used in the invention.

The particles are preferably reduced in size at a temperature which does not significantly degrade the drug substance. Processing temperatures of less than about 30 degrees to about 40 degrees C. are ordinarily preferred. If desired, the processing equipment can be cooled with conventional cooling equipment. The method is conveniently carried out under conditions of ambient temperature and at processing pressures which are safe and effective for the milling process.

Typically, micronized drug dispersions contain a discrete phase of a drug substance as described above having a surface modifier adsorbed on the surface thereof. Useful surface modifiers are believed to include those which physically adhere to the surface of the drug substance but do not chemically bond to the drug. Suitable surface modifiers can preferably be selected from known organic and inorganic pharmaceutical excipients. Such excipients include various polymers, low molecular weight oligomers, natural products and surfactants, such as nonionic and anionic surfactants. Representative examples of excipients include gelatin, casein, lecithin (phosphatides), gum acacia, cholesterol, tragacanth, stearic acid, benzalkonium chloride, calcium stearate, glyceryl monostearate, cetostearl alcohol, cetomacrogol emulsifying wax, sorbitan esters, polyoxyethylene alkyl ethers, e.g., macrogol ethers such as cetomacrogol 1000, polyoxyethylene castor oil derivatives, polyoxyethylene sorbitan fatty acid esters, e.g., the commercially available Tweens (Tween 80), polyethylene glycols, polyoxyethylene stearates, colloidol silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, and polyvinylpyrrolidone (PVP). Most of these excipients are described in detail in the Handbook of Pharmaceutical Excipients, published jointly by the American Pharmaceutical Association and The Pharmaceutical Society of Great Britain, the Pharmaceutical Press, 1986, the disclosure of which is hereby incorporated by reference in its entirety. The surface modifiers are commercially available and/or can be prepared by techniques known in the art.

“Micronized” means to reduce to particles that possess an average diameter of less than about 30 microns in diameter, preferably less than about 20 microns, more preferably less than about 10 microns, and still more preferably less than about 5 microns. The average diameter (e.g., volume diameter) of the particles is typically at least about 400 nm, preferably at least about 500 nm, more preferably at least about 700 nm, such as at least about 800 nm or even at least about 1000 nm. An exemplary preferred formulation of the invention includes particles having an average diameter of about 0.8 (or 1.0) to about 5.0 microns, such as about 0.8 (or 1.0) to about 2 microns. A micronized drug form promotes better and more uniform absorption than forms having a larger average size and a wider distribution of particle sizes. In certain embodiments, a large proportion of the particles have diameters within a defined range. For example, 80% of the particles (e.g., DV10 to DV90) have a diameter of from about 0.1 microns to about 20 microns, such as about 0.2 microns to about 10 microns, particularly about 0.2 microns to about 5 microns or even about 0.2 microns to about 2 microns.

Drugs according to the invention can be purchased in micronized form or can be micronized using conventional micronization equipment, such as the Micron-Master line of micronizers available from The Jet Pulverizer Company (Moorestown, N.J.), or processed by a third-party micronization processor such as Micron Technologies (Exton, Pa.).

Many drug manufacturing, milling and micronizing machines pulverize substances into extremely fine particles, and thus reduce bulk chemicals to the required size for pharmaceutical formulation. Particles may also be micronized by chemical or temperature controlled processes. The primary benefit to micronizing is the increase in solubility/bioavailability due to the increase in surface area. These finished chemicals are combined and processed further in mixing machines. The mixed ingredients may then be mechanically capsulated, pressed into tablets, or made into solutions. As used herein, the term “micronizing” may be considered to refer to the processes of making uniform particle size of a drug, wherein the size desired may be 10 microns or less, and wherein said process may be mechanical, chemical, temperature or pH controlled, or any other commonly known process familiar to one of skill in the art.

Optimization and control of micronizing processes, particularly relating to particle size, are becoming ever more important in the development of pharmaceuticals. Air jet micronization is a well proven technique that consistently produces particles in the 1-30 micron range. Micron Technologies and Jet Pharma are contract micronizers. The primary advantages of air jet micronizers are that particle reduction occurs via particle to particle collisions, with limited reduction from metal to product contact, and no generation of heat. Other advantages include no moving parts and easy to clean surfaces.

The original principles of jet milling are simple. The powder particles are fed into the flat cylindrical milling chamber tangentially through a venturi system by pressurized air or nitrogen. The particles are accelerated in a spiral movement inside the milling chamber by a number of nozzles placed around the periphery of the chamber. The micronizing effect takes place by the collision between the incoming particles and those already accelerated into the spiral path. While centrifugal force retains the larger particles at the periphery of the milling chamber, the smaller particles exit with the exhaust air from the centre of the chamber. The particle size distribution is controlled by adjusting a number of parameters, two of the main ones being pressure and feed rate.

In general, there are two types of air jet micronizers or tangential fluid energy mills, pancake and loop. The primary difference between the two is in overall distribution. Loop mills are excellent choices for cleaning up the tails of the distribution. An additional advantage is that both mill types generate no heat. The mills are available in numerous sizes ranging from 1″, 4″, 6″, 8″, 12″, and 15″ to 20″ and provide flexibility in engineering the desired particle size in ranges of 1-30 micons.

U.S. Pat. Nos. 6,645,466, 6,623,760, 6,555,135, hereby incorporated by reference, describe other micronization procedures.

As used herein, particle size of micronized drug dispersions refers to an average particle size as measured by conventional particle size measuring techniques well known to those skilled in the art, such as sedimentation field flow fractionation, photon correlation spectroscopy, disk centrifugation, and dynamic and static light scattering (e.g, laser diffraction such as Mie scattering).

Another possibility to achieve sustained release kinetics is embedding or encapsulating the active compound into nanoparticles. Nanoparticles can be administrated as powder, as a powder mixture with added excipients or as suspensions. Colloidal suspensions of nanoparticles are preferred because they can easily be administrated through a cannula with small diameter.

Nanoparticles are particles with a diameter from about 5 nm to up to about 1000 nm. The term “nanoparticles” as it is used hereinafter refers to particles formed by a polymeric matrix in which the active compound is dispersed, also known as “nanospheres”, and also refers to nanoparticles which are composed of a core containing the active compound which is surrounded by a polymeric membrane, also known as “nanocapsules”. For administration into the vitreous body of the eye nanoparticles are preferred having a diameter from about 50 nm to about 500 nm, in particular from about 100 nm to about 200 nm.

Nanoparticles can be prepared by in situ polymerization of dispersed monomers or by using preformed polymers. Since polymers prepared in situ are often not biodegradable and/or contain toxicological serious byproducts, nanoparticles from preformed polymers are preferred. Nanoparticles from preformed polymers can be prepared by different techniques, e.g., by emulsion evaporation, solvent displacement, salting-out and by emulsification diffusion.

Emulsion evaporation is the classical technique for preparation of nanoparticles from preformed polymers. According to this technique, the polymer and the active compounds are dissolved in a water-immiscible organic solvent, which is emulsified in an aqueous solution. The crude emulsion is then exposed to a high-energy source such as ultrasonic devices or passed through high pressure homogenizers or microfluidizers to reduce the particle size. Subsequently the organic solvent is removed by heat and/or vacuum resulting in formation of the nanoparticles with a diameter of about 100 nm to about 300 nm. Usually, methylene chloride and chloroform are used as organic solvent because of their water insolubility, good solubilizing properties, easy emulsification and high volatility. These solvents are, however, critical in view of their physiological tolerability. Moreover, the high shear force needed for particle size reduction can lead to damage of polymer and/or the active compound.

The solvent displacement process is described in EP 0 274 961 A1. In this process the active compound and the polymer are dissolved in an organic solvent which is miscible with water in all proportions. This solution is introduced in an aqueous solution containing a stabilizer under gentle agitation resulting in spontaneous formation of nanoparticles. Examples for suitable organic solvents and stabilizer are acetone or ethanol. Advantageously chlorinated solvents and shear stress can be avoided. The mechanism of formation of nanoparticles has been explained by interfacial turbulence generated during solvent displacement (Fessi et al., Int. J. Pharm. 55:R1-R4 (1989)). Recently, a solvent displacement technique was disclosed by WO 97/03657 A1, in which the organic solvent containing the active compound and the polymer is introduced into the aqueous solution without agitation.

The salting-out technique is firstly in WO 88/08011A1. In this technique a solution of a water-insoluble polymer and an active compound in a water-miscible organic solvent, such as acetone, is mixed with a concentrated aqueous viscous solution or gel containing a colloidal stabilizer and a salting-out agent. To the resulting oil-in-water emulsion water is added in a quantity sufficient to diffuse into the aqueous phase and to induce rapid diffusion of the organic solvent into the aqueous phase leading to interfacial turbulence and formation of nanoparticles. The organic solvent and the salting-out agent remaining in the suspension of nanoparticles are subsequently eliminated by repeated washing with water. Alternatively, the solvent and salting-out agent can be eliminated by cross-flow filtration.

In emulsification-diffusion process the polymer is dissolved in a water-saturated partially water-soluble organic solvent. This solution is mixed with an aqueous solution containing a stabilizer resulting in an oil-in-water emulsion. To this emulsion water is added causing the solvent to diffuse into the aqueous external phase accompanied with formation of nanoparticles. During particle formation each emulsion droplet leads to several nanoparticles. As this phenomenon cannot be fully explained by convection effect caused by interfacial turbulence, it has been proposed that diffusion of organic solvent from the droplets of the crude emulsion carries molecules of active compound and polymer phase into the aqueous phase resulting in supersaturated local regions, from which the polymer aggregates in the form of nanoparticles (Quintanar-Guerrero et al., Colloid. Polym. Sci. 275:640-647 (1997)). Advantageously, pharmaceutically acceptable solvents like propylene carbonate or ethyl acetate are used as organic solvents.

With the methods described above, nanoparticles can be formed with various types of polymers. For use in the method of the present invention, nanoparticles made from biocompatible polymers are preferred. The term “biocompatible” refers to material that after introduction into a biological environment has no serious effects to the biological environment. From biocompatible polymers those polymers are especially preferred which are also biodegradable. The term “biodegradable” refers to material that after introduction into a biological environment is enzymatically or chemically degraded into smaller molecules, which can be eliminated subsequently. Examples are polyesters from hydroxycarboxylic acids such as poly(lactic acid) (PLA), poly(glycolic acid) (PGA), polycaprolactone (PCL), copolymers of lactic acid and glycolic acid (PLGA), copolymers of lactic acid and caprolactone, polyepsilon caprolactone, polyhyroxy butyric acid and poly(ortho)esters, polyurethanes, polyanhydrides, polyacetals, polydihydropyrans, polycyanoacrylates, natural polymers such as alginate and other polysaccharides including dextran and cellulose, collagen and albumin.

Additional methods of preparing nanoparticles include the steps of dispersing a therapeutic or diagnostic agent in a liquid dispersion medium and applying mechanical means in the presence of grinding media to reduce the particle size of the therapeutic or diagnostic agent to an effective average particle size of less than about 400 nm. The particles can be reduced in size in the presence of a surface modifier. Alternatively, the particles can be contacted with a surface modifier after attrition.

It is preferred, but not essential, that the particle size of the sirtuin modulator selected be less than about 10 mm as determined by sieve analysis. If the coarse particle size is greater than about 100 mm, then it is preferred that the particles be reduced in size to less than 100 mm using a conventional milling method such as airjet or fragmentation milling.

The sirtuin modulator can then be added to a liquid medium in which it is essentially insoluble to form a premix. The concentration of the therapeutic or diagnostic agent in the liquid medium can vary from about 0.1-60%, and preferably is from 5-30% (w/w). It is preferred, but not essential, that the surface modifier be present in the premix. The concentration of the surface modifier can vary from about 0.1 to about 90%, and preferably is 1-75%, more preferably 20-60%, by weight based on the total combined weight of the sirtuin modulator and surface modifier. The apparent viscosity of the premix suspension is preferably less than about 1000 centipoise.

The premix can be used directly by subjecting it to mechanical means to reduce the average particle size in the dispersion to less than 1000 nm. It is preferred that the premix be used directly when a ball mill is used for attrition. Alternatively, the therapeutic or diagnostic agent and, optionally, the surface modifier, can be dispersed in the liquid medium using suitable agitation, e.g., a roller mill or a Cowles type mixer, until a homogeneous dispersion is observed in which there are no large agglomerates visible to the naked eye. It is preferred that the premix be subjected to such a premilling dispersion step when a recirculating media mill is used for attrition. Alternatively, the therapeutic or diagnostic agnet and, optionally, the surface modifier, can be dispersed in the liquid medium using suitable agitiation, e.g., a roller mill or a Cowles type mixer, until a homogeneous dispersion is observed in which there are no large agglomerates visible to the naked eye. It is preferred that the premix be subjected to such a premilling dispersion step when a recirculating media mill is used for attrition.

The mechanical means applied to reduce the particle size of the sirtuin modulator conveniently can take the form of a dispersion mill. Suitable dispersion mills include a ball mill, an attritor mill, a vibratory mill, and media mills such as a sand mill and a bead mill. A media mill is preferred due to the relatively shorter milling time required to provide the intended result, desired reduction in particle size. For media milling, the apparent viscosity of the premix preferably is from about 100 to about 1000 centipoise. For ball milling, the apparent viscosity of the premix preferably is from about 1 to about 100 centipoise. Such ranges tend to afford an optimal balance between efficient particle fragmentation and media erosion.

The attrition time can vary widely and depends primarily upon the particular mechanical means and processing conditions selected. For ball mills, processing times of up to five days or longer may be required. On the other hand, processing times of less than 1 day (residence times of one minute up to several hours) have provided the desired results using a high shear media mill.

The particles must be reduced in size at a temperature which does not significantly degrade the sirtuin modulator. Processing temperatures of less than about 30-40° C. are ordinarily preferred. If desired, the processing equipment can be cooled with conventional cooling equipment. The method is conveniently carried out under conditions of ambient temperature and at processing pressures which are safe and effective for the milling process. For example, ambient processing pressures are typical of ball mills, attritor mills and vibratory mills. Control of the temperature, e.g., by jacketing or immersion of the milling chamber in ice water are contemplated. Processing pressures from about 1 psi (0.07 kg/cm2) up to about 50 psi (3.5 kg/cm2) are contemplated. Processing pressures from about 10 psi (0.7 kg/cm2) to about 20 psi 1.4 kg/cm2)

The surface modifier, if it was not present in the premix, must be added to the dispersion after attrition in an amount as described for the premix above. Thereafter, the dispersion can be mixed, e.g., by shaking vigorously. Optionally, the dispersion can be subjected to a sonication step, e.g., using an ultrasonic power supply. For example, the dispersion can be subjected to ultrasonic energy having a frequency of 20-80 kHz for a time of about 1 to 120 seconds.

After attrition is completed, the grinding media is separated from the milled particulate product (in either a dry or liquid dispersion form) using conventional separation techniques, such as by filtration, sieving through a mesh screen, and the like.

In a particular method, a sirtuin modulator is prepared in the form of submicron particles by grinding the agent in the presence of a grinding media having a mean particle size of less than about 75 microns.

Another method of forming a nanoparticle dispersion is by microprecipitation. This is a method of preparing stable dispersions of sirtuin modulators in the presence of a surface modifying and colloid stability enhancing surface active agent free of any toxic solvents or solubilized heavy metal inpurities by the following procedural steps:

1. Dissolving the therapeutic or diagnostic agent in aqueous base with stirring,

2. Adding above #1 formulation with stirring to a surface active surfactant (or surface modifiers) solution to form a clear solution, and

3. Neutralizing above formulation #2 with stirring with an appropriate acid solution.

The procedure can be followed by:

4. Removal of formed salt by dialysis or diafiltration and

5. Concentration of dispersion by conventional means.

This microprecipitation process produces a dispersion of a sirtuin activator with Z-average particle diameter less than 400 nm (as measured by photon correlation spectroscopy) that is stable in particle size upon keeping under room temperature or refrigerated conditions. Such dispersions also demonstrate limited particle size growth upon autoclave-decontamination conditions used for standard blood-pool pharmaceutical agents.

In one embodiment, the above procedure is followed with step 4 which comprises removing the formed salts by diafiltration or dialysis. This is done in the case of dialysis by standard dialysis equipment and by diafiltration using standard diafiltration equipment known in the art. Preferably, the final step is concentration to a desired concentration of the agent dispersion. This is done either by diafiltration or evaporation using standard equipment known in this art.

In another embodiment of the microprecipitation process, a crystal growth modifier is used. A crystal growth modifier is defined as a compound that in the co-precipitation process incorporates into the crystal structure of the microprecipitated crystals of the pharmaceutical agent, thereby hindering growth or enlargement of the microcrystalline precipitate, by the so called Ostwald ripening process. A crystal growth modifier (or a CGM) is a chemical that is at least 75% identical in chemical structure to the pharmaceutical agent. By “identical” is meant that the structures are identical atom for atom and their connectivity. Structural identity is charactarized as having 75% of the chemical structure, on a molecular weight basis, identical to the therapeutic or diagnostic agent. The remaining 25% of the structure may be absent or replaced by different chemical structure in the CGM. The crystal growth modifier is dissolved in step #1 above with the therapeutic or diagnostic agent.

Suitable surface modifiers can preferably be selected from known organic and inorganic pharmaceutical excipients. Such excipients include various polymers, low molecular weight oligomers, natural products and surfactants. Preferred surface modifiers include nonionic and ionic surfactants. Representative examples of surface modifiers include gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, e.g., macrogol ethers such as cetomacrogol 1000, polyoxyethylene castor oil derivatives, polyoxyethylene sorbitan fatty acid esters, e.g., the commercially available Tweens™, polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxy propylcellulose, hydroxypropylmethylcellulose phthalate, noncrystalline cellulose, magnesium aluminum silicate, triethanolamine, polyvinyl alcohol, and polyvinylpyrrolidone (PVP). Most of these surface modifiers are known pharmaceutical excipients and are described in detail in the Handbook of Pharmaceutical Excipients, published jointly by the American Pharmaceutical Association and The Pharmaceutical Society of Great Britain, the Pharmaceutical Press, 1986.

Particular surface modifiers include polyvinylpyrrolidone, tyloxapol, poloxamers such as Pluronics™ F68 and F108, which are block copolymers of ethylene oxide and propylene oxide, and polyxamines such as Tetronics™ 908 (also known as Poloxamine 908), which is a tetrafunctional block copolymer derived from sequential addition of propylene oxide and ethylene oxide to ethylenediamine, available from BASF, dextran, lecithin, dialkylesters of sodium sulfosuccinic acid, such as Aerosol OTs™, which is a dioctyl ester of sodium sulfosuccinic acid, available from American Cyanimid, Duponols™ P, which is a sodium lauryl sulfate, available from DuPont, Tritons™ X-200, which is an alkyl aryl polyether sulfonate, available from Rohn and Haas, Tween™ 20 and Tweens™ 80, which are polyoxyethylene sorbitan fatty acid esters, available from ICI Specialty Chemicals; Carbowaxs™ 3550 and 934, which are polyethylene glycols available from Union Carbide; Crodestas™ F-110, which is a mixture of sucrose stearate and sucrose distearate, available from Croda Inc., Crodestas™ SL-40, which is available from Croda, Inc., and SA9OHCO, which is C₁₈H₃₇CH₂(CON(CH₃)CH₂(CHOH)₄(CH₂OH)₂. Surface modifiers which have been found to be particularly useful include Tetronic™ 908, the Tweens™, Pluronics™ F-68 and polyvinylpyrrolidone. Other useful surface modifiers include: decanoyl-N-methylglucamide; n-decyl-beta-D-glucopyranoside; n-decyl-beta-D-maltopyranoside; n-dodecyl-beta-D-glucopyranoside; n-dodecyl-beta-D-maltoside; heptanoyl-N-methylglucamide; n-heptyl-beta-D-glucopyranoside; n-heptyl-beta-D-thioglucoside; n-hexyl-beta-D-glucopyranoside; nonanoyl-N-methylglucamide; n-noyl-beta-D-glucopyranoside; octanoyl-N-methylglucamide; n-octyl-beta-D-glucopyranoside; octyl beta-D-thioglucopyranoside; and the like.

Another useful surface modifier is tyloxapol (a nonionic liquid polymer of the alkyl aryl polyether alcohol type; also known as superinone or triton). Another surface modifier is p-isononylphenoxypoly(glycidol) also known as Olin-1OG™ or Surfactant 10-G, commercially available as 1OG™ from Olin Chemicals, Stamford, Conn.

Two-or more surface modifiers can be used in combination.

Auxiliary surface modifiers can be used to impart resistance to particle aggregation during sterilization and include dioctylsulfosuccinate (DOSS), polyethylene glycol, glycerol, sodium dodecyl sulfate, dodecyl trimethyl ammonium bromide and a charged phospholipid such as dimyristoyl phophatidyl glycerol. Two or more auxiliary surface modifiers can be used in combination.

Further description on preparing nanoparticles can be found, for example, in U.S. Pat. No. 6,264,922, the contents of which are incorporated herein by reference.

Liposomes are a further drug delivery system which is easily injectable. Accordingly, in the method of invention the active compounds can also be administered into the vitreous body of the eye in the form of a liposome delivery system. Liposomes are well-known by a person skilled in the art. Liposomes can be formed from a variety of phospholipids, such as cholesterol, stearylamine of phosphatidylcholines. Liposomes being usable for the method of invention encompass all types of liposomes including, but not limited to, small unilamellar vesicles, large unilamellar vesicles and multilamellar vesicles.

Liposomes are used for a variety of therapeutic purposes, and in particular, for carrying therapeutic agents to target cells. Advantageously, liposome-drug formulations offer the potential of improved drug-delivery properties, which include, for example, controlled drug release. An extended circulation time is often needed for liposomes to reach a target region, cell or site. In particular, this is necessary where the target region, cell or site is not located near the site of administration. For example, when liposomes are administered systemically, it is desirable to coat the liposomes with a hydrophilic agent, for example, a coating of hydrophilic polymer chains such as polyethylene glycol (PEG) to extend the blood circulation lifetime of the liposomes. Such surface-modified liposomes are commonly referred to as “long circulating” or “sterically stabilized” liposomes.

One surface modification to a liposome is the attachment of PEG chains, typically having a molecular weight from about 1000 daltons (Da) to about 5000 Da, and to about 5 mole percent (%) of the lipids making up the liposomes (see, for example, Stealth Liposomes, CRC Press, Lasic, D. and Martin, F., eds., Boca Raton, Fla., (1995)), and the cited references therein. The pharmacokinetics exhibited by such liposomes are characterized by a dose-independent reduction in uptake of liposomes by the liver and spleen via the mononuclear phagocyte system (MPS), and significantly prolonged blood circulation time, as compared to non-surface-modified liposomes, which tend to be rapidly removed from the blood and accumulated in the liver and spleen.

The PEG moiety can have a molecular weight of, for example, 750-20,000 Daltons, such as 1000-10,000 Daltons, in particular 2000-5000 Daltons. In one embodiment, the complex may comprise more than one type of PEG moiety (for example, PEG molecular weight 5K and PEG molecular weight 2K). The PEG moiety may further comprise a suitable functional group, such as, for example, methoxy, N-hydroxyl succinimide (NHS), carbodiimide, etc., for ease of conjugating PEG to the lipid or to the targeting factor. Table 2 of Harasym et al. Advanced Drug Delivery Reviews 32:99-118 (1998) provides examples of suitable functional groups. Functionalized PEG moieties can be purchased from, for example, Shearwater Polymer Inc. (Huntsville, Ala.) and Avanti Polar Lipid Inc. (Alabaster, Ala.). In an exemplary embodiment, the PEG moiety is N-[methoxy(polyethylene glycol)-5K] (PEG_{5K}). Other types of hydrophilic polymers may be substituted for the PEG moiety, including, for example, poloxamer and poloxamine, as described in Feldman et al. (1997) Gene Therapy 4(3):189-198; Lemieux et al. (2000) Gene Therapy 7(11):986-91; Moghimi et al. (2000) Trends In Biotechnology 18:412-420; Torchilin (1998) Journal of Microencapsulation 15(1): 1-19; and Claesson et al. (1996) Colloids & Surfaces A-Physicochemical & Engineering Aspects 112(2):-3, 131-139.

The PEG moiety may be conjugated to a suitable lipid to form a “pegylated lipid”. Preferably, the PEG moiety is covalently attached to the lipid. Suitable lipids include dioleoylphosphatidyl-ethanolamine (DOPE), cholesterol, and ceramides. Lipids comprising a polar end (such as, e.g., phosphatidylethanolamines, including DOPE, DPPE and DSPE), which may be utilized for conjugating to PEG, are preferred for ease of synthesis of pegylated lipids. See Harasym et al., Advanced Drug Delivery Reviews 32:99-118 (1998) for non-limiting examples of suitable functionalized lipids. In a particular embodiment, the lipid is 1,2-distearoyl-sn-glycero-3-phosphotidylethanolamine (DSPE) or dimyristoyl phophatidylethanolamine (DMPE). In a particular embodiment, the pegylated lipid comprises 1,2-distearoyl-sn-glycero-3-phosphatidylethanolamine-N-[methoxy(polyethylene glycol)-5K] (DSPE-PEG_{5K}) or dimyristoyl phosphatidylethanolamine-N-[methoxy(polyethylene glycol)-5K] (DSPE-PEG_{5K}).

The PEG moiety can be conjugated to the lipid by methods known in the art. See, for example, Woodle (1998) Adv. Drug Delivery Reviews 32:139-152 and references cited therein; Haselgruber et al. (1995) Bioconjug Chem 6:242-248; Shahinian et al. (1995) Biochim Biophys Acta 1239:157-167; Zalipsky et al. (1994) FEBS Lett. 353:71-74; Zalipsky et al. (1997) Bioconjug Chem. 8(2):111-118; Zalipsky et al. (1995) Bioconjug Chem. 6:705-708; Hansen et al. (1995) Biochim Biophys Acta. 1239(2):133-44; Allen et al. (1995) Biochim Biophys Acta 1237(2):99-108; Zalipsky (1995) Bioconjug Chem 6(2): 150-65; Zalipsky (1993) Bioconjug Chem 4(4): 296-9; and Zalipsky (1995) in Stealth Liposomes. (Eds: Lasic, D., et al.) CRC Press, Boca Raton, Fla., p. 93-102. Pegylated lipids are also available commercially from, for example, Shearwater Polymer Inc. (Huntsville, Ala.).

It is to be understood that compounds other than lipids, such as, for example, peptides, hydrophobic anchors or polymers, carbohydrates, metals or other ions can be used for conjugating with PEG, provided the compounds anchor PEG to the lipid complex, and allow PEG to be displayed on the surface of the lipid complex.

While not wishing to be bound by theory, the charge shielding effect provided by PEG may enhance the circulatory half-life of the complexes. Shielding may also increase the resistance (decrease the sensitivity) of nucleic acid to degradation, for example by nucleases or other species present in vitro or in vivo (e.g., hyuralonic acid, poly(Asp)) and/or decrease or prevent interactions between individual complex particles or interactions with other species present in vitro or in vivo that may lead to increased complex particle size or aggregation of complex particles. Accordingly, in a preferred embodiment, the complex comprises a neutral surface. In another preferred embodiment, the complex is charge shielded.

In certain embodiments, the complex is shielded to increase the circulatory half-life of the complex or shielded to increase the resistance of nucleic acid to degradation, for example degradation by nucleases.

As used herein, the term “shielding”, and its cognates such as “shielded”, refers to the ability of “shielding moieties” to reduce the non-specific interaction of the complexes described herein with serum complement or with other species present in serum in vitro or in vivo. Shielding moieties may decrease the complex interaction with or binding to these species through one or more mechanisms, including, for example, non-specific steric or non-specific electronic interactions. Examples of such interactions include non-specific electrostatic interactions, charge interactions, Van der Waals interactions, steric-hindrance and the like. For a moiety to act as a shielding moiety, the mechanism or mechanisms by which it may reduce interaction with, association with or binding to the serum complement or other species does not have to be identified. One can determine whether a moiety can act as a shielding moiety by determining whether or to what extent a complex binds serum species.

Other moieties that will act as shielding moieties can be identified by their ability to block binding of serum complement or the serum complement pathway, such as the C3A or C5 proteins of the complement pathway. If a moiety is not recognized by (e.g., does not bind) at least one of the components of serum complement or the serum complement pathway, then the moiety likely acts as a shielding moiety. In particular examples, if a moiety does not bind to or interact with at least one of the C3A or C5 proteins, then the moiety likely is not bound by or does not interact with serum complement.

Incorporation of a moiety which does not bind, associate with, or interact with serum complement or other serum species on the surface of the complexes described herein results in the shielding of the complex. In other words, the components (e.g., lipids) of the complex that would be recognized by or would interact with components of serum are instead shielded from the serum components (e.g., serum proteins, for example, albumin, serum complement, hormones, vitamins, co-factors and others) and therefore are not accessible to serum components and thus are not bound by, associated with, or interacting with these components, including serum complement. The complex therefore can be described as “shielded”. A moiety capable of providing shielding can be termed a “shielding moiety”.

Shielding, as described above, can also be measured by the level of complement opsonization, as described herein. In particular embodiments, the shielding moiety will reduce complement opsonization by approximately 30%, approximately 40%, approximately 50%, approximately 60%, approximately 65%, approximately 70%, approximately 75%, or approximately 80%. In other embodiments, the shielding moiety will reduce complement opsonization by at least 40%, at least 50%, at least 55% or at least 60%.

It should be noted that “shielding moieties” can be multifunctional. For example, a shielding moiety may also function as, for example, a targeting factor. A shielding moiety may also be referred to as multifunctional with respect to the mechanism(s) by which it shields the complex. While not wishing to be limited by proposed mechanism or theory, examples of such a multifunctional shielding moiety are pH sensitive endosomal membrane-disruptive synthetic polymers, such as PPAA or PEAA. Certain poly(alkylacrylic acids) have been shown to disrupt endosomal membranes while leaving the outer cell surface membrane intact (Stayton et al. (2000) J. Controll. Release 65:203-220; Murthy et al. (1999) J. Controll. Release 61:137-143; WO 99/34831), thereby increasing cellular bioavailability and functioning as a targeting factor. However, PPAA reduces binding of serum complement to complexes in which it is incorporated, thus functioning as a shielding moiety.

As will be understood by those of skill in the art, it is important that incorporation of a shielding moiety does not eliminate the complex's ability to be delivered to cells. Therefore, in some embodiments, complexes incorporating a shielding moiety will further comprise a targeting factor. For example, a complex may comprise a cell surface receptor ligand (e.g., folate, an RGD peptide, an LHRH peptide, etc.) that may, for example, be conjugated to a lipid or pegylated lipid and optionally also incorporate PPAA. In certain embodiments, the lipid-targeting factor conjugate is DSPE-PEG_{5K}-RGD or DSPE-PEG_{5K}-Folate.

The amount or ratio of shielding moiety incorporated in a complex formulation can be limited, so as not to eliminate the complex's delivery to cells. Thus in particular examples, the complexes comprise less than about 15%, less than about 12%, less than about 10%, less than about 8%, less than about 7%, less than about 5%, less than about 4%, less than about 3%, or less than about 2% shielding moiety. In particular embodiments, the amount of shielding moiety is about 10%, about 8%, about 5% or about 2%. A complex may also incorporate more than one shielding moiety. In certain embodiments, the amount of shielding moiety is at least 2%, at least 5% or at least 8% or at least 10%.

In certain embodiments, the shielding moiety may be conjugated to another component of the complex, for example a lipid or pegylated lipid. In certain examples, the shielding moiety may be conjugated to a co-lipid or pegylated co-lipid. In other embodiments, the shielding moiety is not conjugated to any other component of the complex.

In particular embodiments, the complex is shielded by incorporation of compounds comprising polyethylene glycol moieties (PEG) or by the incorporation of synthetic polymers. In particular examples of the complexes described herein, the shielded complex may comprise one or more synthetic polymers, including for example, membrane disruptive synthetic polymers, pH sensitive membrane-disruptive synthetic polymers, pH sensitive endosomal membrane-disruptive synthetic polymers, or poly(alkylacrylic acid) polymers. Particular examples of membrane disruptive polymers include poly(alkylacrylic acid) polymers such as poly(ethyl acrylic acid) (PEAA) and poly(propyl acrylic acid) (PPAA).

It is also possible that shielding the complexes may reduce the toxicity of the complexes.

The pegylated lipid and/or targeting factor-pegylated lipid conjugate and/or targeting factor-lipid conjugate may comprise, for example, from about 0.01 to about 30 mol percent of the total lipids, more preferably, from about 1 to about 30 mol percent of the total lipids. The pegylated lipid and/or targeting factor-pegylated lipid conjugate and/or targeting factor-lipid conjugate may comprise, for example, from about 1 to about 20 mol percent, from about 1 to about 10 mol percent of the total lipids, from about 2 to about 5 mol percent, about 1 mol percent, about 2 mol percent, about 3 mol percent, about 4 mol percent, about 5 mol percent, about 10 mol percent, about 15 mol percent or about 20 mol percent of the total lipids. The complex may comprise a pegylated lipid without conjugated targeting factor as well as a targeting factor-pegylated lipid conjugate. The complex may also comprise a targeting factor-pegylated lipid conjugate and a targeting factor-lipid conjugate. The complex may comprise more than one targeting factor-pegylated lipid conjugate or targeting factor-lipid conjugate. The PEG moiety may be the same or different when more than one pegylated lipid is present in the complex. In one non-limiting example, the targeting factor-pegylated lipid conjugate may comprise PEG of 5 KDa molecular weight, and the pegylated lipid without conjugated targeting factor may comprise PEG of 750 Da -2 KDa molecular weight. The complex may also comprise a pegylated lipid and a targeting factor conjugated to a lipid. In one embodiment, the complex comprises a targeting factor-pegylated lipid conjugate and a targeting factor-lipid conjugate. Alternatively, in other embodiments, the complex comprises a targeting factor that is not conjugated to lipid or pegylated lipid, and comprises a pegylated lipid.

Another way to produce a formulation, particularly a solution, of a sirtuin modulator such as resveratrol or a derivative thereof, is through the use of cyclodextrin. By cyclodextrin is meant α-, β-, or γ-cyclodextrin. Cyclodextrins are described in detail in Pitha et al., U.S. Pat. No. 4,727,064, which is incorporated herein by reference. Cyclodextrins are cyclic oligomers of glucose; these compounds form inclusion complexes with any drug whose molecule can fit into the lipophile-seeking cavities of the cyclodextrin molecule.

By amorphous cyclodextrin is meant non-crystalline mixtures of cyclodextrins wherein the mixture is prepared from α-, β-, or γ-cyclodextrin. In general the amorphous cyclodextrin is prepared by non-selective additions, especially alkylation of the desired cyclodextrin species. Reactions are carried out to yield mixtures containing a plurality of components thereby preventing crystallization of the cyclodextrin. Various alkylated and hydroxyalkyl-cyclodextrins can be made and of course will vary, depending upon the starting species of cyclodextrin and the addition agent used. Among the amorphous cyclodextrins suitable for compositions according to the invention are hydroxypropyl, hydroxyethyl, glucosyl, maltosyl and maltotriosyl derivatives of β-cyclodextrin, carboxyamidomethyl-β-cyclodextrin, carboxymethyl-β-cyclodextrin, hydroxypropyl-β-cyclodextrin and diethylamino-β-cyclodextrin. The substituted γ-cyclodextrins may also be suitable, including hydroxypropyl, hydroxyethyl, glucosyl, maltosyl and maltotriosyl derivatives of γ-cyclodextrin.

The cyclodextrin of the compositions according to the invention may be α-, β-, or γ-cyclodextrin. α-cyclodextrin contains six glucopyranose units; γ-cyclodextrin contains seven glucopyranose units; and γ-cyclodextrin contains eight glucopyranose units. The molecule is believed to form a truncated cone having a core opening of 4.7-5.3 angstroms, 6.0-6.5 angstroms, and 7.5-8.3 angstroms in α-, β-, or γ-cyclodextrin respectively. The composition according to the invention may comprise a mixture of two or more of the α-, β-, or γ-cyclodextrins. Typically, however, the composition according to the invention will comprise only one of the α-, β-, or γ-cyclodextrins.

The unmodified α-, β-, or γ-cyclodextrins are less preferred in the compositions according to the invention because the unmodified forms tend to crystallize and are relatively less soluble in aqueous solutions. More preferred for the compositions according to the invention are the α-, β-, and γ-cyclodextrins that are chemically modified or substituted. Chemical substitution at the 2, 3 and 6 hydroxyl groups of the glucopyranose units of the cyclodextrin rings yields increases in solubility of the cyclodextrin compound.

Most preferred cyclodextrins in the compositions according to the invention are amorphous cyclodextrin compounds. By amorphous cyclodextrin is meant non-crystalline mixtures of cyclodextrins wherein the mixture is prepared from α-, β-, or γ-cyclodextrin. In general, the amorphous cyclodextrin is prepared by non-selective alkylation of the desired cyclodextrin species. Suitable alkylation agents for this purpose include but are not limited to propylene oxide, glycidol, iodoacetamide, chloroacetate, and 2-diethylaminoethlychloride. Reactions are carried out to yield mixtures containing a plurality of components thereby preventing crystallization of the cyclodextrin. Various alkylated cyclodextrins can be made and of course will vary, depending upon the starting species of cyclodextrin and the alkylating agent used. Among the amorphous cyclodextrins suitable for compositions according to the invention are hydroxypropyl, hydroxyethyl, glucosyl, maltosyl and maltotriosyl derivatives of β-cyclodextrin, carboxyamidomethyl-β-cyclodextrin, carboxymethyl-β-cyclodextrin, hydroxypropyl-β-cyclodextrin and diethylamino-β-cyclodextrin.

One example of resveratrol dissolved in the presence of a cyclodextrin is provided in Marier et al., J. Pharmacol. Exp. Therap. 302:369-373 (2002), the contents of which are incorporated herein by reference, where a 6 mg/mL solution of resveratrol was prepared using 0.9% saline containing 20% hydroxylpropyl-β-cyclodextrin.

As mentioned above, certain compositions of matter of the invention comprise an aqueous preparation of preferably substituted amorphous cyclodextrin and one or more sirtuin modulators. The relative amounts of sirtuin modulators and cyclodextrin will vary depending upon the relative amount of each of the sirtuin modulators and the effect of the cyclodextrin on the compound. In general, the ratio of the weight of compound of the sirtuin modulators to the weight of cyclodextrin compound will be in a range between 1:1 and 1:100. A weight to weight ratio in a range of 1:5 to 1:50 and more preferably in a range of 1:10 to 1:20 of the compound selected from sirtuin modulators to cyclodextrin are believed to be the most effective for increased circulating availability of the sirtuin modulator.

Preferably, cyclodextrin formulations of a sirtuin modulator (e.g., resveratrol) have at least a 30 mM concentration of the sirtuin modulator, more preferably at least 50 mM. Certain cyclodextrin formulations of the invention contain at least 100 mM, such as at least 125 mM, 150 mM or even 200 mM sirtuin modulator.

Importantly, if the aqueous solution comprising the sirtuin modulators and a cyclodextrin is to be administered parenterally, especially via the intravenous route, a cyclodextrin will be substantially free of pyrogenic contaminants. Various forms of cyclodextrin, such as forms of amorphous cyclodextrin, may be purchased from a number of vendors including Sigma-Aldrich, Inc. (St. Louis, Mo., USA). A method for the production of hydroxypropyl-β-cyclodextrin is disclosed in Pitha et al., U.S. Pat. No. 4,727,064 which is incorporated herein by reference.

To produce the formulations according to the invention, a pre-weighed amount of a cyclodextrin compound, which is substantially pyrogen free is placed in a suitable depyrogenated sterile container. Methods for depyrogenation of containers and closure components are well known to those skilled in the art and are fully described in the United States Pharmacopeia 23 (United States Pharmacopeial Convention, Rockville, Md. USA). Generally, depyrogenation is accomplished by exposing the objects to be depyrogenated to temperatures above 400 degree C. for a period of time sufficient to fully incinerate any organic matter. As measured in U.S.P. Bacterial Endotoxin Units, the formulation will contain no more than 10 Bacterial Endotoxin Units per gram of amorphous cyclodextrin. By substantially pyrogen free is meant that the cyclodextrin contains less than 10 U.S.P. bacterial endotoxin units per gram using the U.S.P. method. Preferably, the cyclodextrin will contain between 0.1 and 5 U.S.P. bacterial endotoxin units per mg, under conditions specified in the United States Pharmacopeia 23.

Sufficient sterile water for injection is added to the substantially pyrogen free amorphous cyclodextrin until the desired concentration of the cyclodextrin is in solution. To this solution a pre-weighed amount of the compound selected from the sirtuin modulators, such as resveratrol, is added with agitation and with additional standing if necessary until it dissolves.

The solution is then filtered through a sterile 0.22 micron filter into a sterile holding vessel and is subsequently filled in sterile depyrogenated vials and is capped. For products that will be stored for long periods of time, a pharmaceutically acceptable preservative may be added to the solution of sirtuin modulator and cyclodextrin prior to filtration, filling and capping or alternatively, may be added sterilely after filtration.

As discussed above, the present invention provides improved water soluble formulations of sirtuin modulators and methods of preparing and employing such formulations. The advantages of these water soluble formulations are that a drug is entrapped in cyclodextrin in dissolved form. These compositions have been observed to provide a very low toxicity form of the pharmacologically active agent that can be delivered in the form by slow infusions or by bolus injection or by other parenteral or oral delivery routes.

Additional description of the use of cyclodextrin for solubilizing compounds can be found in US 2005/0026849, the contents of which are incorporated herein by reference.

Regardless of the route of administration selected, the compounds of the present invention, which may be used in a suitable hydrated form, and/or the pharmaceutical compositions of the present invention, are formulated into pharmaceutically acceptable dosage forms such as described below or by other conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, the route of administration, the time of administration, the rate of excretion of the particular compound being employed, the duration of the treatment, other drugs, compounds and/or materials used in combination with the particular composition employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect, and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, doses (e.g., intravenous, subcutaneous, oral, intraocular) of the compounds of this invention for a patient will range from about 0.0001 to about 100 mg per kilogram of body weight per day. In certain embodiments, doses of the compounds are more than 100 mg per kg of body weight per day. Doses can also be selected to have the biological effect of a particular dose of resveratrol, such as equal to or greater than 18, 20, 25, 30, 35, 40, 50, 60, 75, 100, 150 mg/kg, or more.

If desired, the effective daily dose of the active compound may be administered as two, three, four, five, six or more sub-doses administered separately at appropriate intervals throughout the day, optionally, in unit dosage forms. In certain embodiments, it is preferred that the sirtuin modulator is delivered in a single daily dose.

The term “treatment” is intended to encompass also prophylaxis, therapy and cure. The patient receiving this treatment is any animal in need, including primates, in particular humans, and other mammals such as equines, cattle, swine and sheep; and poultry and pets in general.

A compound of the invention (i.e., sirtuin modulator) can be administered as such or in admixtures with pharmaceutically acceptable and/or sterile carriers and can also be administered in conjunction with other therapeutic agents. Conjoint therapy includes sequential, simultaneous, and separate administration of the active compound in a way that the therapeutical effects of the first administered one is not entirely dissipated when the subsequent is administered.

A sirtuin modulator can be administered in conjunction with a therapy for reducing intraocular pressure. One group of therapies involves blocking aqueous production. For example, topical beta-adrenergic antagonists (timolol and betaxolol) decrease aqueous humor production. Topical timolol causes IOP to fall in 30 minutes with peak effects in 1-2 hours. A reasonable regimen is Timoptic 0.5%, one drop every 30 minutes for 2 doses. The carbonic anhydrase inhibitor, acetazolamide, also decreases aqueous humor production and should be given in conjunction with topical beta-antagonists. An initial dose of 500 mg is administered followed by 250 mg every 6 hours. This medication may be given orally, intramuscularly, or intravenously. In addition, alpha 2-agonists (e.g., Apraclonidine) act by decreasing aqueous humor production. Their effects are additive to topically administered beta-blockers. They have been approved for use in controlling an acute rise in pressure following anterior chamber laser procedures, but has been reported effective in treating acute closed-angle glaucoma. A reasonable regimen is 1 drop every 30 minutes for 2 doses.

A second group of therapies for reducing intraocular pressure involve reducing vitreous volume. Hyperosmotic agents can be used to treat an acute attack. These agents draw water out of the globe by making the blood hyperosmolar. Oral glycerol in a dose of 1 mL/kg in a cold 50% solution (mixed with lemon juice to make it more palatable) often is used. Glycerol is converted to glucose in the liver; persons with diabetes may need additional insulin if they become hyperglycemic after receiving glycerol. Oral isosorbide is a metabolically inert alcohol that also can be used as an osmotic agent for patients with acute angle-closure glaucoma. Usual dose is 100 g taken p.o. (220 cc of a 45% solution). This inert alcohol should not be confused with isosorbide dinitrate, a nitrate-based cardiac medication used for angina and for congestive heart failure. Intravenous mannitol in a dose of 1.0-1.5 mg/kg also is effective and is well tolerated in patients with nausea and vomiting. These hyperosmotic agents should be used with caution in any patient with a history of congestive heart failure.

A third group of therapies involve facilitating aqueous outflow from the eye. Miotic agents pull the iris from the iridocorneal angle and may help to relieve the obstruction of the trabecular meshwork by the peripheral iris. Pilocarpine 2% (blue eyes)-4% (brown eyes) can be administered every 15 minutes for the first 1-2 hours. More frequent administration or higher doses may precipitate a systemic cholinergic crisis. NSAIDS are sometimes used to reduce inflammation.

Exemplary therapeutic agents for reducing intraocular pressure include ALPHAGAN® P (Allergan) (brimonidine tartrate ophthalmic solution), AZOPT® (Alcon) (brinzolamide ophthalmic suspension), BETAGAN® (Allergan) (levobunolol hydrochloride ophthalmic solution, USP), BETIMOL® (Vistakon) (timolol ophthalmic solution), BETOPTIC S® (Alcon) (betaxolol HCl), BRIMONIDINE TARTRATE (Bausch & Lomb), CARTEOLOL HYDROCHLORIDE (Bausch & Lomb), COSOPT® (Merck) (dorzolamide hydrochloride-timolol maleate ophthalmic solution), LUMIGAN® (Allergan) (bimatoprost ophthalmic solution), OPTIPRANOLOL® (Bausch & Lomb) (metipranolol ophthalmic solution), TIMOLOL GFS (Falcon) (timolol maleate ophthalmic gel forming solution), TIMOPTIC® (Merck) (timolol maleate ophthalmic solution), TRAVATAN® (Alcon) (travoprost ophthalmic solution), TRUSOPT® (Merck) (dorzolamide hydrochloride ophthalmic solution) and XALATAN® (Pharmacia & Upjohn) (latanoprost ophthalmic solution).

Drugs currently marketed for glaucoma can be used in combination with sirtuin modulators. An example of a glaucoma drug is DARANIDE®V Tablets (Merck) (Dichlorphenamide).

Drugs currently marketed for optic neuritis can be used in combination with sirtuin modulators. Examples of drugs for optic neuritis include DECADRON® Phosphate Injection (Merck) (Dexamethasone Sodium Phosphate), DEPO-MEDROL® (Pharmacia & Upjohn)(methylprednisolone acetate), HYDROCORTONE® Tablets (Merck) (Hydrocortisone), ORAPRED® (Biomarin) (prednisolone sodium phosphate oral solution) and PEDIAPRED® (Celltech) (prednisolone sodium phosphate, USP).

Drugs currently marketed for CMV Retinopathy can be used in combination with sirtuin modulators. Treatments for CMV retinopathy include CYTOVENE® (ganciclovir capsules) and VALCYTE® (Roche Laboratories) (valganciclovir hydrochloride tablets).

Drugs currently marketed for multiple sclerosis can be used in combination with sirtuin modulators. Examples of such drugs include DANTRIUM® (Procter & Gamble Pharmaceuticals) (dantrolene sodium), NOVANTRONE® (Serono) (mitoxantrone), AVONEX® (Biogen Idec) (Interferon beta-1a), BETASERON® (Berlex) (Interferon beta-1b), COPAXONE® (Teva Neuroscience) (glatiramer acetate injection) and REBIF® (Pfizer) (interferon beta-1a).

In addition, macrolide and/or mycophenolic acid, which has multiple activities, can be co-administered with a sirtuin modulator. Macrolide antibiotics include tacrolimus, cyclosporine, sirolimus, everolimus, ascomycin, erythromycin, azithromycin, clarithromycin, clindamycin, lincomycin, dirithromycin, josamycin, spiramycin, diacetyl-midecamycin, tylosin, roxithromycin, ABT-773, telithromycin, leucomycins, and lincosamide.

The phrase “therapeutically effective amount” as used herein means that amount of a compound, material, or composition comprising a compound of the present invention which is effective for producing some otoprotection, at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its analogs, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, the pharmaceutical preparation is non-pyrogenic, i.e., does not substantially elevate the body temperature of a patient.

As set out above, certain embodiments of the present composition may contain a basic functional group, such as amino or alkylamino, and are, thus, capable of forming pharmaceutically acceptable salts with pharmaceutically acceptable acids. The term “pharmaceutically acceptable salts” in this respect refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19)

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Pharmacological dosages or formulations of the present invention include those suitable for oral, nasal, topical (including buccal and sublingual), rectal, vaginal and/or parenteral administration. The dosages may conveniently be presented in unit dosage form and may be prepared by any methods well known in the art of pharmacy. The amount of active ingredient that can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such as talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Powders comprising micronized drug can be made by spray-drying aqueous dispersions of a micronized drug to form a dry powder which consists of aggregated drug particles. Alternatively, the aqueous dispersion of drug can contain a dissolved diluent, such as lactose or mannitol, which when spray dried forms diluent particles, each of which contains at least one embedded drug particle.

Micronized drug dispersions can also be freeze-dried to obtain powders suitable for formulation into solid dose forms. Such powders comprise aggregated micronized drug particles. Freeze dried powders can also be obtained by freeze drying aqueous dispersions of drug, which additionally contain a dissolved diluent such as lactose or mannitol. In these instances the freeze dried powders consist of particles of diluent, each of which contains at least one embedded drug particle.

Other known methods of processing liquid dispersions, and which can be employed in the present invention, include granulation, including but not limited to high shear granulation, fluid bed granulation, roto granulation, and melt granulation. Additional methods such as spray coating and extrusion spherization can also be used. Any other conventional method for drying or otherwise processing a liquid dispersion can also be used in the invention.

Rapidly disintegrating or dissolving dosage forms are useful for the rapid absorption, particularly buccal and sublingual absorption, of pharmaceutically active agents. Fast melt dosage forms are beneficial to patients, such as aged and pediatric patients, who have difficulty in swallowing typical solid dosage forms, such as caplets and tablets. Additionally, fast melt dosage forms circumvent drawbacks associated with, for example, chewable dosage forms, wherein the length of time an active agent remains in a patient's mouth plays an important role in determining the amount of taste masking and the extent to which a patient may object to throat grittiness of the active agent.

To overcome such problems manufacturers have developed a number of fast melt solid dose oral formulations. These are available from manufacturers including Cima Labs, Fuisz Technologies Ltd., Prographarm, R. P. Scherer, Yamanouchi-Shaklee, and McNeil-PPC, Inc. All of these manufacturers market different types of rapidly dissolving solid oral dosage forms.

Cima Labs markets OraSolv™, which is an effervescent direct compression tablet having an oral dissolution time of five to thirty seconds, and DuraSolv™, which is a direct compression tablet having a taste-masked active agent and an oral dissolution time of 15 to 45 seconds. Cima's U.S. Pat. No. 5,607,697, for “Taste Masking Microparticles for Oral Dosage Forms,” the contents of which are incorporated herein by reference, describes a solid dosage form consisting of coated microparticles that disintegrate in the mouth. The microparticle core of Cima's patented oral dosage form has a pharmaceutical agent and one or more sweet-tasting compounds having a negative heat of solution wherein the sweet-tasting compound can be mannitol, sorbitol, a mixture of an artificial sweetener and menthol, a mixture of sugar and menthol, or methyl salicylate. The microparticle core is coated, at least partially, with a material that retards dissolution in the mouth and masks the taste of the pharmaceutical agent. The microparticles are then compressed to form a tablet. Cima's patent discloses that other excipients can also be added to the tablet formulation.

WO 98/46215 for “Rapidly Dissolving Robust Dosage Form,” the contents of which are incorporated herein by reference, is directed to a hard, compressed, fast melt formulation having an active ingredient and a matrix of at least a non-direct compression filler and lubricant. A non-direct compression filler is typically not free-flowing, in contrast to a direct compression (DC grade) filler, and usually requires additionally processing to form free-flowing granules.

Cima also has U.S. patents and international patent applications directed to effervescent dosage forms (U.S. Pat. Nos. 5,503,846, 5,223,264, and 5,178,878, the contents of each are incorporated herein by reference) and tableting aids for rapidly dissolving dosage forms (U.S. Pat. Nos. 5,401,513 and 5,219,574, the contents of both are incorporated herein by reference), and rapidly dissolving dosage forms for water soluble drugs (WO 98/14179 for “Taste-Masked Microcapsule Composition and Methods of Manufacture”, the contents of which are incorporated herein by reference).

Fuisz Technologies, now part of BioVail, markets Flash Dose™, which is a direct compression tablet containing a processed excipient called Shearform™. Shearform™ is a cotton candy-like substance of mixed polysaccharides converted to amorphous fibers. U.S. patents describing this technology include U.S. Pat. No. 5,871,781 for “Apparatus for Making Rapidly Dissolving Dosage Units;” U.S. Pat. No. 5,869,098 for “Fast-Dissolving Comestible Units Formed Under High-Speed/High-Pressure Conditions;” U.S. Pat. Nos. 5,866,163, 5,851,553, and 5,622,719, all for “Process and Apparatus for Making Rapidly Dissolving Dosage Units and Product Therefrom;” U.S. Pat. No. 5,567,439 for “Delivery of Controlled-Release Systems;” and U.S. Pat. No. 5,587,172 for “Process for Forming Quickly Dispersing Comestible Unit and Product Therefrom,” each of which is incorporated herein by reference.

Prographarm markets Flashtab™, which is a fast melt tablet having a disintegrating agent such as carboxymethyl cellulose, a swelling agent such as a modified starch, and a taste-masked active agent. The tablets have an oral disintegration time of under one minute (U.S. Pat. No. 5,464,632, the contents of which are incorporated herein by reference).

R. P. Scherer markets Zydis™, which is a freeze-dried tablet having an oral dissolution time of 2 to 5 seconds. Lyophilized tablets are costly to manufacture and difficult to package because of the tablets sensitivity to moisture and temperature. U.S. Pat. No. 4,642,903 (R. P. Scherer Corp.), the contents of which are incorporated herein by reference, refers to a fast melt dosage formulation prepared by dispersing a gas throughout a solution or suspension to be freeze-dried. U.S. Pat. No. 5,188,825 (R. P. Scherer Corp.), the contents of which are incorporated herein by reference, refers to freeze-dried dosage forms prepared by bonding or complexing a water-soluble active agent to or with an ion exchange resin to form a substantially water insoluble complex, which is then mixed with an appropriate carrier and freeze dried. U.S. Pat. No. 5,631,023 (R. P. Scherer Corp.), the contents of which are incorporated herein by reference, refers to freeze-dried drug dosage forms made by adding xanthan gum to a suspension of gelatin and active agent. Finally, U.S. Pat. No. 5,827,541 (R. P. Scherer Corp.), the contents of which are incorporated herein by reference, discloses a process for preparing solid pharmaceutical dosage forms of hydrophobic substances. The process involves freeze-drying a dispersion containing a hydrophobic active ingredient and a surfactant, in a non-aqueous phase; and a carrier material, in an aqueous phase.

Yamanouchi-Shaklee markets Wowtab™, which is a tablet having a combination of a low moldability and a high moldability saccharide. U.S. patents covering this technology include U.S. Pat. No. 5,576,014 for “Intrabuccally Dissolving Compressed Moldings and Production Process Thereof,” and U.S. Pat. No. 5,446,464 for “Intrabuccally Disintegrating Preparation and Production Thereof,” both of which are incorporated herein by reference.

Other companies owning rapidly dissolving technology include Janssen Pharmaceutica. U.S. patents assigned to Janssen describe rapidly dissolving tablets having two polypeptide (or gelatin) components and a bulking agent, wherein the two components have a net charge of the same sign, and the first component is more soluble in aqueous solution than the second component. See U.S. Pat. No. 5,807,576 for “Rapidly Dissolving Tablet;” U.S. Pat. No. 5,635,210 for “Method of Making a Rapidly Dissolving Tablet;” U.S. Pat. No. 5,595,761 for “Particulate Support Matrix for Making a Rapidly Dissolving Tablet;” U.S. Pat. No. 5,587,180 for “Process for Making a Particulate Support Matrix for Making a Rapidly Dissolving Tablet;” and U.S. Pat. No. 5,776,491 for “Rapidly Dissolving Dosage Form,” each of which is incorporated herein by reference.

Eurand America, Inc. has U.S. patents directed to a rapidly dissolving effervescent composition having a mixture of sodium bicarbonate, citric acid, and ethylcellulose (U.S. Pat. Nos. 5,639,475 and 5,709,886, the contents of which are incorporated herein by reference).

L.A.B. Pharmaceutical Research owns U.S. patents directed to effervescent-based rapidly dissolving formulations having a pharmaceutically active ingredient and an effervescent couple comprising an effervescent acid and an effervescent base (U.S. Pat. Nos. 5,807,578 and 5,807,577, each of which is incorporated herein by reference).

Schering Corporation has technology relating to buccal tablets having an active agent, an excipient (which can be a surfactant) or at least one of sucrose, lactose, or sorbitol, and either magnesium stearate or sodium dodecyl sulfate (U.S. Pat. Nos. 5,112,616 and 5,073,374, each of which is incorporated herein by reference).

Laboratoire L. LaFon owns technology directed to conventional dosage forms made by lyophilization of an oil-in-water emulsion in which at least one of the two phases contains a surfactant (U.S. Pat. No. 4,616,047, the contents of which are incorporated herein by reference). For this type of formulation, the active ingredient is maintained in a frozen suspension state and is tableted without micronization or compression, as such processes could damage the active agent.

Takeda Chemicals Inc., Ltd. owns technology directed to a method of making a fast dissolving tablet in which an active agent and a moistened, soluble carbohydrate are compression molded into a tablet, followed by drying of the tablets (U.S. Pat. No. 5,501,861, which is incorporated herein by reference).

Finally, Elan's U.S. Pat. No. 6,316,029, for “Rapidly Disintegrating Oral Dosage Form,” the contents of which are incorporated by reference, discloses fast melt dosage forms comprising nanoparticulate active agents.

In one example of fast melt tablet preparation, granules for fast melt tablets made by either the spray drying or pre-compacting processes are mixed with excipients and compressed into tablets using conventional tablet making machinery. The granules can be combined with a variety of carriers including low density, high moldability saccharides, low moldability saccharides, polyol combinations, and then directly compressed into a tablet that exhibits an improved dissolution and disintegration profile.

The tablets according to the present invention typically have a hardness of about 2 to about 6 Strong-Cobb units (scu). Tablets within this hardness range disintegrate or dissolve rapidly when chewed. Additionally, the tablets rapidly disintegrate in water. On average, a typical 1.1 to 1.5 gram tablet disintegrates in 1-3 minutes without stirring. This rapid disintegration facilitates delivery of the active material.

The granules used to make the tablets can be, for example, mixtures of low density alkali earth metal salts or carbohydrates. For example, a mixture of alkali earth metal salts includes a combination of calcium carbonate and magnesium hydroxide. Similarly, a fast melt tablet can be prepared according to the methods of the present invention that incorporates the use of A) spray dried extra light calcium carbonate/maltodextrin, B) magnesium hydroxide and C) a eutectic polyol combination including Sorbitol Instant, xylitol and mannitol. These materials have been combined to produce a low density tablet that dissolves very readily and promotes the fast disintegration of the active ingredient. Additionally, the pre-compacted and spray dried granules can be combined in the same tablet.

For fast melt tablet preparation, a sirtuin modulator useful in the present invention can be in a form such as solid, particulate, granular, crystalline, oily or solution. The sirtuin modulator for use in the present invention may be a spray dried product or an adsorbate that has been pre-compacted to a harder granular form that reduces the medicament taste. A pharmaceutical active ingredient for use in the present invention may be spray dried with a carrier that prevents the active ingredient from being easily extracted from the tablet when chewed.

In addition to being directly added to the tablets of the present invention, the medicament drug itself can be processed by the pre-compaction process to achieve an increased density prior to being incorporated into the formulation.

The pre-compaction process used in the present invention can be used to deliver poorly soluble pharmaceutical materials so as to improve the release of such pharmaceutical materials over traditional dosage forms. This could allow for the use of lower dosage levels to deliver equivalent bioavailable levels of drug and thereby lower toxicity levels of both currently marketed drug and new chemical entities. Poorly soluble pharmaceutical materials can be used in the form of nanoparticles, which are nanometer-sized particles.

In addition to the active ingredient and the granules prepared from low density alkali earth metal salts and/or water soluble carbohydrates, the fast melt tablets can be formulated using conventional carriers or excipients and well established pharmaceutical techniques. Conventional carriers or excipients include, but are not limited to, diluents, binders, adhesives (i.e., cellulose derivatives and acrylic derivatives), lubricants (i.e., magnesium or calcium stearate, vegetable oils, polyethylene glycols, talc, sodium lauryl sulphate, polyoxy ethylene monostearate), disintegrants, colorants, flavorings, preservatives, sweeteners and miscellaneous materials such as buffers and adsorbents.

Additional description of the preparation of fast melt tablets can be found, for example, in U.S. Pat. No. 5,939,091, the contents of which are incorporated herein by reference.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active ingredient.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose analogs, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the composition in the proper medium. Absorption enhancers can also be used to increase the flux of the composition across the skin. The rate of such flux can be controlled by either providing a rate-controlling membrane or dispersing the compound in a polymer matrix or gel.

Ophthalmic formulations, eye ointments, powders, solutions, drops and the like, are also contemplated as being within the scope of this invention. Examples of ophthalmic formulations are described above.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient, or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Inhibition of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

Implantable devices containing a sirtuin modulator are also included in the invention. In one example, the device is bioerodible implant for treating a medical condition of the eye comprising an active agent dispersed within a biodegradable polymer matrix, wherein at least about 75% of the particles of the active agent have a diameter of less than about 10 μm. The bioerodible implant is sized for implantation in an ocular region. Te ocular region can be any one or more of the anterior chamber, the posterior chamber, the vitreous cavity, the choroid, the suprachoroidal space, the conjunctiva, the subconjunctival space, the episcleral space, the intracorneal space, the epicorneal space, the sclera, the pars plana, surgically-induced avascular regions, the macula, and the retina. The biodegradable polymer can be, for example, a poly(lactic-co-glycolic)acid (PLGA) copolymer. The ratio of lactic to glycolic acid monomers in the polymer can be about 50/50 weight percentage. Additionally, the PLGA copolymer can be about 20 to about 90 weight percent of the bioerodible implant. Alternately, the PLGA copolymer can be about 40 percent by weight of the bioerodible implant.

In another example, a drug delivery device is formed, in whole or in part, by co-extruding a drug core and an outer tube. The outer tube may be permeable, semi-permeable, or impermeable to the drug. The drug core may include a polymer matrix which does not significantly affect the release rate of the drug. The outer tube, the polymer matrix of the drug core, or both may be bioerodible. The co-extruded product can be segmented into drug delivery devices. The devices may be left uncoated so that their respective ends are open, or the devices may be coated with, for example, a layer that is permeable to the drug, semi-permeable to the drug, or bioerodible.

In a further example, a surgically implanted intraocular device has a reservoir container having a diffusible wall of polyvinyl alcohol or polyvinyl acetate and containing milligram quantities of a sirtuin modulator. As another example, milligram quantities of agent(s) may be incorporated into a polymeric matrix having dimensions of about 2 mm by 4 mm, and made of a polymer such as polycaprolactone, poly(glycolic) acid, poly(lactic) acid, or a polyanhydride, or a lipid such as sebacic acid. Typically, such devices are implanted on the sclera or in the eye. This is usually accomplished with the patient receiving either a topical or local anesthetic and using a small (3-4 mm incision) made behind the cornea. The matrix, containing the agent(s), is then inserted through the incision and sutured to the sclera using 9-0 nylon.

Additional description of implantable devices can be found, for example, in U.S. Publication Nos. 2004/0009222, 2004/0180075, 2005/0048099, 2005/0064010 and 2005/0025810, the contents of which are incorporated herein by reference.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The addition of the active compound of the invention to animal feed is preferably accomplished by preparing an appropriate feed premix containing the active compound in an effective amount and incorporating the premix into the complete ration.

Alternatively, an intermediate concentrate or feed supplement containing the active ingredient can be blended into the feed. The way in which such feed premixes and complete rations can be prepared and administered are described in reference books (such as “Applied Animal Nutrition”, W.H. Freedman and CO., San Francisco, U.S.A., 1969 or “Livestock Feeds and Feeding” O and B books, Corvallis, Ore., U.S.A., 1977).

The use of compositions of the invention is not limited to treating vision impairment. The compositions of the invention can also be used for treating and/or inhibiting a wide variety of diseases and disorders including, for example, diseases or disorders related to aging or stress, diabetes, obesity, neurodegenerative diseases and neuronal disorders, cardiovascular disease, blood clotting (coagulation) disorders, inflammation, cancer, and/or flushing, etc. Additional exemplary uses of compositions of the invention are disclosed in US Publication 2005/0096256.

### EXEMPLICATION

### Example 1

Preparation of Resveratrol-Cyclodextrin Formulation

100 milligrams of resveratrol are weighed and placed in a 5 mL scintillation tube. 1.5 mL of absolute ethanol is added to the tube and shaken until the resveratrol is completely dissolved. 5 grams of pyrogen free hydroxypropyl-β-cyclodextrin (sold by, Sigma-Aldrich, Inc., St. Louis, Mo., USA) are weighed on an analytical scale and placed in a graduated cylinder. Water is added with shaking until the volume reaches 90 ml. The above ethanolic solution of resveratrol is added to the aqueous solution containing hydroxypropyl-β-cyclodextrin with stirring. Water is added to the clear solution to make the total volume to 100 mL. The solution is sterile-filtered through a 0.22 micron filter. The suspension is frozen below −40 degree C. and is lyophilized. The lyophilized cake is reconstituted with sterile water for injection prior to further use.

### Example 2

Oral and Suppository Formulations of Resveratrol-Cyclodextrin Complex

100 mg of resveratrol is weighed and placed in a sterile test tube. The resveratrol is dissolved in 2-3 mL of purified absolute ethanol. 50 ml of a 9.8% solution of hydroxypropyl-p-cyclodextrin is prepared in a 150 mL sterile beaker and the solution is heated to 70-80 degree Centigrade while stirring on a hot plate. The ethanolic solution of resveratrol is slowly added to the beaker with stirring. The solution is sterile-filtered through a 0.22 μm filter. The solution is frozen below −40° C. and lyophilized. The lyophilized cake is powdered and used for the tablets, capsule and coated pills formulations and the lyophilized powder is denoted as resveratrol-cyclodextrin complex.

### Example 3

Preparation of Tablets

The tablet composition is compounded from the following ingredients: Resveratrol-cyclodextrin complex 6.25 parts; Lactose 79.75 parts; Potato starch 30.00 parts; Gelatin 3.00 parts; Magnesium stearate 1.00 parts; Total 120.0 parts

The resveratrol-cyclodextrin complex is intensively milled with ten times its weight of lactose, the milled mixture is admixed with the remaining amount of the lactose and the potato starch, the resulting. The mixture is moistened with an aqueous 10% solution of the gelatin, the moist mass is formed through a 1.5 mm-mesh screen, and the resulting granulate is dried at 40 degree C. The dry granulate is again passed through a 1 mm-mesh screen, admixed with the magnesium stearate, and the composition is compressed into 120 mg-tablets in a conventional tablet making machine. Each tablet contains 0.125 mg of resveratrol and is an oral dosage unit composition with effective therapeutic action.

### Example 4

Preparation of Coated Pills

The pill core composition is compounded from the ingredients: Resveratrol-cyclodextrin complex 6.25 parts; Lactose 26.25 parts; Corn starch 15.00 parts; Polyvinylpyrrolidone 2.00 parts; Magnesium stearate 0.50 parts; Total 50.0 parts

The resveratrol-cyclodextrin complex is intensively milled with the lactose, the milled mixture is admixed with the corn starch, the mixture is moistened with an aqueous 15% solution of the polyvinylpyrrolidone, the moist mass is forced through a 1 mm-mesh screen, and the resulting granulate is dried at 40 degree C. and again passed through the screen. The dry granulate is admixed with the magnesium stearte, and the resulting composition is compressed into 50 mg-pill cores which are subsequently coated in conventional manner with a thin shell consisting essentially of a mixture of sugar and talcum and finally polished with beeswax. Each coated pill contains 0.125 mg of resveratrol complexed with hydroxypropyl-cyclodextrin and is an oral dosage unit composition with effective therapeutic action.

### Example 5

Preparation of Drop Solution

The solution is compounded from the ingredients: Resveratrol-cyclodextrin complex 0.625 parts; Saccharin sodium 0.3 parts; Sorbic acid 0.1 parts; Ethanol 30.0 parts; Flavoring 1.0 parts; Distilled water q.s. ad 100.0 parts

The resveratrol-cyclodextrin complex and the flavoring are dissolved in the ethanol, and the sorbic acid and the saccharin sodium are dissolved in the distilled water. The two solutions are uniformly admixed with each other, and the mixed solution is filtered until free from suspended matter. 1 ml of the filtrate contains 0.125 mg of the resveratrol and is an oral dosage unit composition with effective therapeutic action.

### Example 6

Preparation of Suppositories

The suppository composition is compounded from the ingredients: Resveratrol-cyclodextrin complex 6.25 parts; Lactose 4.75 parts; Suppository base (e.g. cocoa butter) 1689.0 parts; Total 1700.0 parts

The resveratrol-cyclodextrin complex and the lactose are admixed, and the mixture is milled. The milled mixture is uniformly stirred with the aid of an immersion homogenizer into the suppository base, which had previously been melted and cooled to 40 degree C. The resulting composition is cooled at 37 degree C., and 1700 mg portions thereof are poured into cooled suppository molds and allowed to harden therein. Each suppository contains 0.125 mg of the resveratrol and is rectal dosage unit composition with effective therapeutic action.

### Example 7

Preparation of Capsules

The capsule composition is compounded from the following ingredients: Resveratrol-cyclodextrin complex 6.25 parts; Lactose 94.75 parts Micronized Beta-(1,3/16) Glucan 200.00 parts; (Baker's Yeast) R-Alpha Lipoic Acid 100.00 parts; Total 400.0 parts

The resveratrol-cyclodextrin complex is intensively milled with ten times its weight of lactose, the milled mixture is admixed with the remaining amount of the lactose, the micronized beta-glucan and the R-alpha lipoic acid. The mixed powder is again milled and the composition is filled into 400 mg-capsule in a conventional capsule making machine. Each capsule contains 0.125 mg of resveratrol and is an oral dosage unit composition with effective therapeutic action.

### Example 8

Preparation of Micronized Drug and Drug Suspensions

16 grams of micronized resveratrol was produced by Micron Technologies (Exton, Pa.). The milling equipment used was a Fluid Energy Jet Mill (Subclass: Tangential Jet) with a 4 inch mill size and compressed nitrogen gas/compressed air (dew point>40° C.) as milling gas. The material was manually fed into a hopper and placed on top of the feed tray. The material was drawn into a confined, circular chamber by way of pressurized milling gas. The powder becomes suspended in a high velocity fluid stream in the milling chamber. The mill operates on the principle of impact and attrition due to the high velocity collisions between particles suspended within the fluid stream, causing them to break down into smaller particles. Centrifugal force causes the larger, heavy particles to separate from smaller, lighter particles. The small particles are dragged by the escaping fluid stream towards the center of the mill, where they are discharged into filter bags, and the material is then collected into drums. The large particles are thrown outward where they recirculate and re-collide, causing them to break down.

In-process samples are removed during the course of the micronization and analyzed to ensure that micronization parameters are correct to accomplish the intended degree of particle size reduction. Particle size distribution is measured on a Malvern Mastersizer particle size analyzer. The milling conditions are then adjusted to give material with an acceptable micron size. Final particle size was determined to be 1.12 microns (DV50) (with a range from 0.31 microns (DV10) to 4.64 microns (DV90)) versus intitial particle size of 13.72 microns (DV50) (with a range from 1.92 microns (DV10) to 39.01 microns (DV90)) of unprocessed starting material.

Suspensions of either micronized resveratrol or unprocessed resveratrol were prepared for use in animal studies. Specifically, 20% w/v solutions were prepared in either 2% HPMC (3 cpi)/0.2% DOSS; 0.5% HPMC (4000 cpi); 20% Tween 80; or 0.5% methylcellulose (15 cpi).

### Example 9

Preparation of Resveratrol Cyclodextran Solutions and Lyophilized Powders

Two resveratrol formulations were chosen for evaluation as prototype drug products, resveratrol solutions in either 40% Captisol or 10% Captisol. Specifically, a low concentration resveratrol solution (20 mM or 4.57 mg/mL) in 10% Captisol (w/v) was produced on a 20 mL scale and lyophilized vials produced (1 mL per vial). Lyophilized vials containing 10% Captisol control vehicle were also manufactured. A high concentration resveratrol solution (150 mM) in 40% Captisol (w/v) was produced on a 500 mL scale, and supplied as a lyophilized product (5 mL per vial). Lyophilized vials containing 40% Captisol control vehicle were also manufactured.

The low concentration resveratrol solution was prepared by adding 136.99 mg resveratrol to 30 mL 10% Captisol (CyDex, Lot # CY-04A-05006), and mixed for 1 hour at ambient temperature. The initial solution had a pH of 5.02 and with a concentration of 15.4 mM in resveratrol. The solution was filtered through a 0.20 μm nylon syringe filter (Fisher #09-719C), resulting in a solution at 14.9 mM in resveratrol with a pH of 4.83. The 10% Captisol control vehicle solution had a pH of 4.78 before filtration and a pH of 4.76 after filtration. The filtered solutions were dispensed into vials (1.0 mL solution into 3 mL vial, Wheaton #223684), and lyophilized. At the end of the lyophilization cycle, the vials were sealed under vacuum and a cap crimped over the septa.

The lyophilized resveratrol product was an off-white cake. The material reconstituted in ˜20 seconds after the addition of 1.0 mL water. The reconstituted resveratrol solution was clear, with no visible particulates, having a pH of 5.91, and a measured concentration of 14.4 mM resveratrol. The lyophilized control vehicle product was a white cake. The material reconstituted in ˜20 seconds after the addition of 1.0 mL water. The reconstituted control vehicle was clear, with no visible particulates, and having a pH of 5.82.

The high concentration resveratrol formulation (150 mM SRT501 in 40% Captisol (w/v) on a 500 mL scale) was prepared as follows. A 40% Captisol (w/v) was prepared by placing 560 g of Captisol (CyDex, Lot # CY-04A-05006) into a 4 L beaker. A large stir bar was added to the beaker that was then placed on a large stir plate. In-house Milli-Q water was added to bring the volume in the beaker to ˜1.3 L. The solution was mixed vigorously for 1 hour until clear. At times, a spatula was used to free the stir bar and dislodge undissolved material from the sides of the vessel. The solution was transferred to a 2 L graduated cylinder. Approximately 100 mL of water was used to rinse the beaker and was added to the graduated cylinder, bringing the final volume to 1.4 L. The solution was mixed thoroughly until clear.

The high concentration resveratrol solution was prepared by mixing 600 mL of 40% Captisol in a 1 L beaker with 24.0 g resveratrol and stirring vigorously (the target concentration would be 175 mM resveratrol in 40% Captisol (w/v) if all the material dissolved). The solution was stirred for 35 minutes, followed by sonication for 1 hour. The solution was stirred for an additional 50 minutes, followed by sonication for an additional hour. At this point, solid material was still present in the sample. An aliquot was removed for analysis. The pH was measured at 4.76 and the concentration was measured by HPLC to be 55 mM resveratrol. Because the solution appeared cloudy at this time, three portions were separated to try alternate mixing methods. Approximately 200 mL was transferred to a 500 mL volumetric flask and was sonicated for 45 min, approximately 300 mL was vigorously mixed with an overhead mixer for 90 minutes, and approximately 100 mL was homogenized for 25 minutes. None of the three portions appeared clear. A sample of the homogenized solution was removed for concentration analysis and had a concentration of 74 mM resveratrol. The portions were combined and were stirred at ambient temperature overnight protected from light. The following morning, using an aseptic technique in the laminar flow hood, the solution was sterile filtered via a Nalgene 90 mm filter unit (Cat# 167-0020; 0.02 PES membrane). The concentration was again measured and determined to be 168 mM resveratrol in 40% Captisol.

The control vehicle (40% Captisol (w/v)) was also prepared as follows. 700 mL of 40% Captisol was transferred into a 1 L beaker. The solution was sonicated for a total of 2 hours. The solution was then stored at ambient temperature overnight. Using an aseptic technique in the laminar flow hood, the solution was sterile filtered via a Nalgene 90 mm filter unit (Cat# 167-0020; 0.02 PES membrane).

In the laminar flow hood, 30 mL molded glass vials, which had been sterilized by an autoclave, were filled with 5 mL of control vehicle or resveratrol solution. The vials were lyophilized in three batches according to the previously developed lyophilization cycle. The vials were stoppered under vacuum, sealed, and labeled. The resveratrol/Captisol vials contained a cake with an off-white appearance. The sample quickly reconstituted to a slightly yellow clear solution with the addition of water. 3.7 mL of water should be added to a vial to produce the original concentration of 40% Captisol.

### Example 10

Nicotinamide Riboside is Neuroprotective for Retinal Ganglion Cells During Acute Optic Neuritis

Background

Optic neuritis is an inflammatory disorder of the optic nerve that is commonly associated with the central nervous system autoimmune-mediated demyelinating disease multiple sclerosis (MS). Patients with optic neuritis typically have progressive visual loss over 1-2 weeks, then recover most or all of their vision over several weeks. Over 40% of patients do have some persistent visual changes (decreased acuity, color vision, contrast sensitivity or visual field), and patients with repeated episodes of optic neuritis have increased likelihood of permanent visual loss. Recent studies have suggested that neuronal damage in lesions of MS and optic neuritis are responsible for permanent dysfunction.

Experimental autoimmune encephalomyelitis (EAE) is an animal model of MS induced by immunization with Proteolipid Protein (PLP). Animals mount an immune response resulting in inflammation, demyelination, and neuronal damage in the brain, spinal cord, and optic nerve, similar to MS patients. Optic neuritis induced in EAE mice leads to loss of retinal ganglion cells (RGCs), neurons whose axons form the optic nerve.

Preliminary Studies

Techniques for labeling RGCs and for histological determination of optic neuritis have been refined for use in SJL/J mice with EAE induced by proteolipid protein peptide (PLP). A detailed evaluation of the time course of RGC loss in optic neuritis has been performed and are described below.

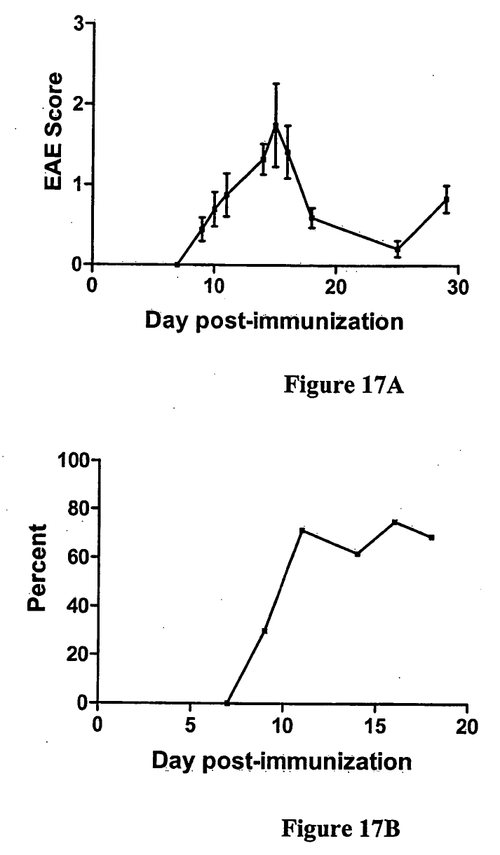

PLP induces a relapsing/remitting course of EAE in SJL/J mice: SJL/J mice were immunized with PLP by subcutaneous injection and observed daily for clinical signs of EAE. Results demonstrate mice develop EAE clinical symptoms as early as day 9 after immunization and clinical symptoms peak by day 14-15 (FIG. 17A). Clinical assessment is on a scale from 0-5 (with “5” being moribund, “4” being quadriplegic through to “0” which is an apparently healthy animal). Clinical EAE score then declines until day 25 when a second relapse of symptoms begins. Mice are considered to have had a relapse if they have an increase by 1 on the clinical scale for two or more days after a period of five or more days of stable or improved appearance.

A high incidence of optic neuritis is detected in EAE mice: SJL/J mice immunized with PLP were sacrificed at various time points. Optic nerves were isolated, fixed, embedded in paraffin, cut and stained with hematoxylin and eosin (H & E). Optic neuritis (presence of inflammatory cell infiltrates) is detected by day 9 after immunization and reaches peak incidence of over 70% of optic nerves by day 11 (FIG. 17B).

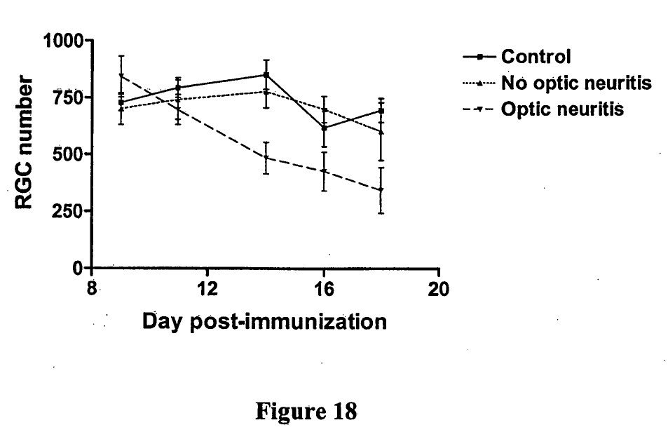

Inflammation precedes RGC loss in eyes with optic neuritis: RGCs were retrogradely labeled with Fluorgold (FG) by stereotactic injection into superior colliculi prior to induction of EAE. Mice were sacrificed at various times points and retinas and optic nerves were isolated. Retinas were whole mounted on glass slides and RGC numbers were counted by fluorescent microscopy. In eyes with optic neuritis, no loss of RGCs is detected at day 9 or 11 after immunization as compared to control eyes or eyes from EAE mice that did not develop optic neuritis (FIG. 18). Significant loss of RGCs is detected by day 14 (43% decrease vs. control) and progresses through day 18 (52% decrease vs. control).

Study outline: The neuroprotective effects of nicotinamide riboside were examined in EAE mice with optic neuritis. 6-8 week old SJL/J mice were labeled with 2.5 μl of 1.25% FG solution injected into the superior colliculi. To induce EAE, mice were immunized several days later with 300 μg PLP emulsified in complete Freund's adjuvant (CFA), and control mice (without EAE) were mock-immunized with phosphate buffered saline (PBS) in CFA. All mice received 200 ng intraperitoneal pertussis toxin (PT) on the day of immunization (day 0) and again on day 2.

Eyes were treated with nicotinamide riboside by intravitreal (ivt) injections with a volume of 0.8 μl/injection of a stock solution of either 0.1 M or 0.4 M nicotinamide riboside in PBS (Groups 2, 4 and 5). This results in an estimated final ocular concentration of nicotinamide riboside of 19 mM or 76 mM. Non-drug treatment control mice received either no ivt injections (Group 1), or mock-injections with PBS (Group 3). Treament with nicotinamide riboside, as well as PBS control injections, were given ivt on days 0, 4, 7 and 11. Mice were scored daily for clinical EAE, and were sacrificed on day 14 by overdose with ketamine and xylazine.

Retinas were dissected and whole-mounted for fluorescent microscopy. RGC numbers were quantified by counting FG-labeled cells in 12 standardized fields in each retina. Optic nerves were dissected and processed for histology. Cut sections stained by H & E were evaluated for the presence of inflammatory cells to determine acute optic neuritis. RGCs were compared between PBS-treated and nicotinamide riboside-treated eyes with optic neuritis to determine whether nicotinamide riboside prevents loss of neurons.

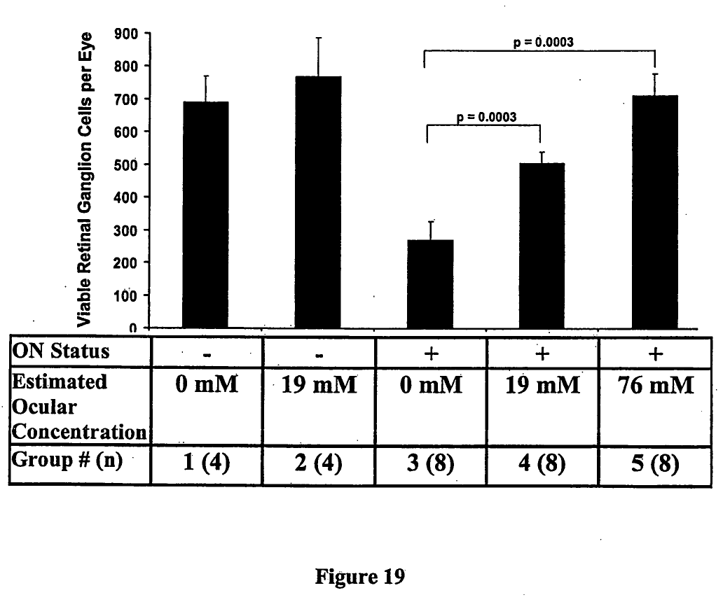

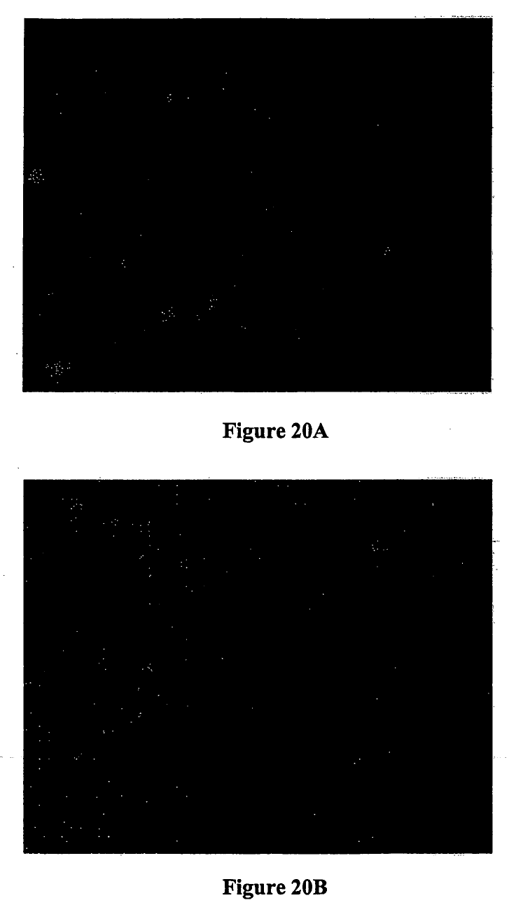

Results: As shown in FIG. 19, there was no difference in RGC numbers between control eyes and non-EAE eyes treated with nicotinamide riboside (Groups 1 and 2). Significant RGC loss occurred in PBS-treated EAE eyes with optic neuritis (268±59 RGCs; Group 3) vs. controls (691±81; Group 1), p<0.01. RGC loss was reduced by 100 mM nicotinamide riboside treatment (505±36; Group 4) and completely blocked by 400 mM nicotinamide riboside treatment (710±67; Group 5), p<0.01. Incidence of optic neuritis and clinical EAE did not differ between nicotinamide riboside treated mice and controls. FIG. 20 shows fluorogold-labeled RGCs (A) of eye with optic neuritis treated with placebo (PBS) (representative of Group 3) and (B) of eye with optic neuritis treated with nicotinamide riboside (representative of Group 5).

Conclusion: Nicotinamide riboside is neuroprotective for RGCs during acute optic neuritis in EAE in a dose-dependent manner. Nicotinamide riboside is not toxic to RGCs, and does not prevent inflammatory cell infiltration. Sirtuin activation has the potential therapeutic role to prevent neurodegeneration in optic neuritis and MS, and may be useful in conjunction with anti-inflammatory therapy.

### Example 11

Resveratrol is Neuroprotective for Retinal Ganglion Cells (RGC) During Acute Optic Neuritis

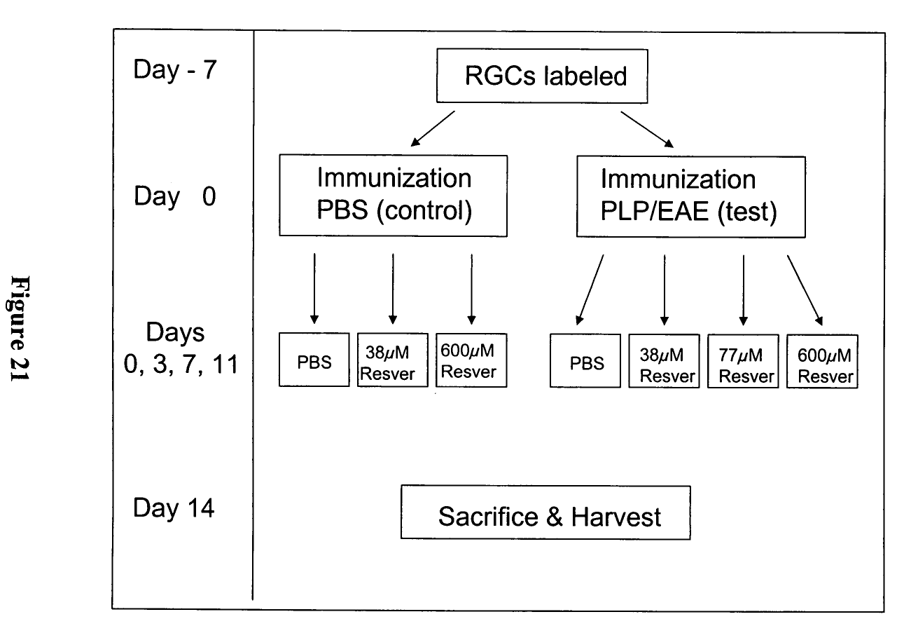

Resveratrol, a second sirtuin activator, was tested in the same experimental autoimmune encephalomyelitis (EAE) optic neuritis model as in the previous example. The experimental design is diagrammed in FIG. 21. 6-8 week old SJL/J mice were labeled with 2.5 μl of 1.25% fluorogold (FG) solution injected into the superior colliculi. To induce EAE, mice were immunized seven days later with 300 μg proteolipid protein (PLP) emulsified in complete Fruend's adjuvant (CFA), and control mice (without EAE) were mock-immunized with phosphate buffered saline (PBS) in CFA. All mice received 200 ng intraperitoneal pertussis toxin (PT) on the day of immunization (day 0) and again on day 3.

Preparation of Test Substance

5 μl of 770 mM resveratrol formulation was dissolved in 495 μl vehicle for a final concentration of 7.7 mM resveratrol (stock solution). Three test doses were diluted:

1) 600 μM resveratrol=10 μl stock solution+118±vehicle;

2) 77 μM resveratrol=10 μl stock solution+990 μl vehicle; and

3) 38 μM resveratrol=200 μl 177 μM resveratrol+200 μl vehicle.

Administration of Test Compounds

Eyes were treated with resveratrol or PBS control by intravitreal (iv) injections with a volume of 0.8 μl/injection. Treament with 38 μM, 77 μM or 600 μM resveratrol, as well as PBS, were given on days 0, 3, 7, and 11.

Evaluation Criteria

Mice were scored daily for clinical EAE on a five point scale: no disease=0; partial tail paralysis=0.5; tail paralysis or waddling gait=1.0; partial tail paralysis and waddling gait=1.5; tail paralysis and waddling gait=2.0; partial limb paralysis=2.5; paralysis of one limb=3.0; paralysis of one limb and partial paralysis of another=3.5; paralysis of two limbs=4.0; moribund state=4.5; death=5.0. Clinical EAE scores of individual mice are shown in Table I.

TABLE I
Peak EAE Score For Individual Mice

| | | | | | | | | | | | |
|---|---|---|---|---|---|---|---|---|---|---|---|
| Control - PBS | 0 | 0 | 0 | 0 | 0 | | | | | | |
| Control - 38 μM | 0 | 0 | 0 | 0 | | | | | | | |
| Resveratrol | | | | | | | | | | | |
| Control - 600 μM | 0 | 0 | 0 | | | | | | | | |
| Resveratrol | | | | | | | | | | | |
| EAE - PBS | 2.5 | 2.5 | 0.5 | 2 | 3 | 1.5 | 2 | 1.5 | 1.5 | 1.0 | 1.0 |
| EAE - 38 μM | 1.5 | 3 | 0 | 5 | 2.5 | 3 | 1 | 1 | | | |
| Resveratrol | | | | | | | | | | | |
| EAE - 77 μM | 2 | 2.5 | 0 | 2 | 3.5 | 2 | 5 | 1.5 | 2.5 | 2 | 2.5 |
| Resveratrol | | | | | | | | | | | |
| EAE - 600 μM | 2.5 | 2 | 5 | 4.5 | 1.5 | 2 | 0.5 | | | | |
| Resveratrol | | | | | | | | | | | |

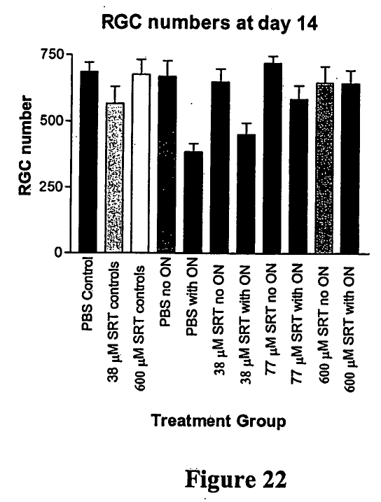

Further results are shown in FIG. 22.

Mice were sacrificed on day 14 by overdose with ketamine and xylazine.

Retinas were diessected and whole-mounted for fluorescent microscopy. RGC numbers were quantified by counting FG-labeled cells in 12 standardized fields in each retina. Optic nerves were dissected and processed for histology. Cut sections stained by H & E were evaluated and scored as follows for the presence of inflammatory cells to determine acute optic neuritis: eyes with no inflammation (i.e. without optic neuritis)=0; mild inflammation=1; moderate inflammation=2; severe inflammation=3. RGCs were compared between PBS-treated and resveratrol-treated eyes with optic neuritis to determine whether resveratrol prevents loss of neurons.

Results

The neuroprotective effects of resveratrol were examined during optic neuritis in EAE, an animal model of MS. RGCs were retrogradely-labelled with FG by injection into superior colliculi. EAE was induced by immunization with PLP in SJL/J mice. Eyes were treated with PBS, 38CM, 77 μM or 600 μM resveratrol by intravitreal injection on days 0 (day of immunization), 3, 7 and 11. Mice were sacrificed on day 14. Optic neuritis was detected by inflammatory cell infiltration of the optic nerve and fluorescent-labelled RGCs were counted. There was no difference in RGC numbers between control eyes and non-EAE eyes treated with resveratol. Significant RGC loss occurred in PBS treated EAE eyes with optic neuritis (385±117 RGCs) vs. controls (686±113; p<0.01). RGC loss was not significantly prevented by 38 μM resveratrol treatment (452±136; p=0.2098). RGC loss was significantly reduced by 77 μM resveratrol treatment (585±198; p=0.0028) and completely blocked by 600 μM resveratrol treatment (644±148; p=0.0001). Incidence of optic neuritis and clinical EAE did not differ between resveratrol treated mice and controls. Statistical analysis was performed using ANOVA. Results demonstrate resveratrol is neuroprotective for RGCs during acute optic neuritis in EAE in a dose-dependent manner. Resveratrol is not toxic to RGCs, and does not prevent inflammatory cell infiltration.

### Example 12

Testing of Neuroprotective Effects of Nicotinamide Riboside and Nicotinamide Mononucleotide in a Retinal Ganglion Cell Injury Model

Summary:

The following example demonstartes the effect of resveratrol, NMN and nicotinamide riboside on ganglion cell survival in the Swiss white mouse retinas after intravitreal NMDA injection.

Administration of Test Compounds:

Stock solutions for administration are nicotinamide riboside (125 mM in water) and NMN (125 mM in water).

Endpoints

RGC density is determined by immunohistochemistry with brn-3 labeled retinal ganglion cells (RGC). RGCs are counted in 12 standard retinal locations per flat mount.

Methods

Test Substance Administration

On days 0, 2 and 4, 2 μl of test substance or vehicle (2% HPMC, 0.2% DOSS) is injected into the intravitreal space of anesthetised (intraperitoneal ketamine, xylazine) to the right eye of all 3-month old adult Swiss white mice (25 to 30 g, n=12 per treatment) using a microsyringe driver attached to a micropipette.

Sham Injections:

Vehicle (2 μl, n=12) is injected on days 0, 2 and 4 to the right eye of all mice using a microsyringe driver attached to a micropipette. Water (n=4) will be injected days 0, 2 and 4 to the right eye of all mice using a microsyringe driver attached to a micropipette to serve as controls for nicotinamide riboside and NMN.

RGC Injury Models

Intravitreal NMDA injection (100 nM in 2 μl) is administered to the right eye of all mice (test substance or sham injected animals) using a microsyringe driver attached to a micropipette. This injection induces reproducible RGC apoptosis, which peaks between 12 and 24 hours after injection.

RGC Density:

This is quantified from retinal flatmounts created 6 days after NMDA injection. RGCs are identified by anti-brn-3 staining³. RGC density is determined for 12 retinal locations per flat mount (3 per quadrant at set distances from the optic nerve head). To generate flatmounts, mice are perfusion fixed with 4% paraformaldehyde, eyes enucleated and fixed overnight in 4% paraformaldehyde. Retinas are then collected and placed onto subbed slides, labeled and counted.

Mouse Summary for Each Test Substance:

Injections are performed to right eyes only (in accordance with ARVO statements for the use of animals in ophthalmic and vision research).

### Example 13

Treatment of Mice and Rats with Micronized Resveratrol Particle Formulations Mouse Dosing

In general, doses as indicated were administered to 3 mice per group and blood was collected at the time points shown. Male C57BL/6 mice, 18-22 grams (Charles River Labs, Willmington, Mass.) were used for all the mouse studies. Samples are sent to Charles River Labs (CRL) for analysis.

Specifically, mice were dosed (one every 2 minutes) either by intraperitoneal injection (IP), subcutaneous injection (SQ), or oral gavage (PO). Mice were sacrificed at appropriate time points using CO₂ overdose (place a mouse in the CO₂ chamber 40 seconds before the time point). Approximately 0.5 ml blood was immediately taken via cardiac stick with a 25 G 1 ml syringe. The needle was removed and the sample was added to a microtainer blood tube with a lithium heparin and plasma separator (Becton Dickinson Catalog Number BD365965). Samples were placed on ice until centrifuged. Plasma was transferred to snap tubes and frozen on dry ice.

Drug stocks used included:

Resveratrol micronized particle suspension (20% w/v; average particle size 1.12 microns, Dv50) in 2% HPMC (3 cpi)+0.2% DOSS;

Resveratrol micronized particle suspension (20% w/v; average particle size 1.12 microns, Dv50) in 0.5% HPMC (4000 cpi);

Resveratrol micronized particle suspension (20% w/v; average particle size 1.12 microns, Dv50) in 20% Tween 80; and

Resveratrol unprocessed particle suspension (20% w/v; average particle size 13.72 microns, Dv50) in 0.5% methylcellulose (15 cpi).

Stocks were diluted in appropriate vehicles to allow dosing of 0.2 ml of a formulation (by injection or gavage) at the indicated dose. For instance, for a 500 mg/kg dose, a 0.2 ml injection of a 50 mg/ml suspension was used. Three mice per group were sacrificed at the following time points: 5, 10, 15, 30, 60, 120, 240, 360, and 720 minutes. Serum values were determined and plotted for all time points where drug could be detected.

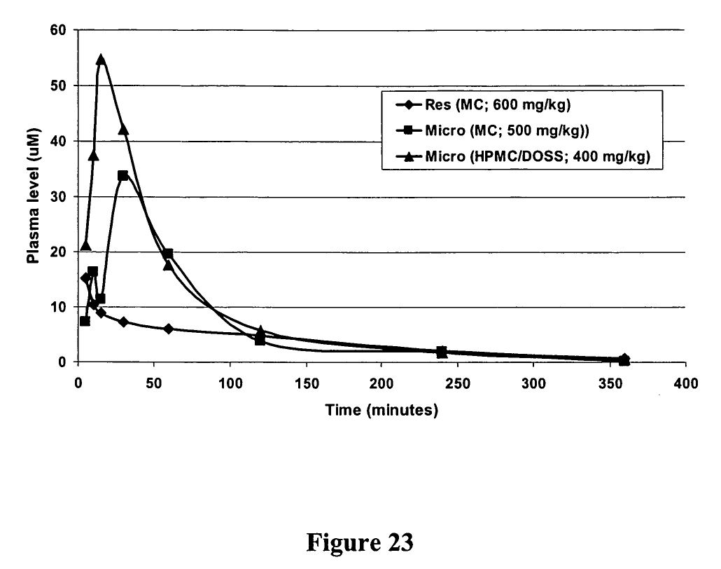

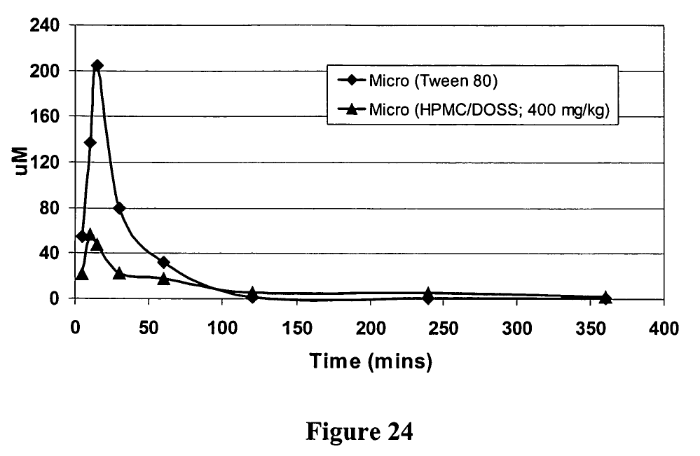

A comparison of the different resveratrol formulations administered by oral gavage is shown in FIGS. 23 and 24. Specifically, a comparison of unprocessed resveratrol in methylcellulose (mean particle size approx. 13 um) to micronized resveratrol (mean particle size approx. 1.0 um) in either HPMC alone or HPMC/DOSS dosed via oral gavage at the indicated doses in mice is shown in FIG. 23. A comparison of micronized resveratrol (Micro; mean particle size approx. 1.0 um) in either Tween 80 or HPMC/DOSS dosed via oral gavage at the indicated doses in mice is shown in FIG. 24.

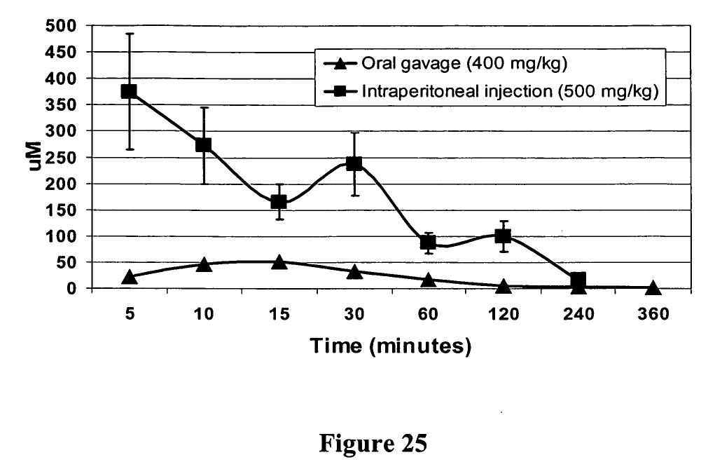

A comparison of micronized resveratrol (mean particle size approx. 1.0 um) in HPMC/DOSS dosed via oral gavage or IP injection at the indicated doses in mice is shown in FIG. 25.

Rat Dosing

In general, doses as indicated were administered to 3 or 4 rats per group and blood was collected at the indicated time points. Male Sprague Dawley rats, about 300 grams (Charles River Labs, Wilmington, Mass.) were used for all the rat studies. Dosing, sample collection and analysis were done at Charles River Labs.

Specifically, rats were dosed either by intraperitoneal injection (IP) or oral gavage (PO). Approxamately 300 to 500 microliters of blood was withdrawn at the indicated times. Samples were placed on ice until centrifugation. Plasma was transferred to a snap tube and frozen on dry ice.

Drug stocks used included:

Resveratrol unprocessed particle suspension (20% w/v; average particle size 13.72 microns, Dv50) in 0.5% methylcellulose (15 cpi) Resveratrol cyclodextran solution (175 mM resveratrol in 40% Captisol, pH approx. 6.0)

Resveratrol dissolved in DMSO (100 mM resveratrol)

Stocks were diluted in appropriate vehicles to allow dosing at indicated doses. Samples were collected at 5, 15, 30, 60, 120, 240, 360, 480, 720 and 1440 minutes for IP dosing and at 15, 30, 60, 120, 240, 360, 480 and 720 minutes for PO dosing. Serum values were determined and plotted for all time points where drug could be detected.

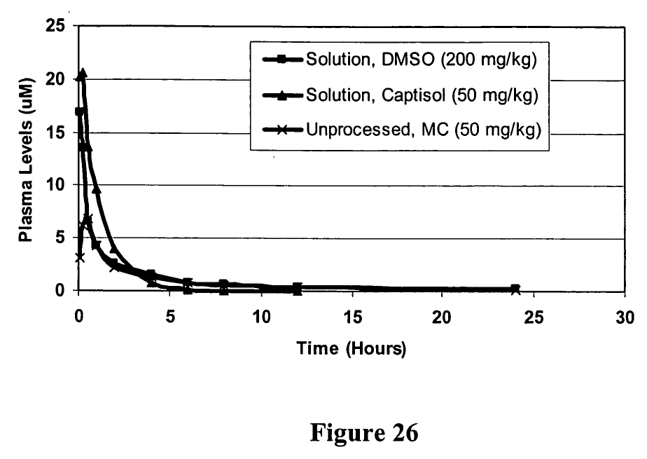

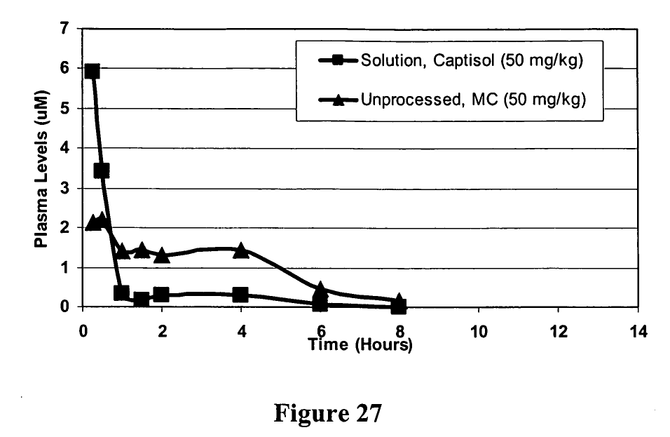

A comparison of various formulations of resveratrol in rats following IP or PO dose administration at the indicated doses in rats is shown in FIG. 26 and FIG. 27 and Tables II and III, respectively.

TABLE II
Comparison of pharmacokinetic parameters for IP dosing
of various resveratrol formulations in rats.

| | DMSO solution | Captisol solution | Unprocessed in |
|---|---|---|---|
| IP Comparison | (200 mg/kg) | (50 mg/kg) | MC (50 mg/kg) |
| Cmax (uM) | 17.0 | 22.0 | 7.1 |
| Tmax (hours) | 0.1 | 0.2 | 0.6 |
| AUC (uM-hours) | 25.0 | 26.4 | 19.9 |
| T_{1/2} (hours) | 0.44 | 1.1 | 2.2 |

TABLE III
Comparison of pharmacokinetic parameters for oral dosing
of various resveratrol formulations in rats.

| | Captisol solution | Unprocessed in |
|---|---|---|
| Oral Comparison | (50 mg/kg) | MC (50 mg/kg) |
| Cmax (uM) | 5.9 | 2.4 |
| Tmax (hours) | 0.3 | 0.4 |
| AUC (uM-hours) | 3.4 | 8.5 |
| T_{1/2} (hours) | 0.5 | 3.9 |

As can be seen, various dosing routes and/or formulations can be used to drive different PK parameters. For instance, with IP dosing, the captisol formulated resveratrol solution achieves a greater Cmax and AUC than the unprocessed resveratrol suspension. The DMSO resveratrol solution gives an intermediate Cmax and AUC. However, the resveratrol suspension showed the longest duration of detectable drug in the plasma as measured by T_{1/2}. With oral dosing, the captisol solution again gave a higher Cmax as compared to oral dosing of the unprocessed resveratrol suspension. However, the resveratrol suspension gave a higher overall AUC and duration of detectable drug in the plasma. Based on these results, resveratrol can be formulated in different ways or delivered by different routes to either maximize absolute plasma levels (Cmax) or duration of exposure (AUC or T_{1/2}).

### INCORPORATION BY REFERENCE

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

Also incorporated by reference are the following: PCT Publications WO 2005/002672; 2005/002555; and 2004/016726 and US Publication 2005/0096256.

1. A composition comprising a cyclodextrin and a sirtuin modulator, or a pharmaceutically acceptable salt, prodrug or a metabolic derivative thereof, wherein when the composition is a liquid and the sirtuin modulator is resveratrol, the composition comprises at least 30 mM of the sirtuin modulator.

2. The compostion of claim 1, wherein the cyclodextrin is a substituted cyclodextrin.

3. The composition of claim 2, wherein the cyclodextrin is substituted on the 2-, 3- or 6-hydroxyl group of a glycopyranose moiety.

4. The composition of claim 2, wherein the cyclodextrin is amorphous.

5. The composition of claim 2, wherein the cyclodextrin is hydroxypropyl-beta-cyclodextrin.

6. The composition of claim 1, wherein the sirtuin modulator is a sirtuin activator.

7. The composition of claim 6, wherein the sirtuin activator is resveratrol, an analog thereof, or a prodrug of resveratrol or the analog.

8. The composition of claim 6, wherein the sirtuin activator is nicotinamide riboside, an analog thereof, or a prodrug of nicotinamide riboside or the analog.

9. The composition of claim 1, wherein the composition is a liquid.

10. The composition of claim 9, wherein the liquid comprises at least 30 mM of the sirtuin modulator.

11. The composition of claim 10, wherein the liquid comprises at least 100 mM of the sirtuin modulator.

12. The composition of claim 1, wherein the composition is a lyophilized powder.

13. A method for treating vision impairment by administering to a patient a therapeutic dosage of a composition comprising cyclodextrin and a sirtuin modulator, or a pharmaceutically acceptable salt, prodrug or a metabolic derivative thereof.

14. A fast melt tablet comprising a sirtuin modulator, or a pharmaceutically acceptable salt, prodrug or a metabolic derivative thereof.

15-20. (canceled)

21. A method for treating vision impairment by administering to a patient a therapeutic dosage of a fast melt tablet comprising a sirtuin modulator, or a pharmaceutically acceptable salt, prodrug or a metabolic derivative thereof.

22. An implanatable device comprising a sirtuin modulator, or a pharmaceutically acceptable salt, prodrug or a metabolic derivative thereof.

23-30. (canceled)

31. A method for treating vision impairment by implanting in a patient a implanatable device comprising a sirtuin modulator, or a pharmaceutically acceptable salt, prodrug or a metabolic derivative thereof.

32. A pharmaceutical composition comprising a micronized sirtuin modulator or a pharmaceutically acceptable salt, prodrug or a metabolic derivative thereof, wherein particles of the micronized sirtuin modulator have an average diameter of less than about 30 microns.

33-38. (canceled)

39. A method for treating vision impairment by administering to a patient a therapeutic dosage of a pharmaceutical composition comprising a micronized sirtuin modulator or a pharmaceutically acceptable salt, prodrug or a metabolic derivative thereof, wherein particles of the micronized sirtuin modulator have an average diameter of less than about 30 microns.
